(19) United States
(12) Patent Application Publication
(10) Pub. No.: US 2025/0034558 A1
(43) Pub. Date: Jan. 30, 2025

(54) COMPOSITIONS AND METHODS FOR TARGETING, EDITING OR MODIFYING HUMAN GENES

(71) Applicants: Artisan Development Labs, Inc., Boulder, CO (US); DOMINGUES PEREIRA, Sara Isabel, Boulder, CO (US)

(72) Inventors: Andrea BARGHETTI, Copenhagen (DK); Roland BAUMGARTNER, Angern an der March (AT); Tanya WARNECKE, Boulder, CO (US); Sara Isabel DOMINGUES PEREIRA, Copenhagen (DK)

(21) Appl. No.: 18/571,700

(22) PCT Filed: Jun. 20, 2022
(86) PCT No.: PCT/US2022/034186

§ 371 Date: Dec. 18, 2023

Related U.S. Application Data
(60) Provisional application No. 63/286,814, filed on Dec. 7, 2021.
(60) Provisional application No. 63/212,189, filed on Jun. 18, 2021.

(51) Int. Cl. C12N 15/11; C12N 9/22; C12N 15/90

(52) U.S. Cl. CPC C12N 15/11; C12N 9/22; C12N 15/907; C12N 2310/20

(57) ABSTRACT

The present invention relates to engineered Clustered Regularly Interspaced Short Palindromic Repeats (CRISPR) systems and corresponding guide RNAs that target specific nucleotide sequences at certain gene loci in the human genome. Also provided are methods of targeting, editing, and/or modifying of the human genes using the engineered CRISPR systems, and compositions and cells comprising the engineered CRISPR systems.

This application claims the benefit of U.S. Provisional Application Nos. 63/212,189 filed Jun. 18, 2021, and 63/286,814, filed Dec. 7, 2021, which applications are incorporated herein by reference.

### BACKGROUND OF THE INVENTION

Recent advances have been made in precise genome targeting technologies. For example, specific loci in genomic DNA can be targeted, edited, or otherwise modified by designer meganucleases, zinc finger nucleases, or transcription activator-like effectors (TALEs). Furthermore, the CRISPR-Cas systems of bacterial and archaeal adaptive immunity have been adapted for precise targeting of genomic DNA in eukaryotic cells. Compared to the earlier generations of genome editing tools, the CRISPR-Cas systems are easy to set up, scalable, and amenable to targeting multiple positions within the eukaryotic genome, thereby providing a major resource for new applications in genome engineering.

Two distinct classes of CRISPR-Cas systems have been identified. Class 1 CRISPR-Cas systems utilize multi-protein effector complexes, whereas class 2 CRISPR-Cas systems utilize single-protein effectors (see, Makarova et al. (2017) CELL, 168: 328). Among the three types of class 2 CRISPR-Cas systems, type II and type V systems typically target DNA and type VI systems typically target RNA (id.). Naturally occurring type II effector complexes consist of Cas9, CRISPR RNA (crRNA), and trans-activating CRISPR RNA (tracrRNA), but the crRNA and tracrRNA can be fused as a single guide RNA in an engineered system for simplicity (see, Wang et al. (2016) ANNU. REV. BIOCHEM., 85: 227). Certain naturally occurring type V systems, such as type V-A, type V-C, and type V-D systems, do not require tracrRNA and use crRNA alone as the guide for cleavage of target DNA (see, Zetsche et al. (2015) CELL, 163: 759; Makarova et al. (2017) CELL, 168: 328).

The CRISPR-Cas systems have been engineered for various purposes, such as genomic DNA cleavage, base editing, epigenome editing, and genomic imaging (see, e.g., Wang et al. (2016) ANNU. REV. BIOCHEM., 85: 227 and Rees et al. (2018) NAT. REV. GENET., 19: 770). Although significant developments have been made, there remains a need for new and useful CRISPR-Cas systems as powerful genome targeting tools.

### SUMMARY OF THE INVENTION

The present invention is based, in part, upon the development of engineered CRISPR-Cas systems (e.g., type V-A CRISPR-Cas systems) that can be used to target, edit, or otherwise modify specific target nucleotide sequences in human APLNR, BBS1, CALR, CD247, CD3D, CD38, CD3E, CD3G, CD40LG, CD52, CD58, COL17A1, CSF2, DEFB134, ERAP1, ERAP2, IFNGR1, IFNGR2, JAK1, JAK2, mir-101-2, MLANA, NLRC5 PSMB5, PSMB8, PSMB9, PTCD2, RFX5, RFXANK, RFXAP, RPL23, SOX10, SRP54, STAT1, Tap1, TAP2, TAPBP, TRBC1, TRBC1_2 (or TRBC1+2), TRBC2, or TWF1 gene. In particular, guide nucleic acids, such as single guide nucleic acids and dual guide nucleic acids, can be designed to hybridize with the selected target nucleotide sequence and activate a Cas nuclease to edit the human genes. CRISPR-Cas systems comprising such guide nucleic acids are also useful for targeting or modifying the human genes.

A CRISPR-Cas system generally comprises a Cas protein and one or more guide nucleic acids (e.g., RNAs). The Cas protein can be directed to a specific location in a double-stranded DNA target by recognizing a protospacer adjacent motif (PAM) in the non-target strand of the DNA, and the one or more guide nucleic acids can be directed to a specific location by hybridizing with a target nucleotide sequence in the target strand of the DNA. Both PAM recognition and target nucleotide sequence hybridization are required for stable binding of a CRISPR-Cas complex to the DNA target and, if the Cas protein has an effector function (e.g., nuclease activity), activation of the effector function. As a result, when creating a CRISPR-Cas system, a guide nucleic acid can be designed to comprise a nucleotide sequence called spacer sequence that hybridizes with a target nucleotide sequence, where target nucleotide sequence is located adjacent to a PAM in an orientation operable with the Cas protein. It has been observed that not all CRISPR-Cas systems designed by these criteria are equally effective. The present invention identifies target nucleotide sequences in particular human genes that can be efficiently edited, and provides CRISPR-Cas systems directed to these target nucleotide sequences.

Accordingly, in one aspect, the present invention provides a guide nucleic acid comprising a targeter stem sequence and a spacer sequence, wherein the spacer sequence comprises a nucleotide sequence listed in Table 1, 2, 3, 4, 5, 6, 7, 8, or 9.

In certain embodiments, the targeter stem sequence comprises a nucleotide sequence of GUAGA. In certain embodiments, the targeter stem sequence is 5′ to the spacer sequence, optionally wherein the targeter stem sequence is linked to the spacer sequence by a linker consisting of 1, 2, 3, 4, or 5 nucleotides.

In certain embodiments, the guide nucleic acid is capable of activating a CRISPR Associated (Cas) nuclease in the absence of a tracrRNA (e.g., the guide nucleic acid being a single guide nucleic acid). In certain embodiments, the guide nucleic acid comprises from 5′ to 3′ a modulator stem sequence, a loop sequence, a targeter stem sequence, and the spacer sequence.

In certain embodiments, the guide nucleic acid is a targeter nucleic acid that, in combination with a modulator nucleic acid, is capable of activating a Cas nuclease. In certain embodiments, the guide nucleic acid comprises from 5′ to 3′ a targeter stem sequence and the spacer sequence.

In certain embodiments, the Cas nuclease is a type V Cas nuclease. In certain embodiments, the Cas nuclease is a type V-A Cas nuclease. In certain embodiments, the Cas nuclease comprises an amino acid sequence at least 80% identical to SEQ ID NO: 1. In certain embodiments, the Cas nuclease is Cpf1. In certain embodiments, the Cas nuclease recognizes a protospacer adjacent motif (PAM) consisting of the nucleotide sequence of TTTN or CTTN.

In certain embodiments, the guide nucleic acid comprises a ribonucleic acid (RNA). In certain embodiments, the guide nucleic acid comprises a modified RNA. In certain embodiments, the guide nucleic acid comprises a combination of RNA and DNA. In certain embodiments, the guide nucleic acid comprises a chemical modification. In certain embodiments, the chemical modification is present in one or more nucleotides at the 5′ end of the guide nucleic acid. In certain embodiments, the chemical modification is present in one or more nucleotides at the 3′ end of the guide nucleic acid. In certain embodiments, the chemical modification is selected from the group consisting of 2′-O-methyl, 2′-fluoro, 2′-O-methoxyethyl, phosphorothioate, phosphorodithioate, pseudouridine, and any combinations thereof.

The present invention also provides an engineered, non-naturally occurring system comprising a guide nucleic acid (e.g., a single guide nucleic acid) disclosed herein. In certain embodiments, the engineered, non-naturally occurring system further comprising the Cas nuclease. In certain embodiments, the guide nucleic acid and the Cas nuclease are present in a ribonucleoprotein (RNP) complex.

The present invention also provides an engineered, non-naturally occurring system comprising the guide nucleic acid (e.g., targeter nucleic acid) disclosed herein, wherein the engineered, non-naturally occurring system further comprises the modulator nucleic acid. In certain embodiments, the engineered, non-naturally occurring system, further comprises the Cas nuclease. In certain embodiments, the guide nucleic acid, the modulator nucleic acid, and the Cas nuclease are present in an RNP complex.

In certain embodiments of the engineered, non-naturally occurring system, the spacer sequence comprises a nucleotide sequence selected from the group consisting of SEQ ID NOs: 201-253, wherein the spacer sequence is capable of hybridizing with the human CSF2 gene. In certain embodiments, when the system is delivered into a population of human cells ex vivo, the genomic sequence at the CSF2 gene locus is edited in at least 1.5% of the cells, or at least 5, 10, 15, 20, 25, 30, 40, 50, 60, 70, 80, 90, or 95% of the cells.

In certain embodiments of the engineered, non-naturally occurring system, the spacer sequence comprises a nucleotide sequence selected from the group consisting of SEQ ID NOs: 254-313, wherein the spacer sequence is capable of hybridizing with the human CD40LG gene. In certain embodiments, when the system is delivered into a population of human cells ex vivo, the genomic sequence at the CD40LG gene locus is edited in at least 1.5% of the cells, or at least 5, 10, 15, 20, 25, 30, 40, 50, 60, 70, 80, 90, or 95% of the cells.

In certain embodiments of the engineered, non-naturally occurring system, the spacer sequence comprises a nucleotide sequence selected from the group consisting of SEQ ID NOs: 314-319 and 329-332, wherein the spacer sequence is capable of hybridizing with the human TRBC1 gene. In certain embodiments, when the system is delivered into a population of human cells ex vivo, the genomic sequence at the TRBC1 gene locus is edited in at least 1.5% of the cells, or at least 5, 10, 15, 20, 25, 30, 40, 50, 60, 70, 80, 90, or 95% of the cells.

In certain embodiments of the engineered, non-naturally occurring system, the spacer sequence comprises a nucleotide sequence selected from the group consisting of SEQ ID NOs: 320-328 and 329-332, wherein the spacer sequence is capable of hybridizing with the human TRBC2 gene. In certain embodiments, when the system is delivered into a population of human cells ex vivo, the genomic sequence at the TRBC2 gene locus is edited in at least 1.5% of the cells, or at least 5, 10, 15, 20, 25, 30, 40, 50, 60, 70, 80, 90, or 95% of the cells.

In certain embodiments of the engineered, non-naturally occurring system, the spacer sequence comprises a nucleotide sequence selected from the group consisting of SEQ ID NOs: 329-332, wherein the spacer sequence is capable of hybridizing with both the human TRBC1 gene and the human TRBC2 gene (TRBC1_2 or TRBC1+2). In certain embodiments, when the system is delivered into a population of human cells ex vivo, the genomic sequence at both the human TRBC1 gene and the human TRBC2 gene locus is edited in at least 1.5% of the cells, or at least 5, 10, 15, 20, 25, 30, 40, 50, 60, 70, 80, 90, or 95% of the cells.

In certain embodiments of the engineered, non-naturally occurring system, the spacer sequence comprises a nucleotide sequence selected from the group consisting of SEQ ID NOs: 333-374, wherein the spacer sequence is capable of hybridizing with the human CD3E gene. In certain embodiments, when the system is delivered into a population of human cells ex vivo, the genomic sequence at the CD3E gene locus is edited in at least 1.5% of the cells, or at least 5, 10, 15, 20, 25, 30, 40, 50, 60, 70, 80, 90, or 95% of the cells.

In certain embodiments of the engineered, non-naturally occurring system, the spacer sequence comprises a nucleotide sequence selected from the group consisting of SEQ ID NOs: 375-411, wherein the spacer sequence is capable of hybridizing with the human CD38 gene. In certain embodiments, when the system is delivered into a population of human cells ex vivo, the genomic sequence at the CD38 gene locus is edited in at least 1.5% of the cells, or at least 5, 10, 15, 20, 25, 30, 40, 50, 60, 70, 80, 90, or 95% of the cells.

In certain embodiments of the engineered, non-naturally occurring system, genomic mutations are detected in no more than 2% of the cells at any off-target loci by CIRCLE-Seq. In certain embodiments, genomic mutations are detected in no more than 1% of the cells at any off-target loci by CIRCLE-Seq.

In another aspect, the present invention provides a human cell comprising an engineered, non-naturally occurring system disclosed herein.

In another aspect, the present invention provides a composition comprising a guide nucleic acid, engineered, non-naturally occurring system, or human cell disclosed herein.

In another aspect, the present invention provides a method of cleaving a target DNA comprising the sequence of a preselected target gene or a portion thereof, the method comprising contacting the target DNA with an engineered, non-naturally occurring system disclosed herein, thereby resulting in cleavage of the target DNA. In certain embodiments, the contacting occurs in vitro. In certain embodiments, the contacting occurs in a cell ex vivo. In certain embodiments, the target DNA is genomic DNA of the cell.

In another aspect, the present invention provides a method of editing human genomic sequence at a preselected target gene locus, the method comprising delivering an engineered, non-naturally occurring system disclosed herein into a human cell, thereby resulting in editing of the genomic sequence at the target gene locus in the human cell. In certain embodiments, the cell is an immune cell. In certain embodiments, the immune cell is a T lymphocyte.

In certain embodiments, the method of editing human genomic sequence at a preselected target gene locus comprises delivering an engineered, non-naturally occurring system disclosed herein into a population of human cells, thereby resulting in editing of the genomic sequence at the target gene locus in at least a portion of the human cells. In certain embodiments, the population of human cells comprises human immune cells. In certain embodiments, the population of human cells is an isolated population of human immune cells. In certain embodiments, the immune cells are T lymphocytes.

In certain embodiments of the method of editing human genomic sequence at a preselected target gene locus, the engineered, non-naturally occurring system is delivered into the cell(s) as a pre-formed RNP complex. In certain embodiments, the pre-formed RNP complex is delivered into the cell(s) by electroporation.

In certain embodiments, the target gene is human CSF2 gene, wherein the spacer sequence comprises a nucleotide sequence selected from the group consisting of SEQ ID NOs: 201-253. In certain embodiments, the genomic sequence at the CSF2 gene locus is edited in at least 1.5% of the human cells, or at least 5, 10, 15, 20, 25, 30, 40, 50, 60, 70, 80, 90, or 95% of the cells.

In certain embodiments, the target gene is human CD40LG gene, wherein the spacer sequence comprises a nucleotide sequence selected from the group consisting of SEQ ID NOs: 254-313. In certain embodiments, the genomic sequence at the CD40LG gene locus is edited in at least 1.5% of the human cells, or at least 5, 10, 15, 20, 25, 30, 40, 50, 60, 70, 80, 90, or 95% of the cells.

In certain embodiments, the target gene is human TRBC1 gene, wherein the spacer sequence comprises a nucleotide sequence selected from the group consisting of SEQ ID NOs: 314-319 and 329-332. In certain embodiments, the genomic sequence at the TRBC1 gene locus is edited in at least 1.5% of the human cells, or at least 5, 10, 15, 20, 25, 30, 40, 50, 60, 70, 80, 90, or 95% of the cells.

In certain embodiments, the target gene is human TRBC2 gene, wherein the spacer sequence comprises a nucleotide sequence selected from the group consisting of SEQ ID NOs: 320-328 and 329-332. In certain embodiments, the genomic sequence at the TRBC2 gene locus is edited in at least 1.5% of the human cells, or at least 5, 10, 15, 20, 25, 30, 40, 50, 60, 70, 80, 90, or 95% of the cells.

In certain embodiments, the target gene is both the human TRBC1 gene and the human TRBC2 gene, wherein the spacer sequence comprises a nucleotide sequence selected from the group consisting of SEQ ID NOs: 329-332. In certain embodiments, the genomic sequence at both the human TRBC1 gene and the human TRBC2 gene locus is edited in at least 1.5% of the human cells, or at least 5, 10, 15, 20, 25, 30, 40, 50, 60, 70, 80, 90, or 95% of the cells.

In certain embodiments, the target gene is human CD3E gene, wherein the spacer sequence comprises a nucleotide sequence selected from the group consisting of SEQ ID NOs: 333-374. In certain embodiments, the genomic sequence at the CD3E gene locus is edited in at least 1.5% of the human cells, or at least 5, 10, 15, 20, 25, 30, 40, 50, 60, 70, 80, 90, or 95% of the cells.

In certain embodiments, the target gene is human CD38 gene, wherein the spacer sequence comprises a nucleotide sequence selected from the group consisting of SEQ ID NOs: 375-411. In certain embodiments, the genomic sequence at the CD38 gene locus is edited in at least 1.5% of the human cells, or at least 5, 10, 15, 20, 25, 30, 40, 50, 60, 70, 80, 90, or 95% of the cells.

In certain embodiments, genomic mutations are detected in no more than 2% of the cells at any off-target loci by CIRCLE-Seq. In certain embodiments, genomic mutations are detected in no more than 1% of the cells at any off-target loci by CIRCLE-Seq.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

FIG. 1A is a schematic representation showing the structure of an exemplary single guide type V-A CRISPR system. FIG. 1B is a schematic representation showing the structure of an exemplary dual guide type V-A CRISPR system.

FIGS. 2A-C are a series of schematic representation showing incorporation of a protecting group (e.g., a protective nucleotide sequence or a chemical modification) (FIG. 2A), a donor template-recruiting sequence (FIG. 2B), and an editing enhancer (FIG. 2C) into a type V-A CRISPR-Cas system. These additional elements are shown in the context of a dual guide type V-A CRISPR system, but it is understood that they can also be present in other CRISPR systems, including a single guide type V-A CRISPR system, a single guide type II CRISPR system, or a dual guide type II CRISPR system.

FIG. 3A shows the knockout efficiency of single guide RNAs targeted human CD38 in pan-T cells as measured by the percentage of cells having one or more insertion or deletion at the target site (% indel).

FIG. 3B shows the knockout efficiency of single guide RNAs targeting human CD38 in pan-T cells as measured by flow cytometry assessing the percent of CD38 negative cells in a population.

FIGS. 4 A-F show the knockout efficiency of single guide RNAs targeting human APLNR, BBS1, CALR, CD247, CD3G, CD52, CD58, COL17A1, DEFB134, ERAP1, ERAP2, IFNGR1, IFNGR2, JAK1, JAK2, mir-101-2, MLANA, PSMB5, PSMB8, PSMB9, PTCD2, RFX5, RFXANK, RFXAP, RPL23, SOX10, SRP54, STAT1, Tap1, TAP2, TAPBP, and TWF1 genes in pan-T cells as measured by the percentage of cells having one or more insertion or deletion at the target site (% indel).

FIG. 5 shows the knockout efficiency of single guide RNAs targeting human CD3D (panel A) and NLRC5 (panel B) genes in pan-T cells as measured by flow cytometry assessing the percent of HLA-I, HLA-II, and TCR negative cells in a population.

FIG. 6 shows percentage of DSG3 positive cells in a population, plotted for various treatment conditions.

FIG. 7 shows Day 7 expansion data for populations transfected under various treatment conditions.

### DETAILED DESCRIPTION OF THE INVENTION

I. Guide Nucleic Acids and Engineered, Non-Naturally Occurring CRISPR-Cas Systems
   A. Cas Proteins
   B. RNA Modifications
II. Methods of Targeting, Editing, and/or Modifying Genomic DNA
   A. Ribonucleoprotein (RNP) Delivery and “Cas RNA” Delivery
   B. CRISPR Expression Systems
   C. Donor Templates
   D. Efficiency and Specificity
   E. Multiplex Methods
III. Pharmaceutical Compositions
IV. Therapeutic Uses
V. Kits
VI. Embodiments
VII. Examples

The present invention is based, in part, upon the development of engineered CRISPR-Cas systems (e.g., type V-A CRISPR-Cas systems) that can be used to target, edit, or otherwise modify specific target nucleotide sequences in human APLNR, BBS1, CALR, CD247, CD3D, CD38, CD3E, CD3G, CD40LG, CD52, CD58, COL17A1, CSF2, DEFB134, ERAP1, ERAP2, IFNGR1, IFNGR2, JAK1, JAK2, mir-101-2, MLANA, NLRC5 PSMB5, PSMB8, PSMB9, PTCD2, RFX5, RFXANK, RFXAP, RPL23, SOX10, SRP54, STAT1, Tap1, TAP2, TAPBP, TRBC1, TRBC1_2 (or TRBC1+2), TRBC2, or TWF1 gene. In particular, guide nucleic acids, such as single guide nucleic acids and dual guide nucleic acids, can be designed to hybridize with the selected target nucleotide sequence and activate a Cas nuclease to edit the human genes. CRISPR-Cas systems comprising such guide nucleic acids are also useful for targeting or modifying the human genes.

A CRISPR-Cas system generally comprises a Cas protein and one or more guide nucleic acids (e.g., RNAs). The Cas protein can be directed to a specific location in a double-stranded DNA target by recognizing a protospacer adjacent motif (PAM) in the non-target strand of the DNA, and the one or more guide nucleic acids can be directed to a specific location by hybridizing with a target nucleotide sequence in the target strand of the DNA. Both PAM recognition and target nucleotide sequence hybridization are required for stable binding of a CRISPR-Cas complex to the DNA target and, if the Cas protein has an effector function (e.g., nuclease activity), activation of the effector function. As a result, when creating a CRISPR-Cas system, a guide nucleic acid can be designed to comprise a nucleotide sequence called spacer sequence that hybridizes with a target nucleotide sequence, where target nucleotide sequence is located adjacent to a PAM in an orientation operable with the Cas protein. It has been observed that not all CRISPR-Cas systems designed by these criteria are equally effective. The present invention identifies target nucleotide sequences in particular human genes that can be efficiently edited, and provides CRISPR-Cas systems directed to these target nucleotide sequences.

Naturally occurring Type V-A, type V-C, and type V-D CRISPR-Cas systems lack a tracrRNA and rely on a single crRNA to guide the CRISPR-Cas complex to the target DNA. Dual guide nucleic acids capable of activating type V-A, type V-C, or type V-D Cas nucleases have been developed, for example, by splitting the single crRNA into a targeter nucleic acid and a modulator nucleic acid. Naturally occurring type V-A Cas proteins comprise a RuvC-like nuclease domain but lack an HNH endonuclease domain, and recognize a 5′ T-rich PAM located immediately upstream from the target nucleotide sequence, the orientation determined using the non-target strand (i.e., the strand not hybridized with the spacer sequence) as the coordinate. The CRISPR-Cas systems cleave a double-stranded DNA to generate a staggered double-stranded break rather than a blunt end. The cleavage site is distant from the PAM site (e.g., separated by at least 10, 11, 12, 13, 14, or 15 nucleotides downstream from the PAM on the non-target strand and/or separated by at least 15, 16, 17, 18, or 19 nucleotides upstream from the sequence complementary to PAM on the target strand).

Naturally occurring type II CRISPR-Cas systems (e.g., CRISPR-Cas9 systems) generally comprise two guide nucleic acids, called crRNA and tracrRNA, which form a complex by nucleotide hybridization. Single guide nucleic acids capable of activating type II Cas nucleases have been developed, for example, by linking the crRNA and the tracrRNA (see, e.g., U.S. Patent Application Publication No. 2014/0242664 and U.S. Pat. No. 10,266,850). Naturally occurring type II Cas proteins comprise a RuvC-like nuclease domain and an HNH endonuclease domain, and recognize a 3′ G-rich PAM located immediately downstream from the target nucleotide sequence, the orientation determined using the non-target strand (i.e., the strand not hybridized with the spacer sequence) as the coordinate. The CRISPR-Cas systems cleave a double-stranded DNA to generate a blunt end. The cleavage site is generally 3-4 nucleotides upstream from the PAM on the non-target strand.

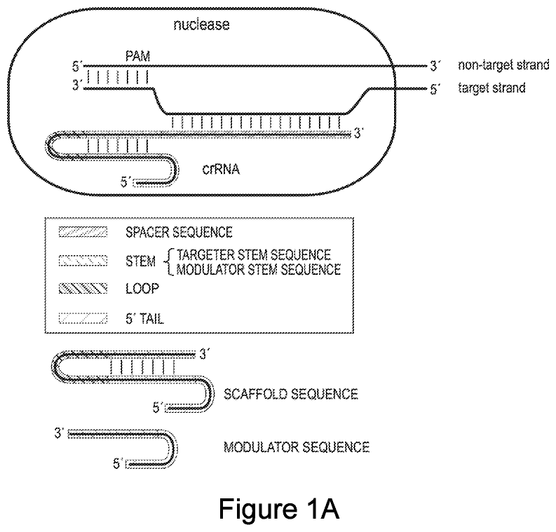

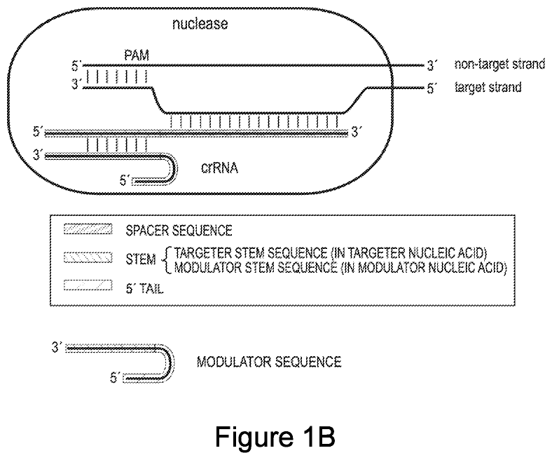

Elements in an exemplary single guide type V-A CRISPR-Cas system are shown in FIG. 1A. The single guide nucleic acid is also called a “crRNA” where it is present in the form of an RNA. It comprises, from 5′ to 3′, an optional 5′ sequence, e.g., a tail sequence, a modulator stem sequence, a loop, a targeter stem sequence complementary to the modulator stem sequence, and a spacer sequence that hybridizes with the target strand of the target DNA. Where a 5′ sequence, e.g., a tail sequence is present, the sequence including the 5′ sequence, e.g., a tail sequence and the modulator stem sequence is also called a “modulator sequence” herein. A fragment of the single guide nucleic acid from the optional 5′ sequence, e.g., a tail sequence to the targeter stem sequence, also called a “scaffold sequence” herein, bind the Cas protein. In addition, the PAM in the non-target strand of the target DNA binds the Cas protein.

Elements in an exemplary dual guide type V-A CRISPR-Cas system are shown in FIG. 1B. The first guide nucleic acid, called “modulator nucleic acid” herein, comprises, from 5′ to 3′, an optional 5′ sequence, e.g., a tail sequence and a modulator stem sequence. Where a 5′ sequence, e.g., a tail sequence, is present, the sequence including the 5′ sequence, e.g., a tail sequence and the modulator stem sequence is also called a “modulator sequence” herein. The second guide nucleic acid, called “targeter nucleic acid” herein, comprises, from 5′ to 3′, a targeter stem sequence complementary to the modulator stem sequence and a spacer sequence that hybridizes with the target strand of the target DNA. The duplex between the modulator stem sequence and the targeter stem sequence, plus the optional 5′ sequence, e.g., a tail sequence, constitute a structure that binds the Cas protein. In addition, the PAM in the non-target strand of the target DNA binds the Cas protein.

The terms “targeter stem sequence” and “modulator stem sequence,” as used herein, refer to a pair of nucleotide sequences in one or more guide nucleic acids that hybridize with each other. When a targeter stem sequence and a modulator stem sequence are contained in a single guide nucleic acid, the targeter stem sequence is proximal to a spacer sequence designed to hybridize with a target nucleotide sequence, and the modulator stem sequence is proximal to the targeter stem sequence. When a targeter stem sequence and a modulator stem sequence are in separate nucleic acids, the targeter stem sequence is in the same nucleic acid as a spacer sequence designed to hybridize with a target nucleotide sequence. In a CRISPR-Cas system that naturally includes separate crRNA and tracrRNA (e.g., a type II system), the duplex formed between the targeter stem sequence and the modulator stem sequence corresponds to the duplex formed between the crRNA and the tracrRNA. In a CRISPR-Cas system that naturally includes a single crRNA but no tracrRNA (e.g., a type V-A system), the duplex formed between the targeter stem sequence and the modulator stem sequence corresponds to the stem portion of a stem-loop structure in the scaffold sequence (also called direct repeat sequence) of the crRNA. It is understood that 100% complementarity is not required between the targeter stem sequence and the modulator stem sequence. In a type V-A CRISPR-Cas system, however, the targeter stem sequence is typically 100% complementary to the modulator stem sequence.

In certain embodiments wherein the target nucleic acid and the modulator nucleic acid comprise a single polynucleotide, a loop motif may exist between the 3′ stem sequence of the targeter nucleic acid and the 5′ stem sequence of the modulator nucleic acid, e.g., a stem loop. In certain embodiments, the loop motif is between 1-11, 2-11, 3-11, 4-11, 5-11, 3-10, 3-9, 3-8, 3-7, 3-6, 1-11, 2-10, 3-9, 4-8, 5-7, 4-6, 1-7, 2-6, 3-5 nucleotides in length. In a preferred embodiment, the loop motif is between 3-5 nucleotides in length. In a separate preferred embodiment, the loop motif is four nucleotides in length. In certain embodiments, the loop motif is 5′-TCTT-3′ or 5′-TATT-3′.

The term “targeter nucleic acid,” as used herein in the context of a dual guide CRISPR-Cas system, can include a nucleic acid comprising (i) a spacer sequence designed to hybridize with a target nucleotide sequence; and (ii) a targeter stem sequence capable of hybridizing with an additional nucleic acid to form a complex, wherein the complex is capable of activating a Cas nuclease (e.g., a type II or type V-A Cas nuclease) under suitable conditions, and wherein the targeter nucleic acid alone, in the absence of the additional nucleic acid, is not capable of activating the Cas nuclease under the same conditions. The term “targeter nucleic acid,” as used herein in the context of a single guide nucleic acid CRISPR-Cas system, can include a nucleic acid comprising (i) a spacer sequence designed to hybridize with a target nucleotide sequence; and (ii) a targeter stem sequence capable of hybridizing with a complementary stem sequence in a modulator nucleic acid that is 5′ to the targeter nucleic acid in the single polynucleotide of the sgNA, wherein the sgNA is capable of activating a Cas nuclease (e.g., a type II or type V-A Cas nuclease).

The term “modulator nucleic acid,” as used herein in connection with a given targeter nucleic acid and its corresponding Cas nuclease, can include a nucleic acid capable of hybridizing with the targeter nucleic acid, to form an intra-polynucleotide hybridized portion in the case of a sgNA, and to form a complex in the case of a dual gNA, wherein the sgNA or complex, but not the modulator nucleic acid alone, is capable of activating the type Cas nuclease under suitable conditions.

The term “suitable conditions,” as used in connection with the definitions of “targeter nucleic acid” and “modulator nucleic acid,” refers to the conditions under which a naturally occurring CRISPR-Cas system is operative, such as in a prokaryotic cell, in a eukaryotic (e.g., mammalian or human) cell, or in an in vitro assay.

The features and uses of the guide nucleic acids and CRISPR-Cas systems are discussed in the following sections.

### I. GUIDE NUCLEIC ACIDS AND ENGINEERED, NON-NATURALLY OCCURRING CRISPR-CAS SYSTEMS

The present invention provides a guide nucleic acid comprising a targeter stem sequence and a spacer sequence, wherein the spacer sequence comprises a nucleotide sequence listed in Tables 1, 2, 3, 4, 5, 6, or 7, or a portion thereof sufficient to hybridize with the corresponding target gene listed in the table. In particular, Table 1 lists the guide nucleic acid, targeting human CSF2 gene, comprising a spacer sequence with SEQ ID NOs: 201-253. Table 2 lists the guide nucleic acid, targeting human CD40LG gene, comprising a spacer sequence with SEQ ID NOs: 254-313. Table 3 lists the guide nucleic acid, targeting human TRBC1 gene, comprising a spacer sequence with SEQ ID NOs: 314-319. Table 4 lists the guide nucleic acid, targeting human TRBC2 gene, comprising a spacer sequence with SEQ ID NOs: 320-328. Table 5 lists the guide nucleic acid, targeting both the human TRBC1 gene and the human TRBC2 gene (TRBC1_2), comprising a spacer sequence with SEQ ID NOs: 329-332. Table 6 lists the guide nucleic acid, targeting human CD3E gene, comprising a spacer sequence with SEQ ID NOs: 333-374. Table 7 lists the guide nucleic acid, targeting human CD38 gene, comprising a spacer sequence with SEQ ID NOs: 375-411. Table 8 lists the guide nucleic acid, targeting human APLNR, BBS1, CALR, CD247, CD3G, CD52, CD58, COL17A1, DEFB134, ERAP1, ERAP2, IFNGR1, IFNGR2, JAK1, JAK2, mir-101-2, MLANA, PSMB5, PSMB8, PSMB9, PTCD2, RFX5, RFXANK, RFXAP, RPL23, SOX10, SRP54, STAT1, Tap1, TAP2, TAPBP, and TWF1 genes, comprising SEQ ID NOs: 412-715. Table 9 lists the guide nucleic acid, targeting human CD3D and NLRC5 genes, comprising a spacer sequence with SEQ ID NOs: 716-744.

In certain embodiments, a guide nucleic acid of the present invention is capable of hybridizing with the genomic locus of the corresponding target gene in the human genome. In certain embodiments, a guide nucleic acid of the present invention, alone of in combination with a modulator nucleic acid, is capable of forming a nucleic acid-guided nuclease complex with a Cas protein. In certain embodiments, a guide nucleic acid of the present invention, alone or in combination with a modulator nucleic acid, is capable of directing a Cas protein to the genomic locus of the corresponding target gene in the human genome. In certain embodiments, a guide nucleic acid of the present invention, alone or in combination with a modulator nucleic acid, is capable of directing a Cas nuclease to the genomic locus of the corresponding target gene in the human genome, thereby resulting in cleavage of the genomic DNA at the genomic locus.

TABLE 1
Selected Spacer Sequences Targeting
Human CSF2 Genes

| | | SEQ |
|---|---|---|
| | | ID |
| crRNA | Spacer Sequence | NO |
| gCSF2_001 | TGAGATGACTTCTACTGTTTC | 201 |
| gCSF2_002 | CCTTTTCTACAGAATGAAACA | 202 |
| gCSF2_003 | CTTTTCTACAGAATGAAACAG | 203 |
| gCSF2_004 | CTACAGAATGAAACAGTAGAA | 204 |
| gCSF2_005 | TACAGAATGAAACAGTAGAAG | 205 |
| gCSF2_006 | CCACAGGAGCCGACCTGCCTA | 206 |
| gCSF2_007 | CACAGGAGCCGACCTGCCTAC | 207 |
| gCSF2_008 | ttatttttctttttttAAAGG | 208 |
| gCSF2_009 | tatttttctttttttAAAGGA | 209 |
| gCSF2_010 | atttttctttttttAAAGGAA | 210 |
| gCSF2_011 | tttttctttttttAAAGGAAA | 211 |
| gCSF2_012 | tctttttttAAAGGAAACTTC | 212 |
| gCSF2_013 | ctttttttAAAGGAAACTTCC | 213 |
| gCSF2_014 | tttttttAAAGGAAACTTCCT | 214 |
| gCSF2_015 | tttAAAGGAAACTTCCTGTGC | 215 |
| gCSF2_016 | ttAAAGGAAACTTCCTGTGCA | 216 |
| gCSF2_017 | tAAAGGAAACTTCCTGTGCAA | 217 |
| gCSF2_018 | AAAGGTGATAATCTGGGTTGC | 218 |
| gCSF2_019 | AAAGGAAACTTCCTGTGCAAC | 219 |
| gCSF2_020 | AAGGAAACTTCCTGTGCAACC | 220 |
| gCSF2_021 | AAACTTTCAAAGGTGATAATC | 221 |
| gCSF2_022 | AAAGTTTCAAAGAGAACCTGA | 222 |
| gCSF2_023 | AAAGAGAACCTGAAGGACTTT | 223 |
| gCSF2_024 | TGCTTGTCATCCCCTTTGACT | 224 |
| gCSF2_025 | ACTGCTGGGAGCCAGTCCAGG | 225 |
| gCSF2_026 | CCTAGGTGGTCAGGCTTGGGG | 226 |
| gCSF2_027 | TGGTCACCATTAATCATTTCC | 227 |
| gCSF2_028 | CTCTGTGTATTTAAGAGCTCT | 228 |
| gCSF2_029 | AGAGCTCTTTTGCCAGTGAGC | 229 |
| gCSF2_030 | ATTCTGTAGAAAAGGAAAATG | 230 |
| gCSF2_031 | ACCTCCAGGTAAGATGCTTCT | 231 |
| gCSF2_032 | CAGAAGCCCCTGCCCTGGGGT | 232 |
| gCSF2_033 | GATGGCACCACACAGGGTTGT | 233 |
| gCSF2_034 | TCTCCAGTCAGCTGGCTGCAG | 234 |
| gCSF2_035 | TCAGCTGAGCGGCCATGGGCA | 235 |
| gCSF2_036 | CCACCTGTCCCCTGGTGACTC | 236 |
| gCSF2_037 | GGGCGCTCACTGTGCCCCGAG | 237 |
| gCSF2_038 | AGGAACAACCCTTGCCCACCC | 238 |
| gCSF2_039 | CTGCTGCCCCCAGCCCCCAGG | 239 |
| gCSF2_040 | TGTGCCAACAGTTATGTAATG | 240 |
| gCSF2_041 | ATCCCAAGGAGTCAGAGCCAC | 241 |
| gCSF2_042 | CCCTCACCTCTGACCTCATTA | 242 |
| gCSF2_043 | CTTGGGTTTGCCCTCACCTCT | 243 |
| gCSF2_044 | CTCTGGCCCCACATGGGGTGC | 244 |
| gCSF2_045 | CTCCCTTCCCGCAGGAAGGAG | 245 |
| gCSF2_046 | TGGCCTTGACTCCACTCCTTC | 246 |
| gCSF2_047 | GTCCCAGGGCAGAGCAGGGCA | 247 |
| gCSF2_048 | ACTGCCCAGAAGGCCAACCTC | 248 |
| gCSF2_049 | TCTACTGCCTCTTAGAACTCA | 249 |
| gCSF2_050 | AAAGGAAACTTCCTGTGCAAt | 250 |
| gCSF2_051 | AAGGAAACTTCCTGTGCAAtC | 251 |
| gCSF2_052 | AAAGGTGATAgTCTGGaTTGC | 252 |
| gCSF2_053 | AAACTTTCAAAGGTGATAgTC | 253 |

TABLE 2
Selected Spacer Sequences Targeting
Human CD40LG Genes

| | | SEQ |
|---|---|---|
| | | ID |
| crRNA | Spacer Sequence | NO |
| gCD40LG_001 | GTTGTATGTTTCGATCATGCT | 254 |
| gCD40LG_002 | AACTTTAACACAGCATGATCG | 255 |
| gCD40LG_003 | ACACAGCATGATCGAAACATA | 256 |
| gCD40LG_004 | ATGCTGATGGGCAGTCCAGTG | 257 |
| gCD40LG_005 | CATGCTGATGGGCAGTCCAGT | 258 |
| gCD40LG_006 | TATGTATTTACTTACTGTTTT | 259 |
| gCD40LG_007 | ATGTATTTACTTACTGTTTTT | 260 |
| gCD40LG_008 | TGTATTTACTTACTGTTTTTC | 261 |
| gCD40LG_009 | CTTACTGTTTTTCTTATCACC | 262 |
| gCD40LG_010 | TCTTATCACCCAGATGATTGG | 263 |
| gCD40LG_011 | CTTATCACCCAGATGATTGGG | 264 |
| gCD40LG_012 | TTATCACCCAGATGATTGGGT | 265 |
| gCD40LG_013 | TGCTGTGTATCTTCATAGAAG | 266 |
| gCD40LG_014 | GCTGTGTATCTTCATAGAAGG | 267 |
| gCD40LG_015 | CTGTGTATCTTCATAGAAGGT | 268 |
| gCD40LG_016 | ATGAATACAAAATCTTCATGA | 269 |
| gCD40LG_017 | CATGAATACAAAATCTTCATG | 270 |
| gCD40LG_018 | TCCTGTGTTGCATCTCTGTAT | 271 |
| gCD40LG_019 | GTATTCATGAAAACGATACAG | 272 |
| gCD40LG_020 | TATTCATGAAAACGATACAGA | 273 |
| gCD40LG_021 | ATCTCCTCACAGTTCAGTAAG | 274 |
| gCD40LG_022 | AATCTCCTCACAGTTCAGTAA | 275 |
| gCD40LG_023 | CCAGTAATTAAGCTGCTTACC | 276 |
| gCD40LG_024 | ACCAGTAATTAAGCTGCTTAC | 277 |
| gCD40LG_025 | AAGGCTTTGTGAAGGTAAGCA | 278 |
| gCD40LG_026 | TTCGTCTCCTCTTTGTTTAAC | 279 |
| gCD40LG_027 | TTTCTTCGTCTCCTCTTTGTT | 280 |
| gCD40LG_028 | CTTTCTTCGTCTCCTCTTTGT | 281 |
| gCD40LG_029 | AGGATATAATGTTAAACAAAG | 282 |
| gCD40LG_030 | GGATATAATGTTAAACAAAGA | 283 |
| gCD40LG_031 | AAAGCTGTTTTCTTTCTTCGT | 284 |
| gCD40LG_032 | CATTTCAAAGCTGTTTTCTTT | 285 |
| gCD40LG_033 | GCATTTCAAAGCTGTTTTCTT | 286 |
| gCD40LG_034 | TGCATTTCAAAGCTGTTTTCT | 287 |
| gCD40LG_035 | AGGATTCTGATCACCTGAAAT | 288 |
| gCD40LG_036 | TGGTTCCATTTCAGGTGATCA | 289 |
| gCD40LG_037 | GGTTCCATTTCAGGTGATCAG | 290 |
| gCD40LG_038 | GTTCCATTTCAGGTGATCAGA | 291 |
| gCD40LG_039 | AGGTGATCAGAATCCTCAAAT | 292 |
| gCD40LG_040 | CTGCTGGCCTCACTTATGACA | 293 |
| gCD40LG_041 | AGCCCACTGTAACACTGTTAC | 294 |
| gCD40LG_042 | CAGCCCACTGTAACACTGTTA | 295 |
| gCD40LG_043 | TCAGCCCACTGTAACACTGTT | 296 |
| gCD40LG_044 | CCTTTCTTTGTAACAGTGTTA | 297 |
| gCD40LG_045 | TTTGTAACAGTGTTACAGTGG | 298 |
| gCD40LG_046 | TAACAGTGTTACAGTGGGCTG | 299 |
| gCD40LG_047 | CAGGGTTACCAAGTTGTTGCT | 300 |
| gCD40LG_048 | CCAGGGTTACCAAGTIGTTGC | 301 |
| gCD40LG_049 | CCATTTTCCAGGGTTACCAAG | 302 |
| gCD40LG_050 | ACGGTCAGCTGTTTCCCATTT | 303 |
| gCD40LG_051 | AACGGTCAGCTGTTTCCCATT | 304 |
| gCD40LG_052 | GGCAGAGGCTGGCTATAAATG | 305 |
| gCD40LG_053 | TAGCCAGCCTCTGCCTAAAGT | 306 |
| gCD40LG_054 | CAGCTCTGAGTAAGATTCTCT | 307 |
| gCD40LG_055 | GCGGAACTGTGGGTATTTGCA | 308 |
| gCD40LG_056 | AATTGCAACCAGGTGCTTCGG | 309 |
| gCD40LG_057 | TCAATGTGACTGATCCAAGCC | 310 |
| gCD40LG_058 | AGTAAGCCAAAGGACGTGAAG | 311 |
| gCD40LG_059 | GCTTACTCAAACTCTGAACAG | 312 |
| gCD40LG_060 | ACTGCTGGCCTCACTTATGAC | 313 |

TABLE 3
Selected Spacer Sequences Targeting
Human TRBC1 Genes

| crRNA | Spacer Sequence | SEQ ID NO |
|---|---|---|
| gTRBC1_001 | CAGAGGACCTGAACAAGGTGT | 314 |
| gTRBC1_002 | CCTCTCCCTGCTTTCTTTCAG | 315 |
| gTRBC1_003 | CTCTCCCTGCTTTCTTTCAGA | 316 |
| gTRBC1_004 | TTTCAGACTGTGGCTTTACCT | 317 |
| gTRBC1_005 | AGACTGTGGCTTTACCTCGGG | 318 |
| gTRBC1_006 | TCTTCTGCAGGTCAAGAGAAA | 319 |

TABLE 4
Selected Spacer Sequences Targeting
Human TRBC2 Genes

| crRNA | Spacer Sequence | SEQ ID NO |
|---|---|---|
| gTRBC2_001 | CAGAGGACCTGAAAAACGTGT | 320 |
| gTRBC2_002 | TCTTCCCCTGTTTTCTTTCAG | 321 |
| gTRBC2_003 | CTTCCCCTGTTTTCTTTCAGA | 322 |
| gTRBC2_004 | TTCCCCTGTTTTCTTTCAGAC | 323 |
| gTRBC2_005 | CTTTCAGACTGTGGCTTCACC | 324 |
| gTRBC2_006 | TTTCAGACTGTGGCTTCACCT | 325 |
| gTRBC2_007 | AGACTGTGGCTTCACCTCCGG | 326 |
| gTRBC2_008 | GAGCTAGCCTCTGGAATCCTT | 327 |
| gTRBC2_009 | GGAGCTAGCCTCTGGAATCCT | 328 |

TABLE 5
Selected Spacer Sequences Targeting
Human TRBC1_2 Genes

| CrRNA | Spacer Sequence | SEQ ID NO |
|---|---|---|
| gTRBC1_2_001 | GGTGTGGGAGATCTCTGCTTC | 329 |
| gTRBC1_2_002 | GGGTGTGGGAGATCTCTGCTT | 330 |
| gTRBC1_2_003 | AGCCATCAGAAGCAGAGATCT | 331 |
| gTRBC1_2_004 | GCCCTATCCTGGGTCCACTCG | 332 |

TABLE 6
Selected Spacer Sequences Targeting
Human CD3E Genes

| crRNA | Spacer Sequence | SEQ ID NO |
|---|---|---|
| gCD3E_1 | CACTCCATCCTACTCACCTGA | 333 |
| gCD3E_2 | tttttCTTATTTATTTTCTAG | 334 |
| gCD3E_3 | ttttCTTATTTATTTTCTAGT | 335 |
| gCD3E_4 | tttCTTATTTATTTTCTAGTT | 336 |
| gCD3E_5 | ttCTTATTTATTTTCTAGTTG | 337 |
| gCD3E_6 | tCTTATTTATTTTCTAGTTGG | 338 |
| gCD3E_7 | CTTATTTATTTTCTAGTTGGC | 339 |
| gCD3E_8 | TTATTTATTTTCTAGTTGGCG | 340 |
| gCD3E_9 | TTTTCTAGTTGGCGTTTGGGG | 341 |
| gCD3E_10 | CTAGTTGGCGTTTGGGGGCAA | 342 |
| gCD3E_11 | TAGTTGGCGTTTGGGGGCAAG | 343 |
| gCD3E_12 | CTTTTCAGGTAATGAAGAAAT | 344 |
| gCD3E_13 | CAGGTAATGAAGAAATGGGTA | 345 |
| gCD3E_14 | AGGTAATGAAGAAATGGGTAA | 346 |
| gCD3E_15 | CTTTTTTCATTTTCAGGTGGT | 347 |
| gCD3E_16 | TTCATTTTCAGGTGGTATTAC | 348 |
| gCD3E_17 | TCATTTTCAGGTGGTATTACA | 349 |
| gCD3E_18 | CATTTTCAGGTGGTATTACAC | 350 |
| gCD3E_19 | ATTTTCAGGTGGTATTACACA | 351 |
| gCD3E_20 | CAGGTGGTATTACACAGACAC | 352 |
| gCD3E_21 | AGGTGGTATTACACAGACACG | 353 |
| gCD3E_22 | CCTTCTTTCTCCCCAGCATAT | 354 |
| gCD3E_23 | TCCCCAGCATATAAAGTCTCC | 355 |
| gCD3E_24 | AGATCCAGGATACTGAGGGCA | 356 |
| gCD3E_25 | tcatTGTGTTGCCATAGTATT | 357 |
| gCD3E_26 | atcatTGTGTTGCCATAGTAT | 358 |
| gCD3E_27 | tatcatTGTGTTGCCATAGTA | 359 |
| gCD3E_28 | tcatcctcatcaccgcctatg | 360 |
| gCD3E_29 | atcatcctcatcaccgcctat | 361 |
| gCD3E_30 | tatcatcctcatcaccgccta | 362 |
| gCD3E_31 | CTCCAATTCTGAAAATTCCTT | 363 |
| gCD3E_32 | CAGAATTGGAGCAAAGTGGTT | 364 |
| gCD3E_33 | AGAATTGGAGCAAAGTGGTTA | 365 |
| gCD3E_34 | CTTCCTCTGGGGTAGCAGACA | 366 |
| gCD3E_35 | ATCTCTACCTGAGGGCAAGAG | 367 |
| gCD3E_36 | TCTCTACCTGAGGGCAAGAGG | 368 |
| gCD3E_37 | TATTCTTGCTCCAGTAGTAAA | 369 |
| gCD3E_38 | CTACTGGAGCAAGAATAGAAA | 370 |
| gCD3E_39 | CCTGCCGCCAGCACCCGCTCC | 371 |
| gCD3E_40 | CCCTCCTTCCTCCGCAGGACA | 372 |
| gCD3E_41 | TATCCCACGTTACCTCATAGT | 373 |
| gCD3E_42 | ACCCCCAGCCCATCCGGAAAG | 374 |

TABLE 7
Selected Spacer Sequences Targeting
Human CD38 Genes

| crRNA | Spacer Sequence | SEQ ID NO |
|---|---|---|
| gCD38_001 | TCCCCGGACACCGGGCTGAAC | 375 |
| gCD38_002 | AGTGTACTTGACGCATCGCGC | 376 |
| gCD38_003 | CCGAGACCGTCCTGGCGCGAT | 377 |
| gCD38_004 | GCAGTCTACATGTCTGAGATA | 378 |
| gCD38_005 | TGTGTTTTATCTCAGACATGT | 379 |
| gCD38_006 | TCTCAGACATGTAGACTGCCA | 380 |
| gCD38_007 | AAATAAATGCACCCTTGAAAG | 381 |
| gCD38_008 | AAGGGTGCATTTATTTCAAAA | 382 |
| gCD38_009 | TTTCAAAACATCCTTGCAACA | 383 |
| gCD38_010 | AAAACATCCTTGCAACATTAC | 384 |
| gCD38_011 | TTCTGCTCCAAAGAAGAATCT | 385 |
| gCD38_012 | TTCTTCCTTAGATTCTTCTTT | 386 |
| gCD38_013 | GAGCAGAATAAAAGATCTGGC | 387 |
| gCD38_014 | TACAAACTATGTCTTTTAGAA | 388 |
| gCD38_015 | TCCAGTCTGGGCAAGATTGAT | 389 |
| gCD38_016 | GAAATAAACTATCAATCTTGC | 390 |
| gCD38_017 | CAGAATACTGAAACAGGGTTG | 391 |
| gCD38_018 | AGTATTCTGGAAAACGGTTTC | 392 |
| gCD38_019 | ACTACTTGGTACTTACCCTGC | 393 |
| gCD38_020 | AGTTTGCAGAAGCTGCCTGTG | 394 |
| gCD38_021 | CAGAAGCTGCCTGTGATGTGG | 395 |
| gCD38_022 | CTGCGGGATCCATTGAGCATC | 396 |
| gCD38_023 | TCAAAGATTTTACTGCGGGAT | 397 |
| gCD38_024 | GGGTTCTTTGTTTCTTCTATT | 398 |
| gCD38_025 | TTTCTTCTATTTTAGCACTTT | 399 |
| gCD38_026 | TTCTATTTTAGCACTTTTGGG | 400 |
| gCD38_027 | GCACTTTTGGGAGTGTGGAAG | 401 |
| gCD38_028 | GGAGTGTGGAAGTCCATAATT | 402 |
| gCD38_029 | CAACCAGAGAAGGTTCAGACA | 403 |
| gCD38_030 | TGGTGGGATCCTGGCATAAGT | 404 |
| gCD38_031 | TTCCCCAGAGACTTATGCCAG | 405 |
| gCD38_032 | CTTATAATCGATTCCAGCTCT | 406 |
| gCD38_033 | CTTTTTTGCTTTCTTGTCATA | 407 |
| gCD38_034 | CTTTCTTGTCATAGACCTGAC | 408 |
| gCD38_035 | ACACACTGAAGAAACTTGTCA | 409 |
| gCD38_036 | TTGTCATAGACCTGACAAGTT | 410 |
| gCD38_037 | TTCAGTGTGTGAAAAATCCTG | 411 |

TABLE 8
Spacer Sequences Targeting Other Human Genes

| | | SEQ ID |
|---|---|---|
| CrRNA | Spacer Sequence | NO |
| gAPLNR_001 | ACAACTACTATGGGGCAGACA | 412 |
| gAPLNR_002 | CAGTCTGTGTACTCACACTCA | 413 |
| gAPLNR_003 | GGAGCAGCCGGGAGAAGAGGC | 414 |
| gAPLNR_004 | GGACCTTCTTCTGCAAGCTCA | 415 |
| gAPLNR_006 | TGGTGCCCTTCACCATCATGC | 416 |
| gAPLNR_007 | GGCGATGAAGAAGTAACAGGT | 417 |
| gAPLNR_008 | CCCTGTGCTGGATGCCCTACC | 418 |
| gAPLNR_009 | ACCTCTTCCTCATGAACATCT | 419 |
| gAPLNR_010 | GACCCCCGCTTCCGCCAGGCC | 420 |
| gAPLNR_011 | TCGTGCATCTGTTCTCCACCC | 421 |
| gBBS1_005 | CATGGGGATGGGGAATACAAG | 422 |
| gBBS1_007 | GGTCATCACCAGTGGTCCTTT | 423 |
| gBBS1_009 | GCCTGGTTCCAAAGGTCTTGT | 424 |
| gBBS1_015 | ACTTAGCTCCAGCTGCAGAAA | 425 |
| gBBS1_016 | CAAATGCCTCCATTTCACTTA | 426 |
| gBBS1_017 | TGCAGCTGGAGCTAAGTGAAA | 427 |
| gBBS1_018 | TAAACCAACACAAGTCCAACT | 428 |
| gBBS1_028 | CACTGTCCACTTCCCTAGGTG | 429 |
| gBBS1_032 | CGTGGATCAGACACTGCGAGA | 430 |
| gBBS1_033 | TCCACCCACCCTCTCCATAGG | 431 |
| gCALR_001 | GATTCGATCCAGCGGGAAGTC | 432 |
| gCALR_006 | CAGACAAGCCAGGATGCACGC | 433 |
| gCALR_011 | ACCGTGAACTGCACCACCAGC | 434 |
| gCALR_012 | CTAATAGTTTGGACCAGACAG | 435 |
| gCALR_013 | GACCAGACAGACATGCACGGA | 436 |
| gCALR_014 | CCACCACCCCCAGGCACACCT | 437 |
| gCALR_015 | CACACCTGTACACACTGATTG | 438 |
| gCALR_017 | AAGCATCAGGATCCTTTATCT | 439 |
| gCALR_019 | TGGGTGGATCCAAGTGCCCTT | 440 |
| gCALR_021 | CTCCAAGTCTCACCTGCCAGA | 441 |
| gCD247_001 | TGAGGGAAAGGACAAGATGAA | 442 |
| gCD247_002 | ACCGCGGCCATCCTGCAGGCA | 443 |
| gCD247_004 | GGATCCAGCAGGCCAAAGCTC | 444 |
| gCD247_005 | GCCTGCTGGATCCCAAACTCT | 445 |
| gCD247_007 | TGTGTTGCAGTTCAGCAGGAG | 446 |
| gCD247_011 | CTAGCAGAGAAGGAAGAACCC | 447 |
| gCD247_012 | ATCCCAATCTCACTGTAGGCC | 448 |
| gCD247_013 | ACTCCCAAACAACCAGCGCCG | 449 |
| gCD247_015 | CTTTCACGCCAGGGTCTCAGT | 450 |
| gCD247_016 | ACGCCAGGGTCTCAGTACAGC | 451 |
| gCD3G_001 | CCGGAGGACAGAGACTGACAT | 452 |
| gCD3G_004 | GCTTCTGCATCACAAGTCAGA | 453 |
| gCD3G_006 | TCTTCAGTTAGGAAGCCGATC | 454 |
| gCD3G_007 | AAGATGGGAAGATGATCGGCT | 455 |
| gCD3G_008 | CACTGATACATCCCTCGAGGG | 456 |
| gCD3G_011 | GTTCAATGCAGTTCTGACACA | 457 |
| gCD3G_012 | CCTACAGTGTGTCAGAACTGC | 458 |
| gCD3G_017 | CCTCTCGACTGGCGAACTCCA | 459 |
| gCD3G_022 | CTTGAAGGTGGCTGTACTGGT | 460 |
| gCD3G_023 | CAGGTACTTTGGCCCAGTCAA | 461 |
| gCD52_1 | CTCTTCCTCCTACTCACCATC | 462 |
| gCD52_10 | TCCTGAGAGTCCAGTTTGTAT | 463 |
| gCD52_4 | GCTGGTGTCGTTTTGTCCTGA | 464 |
| gCD52_9 | TTCGTGGCCAATGCCATAATC | 465 |
| gCD58_004 | CCAACAAATATATGGTGTTGT | 466 |
| gCD58_005 | AAGGCACATTGCTTGGTACAT | 467 |
| gCD58_010 | AAAGAGGTCCTATGGAAAAAA | 468 |
| gCD58_012 | AAAGATGAGAAAGCTCTGAAT | 469 |
| gCD58_018 | GCGATTCCATTTCATACTCAT | 470 |
| gCD58_019 | CAGAGTCTCTTCCATCTCCCA | 471 |
| gCD58_020 | CATTGCTCCATAGGACAATCC | 472 |
| gCD58_023 | AGATGGAAAATGATCTTCCAC | 473 |
| gCD58_028 | TAGGTCATTCAAGACACAGAT | 474 |
| gCD58_033 | GGTATTCTGAAATGTGACAGA | 475 |
| gCOL17A1_005 | TAGTTGTCACTGAAACAGTAA | 476 |
| gCOL17A1_006 | GCATAGCCATTGCTGGTCCCG | 477 |
| gCOL17A1_017 | ACTCCGTCCTCTGGTTGAAGA | 478 |
| gCOL17A1_024 | CAGTGTCAGGCACCTACGATG | 479 |
| gCOL17A1_047 | CTGTTCCATCATTAGCTTCTT | 480 |
| gCOL17A1_054 | AGGTGACATGGGAAGTCCAGG | 481 |
| gCOL17A1_065 | CAAGAAGCAGCAAACTGACCT | 482 |
| gCOL17A1_070 | GGTGACAAAGGACCAATGGGA | 483 |
| gCOL17A1_084 | AGAGGGGTCATCGATGCTCAC | 484 |
| gCOL17A1_094 | ATGCCGGCTCTACTGTACCTT | 485 |
| gDEFB134_001 | CCTGCCAGCACTGGATCCCAA | 486 |
| gDEFB134_004 | CTTTGGGATCCAGTGCTGGCA | 487 |
| gDEFB134_007 | CTTCCAGGTATAAATTCATTA | 488 |
| gDEFB134_008 | TTGTGCATTTCTGATGATAAT | 489 |
| gDEFB134_009 | TAGCATTTCTTGTGCATTTCT | 490 |
| gDEFB134_010 | ACTCTCATAGCATTCAAGTCT | 491 |
| gDEFB134_011 | ACACAGCACTCCAGCTGAAAC | 492 |
| gDEFB134_012 | CTTTGACACAGCACTCCAGCT | 493 |
| gDEFB134_013 | AGCTGGAGTGCTGTGTCAAAG | 494 |
| gDEFB134_014 | TTATGTCAGGGTGCAGGATTT | 495 |
| gERAP1_008 | CATGGATCAAGAGATCATAAT | 496 |
| gERAP1_015 | CAAAAGCACCTACAGAACCAA | 497 |
| gERAP1_029 | AGTCTGTCAGCAAGATAACCA | 498 |
| gERAP1_035 | GGTAGGGGATACGGTATGCTG | 499 |
| gERAP1_037 | AGCATACCGTATCCCCTACCC | 500 |
| gERAP1_039 | CATAGCACCAGACTGAAAGTC | 501 |
| gERAP1_061 | CCTTATCATAAGAAACATCAT | 502 |
| gERAP1_065 | AATGCGTCAGCACTAAGATAC | 503 |
| gERAP1_077 | CCCTAATAACCATCACAGTGA | 504 |
| gERAP1_078 | CTCTAGGAGCATTACCCAGTG | 505 |
| gERAP2_001 | TGTGTGAATTAACCATTGCAG | 506 |
| gERAP2_014 | ATGTATCTTGAATCTTCCTCT | 507 |
| gERAP2_018 | AGTTACCCTGCTCATGAACAA | 508 |
| gERAP2_046 | GAGAGTGGATAGTAGATATCA | 509 |
| gERAP2_048 | ATATCTACTATCCACTCTCCA | 510 |
| gERAP2_099 | ATGTGGACTCAAATGGTTACT | 511 |
| gERAP2_108 | CCTGTCAATCACTGGCTTAAA | 512 |
| gERAP2_118 | GAGCAATATGAACTGTCAATG | 513 |
| gERAP2_134 | ACTTGGGCTCATATGACATAA | 514 |
| gERAP2_261 | TCCTTACCATGTTACTTGTCA | 515 |
| gIFNGR1_004 | TTACAGTGCCTACACCAACTA | 516 |
| gIFNGR1_006 | CCGTAGAGGTAAAGAACTATG | 517 |
| gIFNGR1_008 | GTGTTAAGAATTCAGAATGGA | 518 |
| gIFNGR1_010 | ATGGATCACCAACATGATCAG | 519 |
| gIFNGR1_012 | ACTCTGACCCAAAGAGAATTT | 520 |
| gIFNGR1_021 | GGGATCATAATCGACTTCCTG | 521 |
| gIFNGR1_025 | AGTTGTAACACCCCACACATG | 522 |
| gIFNGR1_042 | GAGACAAAACCTGAATCAAAA | 523 |
| gIFNGR1_049 | AGTAGTAACCAGTCTGAACCT | 524 |
| gIFNGR1_052 | TGGAGTGATCACTCTCAGAAC | 525 |
| gIFNGR2_001 | TCTGTCCCCCTCAAGACCCTC | 526 |
| gIFNGR2_003 | AACTGCACTTGGTAGACAACA | 527 |
| gIFNGR2_005 | CTTCCCAGCACCGACAGTAAA | 528 |
| gIFNGR2_006 | AATGTCACTCTACGCCTTCGA | 529 |
| gIFNGR2_012 | CCAGTAATGGACATAATAACA | 530 |
| gIFNGR2_015 | AGTTATCCAATGAAATGGAGT | 531 |
| gIFNGR2_017 | ATTGGATAACTTAAAACCCTC | 532 |
| gIFNGR2_021 | GTAGCAAGATATGTTGCTTAA | 533 |
| gIFNGR2_026 | GCCTCCACTGAGCTTCAGCAA | 534 |
| gIFNGR2_031 | ACACTCCACCAAGCATCCCAT | 535 |
| gJAK1_002 | CTTCCACAACAGTATCTAAAT | 536 |
| gJAK1_021 | GCTACAAGCGATATATTCCAG | 537 |
| gJAK1_037 | ATTCGAATGACGGTGGAAACG | 538 |
| gJAK1_059 | GCATGAAGCTGATGTTATCCG | 539 |
| gJAK1_074 | GTACACACATTTCCATGGACC | 540 |
| gJAK1_075 | CCAGAGCGTGGTTCCAAAGCT | 541 |
| gJAK1_090 | AGATCAGCTATGTGGTTACCT | 542 |
| gJAK1_100 | CCTTACAAATCTGAACGGCAT | 543 |
| gJAK1_108 | ACCAAAGCAATTGAAACCGAT | 544 |
| gJAK1_111 | GATTGCATTAAACATTCTGGA | 545 |
| gJAK2_009 | GAAGCAGCAATACAGATTTCT | 546 |
| gJAK2_101 | AAGGCGTACGAAGAGAAGTAG | 547 |
| gJAK2_118 | AGATATGTATCTAGTGATCCA | 548 |
| gJAK2_121 | GATCACTAGATACATATCTGA | 549 |
| gJAK2_126 | GCACATACATTCCCATGAATA | 550 |
| gJAK2_132 | AATGCATTCAGGTGGTACCCA | 551 |
| gJAK2_137 | CCACAAAGTGGTACCAAAACT | 552 |
| gJAK2_175 | AAGATAGTCTCGTAAACTTCC | 553 |
| gJAK2_187 | GGTTAACCAAAGTCTTGCCAC | 554 |
| gJAK2_191 | CAGGTATGCTCCAGAATCACT | 555 |
| gmir-101- | GGTTATCATGGTACCGATGCT | 556 |
| 2_001 | | |
| gmir-101- | AGATATACAGCATCGGTACCA | 557 |
| 2_002 | | |
| gmir-101- | TCAATGTGATGGCACCACCAT | 558 |
| 2_003 | | |
| gMLANA_001 | AACTTACTCTTCAGCCGTGGT | 559 |
| gMLANA_002 | TCTATCTCTTGGGCCAGGGCC | 560 |
| gMLANA_003 | GTCTTCTACAATACCAACAGC | 561 |
| gMLANA_004 | CCAACCATCAAGGCTCTGTAT | 562 |
| gMLANA_008 | CATTTCAGGATAAAAGTCTTC | 563 |
| gMLANA_009 | AGGATAAAAGTCTTCATGTTG | 564 |
| gMLANA_010 | CTGTCCCGATGATCAAACCCT | 565 |
| gMLANA_011 | TCTTGAAGAGACACTTTGCTG | 566 |
| gMLANA_012 | ATCATCGGGACAGCAAAGTGT | 567 |
| gMLANA_020 | TCATAAGCAGGTGGAGCATTG | 568 |
| gPSMB5_001 | TGCCCACACTAGACATGGCGC | 569 |
| gPSMB5_002 | GGACTTGGGGGTCGTGCAGAT | 570 |
| gPSMB5_003 | GATTCCTGGCTCTTCTGGGAC | 571 |
| gPSMB5_005 | CTCTGATCTTAACAGTTCCGC | 572 |
| gPSMB5_006 | GAAGCTCATAGATTCGACATT | 573 |
| gPSMB5_007 | GAGGCAGCTGCTACAGAGATG | 574 |
| gPSMB5_008 | TACTGATACACCATGTTGGCA | 575 |
| gPSMB5_010 | CAGGCCTCTACTACGTGGACA | 576 |
| gPSMB5_011 | AGGGGCCACCTTCTCTGTAGG | 577 |
| gPSMB5_012 | AGGGGGTAGAGCCACTATACT | 578 |
| gPSMB8_001 | TCTATGCGATCTCCAGAGCTC | 579 |
| gPSMB8_004 | TCTTATCAGCCCACAGAATTC | 580 |
| gPSMB8_005 | TCCGTCCCCACCCAGGGACTG | 581 |
| gPSMB8_008 | AGTGTCGGCAGCCTCCAAGCT | 582 |
| gPSMB8_010 | ATCTTATAGGGTCCTGGACTC | 583 |
| gPSMB8_011 | CTGAGAGCCGAGTCCCATGTT | 584 |
| gPSMB8_012 | TCATTTGTCCACAGTGTACCA | 585 |
| gPSMB8_013 | ACCCAACCATCTTCCTTCATG | 586 |
| gPSMB8_014 | TCCACAGTGTACCACATGAAG | 587 |
| gPSMB8_015 | TACTTTCACCCAACCATCTTC | 588 |
| gPSMB9_001 | ACGGGGGCGTTGTGATGGGTT | 589 |
| gPSMB9_002 | CTCACCCTGCAGACACTCGGG | 590 |
| gPSMB9_005 | CCTCAGGATAGAACTGGAGGA | 591 |
| gPSMB9_007 | TCACCACATTTGCAGCAGCCA | 592 |
| gPSMB9_009 | GCTGCTGCAAATGTGGTGAGA | 593 |
| gPSMB9_010 | GGAGAAACTCACCTGACCTCC | 594 |
| gPSMB9_011 | ACCTGAGGATCCCTTTCCCAG | 595 |
| gPSMB9_012 | CCAGGTATATGGAACCCTGGG | 596 |
| gPSMB9_014 | TCTATGGTTATGTGGATGCAG | 597 |
| gPSMB9_015 | GCAGTTCATTGCCCAAGATGA | 598 |
| gPTCD2_005 | ACCACATTATCTGTAAGTAGG | 599 |
| gPTCD2_007 | GCTAAAAGATACCTACTTACA | 600 |
| gPTCD2_011 | GTGCCAGAAAGATTACATGCA | 601 |
| gPTCD2_018 | ATTACCAGGTACCATGCAGAG | 602 |
| gPTCD2_026 | TTCTCAGACTCCACATCATTC | 603 |
| gPTCD2_032 | ATCTCTATCAATACTTGCAAA | 604 |
| gPTCD2_033 | GCAGGTGCTTTGCAAGTATTG | 605 |
| gPTCD2_042 | CCTGATTCAGAGCTAATGCCA | 606 |
| gPTCD2_043 | GCTGTGGCATTAGCTCTGAAT | 607 |
| gPTCD2_064 | ATAGCAACGTGTGAGATTTCC | 608 |
| gRFX5_008 | TGTAGCTCAGAGCCAAGTACA | 609 |
| gRFX5_012 | GCAAGATCATCAGAGAGATCT | 610 |
| gRFX5_013 | ACTTGCATCAGATATTGCTAC | 611 |
| gRFX5_015 | GTACTTACACTCTCAGAACCC | 612 |
| gRFX5_016 | AGGATCCGCTCTGCCCAGTCA | 613 |
| gRFX5_017 | GTACCTCTGCAGAAGAGGACG | 614 |
| gRFX5_018 | GATGACCGTTCCCGAGGTGCA | 615 |
| gRFX5_026 | GCTGGTGGAGCCTGCCCACTG | 616 |
| gRFX5_028 | GCATCACTTGCTGTATCCTCT | 617 |
| gRFX5_038 | GCTTCTGCTGCCCTTGATGAC | 618 |
| gRFXANK_001 | CCCATGGAGCTTACCCAGCCT | 619 |
| gREXANK_002 | CCTGCACCCCTGAGCCTGTGA | 620 |
| gRFXANK_003 | CCAGCAGGCAGCTCCCTGAAG | 621 |
| gRFXANK_005 | GAGAGATTGAGACCGTTCGCT | 622 |
| gRFXANK_006 | CCAGGATGTGGGGGTCGGCAC | 623 |
| gRFXANK_007 | TCCTGCCCCTACCCACGACAG | 624 |
| gRFXANK_008 | ACGTGGTTCCCGCGCACAGCG | 625 |
| gRFXANK_009 | CAGCCCGAGGCGCTGACCTCA | 626 |
| gRFXANK_010 | CGGTATCCCAGGGCCACGGCA | 627 |
| gRFXANK_011 | CCTGCCCCATCTCAGTGCAAC | 628 |
| gREXAP_001 | GAGGATCTAGAGGACGAGGAG | 629 |
| gRFXAP_004 | TACTTGTCCTTGTACATCTTG | 630 |
| gRFXAP_005 | CCGCGCTGCCAGTCGAGGCAG | 631 |
| gRFXAP_009 | ACAATGGAGAGTATGTTATCT | 632 |
| gRFXAP_012 | GGGATCGTCCTGCAAGACCTA | 633 |
| gRFXAP_016 | GAACAAGTGTTAAATCAAAAA | 634 |
| gRFXAP_020 | TAAGTCGTTACTAAGAAGTCC | 635 |
| gRFXAP_021 | TGTAAAAATTGCACTACTTCT | 636 |
| gRFXAP_023 | CAGAAACAGCAACAGCTATTA | 637 |
| gRFXAP_025 | GAGCAAAGACAACAGCAGTTT | 638 |
| gRPL23_003 | GCACCAGAGGACCCACCACGT | 639 |
| gRPL23_004 | TATCCACAGGACGTGGTGGGT | 640 |
| gRPL23_008 | TAGGAGCCAAAAACCTGTATA | 641 |
| gRPL23 013 | GTTGTCGAATGACCACTGCTG | 642 |
| gRPL23_014 | TTCTCTCAGTACATCCAGCAG | 643 |
| gRPL23_019 | AAGATAATGCAGGAGTCATAG | 644 |
| gRPL23_021 | CTACCTTTCATCTCGCCTTTA | 645 |
| gRPL23_025 | ATGCAGGTTCTGCCATTACAG | 646 |
| gRPL23_026 | CAAATATACTGGAGAATCATG | 647 |
| gRPL23_027 | CCTTCCCTTTATATCCACAGG | 648 |
| gSOX10_001 | CTGGCGCCGTTGACGCGCACG | 649 |
| gSOX10_002 | TTGTGCTGCATACGGAGCCGC | 650 |
| gSOx10_003 | ATGTGGCTGAGTTGGACCAGT | 651 |
| gSOX10_004 | GCATCCACACCAGGTGGTGAG | 652 |
| gSOx10_005 | ACTACTCTGACCATCAGCCCT | 653 |
| gSOx10_006 | GGGCCGGGACAGTGTCGTATA | 654 |
| gSRP54_011 | TCTTAGTTGCTTCACTAGTTT | 655 |
| gSRP54_020 | GTGGGTGTCCATGCCTTAACT | 656 |
| gSRP54_021 | GCTTGTAGACCCTGGAGTTAA | 657 |
| gSRP54_024 | CCACTCCCTTGCAATCCAACA | 658 |
| gSRP54_029 | TCACCCAGCTAGCATATTATT | 659 |
| gSRP54_030 | ATATGTGCAGACACATTCAGA | 660 |
| gSRP54_064 | ATTGGTACAGGGGAACATATA | 661 |
| gSRP54_087 | GCACCATCCGTACTGTCTAGT | 662 |
| gSRP54_090 | GTAAACAACCAGGAAGAATCC | 663 |
| gSRP54_096 | CCCTCAGGTGGCGACATGTCT | 664 |
| gSRP54_139 | AGGATAACTAACCAAGATCTG | 665 |
| gSTAT1_003 | CATGGGAAAACTGTCATCATA | 666 |
| gSTAT1_005 | TAACCACTGTGCCAGGTACTG | 667 |
| gSTAT1_009 | ATGACCTCCTGTCACAGCTGG | 668 |
| gSTAT1_013 | TTCTAACCACTCAAATCTAGG | 669 |
| gSTAT1_014 | AGGAAGACCCAATCCAGATGT | 670 |
| gSTAT1_026 | TAGTGTATAGAGCATGAAATC | 671 |
| gSTAT1_032 | TGATCACTCTTTGCCACACCA | 672 |
| gSTAT1_102 | CCTGACATCATTCGCAATTAC | 673 |
| gSTAT1_103 | GATACAGATACTTCAGGGGAT | 674 |
| gSTAT1_113 | GTCACCCTTCTAGACTTCAGA | 675 |
| gTap1_011 | GAGTGAAGGTATCGGCTGAGC | 676 |
| gTap1_012 | AGCCCCCAGACCTGGCTATGG | 677 |
| gTap1_016 | AGGAGAAACCTGTCTGGTTCT | 678 |
| gTap1_020 | CTTCTGCCCAAGAAGGTGGGA | 679 |
| gTap1_026 | GGGAAAAGCTGCAAGAAATAA | 680 |
| gTap1_030 | AGGTATGCTGCTGAAAGTGGG | 681 |
| gTap1_033 | TCTGAGGAGCCCACAGCCTTC | 682 |
| gTap1_035 | GGTAGGCAAAGGAGACATCTT | 683 |
| gTap1_036 | CCTACCCAAACCGCCCAGATG | 684 |
| gTap1_039 | GAAGAAGTCTTCAAGAAAATA | 685 |
| gTAP2_004 | GCAGCCCCCACAGCCCTCCCA | 686 |
| gTAP2_008 | AGGTGAGACATTAATCCCTCA | 687 |
| gTAP2_014 | AAGGAAGCCAGTTACTCATCA | 688 |
| gTAP2_027 | CAGACCCTGGTATACATATAT | 689 |
| gTAP2_028 | GCTGTCGGTCCATGTAGGAGA | 690 |
| gTAP2_029 | TCCTACATGGACCGACAGCCA | 691 |
| gTAP2_030 | ACAACCCCCTGCAGAGTGGTG | 692 |
| gTAP2_037 | ATCCAGCAGCACCTGTCCCCC | 693 |
| gTAP2_038 | AGTTGGGCAGGAGCCTGTGCT | 694 |
| gTAP2_040 | TAGAAGATACCTGTGTATATT | 695 |
| gTAPBP_001 | CGCTCGCATCCTCCACGAACC | 696 |
| gTAPBP_002 | GCAGAGGCGGGGAGAGGCACG | 697 |
| gTAPBP_003 | CCTACATGCCCCCCACCTCCG | 698 |
| gTAPBP_004 | GGCTAGAGTGGCGACGCCAGC | 699 |
| gTAPBP_007 | AGGAGGGCACCTATCTGGCCA | 700 |
| gTAPBP_010 | GTCCTCTTTCCCCAGAACCCC | 701 |
| gTAPBP_011 | CCCAGAACCCCCCAAAGTGTC | 702 |
| gTAPBP_012 | AGGGCCCTCCCTTGAGGACAG | 703 |
| gTAPBP_013 | CTGTCTGCCTTTCTTCTGCTT | 704 |
| gTAPBP_016 | CCCACAGCTGTCTACCTGTCC | 705 |
| gTWF1_005 | CACAGCAAGTGAAGATGTTAA | 706 |
| gTWF1_012 | ATAGAGCAACTTGTGATTGGA | 707 |
| gTWF1_015 | CCCCTGTTGGAGGACAAACAA | 708 |
| gTWF1_018 | ATGTGGCCACCTCCAAATTCC | 709 |
| gTWF1_020 | GAGGTGGCCACATTAAAGATG | 710 |
| gTWF1_022 | ATCTGTCGTAGTTCTTCCTCA | 711 |
| gTWF1_051 | CAGATCGAGATAGACAATGGG | 712 |
| gTWF1_053 | TGAAGAAGTACATCCCAAGCA | 713 |
| gTWF1_060 | ATGTGATGACTTTAATCAGTA | 714 |
| gTWF1_101 | AAATAGGTGGGCTACCTTTCT | 715 |

TABLE 9
Spacer Sequences Targeting Human CD3D
and NLRC5 Genes

| | | SEQ ID |
|---|---|---|
| crRNA | Spacer Sequence | NO |
| gCD3D_001 | TCTCTGGCCTGGTACTGGCTA | 716 |
| gCD3D_002 | CCCTTTAGTGAGCCCCTTCAA | 717 |
| gCD3D_003 | GTGAGCCCCTTCAAGATACCT | 718 |
| gCD3D_004 | TGAATTGCAATACCAGCATCA | 719 |
| gCD3D_005 | CCAGGTCCAGTCTTGTAATGT | 720 |
| gCD3D_006 | TCCTTGTATATATCTGTCCCA | 721 |
| gCD3D_007 | GGAGTCTTCTGCTTTGCTGGA | 722 |
| gCD3D_008 | CTGGACATGAGACTGGAAGGC | 723 |
| gCD3D_009 | TCTTCTCCTCTCTTAGCCCCT | 724 |
| gCD3D_010 | CTCCAAGGTGGCTGTACTGAG | 725 |
| gNLRC5_001 | GCTCCTGTAGCGCTGCTGGGC | 726 |
| gNLRC5_002 | GGGAAGGCTGGCATGGGCAAG | 727 |
| gNLRC5_003 | CAGGCCCTGTTCCTTTTTGAA | 728 |
| gNLRC5_004 | AATTCCGCCAGCTCAACTTGA | 729 |
| gNLRC5_005 | ATCTGTACCTGAGCCCTGAAT | 730 |
| gNLRC5_006 | ATGGGCTAGATGAGGCCCTCC | 731 |
| gNLRC5_007 | TCCCATCTCTGCAATGGGACC | 732 |
| gNLRC5_008 | ATGGGCCACGGGTGGAAGAAT | 733 |
| gNLRC5_009 | TCTGTAACTCCACCAGGGCCC | 734 |
| gNLRC5_010 | CATAGAAGATAACCTTCCCTG | 735 |
| gNLRC5_011 | GGGCCACTCACAGCCTGCTGA | 736 |
| gNLRC5_012 | ACCCACCTCAGCCTGCAGGAG | 737 |
| gNLRC5_013 | TTCACCTTGGGGCTGGCCATC | 738 |
| gNLRC5_014 | TTGCTGCCCTGCACCTGATGG | 739 |
| gNLRC5_015 | GTCCGCTGTACCCAGCGGGAA | 740 |
| gNLRC5_016 | GCCCTGTGAGCTTGCGGGTGG | 741 |
| gNLRC5_017 | TGCGGTGAGACTGGCCAGCTC | 742 |
| gNLRC5_018 | CCACTGACCTGCACCGACCTG | 743 |
| gNLRC5_019 | ATGGCTGTCCCCTGGAGCCCC | 744 |

The spacer sequences provided in Tables 1-9 are designed based upon identification of target nucleotide sequences associated with a PAM in a given target gene locus, and are selected based upon the editing efficiency detected in human cells.

To provide sufficient targeting to the target nucleotide sequence, the spacer sequence is generally 16 or more nucleotides in length. In certain embodiments, the spacer sequence is at least 16, 17, 18, 19, 20, 21, 22, 23, 24, 25, 26, 27, 28, 29, 30, 35, 40, 45, 50, or 75 nucleotides in length. In certain embodiments, the spacer sequence is shorter than or equal to 75, 50, 45, 40, 35, 30, 25, 21, or 20 nucleotides in length. Shorter spacer sequence may be desirable for reducing off-target events. Accordingly, in certain embodiments, the spacer sequence is shorter than or equal to 21, 20, 19, 18, or 17 nucleotides. In certain embodiments, the spacer sequence is 17-30 nucleotides in length, e.g., 17-21, 17-22, 17-23, 17-24, 17-25, 17-30, 20-21, 20-22, 20-23, 20-24, 20-25, or 20-30 nucleotides in length, for example 20-22 nucleotides in length, such as 20 or 21 nucleotides in length. In certain embodiments, the spacer sequence is 21 nucleotides in length. In certain embodiments, the spacer sequence is 20 nucleotides in length.

In certain embodiments, the spacer sequence comprises a portion of a spacer sequence listed in any of the Tables 1-9, wherein the portion is 16, 17, 18, 19, or 20 nucleotides in length. In certain embodiments, the spacer sequence comprises nucleotides 1-16, 1-17, 1-18, 1-19, or 1-20 of a spacer sequence listed in any of the Tables 1-9. In specific embodiments, the spacer sequence consists of nucleotides 1-16, 1-17, 1-18, 1-19, or 1-20 of a spacer sequence listed in any of the Tables 1-9.

In certain embodiments, the spacer sequence is 21 nucleotides in length. In certain embodiments, the spacer sequence consists of a spacer sequence shown in any of the Tables 1-9.

In certain embodiments, the spacer sequence, where it is longer than 21 nucleotides in length, comprises a spacer sequence shown in any of the Tables 1-9 and one or more nucleotides. In certain embodiments, the one or more nucleotides are 3′ to the spacer sequence shown in any of the Tables 1-9.

In certain embodiments, the spacer sequence is at least 70%, at least 75%, at least 80%, at least 85%, at least 90%, at least 93%, at least 94%, at least 95%, at least 96%, at least 97%, at least 98%, or at least 99% complementary to the target nucleotide sequence. In certain embodiments, the spacer sequence is 100% complementary to the target nucleotide sequence in the seed region (at least 5 base pairs proximal to the PAM). In certain embodiments, the spacer sequence is 100% complementary to the target nucleotide sequence. The spacer sequences listed in any of the Tables 1-9 are designed to be 100% complementary to the wild-type sequence of the corresponding target gene. Accordingly, it is contemplated that a spacer sequence useful for targeting a gene listed in any of the Tables 1-9 can be at least 70%, at least 75%, at least 80%, at least 85%, at least 90%, at least 93%, at least 94%, at least 95%, at least 96%, at least 97%, at least 98%, or at least 99% identical to a corresponding spacer sequence listed in any of the Tables 1-9, or a portion thereof disclosed herein. In certain embodiments, the spacer sequence is 1, 2, 3, 4, 5, 6, 7, 8, 9, or 10 nucleotides different from a sequence listed in any of the Tables 1-9. In certain embodiments, the spacer sequence is 100% identical to a sequence listed in any of the Tables 1-9 in the seed region (at least 5 base pairs proximal to the PAM). It has been reported that compared to DNA binding, DNA cleavage is less tolerant to mismatches between the spacer sequence and the target nucleotide sequence (see, Klein et al. (2018) CELL REPORTS, 22: 1413). Accordingly, in certain embodiments, a guide nucleic acid to be used with a Cas nuclease comprises a spacer sequence 100% complementary to the target nucleotide sequence. In certain embodiments, a guide nucleic acid to be used with a Cas nuclease comprises a spacer sequence listed in any of the Tables 1-9, or a portion thereof disclosed herein.

The present invention also provides guide nucleic acids targeting human DHODH, PLK1, MVD, TUBB, or U6 gene comprising the spacer sequences provided below in Table 20. DHODH, PLK1, MVD, and TUBB are known to be essential genes. It is contemplated that the guide nucleic acids targeting these genes, particularly the ones that edit the respective genomic locus at height efficiency (e.g., at least 50%, at least 60%, at least 70%, at least 80%, or at least 90%), can be used as positive controls for assessing transfection efficiency and other experimental processes. The spacer sequences targeting U6 in Table 20 are designed to hybridize with the promoter region of human U6 gene and can be used to assess expression of an inserted gene from the endogenous U6 promoter.

### A. Cas Proteins

The guide nucleic acid of the present invention, either as a single guide nucleic acid alone or as a targeter nucleic acid used in combination with a cognate modulator nucleic acid, is capable of binding a CRISPR Associated (Cas) protein. In certain embodiments, the guide nucleic acid, either as a single guide nucleic acid alone or as a targeter nucleic acid used in combination with a cognate modulator nucleic acid, is capable of activating a Cas nuclease.

The terms “CRISPR-Associated protein,” “Cas protein,” and “Cas,” as used interchangeably herein, can include a naturally occurring Cas protein or an engineered Cas protein. Non-limiting examples of Cas protein engineering includes but are not limited to mutations and modifications of the Cas protein that alter the activity of the Cas, alter the PAM specificity, broaden the range of recognized PAMs, and/or reduce the ability to modify one or more off-target loci as compared to a corresponding unmodified Cas. In certain embodiments, the altered activity of the engineered Cas comprises altered ability (e.g., specificity or kinetics) to bind the naturally occurring crRNA or engineered dual guide nucleic acids, altered ability (e.g., specificity or kinetics) to bind the target nucleotide sequence, altered processivity of nucleic acid scanning, and/or altered effector (e.g., nuclease) activity. A Cas protein having the nuclease activity is referred to as a “CRISPR-Associated nuclease” or “Cas nuclease,” as used interchangeably herein.

In certain embodiments, the Cas protein is a type V-A, type V-C, or type V-D Cas protein. In certain embodiments, the Cas protein is a type V-A Cas protein. In other embodiments, the Cas protein is a type II Cas protein, e.g., a Cas9 protein.

In certain embodiments, the Cas nuclease is a type V-A, type V-C, or type V-D Cas nuclease. In certain embodiments, the Cas nuclease is a type V-A Cas nuclease. In other embodiments, the Cas protein is a type II Cas nuclease, e.g., a Cas9 nuclease.

In certain embodiments, the type V-A Cas protein comprises Cpf1. Cpf1 proteins are known in the art and are described in U.S. Pat. Nos. 9,790,490 and 10,113,179. Cpf1 orthologs can be found in various bacterial and archaeal genomes. For example, in certain embodiments, the Cpf1 protein is derived from Francisella novicida U112 (Fn), Acidaminococcus sp. BV3L6 (As), Lachnospiraceae bacterium ND2006 (Lb), Lachnospiraceae bacterium MA2020 (Lb2), Candidatus Methanoplasma termitum (CMt), Moraxella bovoculi 237 (Mb), Porphyromonas crevioricanis (Pc), Prevotella disiens (Pd), Francisella tularensis 1, Francisella tularensis subsp. novicida, Prevotella albensis, Lachnospiraceae bacterium MC2017 1, Butyrivibrio proteoclasticus, Peregrinibacteria bacterium GW2011_GWA2_33_10, Parcubacteria bacterium GW2011_GWC2_44_17, Smithella sp. SCADC, Eubacterium eligens, Leptospira inadai, Porphyromonas macacae, Prevotella bryantii (Pb), Proteocatella sphenisci (Ps), Anaerovibrio sp. RM50 (As2), Moraxella caprae (Mc), Lachnospiraceae bacterium COE1 (Lb3), or Eubacterium coprostanoligenes (Ec).

In certain embodiments, the type V-A Cas protein comprises AsCpf1 or a variant thereof. In certain embodiments, the type V-A Cas protein comprises an amino acid sequence at least 30%, at least 40%, at least 50%, at least 60%, at least 70%, at least 75%, at least 80%, at least 85%, at least 90%, at least 91%, at least 92%, at least 93%, at least 94%, at least 95%, at least 96%, at least 97%, at least 98%, or at least 99% identical to the amino acid sequence set forth in SEQ ID NO: 3. In certain embodiments, the type V-A Cas protein comprises the amino acid sequence set forth in SEQ ID NO: 3.

AsCpf1
(SEQ ID NO: 3)
MTQFEGFTNLYQVSKTLRFELIPQGKTLKHIQEQGFIEEDKARNDHYKELKPIIDRIYKTYADQC
LQLVQLDWENLSAAIDSYRKEKTEETRNALIEEQATYRNAIHDYFIGRTDNLTDAINKRHAEIYK
GLFKAELFNGKVLKQLGTVTTTEHENALLRSEDKETTYFSGFYENRKNVESAEDISTAIPHRIVQ
DNFPKFKENCHIFTRLITAVPSLREHFENVKKAIGIFVSTSIEEVESFPFYNQLLTQTQIDLYNQ
LLGGISREAGTEKIKGLNEVLNLAIQKNDETAHIIASLPHRFIPLFKQILSDRNTLSFILEEFKS
DEEVIQSFCKYKTLLRNENVLETAEALFNELNSIDLTHIFISHKKLETISSALCDHWDTLRNALY
ERRISELTGKITKSAKEKVQRSLKHEDINLQEIISAAGKELSEAFKOKTSEILSHAHAALDQPLP
TTLKKQEEKEILKSQLDSLLGLYHLLDWFAVDESNEVDPEFSARLTGIKLEMEPSLSFYNKARNY
ATKKPYSVEKFKLNFQMPTLASGWDVNKEKNNGAILFVKNGLYYLGIMPKOKGRYKALSFEPTEK
TSEGFDKMYYDYFPDAAKMIPKCSTOLKAVTAHFQTHTTPILLSNNFIEPLEITKEIYDLNNPEK
EPKKFQTAYAKKTGDQKGYREALCKWIDFTRDELSKYTKTTSIDLSSLRPSSQYKDLGEYYAELN
PLLYHISFQRIAEKEIMDAVETGKLYLFQIYNKDFAKGHHGKPNLHTLYWTGLESPENLAKTSIK
LNGQAELFYRPKSRMKRMAHRLGEKMLNKKLKDQKTPIPDTLYQELYDYVNHRLSHDLSDEARAL
LPNVITKEVSHEIIKDRRFTSDKFFFHVPITLNYQAANSPSKENQRVNAYLKEHPETPIIGIDRG
ERNLIYITVIDSTGKILEQRSLNTIQQFDYQKKLDNREKERVAARQAWSVVGTIKDLKQGYLSQV
IHEIVDLMIHYQAVVVLENLNFGFKSKRTGIAEKAVYQQFEKMLIDKLNCLVLKDYPAEKVGGVL
NPYQLTDQFTSFAKMGTQSGELFYVPAPYTSKIDPLTGFVDPFVWKTIKNHESRKHFLEGEDELH
YDVKTGDFILHFKMNRNLSFQRGLPGEMPAWDIVFEKNETQFDAKGTPFIAGKRIVPVIENHRET
GRYRDLYPANELIALLEEKGIVERDGSNILPKLLENDDSHAIDTMVALIRSVLQMRNSNAATGED
YINSPVRDLNGVCFDSRFQNPEWPMDADANGAYHIALKGQLLLNHLKESKDLKLQNGISNODWLA
YIQELRN

In certain embodiments, the type V-A Cas protein comprises LbCpf1 or a variant thereof. In certain embodiments, the type V-A Cas protein comprises an amino acid sequence at least 30%, at least 40%, at least 50%, at least 60%, at least 70%, at least 75%, at least 80%, at least 85%, at least 90%, at least 91%, at least 92%, at least 93%, at least 94%, at least 95%, at least 96%, at least 97%, at least 98%, or at least 99% identical to the amino acid sequence set forth in SEQ ID NO: 4. In certain embodiments, the type V-A Cas protein comprises the amino acid sequence set forth in SEQ ID NO: 4.

LbCpf1
(SEQ ID NO: 4)
MSKLEKFTNCYSLSKTLRFKAIPVGKTQENIDNKRLLVEDEKRAEDYKGVKKLLDRYYLSFINDV
LHSIKLKNLNNYISLERKKTRTEKENKELENLEINLRKEIAKAFKGNEGYKSLEKKDIIETILPE
FLDDKDEIALVNSENGFTTAFTGFFDNRENMESEEAKSTSIAFRCINENLTRYISNMDIFEKVDA
IFDKHEVQEIKEKILNSDYDVEDFFEGEFFNFVLTQEGIDVYNAIIGGFVTESGEKIKGLNEYIN
LYNQKTKQKLPKFKPLYKQVLSDRESLSFYGEGYTSDEEVLEVERNTLNKNSEIFSSIKKLEKLE
KNFDEYSSAGIFVKNGPAISTISKDIFGEWNVIRDKWNAEYDDIHLKKKAVVTEKYEDDRRKSFK
KIGSFSLEQLQEYADADLSVVEKLKEIIIQKVDEIYKVYGSSEKLEDADEVLEKSLKKNDAVVAI
MKDLLDSVKSFENYIKAFFGEGKETNRDESFYGDFVLAYDILLKVDHIYDAIRNYVTQKPYSKDK
FKLYFQNPQFMGGWDKDKETDYRATILRYGSKYYLAIMDKKYAKCLQKIDKDDVNGNYEKINYKL
LPGPNKMLPKVFFSKKWMAYYNPSEDIQKIYKNGTFKKGDMENLNDCHKLIDFFKDSISRYPKWS
NAYDENESETEKYKDIAGFYREVEEQGYKVSFESASKKEVDKLVEEGKLYMFQIYNKDESDKSHG
TPNLHTMYFKLLFDENNHGQIRLSGGAELFMRRASLKKEELVVHPANSPIANKNPDNPKKTTTLS
YDVYKDKRESEDQYELHIPIAINKCPKNIFKINTEVRVLLKHDDNPYVIGIDRGERNLLYIVVVD
GKGNIVEQYSLNEIINNENGIRIKTDYHSLLDKKEKERFEARQNWTSIENIKELKAGYISQVVHK
ICELVEKYDAVIALEDLNSGFKNSRVKVEKQVYQKFEKMLIDKLNYMVDKKSNPCATGGALKGYQ
ITNKFESFKSMSTQNGFIFYIPAWLTSKIDPSTGFVNLLKTKYTSIADSKKFISSEDRIMYVPEE
DLFEFALDYKNFSRTDADYIKKWKLYSYGNRIRIFRNPKKNNVEDWEEVCLTSAYKELENKYGIN
YQQGDIRALLCEQSDKAFYSSFMALMSLMLQMRNSITGRTDVDFLISPVKNSDGIFYDSRNYEAQ
ENAILPKNADANGAYNIARKVLWAIGQFKKAEDEKLDKVKIAISNKEWLEYAQTSVKH

In certain embodiments, the type V-A Cas protein comprises FnCpf1 or a variant thereof. In certain embodiments, the type V-A Cas protein comprises an amino acid sequence at least 30%, at least 40%, at least 50%, at least 60%, at least 70%, at least 75%, at least 80%, at least 85%, at least 90%, at least 91%, at least 92%, at least 93%, at least 94%, at least 95%, at least 96%, at least 97%, at least 98%, or at least 99% identical to the amino acid sequence set forth in SEQ ID NO: 5. In certain embodiments, the type V-A Cas protein comprises the amino acid sequence set forth in SEQ ID NO: 5.

FnCpf1
(SEQ ID NO: 5)
MSIYQEFVNKYSLSKTLRFELIPQGKTLENIKARGLILDDEKRAKDYKKAKQIIDKYHQFFIEEI
LSSVCISEDLLQNYSDVYFKLKKSDDDNLQKDFKSAKDTIKKQISEYIKDSEKFKNLENQNLIDA
KKGQESDLILWLKQSKDNGIELFKANSDITDIDEALEIIKSFKGWTTYFKGEHENRKNVYSSNDI
PTSIIYRIVDDNLPKFLENKAKYESLKDKAPEAINYEQIKKDLAEELTFDIDYKTSEVNQRVESL
DEVFEIANFNNYLNQSGITKENTIIGGKFVNGENTKRKGINEYINLYSQQINDKTLKKYKMSVLF
KQILSDTESKSFVIDKLEDDSDVVTTMQSFYEQIAAFKTVEEKSIKETLSLLEDDLKAQKLDLSK
IYFKNDKSLTDLSQQVEDDYSVIGTAVLEYITQQIAPKNLDNPSKKEQELIAKKTEKAKYLSLET
IKLALEEFNKHRDIDKQCRFEEILANFAAIPMIFDEIAQNKDNLAQISIKYQNQGKKDLLQASAE
DDVKAIKDLLDQTNNLLHKLKIFHISQSEDKANILDKDEHFYLVFEECYFELANIVPLYNKIRNY
ITQKPYSDEKFKLNFENSTLANGWDKNKEPDNTAILFIKDDKYYLGVMNKKNNKIFDDKAIKENK
GEGYKKIVYKLLPGANKMLPKVFFSAKSIKFYNPSEDILRIRNHSTHTKNGSPQKGYEKFEFNIE
DCRKFIDFYKQSISKHPEWKDFGFRESDTQRYNSIDEFYREVENQGYKLTFENISESYIDSVVNQ
GKLYLFQIYNKDFSAYSKGRPNLHTLYWKALFDERNLQDVVYKLNGEAELFYRKQSIPKKITHPA
KEAIANKNKDNPKKESVFEYDLIKDKRFTEDKFFFHCPITINFKSSGANKENDEINLLLKEKAND
VHILSIDRGERHLAYYTLVDGKGNIIKQDTENIIGNDRMKTNYHDKLAAIEKDRDSARKDWKKIN
NIKEMKEGYLSQVVHEIAKLVIEYNAIVVFEDLNFGEKRGREKVEKQVYQKLEKMLIEKLNYLVE
KDNEFDKTGGVLRAYQLTAPFETFKKMGKQTGIIYYVPAGFTSKICPVTGFVNQLYPKYESVSKS
QEFFSKEDKICYNLDKGYFEFSEDYKNFGDKAAKGKWTIASFGSRLINERNSDKNHNWDTREVYP
TKELEKLLKDYSIEYGHGECIKAAICGESDKKFFAKLTSVLNTILQMRNSKIGTELDYLISPVAD
VNGNFFDSRQAPKNMPQDADANGAYHIGLKGLMLLGRIKNNQEGKKLNLVIKNEEYFEFVONRNN

In certain embodiments, the type V-A Cas protein comprises PbCpf1 or a variant thereof. In certain embodiments, the type V-A Cas protein comprises an amino acid sequence at least 30%, at least 40%, at least 50%, at least 60%, at least 70%, at least 75%, at least 80%, at least 85%, at least 90%, at least 91%, at least 92%, at least 93%, at least 94%, at least 95%, at least 96%, at least 97%, at least 98%, or at least 99% identical to the amino acid sequence set forth in SEQ ID NO: 6. In certain embodiments, the type V-A Cas protein comprises the amino acid sequence set forth in SEQ ID NO: 6.

PbCpf1
(SEQ ID NO: 6)
MQINNLKIIYMKFTDETGLYSLSKTLRFELKPIGKTLENIKKAGLLEQDQHRADSYKKVKKIIDE
YHKAFIEKSLSNFELKYQSEDKLDSLEEYLMYYSMKRIEKTEKDKFAKIQDNLRKQIADHLKGDE
SYKTIFSKDLIRKNLPDFVKSDEERTLIKEFKDETTYFKGFYENRENMYSAEDKSTAISHRIIHE
NLPKFVDNINAFSKIILIPELREKLNQIYQDFEEYLNVESIDEIFHLDYFSMVMTQKQIEVYNAI
IGGKSTNDKKIQGLNEYINLYNQKHKDCKLPKLKLLFKQILSDRIAISWLPDNEKDDQEALDSID
TCYKNLLNDGNVLGEGNLKLLLENIDTYNLKGIFIRNDLQLTDISQKMYASWNVIQDAVILDLKK
QVSRKKKESAEDYNDRLKKLYTSQESFSIQYLNDCLRAYGKTENIQDYFAKLGAVNNEHEQTINL
FAQVRNAYTSVQAILTTPYPENANLAQDKETVALIKNLLDSLKRLQRFIKPLLGKGDESDKDERE
YGDFTPLWETLNQITPLYNMVRNYMTRKPYSQEKIKLNFENSTLLGGWDLNKEHDNTAIILRKNG
LYYLAIMKKSANKIFDKDKLDNSGDCYEKMVYKLLPGANKMLPKVFFSKSRIDEFKPSENIIENY
KKGTHKKGANENLADCHNLIDFFKSSISKHEDWSKENFHESDTSSYEDLSDFYREVEQQGYSISE
CDVSVEYINKMVEKGDLYLFQIYNKDFSEFSKGTPNMHTLYWNSLESKENLNNIIYKLNGQAEIF
FRKKSLNYKRPTHPAHQAIKNKNKCNEKKESIFDYDLVKDKRYTVDKFQFHVPITMNFKSTGNTN
INQQVIDYLRTEDDTHIIGIDRGERHLLYLVVIDSHGKIVEQFTLNEIVNEYGGNIYRTNYHDLL
DTREQNREKARESWQTIENIKELKEGYISQVIHKITDLMQKYHAVVVLEDLNMGEMRGRQKVEKQ
VYQKFEEMLINKLNYLVNKKADQNSAGGLLHAYQLTSKFESFQKLGKQSGELFYIPAWNTSKIDP
VTGFVNLEDTRYESIDKAKAFFGKEDSIRYNADKDWFEFAFDYNNFTTKAEGTRINWTICTYGSR
IRTFRNQAKNSQWDNEEIDLTKAYKAFFAKHGINIYDNIKEAIAMETEKSFFEDLLHLLKLTLQM
RNSITGTTTDYLISPVHDSKGNFYDSRICDNSLPANADANGAYNIARKGLMLIQQIKDSTSSNRF
KFSPITNKDWLIFAQEKPYLND

In certain embodiments, the type V-A Cas protein comprises PsCpf1 or a variant thereof. In certain embodiments, the type V-A Cas protein comprises an amino acid sequence at least 30%, at least 40%, at least 50%, at least 60%, at least 70%, at least 75%, at least 80%, at least 85%, at least 90%, at least 91%, at least 92%, at least 93%, at least 94%, at least 95%, at least 96%, at least 97%, at least 98%, or at least 99% identical to the amino acid sequence set forth in SEQ ID NO: 7. In certain embodiments, the type V-A Cas protein comprises the amino acid sequence set forth in SEQ ID NO: 7.

PsCpf1
(SEQ ID NO: 7)
MENFKNLYPINKTLRFELRPYGKTLENFKKSGLLEKDAFKANSRRSMQAIIDEKFKETIEERLKY
TEFSECDLGNMTSKDKKITDKAATNLKKQVILSEDDEIENNYLKPDKNIDALFKNDPSNPVISTE
KGFTTYFVNFFEIRKHIFKGESSGSMAYRIIDENLTTYLNNIEKIKKLPEELKSQLEGIDQIDKL
NNYNEFITQSGITHYNEIIGGISKSENVKIQGINEGINLYCQKNKVKLPRLTPLYKMILSDRVSN
SFVLDTIENDTELIEMISDLINKTEISQDVIMSDIQNIFIKYKQLGNLPGISYSSIVNAICSDYD
NNFGDGKRKKSYENDRKKHLETNVYSINYISELLTDTDVSSNIKMRYKELEQNYQVCKENENATN
WMNIKNIKQSEKINLIKDLLDILKSIQRFYDLEDIVDEDKNPSAEFYTWLSKNAEKLDFEENSVY
NKSRNYLTRKQYSDKKIKLNFDSPTLAKGWDANKEIDNSTIIMRKENNDRGDYDYELGIWNKSTP
ANEKIIPLEDNGLFEKMQYKLYPDPSKMLPKQFLSKIWKAKHPTTPEFDKKYKEGRHKKGPDFEK
EFLHELIDCFKHGLVNHDEKYQDVEGENLRNTEDYNSYTEFLEDVERCNYNLSENKIADTSNLIN
DGKLYVFQIWSKDESIDSKGTKNLNTIYFESLESEENMIEKMEKLSGEAEIFYRPASLNYCEDII
KKGHHHAELKDKEDYPIIKDKRYSQDKFFFHVPMVINYKSEKLNSKSLNNRTNENLGQFTHIIGI
DRGERHLIYLTVVDVSTGEIVEQKHLDEIINTDTKGVEHKTHYLNKLEEKSKTRDNERKSWEAIE
TIKELKEGYISHVINEIQKLQEKYNALIVMENLNYGFKNSRIKVEKQVYQKFETALIKKENYIID
KKDPETYIHGYQLTNPITTLDKIGNQSGIVLYIPAWNTSKIDPVTGFVNLLYADDLKYKNQEQAK
SFIQKIDNIYFENGEFKEDIDESKWNNRYSISKTKWILTSYGTRIQTERNPQKNNKWDSAEYDLT
EEFKLILNIDGTLKSQDVETYKKEMSLFKLMLQLRNSVIGTDIDYMISPVTDKTGTHEDSRENIK
NLPADADANGAYNIARKGIMAIENIMNGISDPLKISNEDYLKYIQNQQE

In certain embodiments, the type V-A Cas protein comprises As2Cpf1 or a variant thereof. In certain embodiments, the type V-A Cas protein comprises an amino acid sequence at least 30%, at least 40%, at least 50%, at least 60%, at least 70%, at least 75%, at least 80%, at least 85%, at least 90%, at least 91%, at least 92%, at least 93%, at least 94%, at least 95%, at least 96%, at least 97%, at least 98%, or at least 99% identical to the amino acid sequence set forth in SEQ ID NO: 8. In certain embodiments, the type V-A Cas protein comprises the amino acid sequence set forth in SEQ ID NO: 8.

As2Cpf1
(SEQ ID NO: 8)
MVAFIDEFVGQYPVSKTLRFEARPVPETKKWLESDQCSVLENDQKRNEYYGVLKELLDDYYRAYI
EDALTSFTLDKALLENAYDLYCNRDTNAFSSCCEKLRKDLVKAFGNLKDYLLGSDQLKDLVKLKA
KVDAPAGKGKKKIEVDSRLINWLNNNAKYSAEDREKYIKAIESFEGFVTYLTNYKQARENMESSE
DKSTAIAFRVIDQNMVTYFGNIRIYEKIKAKYPELYSALKGFEKFFSPTAYSEILSQSKIDEYNY
QCIGRPIDDADEKGVNSLINEYRQKNGIKARELPVMSMLYKQILSDRDNSEMSEVINRNEEAIEC
AKNGYKVSYALFNELLQLYKKIFTEDNYGNIYVKTQPLTELSQALFGDWSILRNALDNGKYDKDI
INLAELEKYESEYCKVLDADDAAKIQDKENLKDYFIQKNALDATLPDLDKITQYKPHLDAMLQAI
RKYKLFSMYNGRKKMDVPENGIDESNEFNAIYDKLSEFSILYDRIRNFATKKPYSDEKMKLSENM
PTMLAGWDYNNETANGCFLFIKDGKYFLGVADSKSKNIFDEKKNPHLLDKYSSKDIYYKVKYKQV
SGSAKMLPKVVFAGSNEKIFGHLISKRILEIREKKLYTAAAGDRKAVAEWIDEMKSAIAIHPEWN
EYFKFKFKNTAEYDNANKFYEDIDKQTYSLEKVEIPTEYIDEMVSQHKLYLFQLYTKDESDKKKK
KGTDNLHTMYWHGVESDENLKAVTEGTQPIIKLNGEAEMEMRNPSIEFQVTHEHNKPIANKNPLN
IKKESVENYDLIKDKRYTERKFYFHCPITLNFRADKPIKYNEKINRFVENNPDVCIIGIDRGERH
LLYYTVINQTGDILEQGSLNKISGSYTNDKGEKVNKETDYHDLLDRKEKGKHVAQQAWETIENIK
ELKAGYLSQVVYKLTQLMLQYNAVIVLENLNVGFKRGRTKVEKQVYQKFEKAMIDKLNYLVEKDR
GYEMNGSYAKGLQLTDKFESEDKIGKQTGCIYYVIPSYTSHIDPKTGFVNLLNAKLRYENITKAQ
DTIRKEDSISYNAKADYFEFAFDYRSFGVDMARNEWVVCTCGDLRWEYSAKTRETKAYSVTDRLK
ELFKAHGIDYVGGENLVSHITEVADKHELSTLLFYLRLVLKMRYTVSGTENENDFILSPVEYAPG
KFFDSREATSTEPMNADANGAYHIALKGLMTIRGIEDGKLHNYGKGGENAAWFKFMQNQEYKNNG

In certain embodiments, the type V-A Cas protein comprises McCpf1 or a variant thereof. In certain embodiments, the type V-A Cas protein comprises an amino acid sequence at least 30%, at least 40%, at least 50%, at least 60%, at least 70%, at least 75%, at least 80%, at least 85%, at least 90%, at least 91%, at least 92%, at least 93%, at least 94%, at least 95%, at least 96%, at least 97%, at least 98%, or at least 99% identical to the amino acid sequence set forth in SEQ ID NO: 9. In certain embodiments, the type V-A Cas protein comprises the amino acid sequence set forth in SEQ ID NO: 9.

McCpf1
(SEQ ID NO: 9)
MLFQDFTHLYPLSKTMRFELKPIGKTLEHIHAKNFLSQDETMADMYQKVKAILDDYHRDEIADMM
GEVKLTKLAEFYDVYLKERKNPKDDGLQKOLKDLQAVLRKEIVKPIGNGGKYKAGYDRLFGAKLE
KDGKELGDLAKFVIAQEGESSPKLAHLAHFEKFSTYFTGFHDNRKNMYSDEDKHTAITYRLIHEN
LPRFIDNLQILATIKQKHSALYDQIINELTASGLDVSLASHLDGYHKLLTQEGITAYNTLLGGIS
GEAGSRKIQGINELINSHHNQHCHKSERIAKLRPLHKQILSDGMGVSFLPSKFADDSEMCQAVNE
FYRHYADVFAKVQSLFDGFDDHQKDGIYVEHKNLNELSKQAFGDFALLGRVLDGYYVDVVNPEEN
ERFAKAKTDNAKAKLTKEKDKFIKGVHSLASLEQAIEHYTARHDDESVQAGKLGQYFKHGLAGVD
NPIQKIHNNHSTIKGFLERERPAGERALPKIKSGKNPEMTQLRQLKELLDNALNVAHFAKLLTTK
TTLDNQDGNFYGEFGALYDELAKIPTLYNKVRDYLSQKPFSTEKYKLNFGNPTLLNGWDLNKEKD
NEGIILQKDGCYYLALLDKAHKKVEDNAPNTGKNVYQKMIYKLLPGPNKMLPKVEFAKSNLDYYN
PSAELLDKYAQGTHKKGNNFNLKDCHALIDFFKAGINKHPEWQHFGFKESPTSSYQDLSDFYREV
EPQGYQVKFVDINADYINELVEQGQLYLFQIYNKDESPKAHGKPNLHTLYFKALFSKDNLANPIY
KLNGEAQIFYRKASLDMNETTIHRAGEVLENKNPDNPKKRQFVYDIIKDKRYTQDKEMLHVPITM
NFGVQGMTIKEFNKKVNQSIQQYDEVNVIGIDRGERHLLYLTVINSKGEILEQRSLNDITTASAN
GTQMTTPYHKILDKREIERLNARVGWGEIETIKELKSGYLSHVVHQISQLMLKYNAIVVLEDLNE
GFKRGRFKVEKQIYQNFENALIKKLNHLVLKDEADDEIGSYKNALQLTNNFTDLKSIGKQTGELF
YVPAWNTSKIDPETGFVDLLKPRYENIAQSQAFFGKEDKICYNADKDYFEFHIDYAKFTDKAKNS
RQIWKICSHGDKRYVYDKTANQNKGATKGINVNDELKSLFARHHINDKQPNLVMDICONNDKEFH
KSLIYLLKTLLALRYSNASSDEDFILSPVANDEGMFENSALADDTQPQNADANGAYHIALKGLWV
LEQIKNSDDLNKVKLAIDNQTWLNFAQNR

In certain embodiments, the type V-A Cas protein comprises Lb3Cpf1 or a variant thereof. In certain embodiments, the type V-A Cas protein comprises an amino acid sequence at least 30%, at least 40%, at least 50%, at least 60%, at least 70%, at least 75%, at least 80%, at least 85%, at least 90%, at least 91%, at least 92%, at least 93%, at least 94%, at least 95%, at least 96%, at least 97%, at least 98%, or at least 99% identical to the amino acid sequence set forth in SEQ ID NO: 10. In certain embodiments, the type V-A Cas protein comprises the amino acid sequence set forth in SEQ ID NO: 10.

Lb3Cpf1
(SEQ ID NO: 10)
MHENNGKIADNFIGIYPVSKTLRFELKPVGKTQEYIEKHGILDEDLKRAGDYKSVKKIIDAYHKY
FIDEALNGIQLDGLKNYYELYEKKRDNNEEKEFQKIQMSLRKQIVKRFSEHPQYKYLFKKELIKN
VLPEFTKDNAEEQTLVKSFQEFTTYFEGFHQNRKNMYSDEEKSTAIAYRVVHQNLPKYIDNMRIF
SMILNTDIRSDLTELENNLKTKMDITIVEEYFAIDGENKVVNQKGIDVYNTILGAFSTDDNTKIK
GLNEYINLYNQKNKAKLPKLKPLFKQILSDRDKISFIPEQFDSDTEVLEAVDMFYNRLLQFVIEN
EGQITISKLLTNFSAYDLNKIYVKNDTTISAISNDLEDDWSYISKAVRENYDSENVDKNKRAAAY
EEKKEKALSKIKMYSIEELNFFVKKYSCNECHIEGYFERRILEILDKMRYAYESCKILHDKGLIN
NISLCQDRQAISELKDELDSIKEVQWLLKPLMIGQEQADKEEAFYTELLRIWEELEPITLLYNKV
RNYVTKKPYTLEKVKLNFYKSTLLDGWDKNKEKDNLGIILLKDGQYYLGIMNRRNNKIADDAPLA
KTDNVYRKMEYKLLTKVSANLPRIFLKDKYNPSEEMLEKYEKGTHLKGENFCIDDCRELIDEFKK
GIKQYEDWGQFDFKESDTESYDDISAFYKEVEHOGYKITFRDIDETYIDSLVNEGKLYLFQIYNK
DESPYSKGTKNLHTLYWEMLESQQNLQNIVYKLNGNAEIFYRKASINQKDVVVHKADLPIKNKDP
QNSKKESMEDYDIIKDKRFTCDKYQFHVPITMNFKALGENHENRKVNRLIHDAENMHIIGIDRGE
RNLIYLCMIDMKGNIVKQISLNEIISYDKNKLEHKRNYHQLLKTREDENKSARQSWQTIHTIKEL
KEGYLSQVIHVITDLMVEYNAIVVLEDLNFGFKQGRQKFERQVYQKFEKMLIDKLNYLVDKSKGM
DEDGGLLHAYQLTDEFKSFKQLGKQSGFLYYIPAWNTSKLDPTTGFVNLFYTKYESVEKSKEFIN
NFTSILYNQEREYFEFLEDYSAFTSKAEGSRLKWTVCSKGERVETYRNPKKNNEWDTQKIDLIFE
LKKLFNDYSISLLDGDLREQMGKIDKADFYKKEMKLFALIVQMRNSDEREDKLISPVLNKYGAFF
ETGKNERMPLDADANGAYNIARKGLWIIEKIKNTDVEQLDKVKLTISNKEWLQYAQEHIL

In certain embodiments, the type V-A Cas protein comprises EcCpf1 or a variant thereof. In certain embodiments, the type V-A Cas protein comprises an amino acid sequence at least 30%, at least 40%, at least 50%, at least 60%, at least 70%, at least 75%, at least 80%, at least 85%, at least 90%, at least 91%, at least 92%, at least 93%, at least 94%, at least 95%, at least 96%, at least 97%, at least 98%, or at least 99% identical to the amino acid sequence set forth in SEQ ID NO: 11. In certain embodiments, the type V-A Cas protein comprises the amino acid sequence set forth in SEQ ID NO: 11.

EcCpf1
(SEQ ID NO: 11)
MDFFKNDMYFLCINGIIVISKLFAYLFLMYKRGVVMIKDNFVNVY
SLSKTIRMALIPWGKTEDNEYKKELLEEDEERAKNYIKVKGYMDE
YHKNFIESALNSVVLNGVDEYCELYFKQNKSDSEVKKIESLEASM
RKQISKAMKEYTVDGVKIYPLLSKKEFIRELLPEFLTQDEEIETL
EQENDESTYFQGEWENRKNIYTDEEKSTGVPYRCINDNLPKFLDN
VKSFEKVILALPQKAVDELNANENGVYNVDVQDVESVDYFNFVLS
QSGIEKYNNIIGGYSNSDASKVQGLNEKINLYNQQIAKSDKSKKL
PLLKPLYKQILSDRSSLSFIPEKFKDDNEVLNSINVLYDNIAESL
EKANDLMSDIANYNTDNIFISSGVAVTDISKKVFGDWSLIRNNWN
DEYESTHKKGKNEEKFYEKEDKEFKKIKSFSVSELQRLANSDLSI
VDYLVDESASLYADIKTAYNNAKDLLSNEYSHSKRLSKNDDAIEL
IKSELDSIKNYEAFLKPLCGTGKEESKDNAFYGAFLECFEEIRQV
DAVYNKVRNHITQKPYSNDKIKLNFQNPQFLAGWDKNKERAYRSV
LLRNGEKYYLAIMEKGKSKLFEDEPEDESSPFEKIDYKLLPEPSK
MLPKVFFATSNKDLENPSDEILNIRATGSFKKGDSENLDDCHKFI
DFYKASIENHPDWSKEDEDESETNDYEDISKFFKEVSDQGYSIGY
RKISESYLEEMVDNGSLYMFQLYNKDESENRKSKGTPNLHTLYFK
MLEDERNLEDVVYKLSGGAEMFYRKPSIDKNEMIVHPKNQPIDNK
NPNNVKKTSTFEYDIVKDMRYTKPQFQLHLPIVLNFKANSKGYIN
DDVRNVLKNSEDTYVIGIDRGERNLVYACVVDGNGKLVEQVPLNV
IEADNGYKTDYHKLLNDREEKRNEARKSWKTIGNIKELKEGYISQ
VVHKICQLVVKYDAVIAMEDLNSGFVNSRKKVEKQVYQKFERMLT
QKLNYLVDKKLDPNEMGGLLNAYQLTNEATKVRNGRQDGIIFYIP
AWLTSKIDPTTGFVNLLKPKYNSVSASKEFFSKEDEIRYNEKENY
FEFSENYDNEPKCNADEKREWTVCTYGDRIRTFRDPENNNKENSE
VVVLNDEFKNLFVEFDIDYTDNLKEQILAMDEKSFYKKLMGLLSL
TLQMRNSISKNVDVDYLISPVKNSNGEFYDSRNYDITSSLPCDAD
SNGAYNIARKGLWAINQIKQADDETKANISIKNSEWLQYAQNCDE
V

In certain embodiments, the type V-A Cas protein is not Cpf1. In certain embodiments, the type V-A Cas nuclease is not AsCpf1.

In certain embodiments, the type V-A Cas protein comprises MAD1, MAD2, MAD3, MAD4, MAD5, MAD6, MAD7, MAD8, MAD9, MAD10, MAD11, MAD12, MAD13, MAD14, MAD15, MAD16, MAD17, MAD18, MAD19, or MAD20, or variants thereof. MAD1-MAD20 are known in the art and are described in U.S. Pat. No. 9,982,279.

In certain embodiments, the type V-A Cas protein comprises MAD7 or a variant thereof. In certain embodiments, the type V-A Cas protein comprises an amino acid sequence at least 30%, at least 40%, at least 50%, at least 60%, at least 70%, at least 75%, at least 80%, at least 85%, at least 90%, at least 91%, at least 92%, at least 93%, at least 94%, at least 95%, at least 96%, at least 97%, at least 98%, or at least 99% identical to the amino acid sequence set forth in SEQ ID NO: 1. In certain embodiments, the type V-A Cas protein comprises the amino acid sequence set forth in SEQ ID NO: 1.

MAD7
(SEQ ID NO: 1)
MNNGTNNFQNFIGISSLQKTLRNALIPTETTQQFIVKNGIIKEDE
LRGENRQILKDIMDDYYRGEISETLSSIDDIDWTSLFEKMEIQLK
NGDNKDTLIKEQTEYRKAIHKKFANDDRFKNMESAKLISDILPEF
VIHNNNYSASEKEEKTQVIKLESRFATSFKDYFKNRANCESADDI
SSSSCHRIVNDNAEIFFSNALVYRRIVKSLSNDDINKISGDMKDS
LKEMSLEEIYSYEKYGEFITQEGISFYNDICGKVNSEMNLYCQKN
KENKNLYKLQKLHKQILCIADTSYEVPYKFESDEEVYQSVNGELD
NISSKHIVERLRKIGDNYNGYNLDKIYIVSKFYESVSQKTYRDWE
TINTALEIHYNNILPGNGKSKADKVKKAVKNDLQKSITEINELVS
NYKLCSDDNIKAETYIHEISHILNNFEAQELKYNPEIHLVESELK
ASELKNVLDVIMNAFHWCSVEMTEELVDKDNNFYAELEEIYDEIY
PVISLYNLVRNYVTQKPYSTKKIKLNEGIPTLADGWSKSKEYSNN
AIILMRDNLYYLGIFNAKNKPDKKIIEGNTSENKGDYKKMIYNLL
PGPNKMIPKVFLSSKTGVETYKPSAYILEGYKQNKHIKSSKDEDI
TECHDLIDYFKNCIAIHPEWKNFGEDESDTSTYEDISGFYREVEL
QGYKIDWTYISEKDIDLLQEKGQLYLFQIYNKDESKKSTGNDNLH
TMYLKNLFSEENLKDIVLKLNGEAEIFFRKSSIKNPIIHKKGSIL
VNRTYEAEEKDQFGNIQIVRKNIPENIYQELYKYFNDKSDKELSD
EAAKLKNVVGHHEAATNIVKDYRYTYDKYELHMPITINFKANKTG
FINDRILQYIAKEKDLHVIGIDRGERNLIYVSVIDTCGNIVEQKS
ENIVNGYDYQIKLKQQEGARQIARKEWKEIGKIKEIKEGYLSLVI
HEISKMVIKYNAIIAMEDLSYGEKKGREKVERQVYQKFETMLINK
LNYLVEKDISITENGGLLKGYQLTYIPDKLKNVGHQCGCIFYVPA
AYTSKIDPTTGFVNIFKFKDLTVDAKREFIKKEDSIRYDSEKNLF
CFTEDYNNFITQNTVMSKSSWSVYTYGVRIKRRFVNGRESNESDT
IDITKDMEKTLEMTDINWRDGHDLRQDIIDYEIVQHIFEIFRLTV
QMRNSLSELEDRDYDRLISPVLNENNIFYDSAKAGDALPKDADAN
GAYCIALKGLYEIKQITENWKEDGKFSRDKLKISNKDWEDFIQNK
RYL

In certain embodiments, the type V-A Cas protein comprises MAD2 or a variant thereof. In certain embodiments, the type V-A Cas protein comprises an amino acid sequence at least 30%, at least 40%, at least 50%, at least 60%, at least 70%, at least 75%, at least 80%, at least 85%, at least 90%, at least 91%, at least 92%, at least 93%, at least 94%, at least 95%, at least 96%, at least 97%, at least 98%, or at least 99% identical to the amino acid sequence set forth in SEQ ID NO: 2. In certain embodiments, the type V-A Cas protein comprises the amino acid sequence set forth in SEQ ID NO: 2.

MAD2
(SEQ ID NO: 2)
MSSLTKFTNKYSKQLTIKNELIPVGKTLENIKENGLIDGDEQLNE
NYQKAKIIVDDELRDFINKALNNTQIGNWRELADALNKEDEDNIE
KLQDKIRGIIVSKFETFDLESSYSIKKDEKIIDDDNDVEEEELDL
GKKTSSFKYIFKKNLFKLVLPSYLKTTNQDKLKIISSEDNESTYF
RGFFENRKNIFTKKPISTSIAYRIVHDNFPKELDNIRCENVWQTE
CPQLIVKADNYLKSKNVIAKDKSLANYFTVGAYDYFLSQNGIDFY
NNIIGGLPAFAGHEKIQGLNEFINQECQKDSELKSKLKNRHAFKM
AVLFKQILSDREKSFVIDEFESDAQVIDAVKNFYAEQCKDNNVIE
NLLNLIKNIAFLSDDELDGIFIEGKYLSSVSQKLYSDWSKLRNDI
EDSANSKQGNKELAKKIKINKGDVEKAISKYEFSLSELNSIVHDN
TKESDLLSCTLHKVASEKLVKVNEGDWPKHLKNNEEKQKIKEPLD
ALLEIYNTLLIFNCKSENKNGNFYVDYDRCINELSSVVYLYNKTR
NYCTKKPYNTDKFKLNENSPQLGEGESKSKENDCLTLLEKKDDNY
YVGIIRKGAKINFDDTQAIADNTDNCIFKMNYFLLKDAKKFIPKC
SIQLKEVKAHEKKSEDDYILSDKEKFASPLVIKKSTFLLATAHVK
GKKGNIKKFQKEYSKENPTEYRNSLNEWIAFCKEFLKTYKAATIF
DITTLKKAEEYADIVEFYKDVDNLCYKLEFCPIKTSFIENLIDNG
DLYLFRINNKDESSKSTGTKNLHTLYLQAIFDERNLNNPTIMLNG
GAELFYRKESIEQKNRITHKAGSILVNKVCKDGTSLDDKIRNEIY
QYENKFIDTLSDEAKKVLPNVIKKEATHDITKDKRFTSDKFFFHC
PLTINYKEGDTKQFNNEVLSFLRGNPDINIIGIDRGERNLIYVTV
INQKGEILDSVSENTVINKSSKIEQTVDYEEKLAVREKERIEAKR
SWDSISKIATLKEGYLSAIVHEICLLMIKHNAIVVLENLNAGFKR
IRGGLSEKSVYQKFEKMLINKLNYFVSKKESDWNKPSGLLNGLQL
SDQFESFEKLGIQSGFIFYVPAAYTSKIDPTTGFANVLNLSKVRN
VDAIKSFFSNFNEISYSKKEALFKFSEDLDSLSKKGFSSFVKESK
SKWNVYTFGERIIKPKNKQGYREDKRINLTFEMKKLLNEYKVSED
LENNLIPNLTSANLKDTFWKELFFIFKTTLQLRNSVINGKEDVLI
SPVKNAKGEFFVSGTHNKTLPQDCDANGAYHIALKGLMILERNNL
VREEKDTKKIMAISNVDWFEYVQKRRGVL

In certain embodiments, the type V-A Cas protein comprises Csm1. Csm1 proteins are known in the art and are described in U.S. Pat. No. 9,896,696. Csm1 orthologs can be found in various bacterial and archaeal genomes. For example, in certain embodiments, the Csm1 protein is derived from Smithella sp. SCADC (Sm), Sulfuricurvum sp. (Ss), or Microgenomates (Roizmanbacteria) bacterium (Mb).

In certain embodiments, the type V-A Cas protein comprises SmCsm1 or a variant thereof. In certain embodiments, the type V-A Cas protein comprises an amino acid sequence at least 30%, at least 40%, at least 50%, at least 60%, at least 70%, at least 75%, at least 80%, at least 85%, at least 90%, at least 91%, at least 92%, at least 93%, at least 94%, at least 95%, at least 96%, at least 97%, at least 98%, or at least 99% identical to the amino acid sequence set forth in SEQ ID NO: 12. In certain embodiments, the type V-A Cas protein comprises the amino acid sequence set forth in SEQ ID NO: 12.

SmCsm1
(SEQ ID NO: 12)
MEKYKITKTIRFKLLPDKIQDISRQVAVLQNSTNAEKKNNLLRLV
QRGQELPKLLNEYIRYSDNHKLKSNVTVHFRWLRLFTKDLFYNWK
KDNTEKKIKISDVVYLSHVFEAFLKEWESTIERVNADCNKPEESK
TRDAEIALSIRKLGIKHQLPFIKGFVDNSNDKNSEDTKSKLTALL
SEFEAVLKICEQNYLPSQSSGIAIAKASFNYYTINKKQKDFEAEI
VALKKQLHARYGNKKYDQLLRELNLIPLKELPLKELPLIEFYSEI
KKRKSTKKSEFLEAVSNGLVEDDLKSKFPLFQTESNKYDEYLKLS
NKITQKSTAKSLLSKDSPEAQKLQTEITKLKKNRGEYFKKAFGKY
VQLCELYKEIAGKRGKLKGQIKGIENERIDSQRLQYWALVLEDNL
KHSLILIPKEKTNELYRKVWGAKDDGASSSSSSTLYYFESMTYRA
LRKLCFGINGNTFLPEIQKELPQYNQKEFGEFCFHKSNDDKEIDE
PKLISFYQSVLKTDFVKNTLALPQSVENEVAIQSFETRQDFQIAL
EKCCYAKKQIISESLKKEILENYNTQIFKITSLDLQRSEQKNLKG
HTRIWNRFWTKQNEEINYNLRLNPEIAIVWRKAKKTRIEKYGERS
VLYEPEKRNRYLHEQYTLCTTVTDNALNNEITFAFEDTKKKGTEI
VKYNEKINQTLKKEFNKNQLWFYGIDAGEIELATLALMNKDKEPQ
LFTVYELKKLDFFKHGYIYNKERELVIREKPYKAIQNLSYFLNEE
LYEKTERDGKENETYNELFKEKHVSAIDLTTAKVINGKIILNGDM
ITELNLRILHAQRKIYEELIENPHAELKEKDYKLYFEIEGKDKDI
YISRLDFEYIKPYQEISNYLFAYFASQQINEAREEEQINQTKRAL
AGNMIGVIYYLYQKYRGIISIEDLKQTKVESDRNKFEGNIERPLE
WALYRKFQQEGYVPPISELIKLRELEKEPLKDVKQPKYENIQQFG
IIKFVSPEETSTTCPKCLRRFKDYDKNKQEGFCKCQCGEDTRNDL
KGFEGLNDPDKVAAFNIAKRGFEDLQKYK

In certain embodiments, the type V-A Cas protein comprises SsCsm1 or a variant thereof. In certain embodiments, the type V-A Cas protein comprises an amino acid sequence at least 30%, at least 40%, at least 50%, at least 60%, at least 70%, at least 75%, at least 80%, at least 85%, at least 90%, at least 91%, at least 92%, at least 93%, at least 94%, at least 95%, at least 96%, at least 97%, at least 98%, or at least 99% identical to the amino acid sequence set forth in SEQ ID NO: 13. In certain embodiments, the type V-A Cas protein comprises the amino acid sequence set forth in SEQ ID NO: 13.

SsCsm1
(SEQ ID NO: 13)
MLHAFTNQYQLSKTLRFGATLKEDEKKCKSHEELKGFVDISYENM
KSSATIAESLNENELVKKCERCYSEIVKFHNAWEKIYYRTDQIAV
YKDFYRQLSRKARFDAGKQNSQLITLASLCGMYQGAKLSRYITNY
WKDNITRQKSFLKDESQQLHQYTRALEKSDKAHTKPNLINENKTE
MVLANLVNEIVIPLSNGAISFPNISKLEDGEESHLIEFALNDYSQ
LSELIGELKDAIATNGGYTPFAKVTLNHYTAEQKPHVFKNDIDAK
IRELKLIGLVETLKGKSSEQIEEYESNLDKESTYNDRNQSVIVRT
QCFKYKPIPELVKHQLAKYISEPNGWDEDAVAKVLDAVGAIRSPA
HDYANNQEGFDLNHYPIKVAFDYAWEQLANSLYTTVTFPQEMCEK
YLNSIYGCEVSKEPVFKFYADLLYIRKNLAVLEHKNNLPSNQEEF
ICKINNTFENIVLPYKISQFETYKKDILAWINDGHDHKKYTDAKQ
QLGFIRGGLKGRIKAEEVSQKDKYGKIKSYYENPYTKLINEFKQI
SSTYGKTFAELRDKEKEKNEITKITHEGIIIEDKNRDRYLLASEL
KHEQINHVSTILNKLDKSSEFITYQVKSLTSKTLIKLIKNHTTKK
GAISPYADFHTSKTGENKNEIEKNWDNYKREQVLVEYVKDCLTDS
TMAKNQNWAEFGWNFEKCNSYEDIEHEIDQKSYLLQSDTISKQSI
ASLVEGGCLLLPIINQDITSKERKDKNQFSKDWNHIFEGSKEFRL
HPEFAVSYRTPIEGYPVQKRYGRLQFVCAFNAHIVPQNGEFINLK
KQIENENDEDVQKRNVTEENKKVNHALSDKEYVVIGIDRGLKQLA
TLCVLDKRGKILGDFEIYKKEFVRAEKRSESHWEHTQAETRHILD
LSNLRVETTIEGKKVLVDQSLTLVKKNRDTPDEEATEENKQKIKL
KQLSYIRKLQHKMQTNEQDVLDLINNEPSDEEFKKRIEGLISSFG
EGQKYADLPINTMREMISDLQGVIARGNNQTEKNKIIELDAADNL
KQGIVANMIGIVNYIFAKYSYKAYISLEDLSRAYGGAKSGYDGRY
LPSTSQDEDVDFKEQQNQMLAGLGTYQFFEMQLLKKLQKIQSDNT
VLRFVPAFRSADNYRNILRLEETKYKSKPFGVVHFIDPKFTSKKC
PVCSKTNVYRDKDDILVCKECGFRSDSQLKERENNIHYIHNGDDN
GAYHIALKSVENLIQMK

In certain embodiments, the type V-A Cas protein comprises MbCsm1 or a variant thereof. In certain embodiments, the type V-A Cas protein comprises an amino acid sequence at least 30%, at least 40%, at least 50%, at least 60%, at least 70%, at least 75%, at least 80%, at least 85%, at least 90%, at least 91%, at least 92%, at least 93%, at least 94%, at least 95%, at least 96%, at least 97%, at least 98%, or at least 99% identical to the amino acid sequence set forth in SEQ ID NO: 14. In certain embodiments, the type V-A Cas protein comprises the amino acid sequence set forth in SEQ ID NO: 14.

MbCsm1
(SEQ ID NO: 14)
MEIQELKNLYEVKKTVRFELKPSKKKIFEGGDVIKLQKDFEKVQK
FFLDIFVYKNEHTKLEFKKKREIKYTWLRTNTKNEFYNWRGKSDT
GKNYALNKIGFLAEEILRWLNEWQELTKSLKDLTQREEHKQERKS
DIAFVLRNELKRQNLPFIKDFFNAVIDIQGKQGKESDDKIRKFRE
EIKEIEKNLNACSREYLPTQSNGVLLYKASFSYYTLNKTPKEYED
LKKEKESELSSVLLKEIYRRKRENRTTNQKDTLFECTSDWLVKIK
LGKDIYEWTLDEAYQKMKIWKANQKSNFIEAVAGDKLTHQNFRKQ
FPLEDASDEDFETFYRLTKALDKNPENAKKIAQKRGKFFNAPNET
VQTKNYHELCELYKRIAVKRGKIIAEIKGIENEEVQSQLLTHWAV
IAEERDKKFIVLIPRKNGGKLENHKNAHAFLQEKDRKEPNDIKVY
HFKSLTLRSLEKLCFKEAKNTFAPEIKKETNPKIWFPTYKQEWNS
TPERLIKFYKQVLQSNYAQTYLDLVDEGNLNTFLETHFTTLEEFE
SDLEKTCYTKVPVYFAKKELETFADEFEAEVFEITTRSISTESKR
KENAHAEIWRDEWSRENEEENHITRLNPEVSVLYRDEIKEKSNTS
RKNRKSNANNRESDPRETLATTITLNADKKKSNLAFKTVEDINIH
IDNENKKESKNESGEWVYGIDRGLKELATLNVVKESDVKNVFGVS
QPKEFAKIPIYKLRDEKAILKDENGLSLKNAKGEARKVIDNISDV
LEEGKEPDSTLFEKREVSSIDLTRAKLIKGHIISNGDQKTYLKLK
ETSAKRRIFELFSTAKIDKSSQFHVRKTIELSGTKIYWLCEWQRQ
DSWRTEKVSLRNTLKGYLQNLDLKNRFENIETIEKINHLRDAITA
NMVGILSHLQNKLEMQGVIALENLDTVREQSNKKMIDEHFEQSNE
HVSRRLEWALYCKFANTGEVPPQIKESIFLRDEFKVCQIGILNFI
DVKGTSSNCPNCDQESRKTGSHFICNFQNNCIFSSKENRNLLEQN
LHNSDDVAAFNIAKRGLEIVKV

In certain embodiments, the type V-A Cas nuclease comprises an ART nuclease or a variant thereof. In general, such nucleases sequences have <60% AA sequence similarity to Cas12a, <60% AA sequence similarity to a positive control nuclease, and >80% query cover. In certain embodiments, the Type V-A nuclease comprises an ART1, ART2, ART3, ART4, ART5, ART6, ART7, ART8, ART9, ART10, ART11, ART12, ART13, ART14, ART15, ART16, ART17, ART18, ART19, ART20, ART21, ART22, ART23, ART24, ART25, ART26, ART27, ART28, ART28, ART30, ART31, ART32, ART33, ART34, ART35, or ART11* (i.e., ART11_L679F, i.e., ART11 wherein leucine (L) at amino acid position 679 is replaced with phenylalanine (F)) nuclease, as shown in Table 10. In certain embodiments, the type V-A Cas protein comprises an amino acid sequence at least 30%, at least 40%, at least 50%, at least 60%, at least 70%, at least 75%, at least 80%, at least 85%, at least 90%, at least 91%, at least 92%, at least 93%, at least 94%, at least 95%, at least 96%, at least 97%, at least 98%, or at least 99% identical to the amino acid sequence designated for the individual ART nuclease as shown in Table 10. In certain embodiments, provided is a nucleic acid-guided nuclease comprising a nucleic acid-guided nuclease polypeptide having at least 85% identity to an amino acid sequence represented by SEQ ID NOs: 950-984 or a nucleic acid encoding a nucleic acid-guided nuclease polypeptide comprising at least 85% identity with the polynucleotide represented by SEQ ID NOs: 808-949. In certain embodiments, provided is a nucleic acid-guided nuclease comprising a polypeptide having at least 90% identity to the amino acid sequence represented by SEQ ID NOs: 950-958, 968-970, 972, 973, 976, 978-982, or 984, wherein the polypeptide does not contain a peptide motif of YLFQIYNKDF (SEQ ID NO: 806). In certain embodiments, provided is a nucleic acid-guided nuclease comprising a nucleic acid encoding a polypeptide having at least 90% identity to nucleic acids represented by SEQ ID NOs: 808-845 wherein an encoded polypeptide does not contain a peptide motif of YLFQIYNKDF (SEQ ID NO: 806). In certain embodiments, provided is a nucleic acid-guided nuclease wherein the polypeptide comprises at least 90% identity with the amino acid sequence represented by SEQ ID NOs: 950, 951, 954, 955, 957, or 958. In certain embodiments, provided is a nucleic acid-guided nuclease, wherein the polypeptide comprises a polypeptide comprising at least 90% identity with the amino acid sequence represented by SEQ ID NO: 951.

TABLE 10
Exemplary ART nucleases

| | | SEQ ID | SEQ ID | | |
|---|---|---|---|---|---|
| | | NO | NO | | % AA |
| | | corre- | corre- | | to |
| | | sponding | sponding | % AA | positive |
| | Protein | to Amino | to nucleic | to Cpf1 | control |
| ART | Reference | Acid | acid | (<80% | (<60% |
| Name | Number | sequences | sequence | desired) | desired) |
| ART1 | WP_118425113.1 | 950 | 808 | 30.838 | 32.54 |
| ART2 | WP_137013028.1 | 951 | 812 | 34.189 | 33.07 |
| ART3 | WP_073043853.1 | 952 | 818 | 35.982 | 36.72 |
| ART4 | WP_118734405.1 | 953 | 822 | 30.519 | 51.64 |
| ART5 | WP_146683785.1 | 954 | 826 | 30.114 | 32.31 |
| ART6 | WP_117882263.1 | 955 | 830 | 29.421 | 33.49 |
| ART7 | OYP43732.1 | 956 | 834 | 26.323 | 28.64 |
| ART8 | TSC78600.1 | 957 | 838 | 25.379 | 23.01 |
| ART9 | WP_094390816.1 | 958 | 842 | 26.323 | 28.62 |
| ART10 | WP_104505765.1 | 959 | 846 | 31.291 | 32.59 |
| ART11 | WP_151622887.1 | 960 | 850 | 30.654 | 35.55 |
| ART12 | HAW84277.1 | 961 | 854 | 34.872 | 31.33 |
| ART13 | WP_119227726.1 | 962 | 858 | 34.993 | 31.55 |
| ART14 | WP_118080156.1 | 963 | 862 | 32.551 | 35.33 |
| ART15 | WP_046700744.1 | 964 | 866 | 31.456 | 33.92 |
| ART16 | WP_115247861.1 | 965 | 870 | 31.136 | 34.25 |
| ART17 | WP_062499108.1 | 966 | 874 | 31.136 | 34.17 |
| ART18 | WP_154326953.1 | 967 | 878 | 31.113 | 33.28 |
| ART19 | WP_117747221.1 | 968 | 882 | 30.764 | 32.47 |
| ART20 | WP_118211091.1 | 969 | 886 | 30.986 | 32.29 |
| ART21 | WP_118163031.1 | 970 | 890 | 31.134 | 32.54 |
| ART22 | WP_115006085.1 | 971 | 894 | 30.044 | 31.55 |
| ART23 | HCS95801.1 | 972 | 898 | 30.37 | 51.64 |
| ART24 | WP_089541090.1 | 973 | 902 | 30.933 | 33.11 |
| ART25 | WP_120123115.1 | 974 | 906 | 29.978 | 48.88 |
| ART26 | WP_117874294.1 | 975 | 910 | 29.904 | 48.49 |
| ART27 | WP_117951432.1 | 976 | 904 | 29.421 | 33.03 |
| ART28 | WP_108977930.1 | 977 | 918 | 32.099 | 32.69 |
| ART29 | WP_117886476.1 | 978 | 922 | 29.643 | 33.41 |
| ART30 | WP_101070975.1 | 979 | 926 | 29.027 | 32.95 |
| ART31 | WP_117949317.1 | 980 | 930 | 29.198 | 33.18 |
| ART32 | WP_118128310.1 | 981 | 934 | 29.198 | 33.18 |
| ART33 | WP_138157649.1 | 982 | 938 | 27.273 | 29.89 |
| ART34 | WP_135764749.1 | 983 | 942 | 27.004 | 25 |
| ART35 | OYP46450.1 | 984 | 946 | 26.709 | 29.51 |

In certain embodiments, the type V-A Cas nuclease comprises an ABW nuclease or a variant thereof. See International (PCT) Publication No. WO2021/108324. Exemplary amino acid and nucleic acid sequences are shown in Table 11. In certain embodiments, the Type V-A nuclease comprises an ABW1, ABW2, ABW3, ABW4, ABW5, ABW6, ABW7, ABW8, or ABW9 nuclease, as shown in Table 11. In certain embodiments, the type V-A Cas protein comprises an amino acid sequence at least 30%, at least 40%, at least 50%, at least 60%, at least 70%, at least 75%, at least 80%, at least 85%, at least 90%, at least 91%, at least 92%, at least 93%, at least 94%, at least 95%, at least 96%, at least 97%, at least 98%, or at least 99% identical to the amino acid sequence designated for the individual ABW nuclease as shown in Table 11.

TABLE 11
Sequences of exemplary engineered ABW nucleases

| | Engineered | |
|---|---|---|
| | Amino Acid | Engineered |
| | Sequence | Nucleotide Sequence |
| ABW1 | MGHHHHHHSSGLVPRGSG | ATGGGCCACCATCATCATCATCATAGCAGCGGCCTG |
| | TMAAFDKFIHQYQVSKTL | GTGCCGCGCGGCAGCGGTACCATGGCGGCGTTCGAT |
| | RFALIPQGKTLENTKNNV | AAGTTCATCCATCAATATCAAGTAAGCAAAACCCTC |
| | LQEDDERQKNYEKVKPIL | CGTTTTGCACTTATTCCGCAGGGGAAAACCTTGGAG |
| | DRIYKVFAEESLKDCSVD | AATACAAAAAATAACGTACTCCAGGAAGATGATGAG |
| | WNDLNACLDAYQKNPSAD | CGTCAGAAAAATTACGAAAAAGTCAAACCTATCCTT |
| | KRQKVKAAQDALRDEIAG | GATCGTATTTATAAGGTATTCGCTGAGGAAAGCCTG |
| | YFTGKQYANGKNKNAVKE | AAAGATTGCAGCGTTGACTGGAATGACCTCAATGCA |
| | KEQAELYKDIFSKKIFDG | TGTCTGGATGCTTACCAAAAAAATCCTAGCGCGGAT |
| | TVTNNKLPQVNLSAEETE | AAGCGTCAGAAGGTGAAAGCCGCGCAGGACGCGTTG |
| | LLGCFDKFTTYFVGFYQN | CGGGACGAAATTGCCGGTTATTTTACAGGGAAACAA |
| | RENVESGEDIATAIPHRI | TACGCGAACGGGAAGAACAAAAATGCCGTTAAGGAG |
| | VQDNFPKFRENCRIYQDL | AAAGAGCAGGCAGAATTGTATAAGGATATCTTTAGC |
| | IKNEPALKPLLQQAAAAV | AAAAAGATCTTTGATGGGACCGTAACGAACAACAAA |
| | MAQNPKGIYQPRKSLDDI | TTGCCACAGGTCAACCTTTCAGCCGAAGAAACAGAG |
| | FVIPFYNHLLLQDDIDYF | TTATTAGGCTGTTTTGATAAATTCACAACATATTTC |
| | NQILGGISGAAGQKKIQG | GTCGGCTTTTACCAGAACCGTGAGAACGTATTTTCA |
| | LNETINLFMQQHPQEADK | GGGGAGGATATTGCTACAGCTATTCCGCATCGGATC |
| | LKKKKIRHRFIPLYKQIL | GTCCAGGATAATTTTCCTAAATTCCGGGAAAACTGT |
| | SDRTSFSFIPEAFSNSQE | CGGATTTATCAGGACTTAATCAAAAATGAACCTGCC |
| | ALDGIETFKKSLKKNDTE | CTTAAACCGCTGCTTCAGCAAGCAGCGGCCGCGGTG |
| | GALERLIQNLASLDLKYV | ATGGCCCAGAATCCAAAGGGGATCTATCAACCACGT |
| | YLSNKKVNEISQALYGEW | AAGAGTCTGGACGATATTTTTGTCATTCCGTTTTAT |
| | HCIQDVLKQDESLESLIQ | AACCATCTCCTCTTACAGGATGATATTGATTATTTC |
| | INPQNSSNGFLATLTDEG | AATCAAATCTTAGGCGGCATTTCGGGGGCAGCCGGT |
| | KKRISQCRNVLGNPLPVK | CAGAAAAAAATCCAGGGTTTAAATGAAACAATTAAT |
| | LADDQDKAQVKNQLDTLL | CTGTTTATGCAACAGCACCCACAAGAAGCCGATAAG |
| | AAVHYLEWFKADPDLETD | TTAAAGAAAAAAAAGATTCGTCATCGGTTTATTCCG |
| | PNFTVPFEKIWEELVPLL | CTGTATAAACAAATTCTCTCTGACCGTACGTCTTTC |
| | SLYSKVRNFVTKKPYSTA | TCGTTCATCCCTGAAGCTTTTTCCAATTCTCAGGAA |
| | KFKLNFANPTLADGWDIH | GCGTTAGACGGCATTGAGACATTCAAAAAGTCTCTT |
| | KESDNGALLFEKGGLYYL | AAGAAGAATGACACATTCGGCGCGTTGGAGCGGCTG |
| | GIMNPKDKPNFKSYQGAE | ATTCAAAATCTTGCTTCCCTGGACCTGAAATACGTG |
| | PYYQKMVYRFFPDCSKTI | TATTTATCGAACAAGAAGGTCAATGAGATTTCGCAG |
| | PKCSTQRKDVKKYFEDHP | GCATTATACGGCGAATGGCACTGCATCCAAGACGTC |
| | QATSYQIHDSKKEKFRQD | CTCAAGCAAGATTTCAGCCTTGAGAGCCTGATCCAG |
| | FFEIPREIYELNNTTYGT | ATCAACCCACAAAATTCTAGCAATGGTTTCCTGGCC |
| | GKSKYKKFQTQYYQKTQD | ACACTTACCGACGAAGGCAAGAAACGTATCTCCCAA |
| | KSGYQKALRKWIDESKKF | TGTCGTAACGTACTGGGGAATCCTCTTCCAGTCAAG |
| | LQTYVSTSIFDEKGLRPS | CTTGCGGATGATCAAGACAAAGCGCAAGTCAAAAAC |
| | KDYQDLGEFYKDVNSRCY | CAATTGGATACATTACTGGCTGCTGTACACTATCTC |
| | RVTFEKIRVQDIHEAVKN | GAGTGGTTCAAGGCAGATCCAGACCTGGAAACAGAC |
| | GQLYLFQLYNKDESPKSH | CCTAACTTCACTGTTCCTTTCGAAAAGATCTGGGAG |
| | GLPNLHTLYWKAVEDPEN | GAATTGGTTCCTTTACTTTCACTGTACTCTAAAGTT |
| | LKDPIVKLNGQAELFYRP | CGGAATTTTGTTACAAAGAAGCCATATTCTACAGCT |
| | KSNMQIIQHKTGEEIVNK | AAATTTAAACTGAACTTTGCTAACCCGACATTAGCG |
| | KLKDGTPVPDDIYREISA | GATGGGTGGGATATTCACAAGGAAAGTGATAACGGC |
| | YVQGKCQGNLSPEAEKWL | GCGCTCCTGTTTGAAAAGGGTGGTTTGTATTACTTG |
| | PSVTIKKAAHDITKDRRF | GGTATCATGAACCCTAAAGATAAGCCTAATTTTAAA |
| | TEDKFFFHVPITLNYQSS | TCCTATCAGGGTGCAGAGCCATACTATCAGAAGATG |
| | GKPTAFNSQVNDELTEHP | GTGTACCGTTTTTTTCCTGACTGTTCGAAGACCATC |
| | ETNIIGIDRGERNLIYAV | CCAAAATGCAGCACCCAACGTAAGGATGTAAAAAAG |
| | VITPDGKILEQKSENVIH | TACTTCGAAGACCACCCTCAAGCGACCTCATACCAG |
| | DEDYHESLSQREKQRVAA | ATCCACGACTCAAAGAAAGAGAAGTTTCGTCAGGAT |
| | RQAWTAIGRIKDLKEGYL | TTTTTTGAGATCCCTCGGGAGATTTACGAGCTTAAT |
| | SLVVHEIAQMMIKYQAVV | AACACCACATACGGCACAGGTAAGTCTAAATATAAA |
| | VLENLNTGFKRVRGGISE | AAATTCCAGACCCAGTATTACCAGAAGACTCAGGAT |
| | KAVYQQFEKMLIEKLNEL | AAGTCAGGCTATCAGAAAGCACTTCGCAAATGGATT |
| | VFKDRAINQEGGVLKAYQ | GACTTTTCCAAAAAGTTTCTTCAAACATACGTCAGT |
| | LTDSFTSFAKLGNQSGEL | ACTTCCATTTTTGATTTCAAAGGTCTCCGTCCTTCG |
| | FYIPSAYTSKIDPGTGFV | AAGGATTATCAGGACTTAGGCGAGTTCTATAAAGAC |
| | DPFIWSHVTASEENRNEF | GTTAATTCGCGTTGTTACCGTGTGACGTTCGAGAAA |
| | LKGFDSLKYDAQSSAFVL | ATTCGCGTACAGGACATCCACGAAGCAGTCAAAAAT |
| | HFKMKSNKQFQKNNVEGF | GGGCAACTGTATCTCTTCCAATTATATAATAAGGAC |
| | MPEWDICFEKNEEKISLQ | TTCTCACCTAAAAGCCATGGGTTGCCTAATCTTCAC |
| | GSKYTAGKRIIFDSKKKQ | ACTCTCTATTGGAAAGCCGTGTTCGATCCTGAGAAC |
| | YMECFPQNELMKALQDVG | TTGAAGGACCCTATCGTAAAACTTAATGGCCAAGCT |
| | ITWNTGNDIWQDVLKQAS | GAGTTATTCTATCGGCCGAAATCCAACATGCAAATC |
| | TDTGERHRMINLIRSVLQ | ATCCAACATAAGACCGGGGAGGAGATTGTGAACAAA |
| | MRSSNGATGEDYINSPVM | AAGCTGAAGGACGGCACCCCGGTTCCTGATGATATC |
| | DLDGRFFDTRAGIRDLPL | TACCGCGAAATCAGTGCTTACGTCCAGGGGAAATGT |
| | DADANGAYHIALKGRMVL | CAAGGCAACTTATCCCCGGAGGCAGAGAAGTGGCTC |
| | ERIRSQKNTAIKNTDWLY | CCAAGTGTCACAATCAAGAAAGCCGCCCATGATATC |
| | AIQEERNGAPKRPAATKK | ACAAAGGATCGTCGCTTTACCGAAGATAAGTTTTTC |
| | AGQAKKKKASGSGAGSPK | TTTCATGTCCCTATTACACTGAACTATCAGAGTTCA |
| | KKRKVEDPKKKRKV | GGCAAGCCGACGGCATTCAACTCGCAAGTAAACGAT |
| | (SEQ ID NO: 789) | TTCTTGACCGAGCACCCTGAGACAAATATCATCGGC |
| | | ATTGATCGGGGTGAACGTAACTTGATTTATGCCGTT |
| | | GTAATCACTCCAGATGGCAAGATTCTCGAACAGAAA |
| | | TCTTTTAACGTGATCCACGACTTTGATTATCATGAA |
| | | TCCCTGTCCCAGCGGGAAAAACAGCGGGTAGCAGCG |
| | | CGTCAGGCTTGGACAGCGATTGGTCGCATCAAGGAT |
| | | CTCAAGGAAGGTTACCTGTCGCTTGTGGTGCACGAA |
| | | ATTGCTCAAATGATGATCAAATACCAAGCAGTCGTC |
| | | GTATTAGAAAACCTCAACACGGGCTTTAAGCGTGTG |
| | | CGCGGTGGTATCAGTGAGAAGGCCGTCTACCAACAG |
| | | TTCGAAAAAATGTTGATTGAAAAATTGAACTTCCTG |
| | | GTATTTAAAGATCGGGCAATCAATCAGGAAGGCGGG |
| | | GTTCTCAAAGCTTACCAGCTGACAGACTCGTTTACG |
| | | TCTTTTGCAAAGTTAGGTAACCAGTCCGGTTTCCTG |
| | | TTCTACATCCCGTCCGCCTACACCAGCAAAATCGAC |
| | | CCTGGTACGGGCTTCGTCGATCCTTTTATCTGGTCT |
| | | CACGTGACCGCTTCTGAGGAAAATCGGAATGAATTT |
| | | TTAAAGGGCTTTGATAGCTTGAAATATGACGCCCAA |
| | | TCATCCGCCTTTGTACTGCATTTCAAGATGAAATCC |
| | | AATAAGCAATTTCAGAAGAACAATGTTGAAGGTTTC |
| | | ATGCCGGAATGGGATATCTGCTTCGAGAAAAACGAG |
| | | GAAAAGATTTCCTTGCAGGGTAGTAAGTATACAGCC |
| | | GGTAAACGCATTATTTTCGACTCCAAAAAGAAGCAA |
| | | TACATGGAGTGCTTCCCGCAGAATGAGCTCATGAAA |
| | | GCACTGCAGGACGTAGGCATCACCTGGAACACGGGC |
| | | AACGATATCTGGCAGGATGTCCTTAAACAAGCGAGC |
| | | ACAGATACAGGGTTTCGTCACCGGATGATCAACCTG |
| | | ATCCGTTCAGTGCTCCAGATGCGGTCCAGTAATGGT |
| | | GCGACCGGGGAGGATTACATCAATTCACCTGTGATG |
| | | GATCTGGACGGCCGTTTTTTCGACACTCGGGCGGGG |
| | | ATTCGTGATCTGCCATTGGATGCCGACGCCAACGGC |
| | | GCATACCACATCGCTTTAAAAGGGCGTATGGTACTC |
| | | GAACGCATTCGCTCCCAAAAGAATACCGCGATTAAG |
| | | AACACTGACTGGTTATACGCAATCCAAGAGGAACGT |
| | | AACGGCGCGCCAAAAAGGCCGGCGGCCACGAAAAAG |
| | | GCCGGCCAGGCAAAAAAGAAAAAGGCTAGCGGCAGC |
| | | GGCGCCGGATCCCCAAAGAAGAAAAGGAAGGTTGAA |
| | | GACCCCAAGAAAAAGAGGAAGGTGTGATAA (SEQ |
| | | ID NO: 790) |
| ABW2 | MGHHHHHHSSGLVPRGSG | ATGGGCCACCATCATCATCATCATAGCAGCGGCCTG |
| | TMKEFTNQYSLTKTLRFE | GTGCCGCGCGGCAGCGGTACCATGAAGGAGTTTACC |
| | LRPVGETAEKIEDFKSGG | AACCAATATTCCTTAACCAAGACCCTGCGGTTCGAG |
| | LKQTVEKDRERTEAYKQL | TTGCGGCCAGTCGGCGAAACAGCAGAAAAGATCGAA |
| | KEVIDSYHRDFIEQAFAR | GATTTTAAATCGGGCGGGCTCAAGCAAACAGTGGAA |
| | QQTLSEEDFKQTYQLYKE | AAGGATCGTGAGCGTACAGAAGCGTATAAGCAGTTG |
| | AQKEKDGETLTKQYEHLR | AAAGAGGTTATTGACTCCTATCATCGTGACTTCATT |
| | KKIAAMESKATKEWAVMG | GAGCAAGCTTTTGCGCGCCAGCAGACGCTGTCCGAG |
| | ENNELIGKNKESKLYQWL | GAGGATTTTAAACAAACATATCAACTGTACAAAGAG |
| | EKNYRAGRIEKEEFDHNA | GCCCAGAAAGAGAAGGATGGGGAAACATTAACAAAG |
| | GLIEYFEKFSTYFVGEDK | CAGTACGAGCATTTACGGAAGAAAATCGCAGCTATG |
| | NRANMYSKEAKATAISER | TTCAGCAAGGCTACGAAGGAATGGGCCGTTATGGGG |
| | TINENMVKHEDNCQRLEK | GAGAATAACGAATTGATCGGGAAAAACAAAGAGTCA |
| | IKSKYPDLAEELKDFEEF | AAGTTGTATCAGTGGCTGGAGAAGAACTACCGCGCA |
| | FKPSYFINCMNQSGIDYY | GGTCGCATCGAAAAAGAGGAATTCGACCATAATGCG |
| | NISAIGGKDEKDQKANMK | GGCTTAATCGAATACTTCGAGAAATTTTCCACATAT |
| | INLFTQKNHLKGSDKPPF | TTCGTAGGTTTTGACAAAAATCGTGCGAATATGTAT |
| | FAKLYKQILSDREKSVVI | TCAAAGGAGGCAAAGGCGACCGCAATTTCCTTCCGG |
| | DEFEKDSELTEALKNVES | ACGATTAATGAGAACATGGTCAAGCATTTCGATAAT |
| | KDGLINEEFFTKLKSALE | TGCCAGCGGCTCGAGAAGATTAAATCTAAATATCCT |
| | NEMLPEYQGQLYIRNAFL | GATTTGGCCGAGGAGCTGAAGGATTTTGAGGAGTTT |
| | TKISANIWGSGSWGIIKD | TTTAAACCTAGCTATTTCATTAATTGTATGAATCAA |
| | AVTQAAENNFTRKSDKEK | TCGGGTATCGACTACTACAATATCAGCGCGATCGGC |
| | YAKKDFYSIAELQQAIDE | GGTAAGGATGAAAAGGATCAGAAAGCGAATATGAAG |
| | YIPTLENGVQNASLIEYE | ATCAACCTTTTCACGCAAAAAAATCATTTAAAGGGC |
| | RKMNYKPRGSEEDAGLIE | AGTGATAAACCACCATTTTTTGCTAAGCTCTACAAG |
| | EINNNLRQAGIVLNQAEL | CAAATTTTGAGTGACCGGGAGAAGTCCGTGGTAATC |
| | GSGKQREENIEKIKNLLD | GACGAGTTCGAAAAGGACAGCGAATTGACAGAGGCA |
| | SVLNLERELKPLYLEKEK | CTCAAAAACGTGTTTTCCAAGGACGGTTTGATCAAT |
| | MRPKAANLNKDFCESEDP | GAGGAGTTTTTTACAAAGTTAAAAAGTGCATTAGAA |
| | LYEKLKTFFKLYNKVRNY | AATTTTATGTTGCCTGAATATCAAGGTCAACTCTAC |
| | ATKKPYSKDKFKINEDTA | ATCCGTAACGCTTTCCTTACGAAGATCAGCGCAAAC |
| | TLLYGWSLDKETANLSVI | ATTTGGGGCTCTGGTTCTTGGGGCATCATCAAGGAC |
| | FRKREKFYLGIINRYNSQ | GCAGTTACCCAGGCTGCGGAAAACAATTTCACGCGT |
| | IFNYKIAGSESEKGLERK | AAGTCTGACAAGGAAAAGTATGCCAAGAAAGACTTC |
| | RSLQQKVLAEEGEDYFEK | TATTCCATTGCTGAACTCCAGCAGGCTATTGATGAA |
| | MVYHLLLGASKTIPKCST | TACATTCCTACTCTGGAGAACGGGGTTCAAAACGCA |
| | QLKEVKAHFQKSSEDYII | TCACTCATCGAGTACTTTCGCAAAATGAATTACAAA |
| | QSKSFAKSLTLTKEIFDL | CCACGCGGTTCTGAAGAAGACGCAGGCTTGATCGAA |
| | NNLRYNTETGEISSELSD | GAAATTAATAACAACCTGCGTCAGGCTGGGATCGTC |
| | TYPKKFQKGYLTQTGDVS | CTGAATCAAGCCGAGCTGGGGTCTGGTAAGCAGCGG |
| | GYKTALHKWIDECKEFLR | GAAGAGAATATTGAAAAAATTAAGAACTTATTAGAT |
| | CYRNTEIFTFHFKDTKEY | TCGGTTTTGAATCTCGAACGTTTCTTAAAGCCACTT |
| | ESLDEFLKEVDSSGYEIS | TACTTGGAGAAAGAGAAAATGCGTCCAAAAGCTGCT |
| | FDKIKASYINEKVNAGEL | AACCTGAATAAGGATTTTTGTGAGTCATTTGATCCA |
| | YLFEIYNKDFSEYSKGKP | CTTTACGAGAAACTGAAAACGTTTTTCAAGCTCTAC |
| | NLHTIYWKSLFETQNLLD | AATAAAGTACGTAACTACGCAACAAAGAAACCATAC |
| | KTAKLNGKAEIFFRPRSI | TCAAAGGACAAATTTAAGATCAATTTTGATACCGCT |
| | KHNDKIIHRAGETLKNKN | ACGTTATTATATGGGTGGAGTTTGGATAAGGAAACC |
| | PLNEKPSSREDYDITKDR | GCGAATCTCAGCGTCATTTTCCGTAAACGCGAAAAA |
| | RFTKDKFFLHCPITLNEK | TTCTATTTGGGTATCATCAACCGGTACAATAGCCAG |
| | QDKPVRFNEQVNLYLKDN | ATTTTCAATTATAAGATTGCGGGCAGTGAGAGCGAG |
| | PDVNIIGIDRGERHLLYY | AAAGGGTTAGAGCGTAAGCGGTCGCTGCAGCAAAAG |
| | TLINQNGEILQQGSLNRI | GTGCTTGCAGAGGAGGGTGAAGATTATTTTGAGAAA |
| | GEEESRPTDYHRLLDERE | ATGGTATACCACCTGCTGCTTGGCGCGTCGAAAACT |
| | KQRQQARETWKAVEGIKD | ATTCCGAAATGCTCGACACAGTTGAAAGAAGTAAAA |
| | LKAGYLSRVVHKLAGLMV | GCACACTTTCAAAAGTCATCAGAAGATTATATTATC |
| | QNNAIVVLEDLNKGEKRG | CAATCCAAATCATTTGCAAAGTCATTAACATTAACA |
| | RFAVEKQVYQNFEKALIQ | AAAGAGATCTTTGACTTAAATAATCTGCGGTATAAC |
| | KLNYLVFKEVNSKDAPGH | ACAGAAACGGGCGAAATTAGTTCCGAGCTTTCTGAT |
| | YLKAYQLTAPFISFEKLG | ACATATCCGAAGAAGTTCCAGAAGGGGTATCTCACA |
| | TQSGELFYVRAWNTSKID | CAAACAGGCGACGTTTCGGGTTACAAAACTGCTCTG |
| | PATGFTDQIKPKYKNQKQ | CATAAGTGGATTGATTTCTGCAAAGAGTTCTTGCGT |
| | AKDFMSSFDSVRYNRKEN | TGCTATCGTAATACGGAGATCTTCACGTTCCATTTC |
| | YFEFEADFEKLAQKPKGR | AAGGACACGAAGGAGTACGAGTCGTTAGATGAGTTC |
| | TRWTICSYGQERYSYSPK | TTGAAAGAAGTGGATAGTTCAGGTTATGAGATTTCA |
| | ERKFVKHNVTQNLAELEN | TTCGATAAGATCAAAGCCTCTTATATCAACGAGAAG |
| | SEGISEDSGQCFKDEILK | GTTAATGCAGGCGAGCTGTACTTGTTCGAGATCTAT |
| | VEDASFFKSIIFNLRLLL | AATAAAGATTTCTCCGAGTATTCCAAAGGTAAGCCA |
| | KLRHTCKNAEIERDFIIS | AATCTGCATACCATTTATTGGAAAAGTCTCTTCGAG |
| | PVKGNNSSFFDSRIAEQE | ACTCAAAACTTGCTGGATAAAACAGCGAAACTCAAC |
| | NITSIPQNADANGAYNIA | GGCAAGGCAGAGATCTTCTTCCGGCCACGTTCGATC |
| | LKGLMNLHNISKDGKAKL | AAACACAACGACAAAATCATCCACCGTGCGGGCGAA |
| | IKDEDWIEFVQKRKFAAA | ACACTTAAGAATAAAAACCCGCTCAATGAAAAGCCT |
| | KRPAATKKAGQAKKKKAS | AGTTCGCGTTTCGATTACGATATTACGAAAGATCGT |
| | GSGAGSPKKKRKVEDPKK | CGTTTTACGAAAGACAAATTTTTTTTACACTGCCCT |
| | KRKV (SEQ ID NO: | ATTACGTTAAACTTTAAGCAGGACAAGCCTGTTCGC |
| | 16) | TTTAATGAACAAGTCAACTTATACTTAAAAGACAAT |
| | | CCAGACGTGAATATTATCGGTATCGATCGTGGTGAG |
| | | CGTCACTTGCTTTATTACACTTTGATCAATCAGAAT |
| | | GGTGAGATCTTACAGCAGGGTTCACTTAATCGCATT |
| | | GGTGAGGAAGAATCTCGGCCTACGGACTACCATCGG |
| | | TTACTCGATGAGCGTGAAAAGCAGCGTCAACAAGCA |
| | | CGGGAGACGTGGAAAGCAGTAGAAGGGATTAAGGAC |
| | | TTAAAAGCTGGGTATCTTTCACGGGTTGTACATAAA |
| | | CTTGCAGGTTTAATGGTACAAAACAACGCAATTGTC |
| | | GTTCTGGAAGATCTTAACAAGGGTTTTAAGCGCGGT |
| | | CGTTTCGCTGTTGAGAAACAGGTGTACCAGAACTTC |
| | | GAAAAAGCACTTATTCAAAAGCTTAACTATTTAGTG |
| | | TTCAAGGAGGTCAACTCTAAAGACGCCCCTGGCCAC |
| | | TATTTGAAGGCATATCAGCTTACGGCCCCTTTCATC |
| | | TCGTTCGAAAAATTGGGTACTCAGAGCGGTTTCCTT |
| | | TTTTATGTGCGCGCATGGAATACCTCGAAGATCGAC |
| | | CCGGCGACGGGTTTTACCGACCAAATCAAACCAAAG |
| | | TATAAAAACCAAAAACAAGCTAAAGACTTCATGTCA |
| | | AGCTTCGACTCTGTCCGGTACAACCGCAAGGAAAAT |
| | | TATTTTGAATTCGAGGCGGACTTTGAAAAACTGGCA |
| | | CAGAAACCTAAGGGGCGCACCCGCTGGACGATTTGT |
| | | TCCTATGGCCAGGAACGGTACTCTTACTCCCCAAAA |
| | | GAACGGAAGTTTGTAAAGCACAACGTTACACAAAAT |
| | | CTTGCTGAGCTTTTTAATTCAGAGGGTATCTCGTTC |
| | | GACTCCGGGCAGTGTTTCAAGGATGAGATCCTGAAG |
| | | GTCGAGGATGCCAGTTTCTTTAAGTCTATTATTTTC |
| | | AATCTTCGCCTCCTTCTCAAGCTTCGTCACACTTGC |
| | | AAGAACGCCGAGATCGAACGTGATTTCATCATTTCT |
| | | CCTGTCAAGGGGAACAATTCGTCCTTTTTTGACTCC |
| | | CGTATTGCCGAACAAGAAAATATCACCAGCATTCCA |
| | | CAGAATGCTGATGCAAACGGTGCATACAACATCGCG |
| | | CTGAAGGGCCTGATGAACCTCCATAATATCTCTAAG |
| | | GACGGCAAGGCAAAATTAATTAAGGATGAAGATTGG |
| | | ATCGAATTTGTCCAAAAACGCAAGTTCGCGGCCGCA |
| | | AAAAGGCCGGCGGCCACGAAAAAGGCCGGCCAGGCA |
| | | AAAAAGAAAAAGGCTAGCGGCAGCGGCGCCGGATCC |
| | | CCAAAGAAGAAAAGGAAGGTTGAAGACCCCAAGAAA |
| | | AAGAGGAAGGTGTGATAA (SEQ ID NO: 17) |
| ABW3 | MGHHHHHHSSGLVPRGSL | ATGGGCCACCATCATCATCATCATAGCAGCGGCCTG |
| | QMKTLSDFTNLFPLSKTL | GTGCCGCGCGGCAGCCTGCAGATGAAGACCTTGTCT |
| | RFKLIPIGNTLKNIEASG | GATTTTACCAATCTGTTCCCTTTATCTAAGACTCTC |
| | ILDEDRHRAESYVKVKAI | CGTTTCAAGCTGATTCCAATCGGCAACACGCTCAAG |
| | IDEYHKAFIDRVLSDTCL | AACATTGAAGCTAGTGGCATCCTTGACGAGGATCGC |
| | QTESIGKHNSLEEFFFYY | CACCGCGCGGAGTCCTATGTCAAGGTCAAGGCCATC |
| | QIGAKSEQQKKTEKKIQD | ATCGACGAATATCATAAAGCTTTCATCGATCGGGTC |
| | ALRKQIADSLTKDKHESR | CTGTCGGATACTTGCCTCCAGACGGAATCTATCGGC |
| | IDKKELIQEDLIQFVRDG | AAACACAACAGTCTCGAGGAATTCTTTTTCTACTAC |
| | EDAAEKTSLISEFQNFTV | CAAATTGGTGCAAAAAGTGAACAGCAGAAAAAGACG |
| | YFTGFHENRQNMYSPDEK | TTTAAAAAGATTCAAGACGCCTTGCGCAAACAAATC |
| | STAIAYRLINENLPKFVD | GCAGATAGCCTCACCAAGGACAAACATTTTTCACGG |
| | NMKVEDRIAASELASCED | ATTGATAAAAAAGAATTGATCCAAGAGGATTTGATC |
| | ELYHNFEEYLQVERLHDI | CAGTTTGTGCGCGATGGGGAGGATGCCGCTGAAAAG |
| | FSLDYFNLLLTQKHIDVY | ACGTCTCTGATTTCCGAATTTCAAAATTTCACAGTT |
| | NALIGGKATETGEKIKGL | TATTTTACCGGGTTTCATGAGAATCGCCAGAACATG |
| | NEYINLYNQRHKQEKLPK | TACAGTCCGGACGAGAAGTCCACGGCCATCGCATAT |
| | FKMLFKQILTDREAISWL | CGCTTAATTAACGAGAATCTCCCAAAATTCGTAGAC |
| | PRQFDDNSQLLSAIEQCY | AACATGAAAGTTTTTGACCGTATCGCGGCGTCCGAA |
| | NHLSTYTLKDGSLKYLLE | TTGGCATCGTGTTTCGACGAATTATACCACAACTTC |
| | NLHTYDTEKIFIRNDSLL | GAGGAATACCTCCAAGTGGAGCGGTTACATGATATC |
| | TEISQRHYGSWSILPEAI | TTTAGTTTGGACTATTTCAATCTGCTTCTCACGCAG |
| | KRHLERANPQKRRETYEA | AAACATATCGACGTCTATAATGCTCTGATCGGTGGG |
| | YQSRIEKAFKAYPGESIA | AAGGCAACCGAAACCGGGGAAAAGATCAAGGGCTTA |
| | FLNGCLTETGKESPSIES | AATGAATACATCAATCTCTACAATCAACGTCACAAG |
| | YFESLGAVETETSQQENW | CAGGAAAAACTGCCAAAATTCAAGATGTTATTCAAG |
| | FARIANAYTDFREMQNRL | CAAATTCTTACCGACCGTGAGGCAATCAGCIGGTTG |
| | HATDVPLAQDAEAVARIK | CCACGCCAATTTGACGATAATAGTCAGTTACTCTCA |
| | KLLDALKGLQLFIKPLLD | GCCATTGAACAGTGTTATAACCACCTTTCGACCTAC |
| | TGEEAEKDERFYGDFTEF | ACACTCAAGGATGGGTCACTCAAATACCTGTTAGAA |
| | WNELDTITPLYNMVRNYL | AACCTGCATACATACGATACTGAAAAGATCTTCATC |
| | TRKPYSEEKIKLNFQNPT | CGCAATGACAGTTTACTTACGGAAATCTCCCAACGG |
| | LLNGWDLNKEVDNTSVIL | CATTACGGTTCGTGGTCGATTTTACCAGAAGCTATC |
| | RRNGRYYLAIMHRNHRRV | AAACGTCATCTCGAGCGCGCGAACCCGCAAAAACGG |
| | FSQYPGTERGDCYEKMEY | CGCGAAACATACGAGGCCTATCAATCTCGCATTGAG |
| | KLLPGANKMLPKVFFSKS | AAGGCCTTTAAGGCATATCCGGGGTTTTCAATTGCT |
| | RIDEFNPSEELLARYQQG | TTCCTCAATGGGTGTTTAACAGAGACAGGTAAGGAG |
| | THKKGENENLHDCHALID | TCGCCATCCATCGAAAGCTATTTTGAAAGTCTGGGT |
| | FFKDSIEKHEEWRNFHFK | GCTGTCGAAACAGAGACCTCTCAGCAGGAAAACTGG |
| | FSDTSSYTDMSGFYREIE | TTTGCCCGCATCGCAAACGCTTATACGGACTTTCGT |
| | TQGYKLSFVPVACEYIDE | GAAATGCAAAATCGGCTGCACGCCACTGACGTGCCG |
| | LVRDGKIFLFQIYNKDES | TTGGCTCAAGACGCTGAGGCAGTGGCCCGGATCAAG |
| | TYSKGKPNMHTLYWEMLF | AAGCTGTTAGATGCACTGAAAGGCCTGCAATTATTC |
| | DERNLMNVVYKLNGQAEI | ATTAAGCCTCTTTTGGATACTGGCGAAGAAGCAGAG |
| | FFRKASLSARHPEHPAGL | AAAGATGAACGGTTCTATGGGGACTTTACCGAATTC |
| | PIKKKQAPTEESCFPYDL | TGGAACGAGTTAGACACTATCACGCCATTGTACAAT |
| | IKNKRYTVDQFQFHVPIT | ATGGTACGGAACTATCTCACGCGTAAGCCTTATAGT |
| | INFKATGTSNINPSVTDY | GAAGAAAAAATCAAGCTCAATTTCCAGAATCCGACA |
| | IRTADDLHIIGIDRGERH | TTACTGAACGGTTGGGATTTGAACAAAGAGGTAGAT |
| | LLYLVVIDSQGRICEQFS | AATACATCTGTCATCCTCCGCCGGAATGGTCGTTAT |
| | LNEIVTQYQGHQYRTDYH | TATCTTGCCATCATGCACCGCAACCACCGGCGTGTA |
| | ALLQKKEDERQKARQSWQ | TTTTCACAGTATCCAGGCACAGAACGTGGCGATTGT |
| | SIENIKELKEGYLSQVVH | TATGAGAAAATGGAATATAAACTGCTTCCGGGCGCC |
| | KVSELMIKYKAIVVLEDL | AACAAGATGCTCCCAAAAGTCTTCTTCTCTAAATCA |
| | NAGFKRSRQKVEKQVYQK | CGCATCGATGAATTCAACCCTAGCGAAGAATTATTA |
| | FEKMLIDKLNYLVEKTAE | GCACGTTACCAGCAAGGTACCCACAAGAAGGGTGAG |
| | ADQPGGLLHAYQLTNKFE | AATTTTAATTTACACGACTGCCATGCCTTGATTGAT |
| | SFKKMGKQSGELFYIPAW | TTTTTTAAAGACTCTATTGAGAAACATGAAGAATGG |
| | NTSKIDPTTGFVNLEDTR | CGTAACTTTCATTTTAAATTTAGTGATACGTCCAGT |
| | YENVDKSRAFFGKEDSIR | TACACCGACATGAGCGGCTTTTATCGTGAAATCGAA |
| | YRADKGTFEWTEDYNNFH | ACACAGGGTTACAAGTIGTCATTTGTGCCAGTGGCG |
| | KKAEGTRSSWCLSSHGNR | TGTGAATACATCGATGAGTTGGTACGTGATGGCAAA |
| | VRTERNPAKNNQWDNEEI | ATCTTTTTGTTCCAGATCTATAATAAGGACTTTTCG |
| | DLTQAFRDLFEAWGIEIT | ACCTACTCTAAGGGCAAGCCAAATATGCACACTCTT |
| | SNLKEAICNQSEKKFFSE | TATTGGGAAATGCTTTTCGACGAGCGGAACCTGATG |
| | LFELFKLMIQLRNSVTGT | AACGTGGTGTATAAACTCAATGGCCAAGCAGAGATC |
| | NIDYMVSPVENHYGTFED | TTTTTTCGTAAAGCATCACTGAGCGCACGTCACCCT |
| | SRTCDSSLPANADANGAY | GAGCACCCGGCAGGGTTGCCAATTAAAAAAAAACAG |
| | NIARKGLMLARRIQATPE | GCCCCGACGGAAGAATCTTGTTTCCCATATGATCTC |
| | NDPISLTLSNKEWLRFAQ | ATTAAGAATAAGCGGTATACAGTTGACCAGTTTCAG |
| | GLDETTTYEAAAKRPAAT | TTTCACGTGCCAATTACTATTAATTTTAAAGCAACT |
| | KKAGQAKKKKASGSGAGS | GGGACTTCAAATATCAACCCGTCGGTCACTGATTAT |
| | PKKKRKVEDPKKKRKV | ATTCGTACGGCCGATGACCTCCATATCATTGGCATT |
| | (SEQ ID NO: 29) | GATCGCGGTGAGCGCCATTTACTTTATTTAGTGGTG |
| | | ATTGACTCACAAGGGCGCATCTGTGAACAGTTTTCC |
| | | TTAAACGAGATCGTAACGCAATACCAAGGTCACCAG |
| | | TACCGTACAGATTATCATGCTCTCTTGCAGAAAAAA |
| | | GAGGATGAACGGCAAAAAGCTCGCCAGTCTTGGCAA |
| | | TCGATCGAAAACATCAAGGAATTAAAAGAGGGGTAT |
| | | CTGAGCCAAGTAGTGCACAAGGTTTCTGAACTGATG |
| | | ATCAAATATAAAGCAATTGTGGTGTTGGAAGATTTA |
| | | AATGCTGGGTTCAAGCGGAGTCGGCAGAAGGTTGAA |
| | | AAGCAAGTGTATCAAAAATTTGAGAAGATGCTGATC |
| | | GACAAACTTAACTATCTTGTGTTCAAGACCGCAGAA |
| | | GCTGACCAACCTGGCGGCCTCCTGCACGCATACCAA |
| | | TTAACAAATAAATTTGAGTCATTCAAGAAAATGGGG |
| | | AAGCAAAGTGGCTTCCTCTTCTACATTCCTGCATGG |
| | | AACACGTCTAAAATCGACCCGACCACGGGCITTGTC |
| | | AACCTTTTTGATACCCGGTATGAGAACGTAGACAAA |
| | | TCCCGTGCCTTCTTCGGCAAATTCGATAGCATCCGC |
| | | TACCGTGCGGACAAGGGCACGTTCGAGTGGACGTTC |
| | | GATTATAATAACTTTCACAAAAAGGCCGAAGGTACG |
| | | CGGTCGAGCTGGTGTTTGTCTTCTCATGGTAACCGG |
| | | GTCCGTACTTTCCGCAATCCTGCGAAAAACAACCAA |
| | | TGGGACAACGAAGAGATCGACTTAACACAAGCGTTC |
| | | CGCGATCTGTTTGAAGCTTGGGGGATCGAGATCACT |
| | | TCGAACTTAAAAGAGGCCATTTGCAACCAGTCTGAG |
| | | AAGAAATTCTTTTCTGAGCTTTTCGAACTGTTCAAA |
| | | CTTATGATCCAGCTGCGGAACTCAGTGACAGGCACG |
| | | AATATCGACTATATGGTGAGCCCAGTCGAGAATCAC |
| | | TACGGCACGTTCTTCGATTCGCGCACATGCGATTCG |
| | | TCTCTGCCGGCTAACGCTGACGCTAATGGTGCTTAT |
| | | AATATTGCCCGTAAGGGGTTAATGCTGGCTCGCCGC |
| | | ATTCAGGCTACCCCTGAGAATGATCCGATCTCCTTA |
| | | ACATTGAGCAACAAAGAGTGGTTACGCTTTGCACAG |
| | | GGGCTCGATGAGACAACAACCTACGAGGCGGCCGCA |
| | | AAAAGGCCGGCGGCCACGAAAAAGGCCGGCCAGGCA |
| | | AAAAAGAAAAAGGCTAGCGGCAGCGGCGCCGGATCC |
| | | CCAAAGAAGAAAAGGAAGGTTGAAGACCCCAAGAAA |
| | | AAGAGGAAGGTGTGATAA (SEQ ID NO: 30) |
| ABW4 | MGHHHHHHSSGLVPRGSG | ATGGGCCACCATCATCATCATCATAGCAGCGGCCTG |
| | TMKNMESFINLYPVSKTL | GTGCCGCGCGGCAGCGGTACCATGAAGAACATGGAG |
| | RFELKPIGKTLETFSRWI | TCTTTTATTAATTTATATCCGGTTTCGAAAACTTTA |
| | EELKEKEAIELKETGNLL | CGTTTTGAGTTAAAGCCTATTGGCAAAACACTCGAA |
| | AQDEHRAESYKKVKKILD | ACTTTCTCCCGCTGGATCGAAGAGTTGAAAGAGAAA |
| | EYHKWFITESLQNTKLNG | GAGGCTATTGAGCTGAAAGAAACTGGCAACCTGTTG |
| | LDVFYHNYMLPKKEDHEK | GCGCAGGATGAGCATCGGGCCGAGTCTTATAAGAAG |
| | KAFASCQDNLRKQIVNAF | GTCAAAAAAATTCTTGACGAATATCATAAATGGTTC |
| | RQETGLENKLSGKELFKD | ATCACTGAAAGCCTCCAGAACACAAAGTTAAATGGG |
| | SKEEVALLKAIVPYEDNK | TTGGACGTTTTTTATCATAACTATATGCTCCCGAAG |
| | TLENIGVKSNEGALLLIE | AAAGAGGACCATGAGAAGAAAGCTTTTGCTTCGTGT |
| | EFKDETTYFGGFHENRKN | CAAGATAATCTCCGTAAGCAAATTGTAAACGCGTTT |
| | MYSDEAKSTAVAFRLIHE | CGTCAAGAAACCGGTTTATTTAACAAACTGTCAGGC |
| | NLPRFIDNKKVFEEKIMN | AAAGAACTGTTTAAAGATTCGAAGGAAGAGGTTGCA |
| | SELKDKFPEILKELEQIL | CTGTTGAAAGCCATTGTACCGTATTTCGATAACAAG |
| | QVNEIEEMFQLDYENDTL | ACTCTGGAAAACATTGGTGTTAAGAGTAATGAAGGG |
| | IQNGIDVYNHLIGGYAEE | GCTCTCCTTTTAATTGAAGAGTTCAAGGATTTTACC |
| | GKKKIQGLNEHINLYNQI | ACGTATTTCGGTGGCTTCCATGAGAATCGCAAAAAT |
| | QKEKNKRIPRLKPLYKQI | ATGTATAGCGACGAAGCAAAATCAACAGCGGTTGCC |
| | LSDRETASFVIEAFENDG | TTTCGTCTTATTCACGAAAATTTGCCGCGCTTCATT |
| | ELLESLEKSYRLLQQEVF | GACAATAAGAAGGTCTTCGAAGAGAAAATCATGAAT |
| | TPEGKEGLANLLAAIAES | AGTGAATTAAAGGATAAATTTCCAGAGATTTTGAAG |
| | ETHKIFLKNDLGLTEISQ | GAGCTGGAACAGATTCTGCAAGTCAACGAGATTGAA |
| | QIYESWSLIEEAWNKQYD | GAGATGTTTCAGCTCGACTATTTTAACGACACATTG |
| | NKQKKVTETETYVDNRKK | ATCCAGAATGGCATCGATGTCTATAACCATTIGATC |
| | AFKSIKSFSIAEVEEWVK | GGCGGCTACGCCGAGGAAGGCAAGAAAAAAATTCAA |
| | ALGNEKHKGKSVATYFKS | GGGCTTAACGAGCATATTAACCTCTATAACCAGATC |
| | LGKTDEKVSLIEQVENNY | CAGAAGGAGAAGAATAAGCGTATCCCGCGGCTGAAA |
| | NIIKDLLNTPYPPSKDLA | CCACTCTATAAGCAAATTTTGAGTGATCGCGAAACC |
| | QQKDDVEKIKNYLDSLKA | GCCTCATTTGTTATCGAGGCGTTTGAGAACGATGGC |
| | LQRFIKPLLGSGEESDKD | GAGTTATTAGAATCATTGGAGAAGTCATATCGCTTA |
| | AHFYGEFTAFWDVLDKVT | CTGCAGCAGGAGGTCTTTACGCCTGAAGGTAAAGAA |
| | PLYNKVRNYMTKKPYSTE | GGTCTGGCGAATTTACTCGCAGCAATCGCTGAAAGC |
| | KFKLNFENSYFLNGWAQD | GAGACACACAAGATCTTTCTGAAGAACGACTTGGGT |
| | YETKAGLIFLKDGNYFLA | CTCACCGAGATCTCTCAACAAATTTATGAATCATGG |
| | INNKKLDEKEKKQLKTNY | TCGCTGATTGAAGAGGCATGGAATAAACAATATGAC |
| | EKNPAKRIILDFQKPDNK | AACAAACAGAAGAAAGTTACGGAGACAGAGACATAT |
| | NIPRLFIRSKGDNFAPAV | GTGGACAATCGGAAAAAGGCTTTCAAGTCCATCAAG |
| | EKYNLPISDVIDIYDEGK | AGCTTTAGCATCGCAGAGGTTGAGGAATGGGTGAAA |
| | FKTEYRKINEPEYLKSLH | GCACTTGGGAATGAGAAACACAAGGGCAAAAGCGTG |
| | KLIDYFKLGESKHESYKH | GCAACCTATTTTAAAAGTCTCGGGAAGACTGACGAA |
| | YSFSWKKTHEYENIAQFY | AAAGTTAGCCTTATTGAACAGGTAGAGAACAATTAT |
| | HDVEVSCYQVLDENINWD | AATATCATCAAGGACCTTTTGAACACACCGTATCCT |
| | SLMEYVEQNKLYLFQIYN | CCTTCGAAGGACTTGGCCCAGCAAAAAGATGACGTT |
| | KDFSPNSKGTPNMHTLYW | GAAAAAATCAAAAATTATTTGGACTCTCTGAAGGCC |
| | KMLFNPDNLKDVVYKLNG | CTCCAGCGGTTCATTAAGCCATTGTTGGGTAGCGGG |
| | QAEVFYRKASIKKENKIV | GAGGAATCCGATAAAGATGCGCACTTTTATGGTGAG |
| | HKANDPIDNKNELNKKKQ | TTTACCGCTTTCTGGGATGTGCTCGACAAAGTAACC |
| | NTFEYDIVKDKRYTVDKF | CCACTCTACAATAAAGTCCGCAACTATATGACTAAG |
| | QFHVPITLNFKAEGLNNL | AAACCTTATAGCACAGAGAAATTTAAGCTGAATTTT |
| | NSKVNEYIKECDDLHIIG | GAAAATAGTTACTTTTTGAATGGTTGGGCACAGGAC |
| | IDRGERHLLYLSLIDMKG | TACGAGACAAAAGCGGGGCTTATCTTCTTGAAGGAC |
| | NIVKQFSLNEIVNEHKGN | GGCAATTACTTCCTTGCCATCAATAATAAGAAATTA |
| | TYRTNYHNLLDKREKERE | GATGAAAAGGAGAAAAAACAGCTCAAGACTAATTAT |
| | KERESWKTIETIKELKEG | GAGAAGAATCCTGCGAAGCGTATCATCTTAGACTTT |
| | YISQVVHKITQLMIEYNA | CAGAAGCCAGACAATAAGAACATTCCTCGCTTGTTC |
| | IVVLEDLNFGFKRGRFKV | ATTCGCAGTAAAGGCGACAATTTCGCTCCTGCAGTA |
| | EKQVYQKFEKMLIDKLNY | GAAAAGTATAATCTTCCGATCTCTGACGTTATTGAC |
| | LVDKKKEANESGGTLKAY | ATCTATGACGAGGGGAAGTTTAAGACTGAGTATCGC |
| | QLTDSYADFMKYKKKQCG | AAAATTAACGAGCCGGAATATCTCAAATCTCTCCAT |
| | FLFYVPAWNTSKIDPTTG | AAGCTGATTGACTACTTCAAACTTGGGTTCTCCAAG |
| | FVNLFDTHYVNVSKAQEF | CATGAATCCTACAAGCATTATTCTTTTTCATGGAAG |
| | FSKEKSIRYNAANNYFEF | AAAACACATGAGTATGAGAACATCGCCCAGTTTTAC |
| | EVTDYFSFSGKAEGTKQN | CACGACGTGGAGGTCTCTTGCTATCAGGTGCTCGAC |
| | WIICTHGTRIINFRNPEK | GAAAATATTAACTGGGATTCCCTCATGGAGTATGTA |
| | NSQWDNKEVVITDEFKKL | GAACAGAACAAATTGTACTTGTTCCAGATTTATAAC |
| | FEKHGIDYKNSSDLKGQI | AAAGACTTCTCCCCAAACTCGAAAGGCACTCCGAAT |
| | ASQSEKAFFHNEKKDTKD | ATGCACACTTTGTACTGGAAGATGTTGTTTAATCCG |
| | PDGLLQLFKLALQMRNSF | GATAATCTTAAGGACGTGGTCTATAAGCTGAACGGT |
| | IKSEEDYLVSPVMNDEGE | CAGGCTGAAGTATTCTACCGGAAGGCGAGTATTAAG |
| | FFDSRKAQPNQPENADAN | AAAGAAAACAAGATTGTCCACAAGGCGAACGACCCT |
| | GAYNIAMKGKWVVKQIRE | ATTGACAATAAAAACGAGTTGAATAAGAAAAAGCAA |
| | SEDLDKLKLAISNKEWLN | AATACATTTGAATACGACATCGTCAAAGATAAACGG |
| | FAQRSAAAKRPAATKKAG | TATACAGTGGATAAGTTTCAATTCCATGTTCCTATC |
| | QAKKKKASGSGAGSPKKK | ACGCTCAACTTTAAAGCTGAAGGCCTGAATAACTTG |
| | RKVEDPKKKRKV | AATAGCAAAGTTAACGAATACATCAAAGAGTGTGAC |
| | (SEQ ID NO: 42) | GACCTTCACATTATTGGCATCGACCGGGGTGAACGG |
| | | CACCTCTTGTATCTGAGCCTCATCGATATGAAAGGT |
| | | AACATTGTAAAGCAATTTAGTCTTAACGAGATCGTT |
| | | AATGAGCACAAGGGGAACACGTACCGCACGAACTAT |
| | | CATAACCTCTTGGACAAACGTGAAAAGGAACGTGAA |
| | | AAAGAGCGCGAGTCATGGAAAACCATTGAGACCATC |
| | | AAAGAGCTGAAAGAAGGCTATATTAGTCAAGTAGTA |
| | | CATAAAATCACTCAGTTAATGATCGAATATAATGCG |
| | | ATCGTTGTACTCGAAGACCTGAATTTCGGCTTCAAA |
| | | CGCGGCCGGTTCAAGGTGGAGAAGCAAGTGTATCAA |
| | | AAATTTGAGAAGATGTTAATTGATAAACTGAACTAC |
| | | TTGGTCGATAAGAAGAAGGAAGCCAATGAGAGTGGC |
| | | GGGACACTCAAAGCCTACCAGCTTACCGATAGTTAC |
| | | GCTGACTTCATGAAGTACAAGAAAAAGCAATGCGGC |
| | | TTCCTGTTTTATGTCCCGGCCTGGAACACTTCCAAA |
| | | ATCGATCCTACTACTGGGTTCGTGAATCTGTTTGAC |
| | | ACACATTATGTCAATGTTAGTAAGGCCCAGGAATTT |
| | | TTCTCGAAATTCAAGTCAATTCGCTACAACGCGGCC |
| | | AACAACTATTTCGAGTTTGAAGTAACAGATTATTTT |
| | | TCCTTCAGTGGTAAAGCTGAGGGCACCAAGCAGAAT |
| | | TGGATCATTTGCACCCATGGCACCCGCATTATCAAT |
| | | TTTCGTAACCCGGAAAAAAATTCGCAGTGGGATAAT |
| | | AAGGAAGTAGTGATCACAGATGAATTCAAGAAACTG |
| | | TTTGAGAAGCACGGCATTGACTACAAAAATAGTTCC |
| | | GACCTCAAGGGGCAGATCGCCTCTCAATCGGAGAAG |
| | | GCGTTTTTTCATAACGAAAAAAAAGATACAAAGGAC |
| | | CCAGATGGCCTTCTGCAGCTTTTTAAACTGGCGCTG |
| | | CAGATGCGGAACTCTTTCATTAAGAGCGAAGAGGAC |
| | | TACTTAGTATCTCCTGTGATGAACGACGAAGGTGAA |
| | | TTCTTTGACTCGCGCAAAGCCCAGCCTAATCAGCCA |
| | | GAGAACGCTGATGCTAATGGGGCGTACAATATTGCA |
| | | ATGAAAGGGAAATGGGTTGTTAAGCAAATCCGCGAA |
| | | TCGGAGGACCTCGACAAGCTGAAACTGGCAATCTCA |
| | | AATAAAGAATGGTTGAACTTCGCCCAGCGCTCCGCG |
| | | GCCGCAAAAAGGCCGGCGGCCACGAAAAAGGCCGGC |
| | | CAGGCAAAAAAGAAAAAGGCTAGCGGCAGCGGCGCC |
| | | GGATCCCCAAAGAAGAAAAGGAAGGTTGAAGACCCC |
| | | AAGAAAAAGAGGAAGGTGTGATAA(SEQ ID NO: |
| | | 43) |
| ABW5 | MGHHHHHHSSGLVPRGSG | ATGGGCCACCATCATCATCATCATAGCAGCGGCCTG |
| | TMKNILEQFVGLYPLSKT | GTGCCGCGCGGCAGCGGTACCATGAAGAACATCTTA |
| | LRFELKPLGKTLEHIEKK | GAGCAGTTTGTCGGCTTATATCCGTTGTCTAAAACA |
| | GLIAQDEQRAEEYKLVKD | CTTCGGTTTGAGCTTAAACCTTTGGGTAAGACGTTG |
| | IIDRYHKAFIHMCLKHFK | GAACATATTGAGAAAAAAGGCTTGATTGCCCAAGAC |
| | LKMYSEQGYDSLEEYRKL | GAACAGCGGGCGGAGGAGTACAAATTGGTTAAAGAT |
| | ASISKRNEKEEQQFDKVK | ATTATTGATCGCTACCACAAGGCTTTTATTCATATG |
| | ENLRKQIVDAFKNGGSYD | TGCTTAAAACATTTTAAGCTCAAGATGTACAGTGAA |
| | DLFKKELIQKHLPRFIEG | CAAGGGTATGATAGCTTGGAGGAGTACCGCAAGCTT |
| | EEEKRIVDNENKFTTYFT | GCGTCAATTTCCAAACGCAACGAGAAAGAGGAGCAG |
| | GEHENRKNMYSDEKESTA | CAATTTGACAAAGTCAAGGAAAATCTTCGTAAGCAA |
| | IAYRLIHENLPLELDNMK | ATTGTCGACGCGTTTAAAAATGGCGGGAGTTATGAT |
| | SFAKIAESEVAARFTEIE | GATCTGTTTAAGAAAGAATTGATCCAGAAACACCTC |
| | TAYRTYLNVEHISELFTL | CCACGTTTTATTGAGGGTGAAGAAGAAAAACGTATC |
| | DYFSTVLTQEQIEVYNNI | GTTGACAACTTCAACAAGTTCACGACCTATTTTACT |
| | IGGRVDDDNVKIQGLNEY | GGTTTTCATGAAAATCGCAAGAATATGTATAGTGAC |
| | VNLYNQQQKDRSKRLPLL | GAAAAGGAATCGACGGCTATTGCTTATCGTCTCATT |
| | KSLYKMILSDRIAISWLP | CACGAAAACTTGCCATTGTTTTTGGATAACATGAAG |
| | EEFKSDKEMIEAINNMHD | AGCTTCGCTAAGATCGCCGAATCGGAAGTGGCTGCT |
| | DLKDILAGDNEDSLKSLL | CGTTTTACCGAAATCGAAACCGCTTACCGGACATAC |
| | QHIGQYDLSKIYIANNPG | TTGAACGTAGAACACATTAGTGAACTGTTCACCCTC |
| | LTDISQQMFGCYDVFTNG | GACTATTTTAGCACGGTTTTGACGCAAGAACAAATC |
| | IKQELRNSITPSKKEKAD | GAAGTATATAATAACATTATCGGCGGGCGCGTCGAC |
| | NEIYEERINKMFKSEKSF | GACGACAACGTAAAGATCCAAGGGTTGAATGAGTAC |
| | SIAYLNSLPHPKTDAPQK | GTAAATTTATATAATCAGCAGCAGAAGGACCGGTCT |
| | NVEDYFALLGTCNQNDEQ | AAGCGCTTACCGCTTCTTAAGTCCCTCTACAAAATG |
| | PINLFAQIEMARLVASDI | ATCTTATCCGATCGTATTGCAATTTCGTGGTTACCT |
| | LAGRHVNLNQSENDIKLI | GAGGAGTTCAAATCCGATAAGGAGATGATTGAAGCA |
| | KDLLDAYKALQHFVKPLL | ATTAACAACATGCATGACGACCTGAAGGACATTCTG |
| | GSGDEAEKDNEFDARLRA | GCAGGCGACAACGAAGACTCGCTTAAGTCCTTACTG |
| | AWNALDIVTPLYNKVRNW | CAGCATATTGGCCAATACGATCTCTCGAAAATCTAC |
| | LTRKPYSTEKIKLNFENA | ATTGCGAACAATCCGGGCCTGACAGATATCTCACAA |
| | QLLGGWDQNKEPDCTSVL | CAAATGTTCGGGTGTTATGACGTCTTTACTAATGGG |
| | LRKDGMYYLAIMDKKANH | ATCAAGCAGGAGCTCCGGAACAGTATTACCCCTTCA |
| | AFDCDCLPSDGACFEKID | AAAAAGGAGAAAGCCGATAACGAAATCTACGAGGAG |
| | YKLLPGANKMLPKVFFSK | CGGATTAACAAAATGTTTAAAAGTGAGAAGAGTTTC |
| | SRIKEFSPSESIIAAYKK | TCAATTGCCTACCTGAATTCGTTGCCGCACCCAAAG |
| | GTHKKGPNFSLSDCHRLI | ACGGATGCGCCTCAAAAAAATGTTGAGGATTATTTT |
| | DFFKASIDKHEDWSKERF | GCTCTCCTGGGGACTTGCAATCAAAACGATGAACAG |
| | RESDTKTYEDISGFYREV | CCGATTAATTTGTTTGCCCAAATTGAGATGGCACGC |
| | EQQGYMLGFRKVSEAFVN | TTAGTCGCCTCTGATATTCTCGCAGGCCGGCACGTT |
| | KLVDEGKLYLFHIWNKDE | AATTTGAACCAATCTGAGAATGATATCAAGTTAATC |
| | SKHSKGTPNLHTIYWKML | AAGGATCTGTTAGATGCTTACAAGGCTCTGCAGCAT |
| | FDEKNLTDVIYKLNGQAE | TTCGTCAAACCACTCCTTGGCTCGGGTGACGAGGCT |
| | VFYRKKSLDLNKTTTHKA | GAGAAAGATAACGAGTTCGATGCACGCCTCCGTGCG |
| | HAPITNKNTQNAKKGSVE | GCTTGGAATGCGTTGGACATTGTTACACCACTCTAT |
| | DYDIIKNRRYTVDKFQFH | AACAAGGTTCGGAACTGGCTGACCCGCAAACCATAT |
| | VPITLNFKATGRNYINEH | TCTACAGAAAAAATCAAGCTTAATTTCGAAAACGCC |
| | TQEAIRNNGIEHIIGIDR | CAACTTCTGGGGGGTTGGGATCAGAACAAAGAACCG |
| | GERHLLYLSLIDLKGNIV | GATTGCACATCAGTCCTCCTTCGGAAGGATGGGATG |
| | KQMTLNDIVNEYNGRTYA | TACTATTTAGCGATCATGGATAAAAAGGCGAATCAC |
| | TNYKDLLATREGERTDAR | GCCTTTGACTGTGACTGCTTACCGTCTGACGGGGCC |
| | RNWQKIENIKEIKEGYLS | TGTTTCGAGAAAATTGACTACAAGCTGCTCCCGGGC |
| | QVVHILSKMMVDYKAIVV | GCGAATAAAATGTTGCCGAAAGTTTTTTTTTCTAAA |
| | LEDLNTGEMRNRQKIERQ | AGCCGCATCAAAGAATTTTCCCCTTCGGAATCGATC |
| | VYEKFEKMLIDKLNCYVD | ATCGCTGCTTATAAAAAGGGGACTCATAAAAAAGGG |
| | KQKDADETGGALHPLQLT | CCGAATTTCAGTCTCTCTGATTGTCATCGCTTGATT |
| | NKFESFRKLGKQSGWLFY | GACTTTTTTAAGGCTAGCATTGATAAGCACGAAGAT |
| | IPAWNTSKIDPVTGFVNM | TGGTCAAAATTTCGTTTTCGCTTCTCAGATACCAAA |
| | LDTRYENADKARCFFSKF | ACGTATGAAGACATCAGTGGTTTCTACCGTGAAGTA |
| | DSIRYNADKDWFEFAMDY | GAACAGCAAGGCTATATGCTGGGTTTTCGTAAAGTC |
| | SKFTDKAKDTYTWWTLCS | TCTGAGGCCTTTGTGAATAAACTCGTTGATGAAGGT |
| | YGTRIKTERNPAKNNLWD | AAGTTATACTTATTCCATATCTGGAACAAAGACTTT |
| | NEEVVLTDEFKKVFAAAG | AGTAAGCACTCCAAAGGTACACCTAATCTCCACACT |
| | IDVHENLKEAICALTDKK | ATTTATTGGAAAATGCTCTTCGATGAGAAAAATCTC |
| | YLEPLMRLMTLLVQMRNS | ACTGACGTCATCTACAAACTGAATGGGCAGGCTGAA |
| | ATNSETDYLLSPVADESG | GTATTCTACCGTAAAAAAAGTCTGGATCTTAATAAG |
| | MFYDSREGKETLPKDADA | ACAACTACTCACAAGGCACATGCCCCAATCACCAAT |
| | NGAYNIARKGLWTIRRIQ | AAAAATACCCAAAACGCAAAGAAGGGTAGTGTTTTC |
| | ATNCEEKVNLVLSNREWL | GATTACGATATCATCAAAAATCGTCGCTACACAGTG |
| | QFAQQKPYLNDAAAKRPA | GACAAATTCCAGTTCCACGTCCCTATCACCTTAAAT |
| | ATKKAGQAKKKKASGSGA | TTTAAGGCAACAGGTCGTAATTACATTAATGAGCAC |
| | GSPKKKRKVEDPKKKRKV | ACTCAAGAGGCAATCCGTAATAATGGCATCGAACAT |
| | (SEQ ID NO: 55) | ATCATTGGCATCGACCGTGGGGAGCGTCACTTGCTT |
| | | TACTTGTCGCTCATTGATCTGAAGGGTAATATCGTC |
| | | AAGCAGATGACCCTTAATGATATTGTCAATGAATAT |
| | | AATGGTCGGACTTATGCGACGAACTACAAGGACTTG |
| | | CTGGCAACACGGGAGGGTGAGCGTACGGACGCTCGG |
| | | CGCAACTGGCAGAAGATTGAAAATATTAAAGAAATC |
| | | AAGGAAGGTTACCTTAGCCAGGTGGTGCACATCTTG |
| | | AGTAAAATGATGGTCGACTACAAGGCTATCGTTGTT |
| | | CTGGAAGACTTGAATACAGGCTTCATGCGGAATCGT |
| | | CAAAAAATCGAACGTCAAGTATATGAGAAGTTCGAA |
| | | AAAATGTTAATTGACAAGCTGAACTGCTATGTTGAC |
| | | AAACAAAAGGATGCTGACGAGACGGGCGGTGCCCTC |
| | | CACCCGCTGCAGCTGACAAACAAATTTGAGTCGTTT |
| | | CGTAAGTTAGGTAAGCAGAGTGGTTGGCTTTTTTAC |
| | | ATCCCAGCATGGAACACTTCGAAAATCGACCCAGTT |
| | | ACTGGGTTCGTGAACATGTTAGACACGCGCTACGAG |
| | | AACGCCGATAAGGCGCGGTGTTTCTTCTCGAAATTC |
| | | GATTCCATCCGGTATAACGCTGACAAAGATTGGTTT |
| | | GAGTTTGCTATGGATTACAGTAAGTTCACTGATAAA |
| | | GCGAAAGATACTTACACGTGGTGGACTCTGTGTTCC |
| | | TATGGGACGCGTATTAAAACTTTTCGTAATCCGGCT |
| | | AAGAATAATTTGTGGGATAATGAGGAGGTTGTCCTT |
| | | ACTGATGAGTTCAAGAAAGTTTTCGCAGCGGCAGGT |
| | | ATTGATGTCCATGAGAACCTTAAGGAAGCGATCTGT |
| | | GCTCTGACAGATAAAAAGTATCTTGAACCACTCATG |
| | | CGTCTCATGACCCTGCTCGTTCAAATGCGGAACTCT |
| | | GCTACTAACTCCGAAACAGACTATTTACTTTCACCA |
| | | GTTGCTGACGAGTCAGGGATGTTCTATGACTCCCGC |
| | | GAAGGGAAGGAAACACTGCCAAAAGATGCGGACGCC |
| | | AACGGTGCATATAACATTGCCCGTAAGGGCCTCTGG |
| | | ACCATCCGGCGGATTCAAGCCACCAACTGTGAGGAG |
| | | AAAGTTAACTTAGTCCTCAGTAATCGTGAATGGTTG |
| | | CAGTTTGCCCAGCAGAAACCATATCTGAATGATGCG |
| | | GCCGCAAAAAGGCCGGCGGCCACGAAAAAGGCCGGC |
| | | CAGGCAAAAAAGAAAAAGGCTAGCGGCAGCGGCGCC |
| | | GGATCCCCAAAGAAGAAAAGGAAGGTTGAAGACCCC |
| | | AAGAAAAAGAGGAAGGTGTGATAA (SEQ ID NO: |
| | | 56) |
| ABW 6 | MGHHHHHHSSGLVPRGSG | ATGGGCCACCATCATCATCATCATAGCAGCGGCCTG |
| | TMIYRENFKRKKEKIEMN | GTGCCGCGCGGCAGCGGTACCATGATCTACCGTGAG |
| | TGFNDFTNLSSVIKTLCN | AATTTTAAGCGGAAAAAGGAGAAGATTGAAATGAAC |
| | RLIPTEITAKYIKEHGVI | ACTGGGTTTAATGACTTCACTAATTTGAGTTCCGTG |
| | EADQERNMMSQELKNILN | ACCAAGACGTTATGCAACCGGTTGATCCCAACAGAA |
| | DFYRSFLNENLVKVHELD | ATTACCGCAAAGTACATTAAGGAGCATGGGGTAATT |
| | FKPLFTEMKKYLETKDNK | GAGGCGGACCAAGAACGGAACATGATGAGTCAAGAG |
| | EALEKAQDDMRKAIHDIF | CTGAAAAATATCTTGAATGACTTTTACCGGAGTTTC |
| | ESDDRYKKMFKAEITASI | CTGAACGAGAACCTTGTGAAGGTGCACGAACTTGAT |
| | LPEFILHNGAYSAEEKEE | TTCAAGCCGTTATTCACCGAGATGAAAAAGTACCTC |
| | KMQVVKMENGEMTSFSAF | GAAACAAAAGATAACAAGGAAGCACTCGAAAAGGCC |
| | FTNRENCESKEKISSSAC | CAGGACGACATGCGGAAGGCAATCCATGATATCTTT |
| | YRIVDDNAKIHEDNIRIY | GAAAGTGATGACCGCTACAAAAAAATGTTCAAGGCT |
| | KNIANKEDYEIEMIEKIE | GAGATCACGGCGTCGATTTTGCCTGAATTCATTCTT |
| | EAAGGADIRNIFSYNEDH | CATAACGGGGCATATTCAGCCGAAGAAAAGGAGGAG |
| | FAFNHFVSQDDISFYNYV | AAAATGCAAGTAGTCAAGATGTTCAATGGCTTTATG |
| | VGGINKEMNLYCQATKEK | ACGTCTTTCTCAGCATTCTTTACGAATCGTGAGAAT |
| | LSPYKLRHLHKQILCIEE | TGTTTCTCCAAAGAAAAGATCAGCTCCTCCGCATGT |
| | SLYDVPAKFNCDEDVYAA | TACCGTATTGTTGATGACAACGCGAAAATCCATTTC |
| | VNDFLNNVRTKSVIERLQ | GATAACATTCGTATTTATAAAAATATCGCCAACAAG |
| | MLGKNADSYDLDKIYISK | TTCGATTATGAAATTGAAATGATCGAGAAGATCGAA |
| | KHFTNISQTLYRDESVIN | GAGGCGGCGGGGGGTGCCGACATTCGTAATATCTTC |
| | TALTMSYIDTLPGKGKTK | TCGTACAACTTTGACCACTTTGCATTCAATCATTTC |
| | EKKAASMAKNTELISLGE | GTTAGTCAAGATGATATCTCATTCTACAATTATGTT |
| | IDKLVDKYNLCPDKAAST | GTTGGTGGTATTAACAAGTTTATGAACTTGTATTGT |
| | RSLIRSISDIVADYKANP | CAAGCCACCAAAGAGAAATTATCGCCTTATAAACTG |
| | LTMNSGIPLAENETEIAV | CGTCACCTTCACAAACAGATTCTGTGTATTGAGGAA |
| | LKEAIEPEMDIFRWCAKE | AGCCTCTATGACGTGCCAGCGAAGTTTAATTGTGAT |
| | KTDEPVDKDTDFYTELED | GAGGACGTATATGCAGCTGTCAACGATTTTCTTAAT |
| | INDEIHSIVSLYNRTRNY | AACGTTCGGACGAAATCAGTAATTGAACGCTTGCAA |
| | VTKKPYNTDKFGLYFGTS | ATGCTCGGCAAAAATGCAGACAGTTACGACCTGGAT |
| | SFASGWSESKEFTNNAIL | AAAATTTATATCTCTAAAAAGCACTTCACCAATATC |
| | LAKDDKFYLGVFNAKNKP | TCTCAAACTTTATATCGCGACTTCTCTGTGATCAAC |
| | AKSIIKGHDTIQDGDYKK | ACTGCCCTCACTATGTCTTATATCGATACTCTTCCG |
| | MVYSLLTGPNKMLPHMFI | GGTAAGGGGAAAACCAAGGAAAAAAAGGCAGCATCG |
| | SSSKAVPVYGLTDELLSD | ATGGCCAAAAACACCGAACTTATTTCGTTAGGCGAA |
| | YKKGRHLKTSKNFDIDYC | ATTGATAAGTTGGTGGATAAATATAACCTCTGTCCA |
| | HKLIDYFKHCLALYTDWD | GATAAGGCAGCTAGCACTCGTAGCCTCATTCGGTCT |
| | CFNFKFSDTESYNDIGEF | ATTAGCGACATCGTCGCTGACTACAAGGCAAACCCT |
| | YKEVAEQGYYMNWTYIGS | CTTACAATGAATAGTGGGATTCCGTTGGCAGAGAAC |
| | DDIDSLQENGQLYLFQIY | GAGACAGAAATCGCGGTGTTAAAAGAGGCGATCGAG |
| | NKDESEKSFGKPSKHTAI | CCTTTTATGGATATCTTCCGGTGGTGTGCTAAGTTT |
| | LRSLESDENVADPVIKLC | AAAACCGACGAGCCTGTCGATAAGGATACAGATTTC |
| | GGTEVFFRPKSIKTPVVH | TACACGGAGTTAGAAGACATTAACGATGAAATCCAT |
| | KKGSILVSKTYNAQEMDE | AGTATTGTCAGTCTTTATAACCGGACCCGGAATTAT |
| | NGNIITVRKCVPDDVYME | GTCACTAAAAAGCCGTACAACACAGATAAGTTCGGT |
| | LYGYYNNSGTPLSAEALK | CTGTATTTTGGCACTTCGTCGTTCGCATCGGGTTGG |
| | YKDIVDHRTAPYDIIKDR | AGCGAGAGCAAAGAGTTTACTAACAACGCAATTTTG |
| | RYTEDEFFINMPVSLNYK | TTAGCCAAGGATGACAAGTTTTACCTCGGCGTGTTC |
| | AENRRVNVNEMALKYIAQ | AACGCAAAAAACAAGCCAGCAAAATCGATTATCAAA |
| | TKDTYIIGIDRGERNLLY | GGGCATGACACAATCCAAGATGGTGATTATAAGAAA |
| | VSVIDTDGNIVEQKSLNI | ATGGTGTATTCACTGCTCACCGGGCCAAATAAGATG |
| | INNVDYQAKLKQVEIMRK | CTTCCTCACATGTTTATCTCGAGCAGTAAAGCGGTT |
| | LARQNWKQGVKIADLKKG | CCTGTTTACGGGCTCACTGACGAGCTTCTCAGCGAC |
| | YLSQAVHEVAELVIKYNG | TATAAGAAAGGTCGCCACCTTAAGACATCCAAGAAT |
| | IVVMEDLNSREKEKRSKI | TTCGACATTGATTACTGTCACAAACTTATCGATTAC |
| | ERGVYQQFETSLIKTLNY | TTCAAACATTGTCTCGCTTTGTATACTGATTGGGAT |
| | LTFKDRKPLEAGGIANGY | TGCTTCAACTTCAAATTCTCTGATACGGAGTCCTAC |
| | QLTYIPESLKNVGSQCGC | AATGATATCGGCGAGTTCTACAAAGAGGTTGCCGAG |
| | ILYVPAAYTSKIDPTTGF | CAAGGCTACTACATGAACTGGACATATATCGGGTCG |
| | VTLFKFKDISSEKAKTDE | GACGATATCGATTCGCTGCAGGAAAACGGCCAGCTC |
| | IGREDCIRYDAEKDLFAF | TATCTTTTTCAAATTTATAACAAAGATTTCAGCGAA |
| | EFDYDNFETYETCARTKW | AAGTCATTCGGTAAACCGTCTAAACATACGGCCATC |
| | CAYTYGTRVKKTERNRKF | CTGCGTAGCTTATTCAGCGATGAAAACGTGGCCGAC |
| | VSEVIIDITEEIKKTLAA | CCAGTCATTAAACTGTGTGGGGGGACCGAAGTTTTT |
| | TDINWIDSHDIKQEIIDY | TTCCGGCCGAAGTCTATTAAGACACCAGTAGTACAT |
| | ALSSHIFEMEKLTVQMRN | AAAAAAGGCAGCATCCTCGTATCCAAAACCTATAAC |
| | SLCESKDREYDKFVSPIL | GCACAAGAAATGGACGAGAATGGTAATATCATCACC |
| | NASGKFFDTDAADKSLPI | GTGCGGAAGTGTGTTCCAGACGACGTCTATATGGAG |
| | EADANDAYGIAMKGLYNV | CTCTACGGCTATTACAACAACTCTGGGACGCCTCTG |
| | LQVKNNWAEGEKFKESRL | TCCGCCGAAGCTTTGAAATACAAGGATATTGTGGAC |
| | SNEDWENFMQKRAAAKRP | CACCGCACGGCTCCGTACGACATTATCAAGGACCGG |
| | AATKKAGQAKKKKASGSG | CGTTACACCGAAGACGAATTTTTCATCAACATGCCG |
| | AGSPKKKRKVEDPKKKRK | GTGTCATTGAATTATAAAGCGGAAAACCGCCGTGTT |
| | V (SEQ ID NO: 68) | AATGTGAACGAAATGGCCTTAAAATACATCGCACAG |
| | | ACCAAGGACACCTACATCATTGGCATCGATCGGGGC |
| | | GAACGTAATCTGTTGTATGTGAGCGTTATCGATACT |
| | | GACGGCAATATCGTTGAGCAAAAGAGTCTCAATATC |
| | | ATCAATAACGTGGATTATCAAGCCAAATTAAAGCAA |
| | | GTGGAAATCATGCGTAAACTGGCCCGTCAGAATTGG |
| | | AAGCAGGGGGTAAAGATTGCAGACCTGAAAAAGGGC |
| | | TACCTGTCACAAGCGGTACATGAAGTCGCGGAACTT |
| | | GTAATTAAATACAACGGGATTGTTGTAATGGAGGAC |
| | | TTAAACTCCCGCTTCAAAGAGAAGCGTTCTAAAATT |
| | | GAACGCGGCGTCTACCAACAGTTTGAGACATCATTA |
| | | ATCAAGACATTGAATTATTTGACGTTCAAAGATCGC |
| | | AAACCGTTAGAAGCCGGGGGCATTGCGAATGGTTAT |
| | | CAATTAACTTATATTCCGGAGTCTCTTAAAAATGTG |
| | | GGCTCTCAGTGCGGCTGTATCTTGTATGTGCCAGCA |
| | | GCCTACACCTCGAAGATCGACCCTACCACTGGTTTC |
| | | GTCACCTTGTTCAAATTCAAAGACATTTCGAGCGAG |
| | | AAAGCTAAAACGGATTTTATTGGTCGGTTCGACTGC |
| | | ATCCGTTATGATGCAGAAAAGGACCTTTTCGCATTT |
| | | GAATTCGATTATGACAACTTTGAGACTTATGAGACT |
| | | TGTGCGCGTACCAAATGGTGTGCATATACATACGGG |
| | | ACTCGGGTGAAGAAAACTTTCCGGAATCGGAAATTC |
| | | GTGTCAGAGGTGATCATCGACATCACTGAAGAGATC |
| | | AAGAAGACCCTTGCAGCGACCGATATTAATTGGATT |
| | | GACAGTCACGACATCAAACAAGAGATCATCGACTAT |
| | | GCCCTTAGCAGCCATATTTTTGAAATGTTCAAATTA |
| | | ACGGTACAGATGCGTAACAGCCTTTGCGAGAGTAAA |
| | | GATCGCGAGTACGACAAGTTCGTCTCACCTATTCTC |
| | | AACGCGTCGGGCAAATTTTTCGACACCGATGCCGCT |
| | | GATAAAAGTCTGCCTATTGAAGCTGATGCGAACGAT |
| | | GCGTATGGTATTGCTATGAAAGGGTTGTATAATGTT |
| | | TTACAAGTCAAAAACAACTGGGCGGAGGGCGAGAAA |
| | | TTTAAGTTCTCCCGTTTAAGCAACGAAGATTGGTTC |
| | | AACTTCATGCAAAAGCGGGCGGCCGCAAAAAGGCCG |
| | | GCGGCCACGAAAAAGGCCGGCCAGGCAAAAAAGAAA |
| | | AAGGCTAGCGGCAGCGGCGCCGGATCCCCAAAGAAG |
| | | AAAAGGAAGGTTGAAGACCCCAAGAAAAAGAGGAAG |
| | | GTGTGATAA (SEQ ID NO: 69) |
| ABW 7 | MGHHHHHHSSGLVPRGSL | ATGGGCCACCATCATCATCATCATAGCAGCGGCCTG |
| | QMTMDYGNGQFERRAPLT | GTGCCGCGCGGCAGCCTGCAGATGACAATGGATTAC |
| | KTITLRLKPIGETRETIR | GGTAACGGTCAATTTGAGCGGCGCGCCCCGCTCACC |
| | EQKLLEQDAAFRKLVETV | AAGACAATCACTCTCCGGTTGAAACCGATCGGGGAG |
| | TPIVDDCIRKIADNALCH | ACCCGTGAGACGATTCGCGAGCAAAAGCTCCTCGAA |
| | FGTEYDESCLGNAISKND | CAAGATGCTGCATTCCGTAAACTTGTTGAAACTGTC |
| | SKAIKKETEKVEKLLAKV | ACCCCTATCGTGGATGATTGTATCCGGAAAATTGCT |
| | LTENLPDGLRKVNDINSA | GACAACGCTTTGTGTCATTTTGGCACGGAATATGAT |
| | AFIQDTLTSFVQDDADKR | TTCTCCTGTTTAGGTAATGCCATCTCAAAAAATGAC |
| | VLIQELKGKTVLMQRFLT | AGCAAAGCGATTAAGAAAGAGACCGAAAAAGTAGAG |
| | TRITALTVWLPDRVFENF | AAGCTGTTGGCCAAGGTTCTGACAGAGAACTTGCCA |
| | NIFIENAEKMRILLDSPL | GACGGTCTGCGTAAAGTCAACGATATTAACAGCGCG |
| | NEKIMKEDPDAEQYASLE | GCTTTTATTCAGGACACACTGACATCATTCGTCCAG |
| | FYGQCLSQKDIDSYNLII | GACGATGCTGACAAACGTGTGTTAATTCAAGAGTTA |
| | SGIYADDEVKNPGINEIV | AAGGGCAAAACTGTGTTAATGCAACGCTTTTTAACA |
| | KEYNQQIRGDKDESPLPK | ACCCGGATTACTGCATTGACTGTATGGCTCCCTGAC |
| | LKKLHKQILMPVEKAFFV | CGGGTGTTTGAGAACTTCAACATTTTTATCGAAAAT |
| | RVLSNDSDARSILEKILK | GCTGAAAAGATGCGCATCTTGCTCGACTCACCATTG |
| | DTEMLPSKIIEAMKEADA | AATGAAAAGATCATGAAGTTCGATCCGGATGCTGAA |
| | GDIAVYGSRLHELSHVIY | CAATACGCGAGTTTGGAATTCTATGGTCAATGTCTG |
| | GDHGKLSQIIYDKESKRI | TCCCAGAAGGATATTGATTCGTACAACCTCATCATT |
| | SELMETLSPKERKESKKR | TCCGGGATTTATGCCGATGATGAGGTCAAGAACCCA |
| | LEGLEEHIRKSTYTEDEL | GGTATCAATGAAATTGTTAAGGAATACAACCAGCAA |
| | NRYAEKNVMAAYIAAVEE | ATTCGCGGGGATAAGGATGAGTCACCTTTACCTAAA |
| | SCAEIMRKEKDLRTLLSK | CTGAAAAAGTTGCATAAACAAATTTTGATGCCTGTC |
| | EDVKIRGNRHNTLIVKNY | GAGAAGGCATTTTTCGTTCGGGTACTCAGTAATGAT |
| | FNAWTVERNLIRILRRKS | TCTGATGCTCGTTCAATTTTAGAAAAAATCTTGAAG |
| | EAEIDSDFYDVLDDSVEV | GATACTGAGATGTTGCCTTCTAAGATCATTGAAGCG |
| | LSLTYKGENLCRSYITKK | ATGAAAGAAGCAGACGCTGGGGACATCGCTGTATAT |
| | IGSDLKPEIATYGSALRP | GGTTCACGTTTGCACGAGTTAAGCCACGTAATCTAT |
| | NSRWWSPGEKFNVKFHTI | GGCGATCACGGGAAGCTCTCTCAGATTATCTATGAT |
| | VRRDGRLYYFILPKGAKP | AAGGAGTCGAAACGCATCAGCGAGCTCATGGAAACG |
| | VELEDMDGDIECLQMRKI | TTATCGCCTAAGGAGCGCAAAGAGTCAAAGAAACGC |
| | PNPTIFLPKLVFKDPEAF | TTGGAGGGTCTGGAAGAACATATCCGGAAGTCGACA |
| | FRDNPEADEFVELSGMKA | TATACCTTCGACGAGCTTAATCGTTATGCGGAAAAG |
| | PVTITRETYEAYRYKLYT | AACGTCATGGCTGCCTACATCGCGGCCGTGGAGGAA |
| | VGKLRDGEVSEEEYKRAL | AGCTGCGCCGAAATTATGCGTAAGGAGAAGGACTTA |
| | LQVLTAYKEFLENRMIYA | CGCACGCTTCTTAGTAAGGAGGATGTCAAGATTCGT |
| | DLNFGFKDLEEYKDSSEF | GGTAATCGCCACAATACGTTAATTGTTAAGAACTAC |
| | IKQVETHNTFMCWAKVSS | TTCAATGCCTGGACTGTCTTCCGGAATTTGATCCGC |
| | SQLDDLVKSGNGLLFEIW | ATCCTCCGGCGGAAATCCGAGGCGGAGATCGACTCA |
| | SERLESYYKYGNEKVLRG | GATTTCTATGACGTCTTGGATGACTCTGTGGAAGTT |
| | YEGVLLSILKDENLVSMR | TTATCGCTCACATATAAAGGTGAAAACTTGTGCCGG |
| | TLLNSRPMLVYRPKESSK | TCTTACATTACGAAGAAGATCGGGAGCGATTTAAAG |
| | PMVVHRDGSRVVDREDKD | CCAGAGATTGCTACCTATGGTTCCGCCTTGCGCCCT |
| | GKYIPPEVHDELYRFENN | AATTCACGGTGGTGGTCACCGGGCGAGAAGTTTAAC |
| | LLIKEKLGEKARKILDNK | GTAAAGTTCCACACCATTGTTCGCCGGGACGGTCGC |
| | KVKVKVLESERVKWSKFY | CTTTATTATTTCATCTTGCCGAAAGGTGCCAAACCT |
| | DEQFAVTFSVKKNADCLD | GTCGAGCTCGAAGATATGGATGGGGACATCGAATGC |
| | TTKDLNAEVMEQYSESNR | TTGCAAATGCGCAAGATTCCGAATCCGACTATTTTC |
| | LILIRNTTDILYYLVLDK | CTTCCAAAATTGGTTTTCAAGGACCCAGAGGCCTTC |
| | NGKVLKQRSLNIINDGAR | TTCCGCGACAATCCAGAGGCAGATGAATTCGTTTTT |
| | DVDWKERFRQVTKDRNEG | CTTTCGGGTATGAAAGCTCCAGTGACCATCACGCGT |
| | YNEWDYSRTSNDLKEVYL | GAAACCTATGAGGCGTATCGCTACAAACTTTATACA |
| | NYALKEIAEAVIEYNAIL | GTTGGGAAGTTACGCGACGGTGAAGTGAGCGAAGAA |
| | IIEKMSNAFKDKYSELDD | GAGTATAAACGTGCGTTGTTACAAGTATTGACCGCC |
| | VTFKGFETKLLAKLSDLH | TATAAGGAATTCTTAGAGAATCGGATGATCTACGCA |
| | FRGIKDGEPCSFTNPLQL | GATCTGAACTTTGGCTTTAAAGATCTCGAAGAATAC |
| | CQNDSNKILQDGVIFMVP | AAAGACTCGTCAGAATTTATCAAACAAGTCGAAACT |
| | NSMTRSLDPDTGFIFAIN | CACAACACTTTTATGTGCTGGGCTAAGGTCAGTAGC |
| | DHNIRTKKAKLNELSKED | AGTCAGCTCGACGACCTGGTCAAGAGCGGGAACGGG |
| | QLKVSSEGCLIMKYSGDS | TTACTGTTCGAAATCTGGTCAGAACGGTTGGAGTCC |
| | LPTHNTDNRVWNCCCNHP | TATTACAAATATGGCAACGAGAAGGTGCTGCGTGGG |
| | ITNYDRETKKVEFIEEPV | TACGAGGGCGTTCTTTTGAGTATCCTTAAGGACGAG |
| | EELSRVLEENGIETDTEL | AACCTCGTGAGCATGCGGACGCTGCTTAATTCTCGG |
| | NKLNERENVPGKVVDAIY | CCGATGCTCGTCTACCGCCCTAAAGAATCATCCAAG |
| | SLVLNYLRGTVSGVAGQR | CCGATGGTCGTTCACCGGGACGGTAGCCGCGTCGTT |
| | AVYYSPVTGKKYDISFIQ | GATCGGTTCGATAAGGATGGGAAGTATATTCCACCA |
| | AMNLNRKCDYYRIGSKER | GAGGTACACGACGAATTATACCGGTTCTTTAACAAT |
| | GEWTDFVAQLINAAAKRP | TTGCTTATTAAGGAAAAGCTCGGCGAGAAAGCGCGC |
| | AATKKAGQAKKKKASGSG | AAAATTTTAGACAACAAAAAAGTAAAAGTAAAGGTA |
| | AGSPKKKRKVEDPKKKRK | TTGGAATCTGAACGTGTAAAGTGGTCAAAGTTTTAT |
| | V (SEQ ID NO: 81) | GATGAACAGTTTGCAGTTACATTCTCTGTTAAAAAG |
| | | AATGCAGACTGTCTGGATACCACGAAAGATCTCAAT |
| | | GCCGAAGTTATGGAGCAGTATTCCGAATCGAACCGG |
| | | CTTATCCTGATCCGCAATACCACTGACATCTTGTAT |
| | | TATCTTGTACTTGATAAGAATGGGAAAGTGCTGAAA |
| | | CAACGCTCATTGAATATCATTAACGACGGGGCTCGC |
| | | GACGTTGATTGGAAAGAGCGTTTTCGGCAGGTAACA |
| | | AAAGATCGTAACGAAGGCTATAACGAGTGGGACTAC |
| | | TCGCGGACTAGCAACGATTTGAAAGAGGTCTATCTG |
| | | AATTATGCATTGAAGGAGATTGCCGAAGCGGTAATC |
| | | GAATACAACGCAATTTTGATTATTGAAAAAATGTCG |
| | | AATGCCTTCAAGGATAAGTACTCCTTTTTGGATGAT |
| | | GTTACCTTCAAAGGTTTTGAGACCAAACTTCTTGCG |
| | | AAGCTCTCTGACTTGCATTTCCGGGGTATTAAAGAT |
| | | GGGGAGCCATGTTCGTTTACGAACCCGTTACAGTTA |
| | | TGTCAGAACGACTCAAACAAAATTTTACAAGACGGT |
| | | GTGATTTTCATGGTCCCTAACAGCATGACGCGCAGT |
| | | CTGGACCCTGACACTGGGTTCATTTTTGCGATTAAC |
| | | GATCACAACATCCGCACTAAGAAAGCGAAGTTAAAC |
| | | TTCCTTAGTAAATTCGATCAGCTGAAAGTGTCATCA |
| | | GAGGGCTGTTTAATCATGAAATATTCGGGGGACTCC |
| | | CTTCCTACACACAACACAGATAATCGTGTATGGAAC |
| | | TGTTGTTGCAATCACCCGATCACCAACTACGACCGC |
| | | GAGACGAAAAAGGTCGAATTCATCGAGGAGCCAGTG |
| | | GAAGAGTTGAGTCGCGTCTTAGAAGAGAATGGGATT |
| | | GAGACAGATACGGAACTTAACAAGCTTAACGAGCGC |
| | | GAGAATGTTCCGGGCAAGGTAGTAGATGCCATCTAT |
| | | TCTCTGGTGTTGAATTACTTGCGTGGTACCGTGTCC |
| | | GGCGTTGCAGGCCAACGGGCGGTCTACTATTCCCCT |
| | | GTGACGGGGAAAAAATATGATATTTCGTTTATCCAA |
| | | GCAATGAATCTGAATCGTAAGTGCGATTACTACCGG |
| | | ATCGGGAGCAAAGAACGCGGCGAATGGACGGATTTT |
| | | GTAGCGCAGTTAATTAACGCGGCCGCAAAAAGGCCG |
| | | GCGGCCACGAAAAAGGCCGGCCAGGCAAAAAAGAAA |
| | | AAGGCTAGCGGCAGCGGCGCCGGATCCCCAAAGAAG |
| | | AAAAGGAAGGTTGAAGACCCCAAGAAAAAGAGGAAG |
| | | GTGTGATAA (SEQ ID NO: 82) |
| ABW8 | MGHHHHHHSSGLVPRGSG | ATGGGCCACCATCATCATCATCATAGCAGCGGCCTG |
| | TMCYDLNNIKTKLREREV | GTGCCGCGCGGCAGCGGTACCATGTGCTACGACTTA |
| | ETMGNNMDNSFEPFIGGN | AACAACATCAAGACAAAGTTACGTGAACGCGAAGTC |
| | SVSKTLRNELRVGSEYTG | GAAACTATGGGCAATAACATGGATAATAGCTTCGAG |
| | KHIKECAIIAEDAVKAEN | CCTTTTATTGGCGGTAATAGTGTCTCTAAAACACTT |
| | QYIVKEMMDDFYRDFINR | CGGAATGAGCTGCGTGTAGGTTCCGAATATACTGGT |
| | KLDALQGINWEQLEDIMK | AAACACATTAAAGAGTGCGCGATCATTGCAGAGGAC |
| | KAKLDKSNKVSKELDKIQ | GCCGTGAAGGCGGAGAACCAGTACATCGTAAAAGAG |
| | ESTRKEIGKIFSSDPIYK | ATGATGGACGACTTTTACCGTGACTTCATTAATCGC |
| | DMLKADMISKILPEYIVD | AAACTTGACGCCTTGCAGGGTATTAATTGGGAGCAG |
| | KYGDAASRIEAVKVFYGF | CTTTTTGACATTATGAAGAAGGCGAAATTGGATAAG |
| | SGYFIDEWASRKNVESDK | TCGAATAAAGTCAGCAAAGAGTTAGACAAGATTCAA |
| | NIASAIPHRIVNVNARIH | GAGTCTACGCGGAAAGAAATCGGGAAAATCTTCTCA |
| | LDNITAFNRIAEIAGDEV | TCCGATCCAATCTATAAAGACATGCTCAAAGCGGAC |
| | AGIAEDACAYLQNMSLED | ATGATCAGCAAAATTCTGCCAGAGTATATTGTCGAC |
| | VFTGACYGEFICQKDIDR | AAATACGGTGATGCAGCCTCGCGGATCGAAGCTGTA |
| | YNNICGVINQHMNQYCQN | AAGGTGTTTTACGGCTTTTCGGGTTATTTTATCGAC |
| | KKISRSKFKMERLHKQIL | TTCTGGGCATCGCGCAAGAACGTCTTCTCAGATAAG |
| | CRSESGFEIPIGFQTDGE | AACATCGCGTCGGCCATTCCGCACCGGATTGTCAAT |
| | VIDAINSESTILEEKDIL | GTGAACGCTCGGATCCATCTGGACAACATCACGGCC |
| | DRLRTLSQEVTGYDMERI | TTCAACCGTATCGCAGAAATTGCAGGGGATGAAGTC |
| | YVSSKAFESVSKYIDHKW | GCCGGCATTGCTGAAGATGCTTGTGCTTACCTGCAG |
| | DVIASSMYNYFSGAVRGK | AATATGAGCTTAGAGGATGTATTCACGGGGGCCTGC |
| | DDKKDVKIQTEIKKIKSC | TACGGTGAGTTCATCTGTCAGAAGGATATTGATCGT |
| | SLLDLKKLVDMYYKMDGM | TACAATAACATTTGCGGTGTTATCAACCAGCACATG |
| | CLEHEATEYVAGITEILV | AATCAATACTGCCAAAACAAAAAGATCTCACGCTCA |
| | DENYKTFDMDDSVKMIQN | AAATTTAAGATGGAACGTCTGCACAAACAGATCTTA |
| | EHMINEIKEYLDTYMSIY | TGTCGCTCTGAGAGTGGTTTTGAGATCCCGATTGGG |
| | HWAKDEMIDELVDRDMEF | TTTCAAACCGACGGGGAGGTAATCGATGCTATCAAC |
| | YSELDEIYYDLSDIVPLY | TCCTTTTCTACGATTCTTGAAGAGAAAGATATCTTG |
| | NKVRNYVTQKPYSQDKIK | GATCGTCTGCGCACTTTGTCGCAGGAGGTAACAGGT |
| | LNFGSPTLANGWSKSKEF | TATGACATGGAGCGTATCTATGTAAGTTCCAAGGCG |
| | DNNVVVLLRDEKIYLAIL | TTTGAGTCTGTATCAAAGTACATCGATCACAAATGG |
| | NVGNKPSKDIMAGEDRRR | GACGTAATTGCTTCTTCCATGTACAATTACTTTTCT |
| | SDTDYKKMNYYLLPGASK | GGGGCTGTTCGTGGGAAGGACGACAAGAAAGATGTC |
| | TLPHVFISSNAWKKSHGI | AAGATTCAGACGGAAATTAAAAAGATTAAGTCATGT |
| | PDEIMYGYNQNKHLKSSP | TCGTTATTGGACCTCAAAAAGCTGGTAGATATGTAT |
| | NFDLEFCRKLIDYYKECI | TATAAAATGGATGGGATGTGTTTAGAGCACGAAGCG |
| | DSYPNYQIENFKFAATET | ACGGAGTACGTGGCAGGTATTACGGAGATCCTGGTT |
| | YNDISEFYKDVERQGYKI | GACTTTAACTATAAGACCTTCGACATGGATGATTCC |
| | EWSYISEDDINQMDRDGQ | GTTAAGATGATTCAAAATGAGCACATGATTAATGAA |
| | IYLFQIYNKDFAPNSKGM | ATTAAAGAATATTTAGATACCTATATGTCTATCTAT |
| | QNLHTLYLKNIFSEENLS | CATTGGGCGAAGGACTTTATGATCGATGAGCTCGTA |
| | DVVIKLNGEAELFFRKSS | GATCGCGACATGGAATTCTACAGTGAGCTCGATGAA |
| | IQHKRGHKKGSVLVNKTY | ATCTATTATGATTTGTCCGACATCGTACCACTGTAT |
| | KTTEKTENGQGEIEVIES | AATAAAGTCCGCAACTACGTCACGCAAAAACCGTAT |
| | VPDQCYLELVKYWSEGGV | TCCCAGGATAAAATCAAGTTAAACTTTGGCAGCCCA |
| | GQLSEEASKYKDKVSHYA | ACCTTAGCAAACGGTTGGAGCAAGTCGAAAGAATTT |
| | ATMDIVKDRRYTEDKFFI | GATAACAACGTTGTAGTATTGTTGCGTGACGAAAAG |
| | HMPITINFKADNRNNVNE | ATTTATCTGGCCATCTTAAATGTGGGGAATAAACCG |
| | KVLKFIAENDDLHVIGID | TCAAAGGATATCATGGCGGGCGAAGACCGTCGTCGC |
| | RGERNLLYVSVIDSRGRI | TCCGATACTGATTACAAGAAAATGAATTACTATCTG |
| | VEQKSENIVENYESSKNV | CTCCCTGGGGCAAGCAAAACCCTGCCACACGTTTTT |
| | IRRHDYRGKLVNKEHYRN | ATCTCTTCAAATGCATGGAAGAAATCCCACGGTATC |
| | EARKSWKEIGKIKEIKEG | CCTGACGAGATTATGTACGGCTATAACCAAAATAAG |
| | YLSQVIHEISKLVLKYNA | CATTTAAAATCTTCGCCAAACTTCGACTTAGAGTTT |
| | IIVMEDLNYGFKRGREKV | TGTCGCAAGCTGATCGATTATTACAAAGAATGTATT |
| | ERQVYQKFETMLINKLAY | GACAGCTATCCTAACTATCAGATCTTCAATTTCAAA |
| | LVDKSRAVDEPGGLLKGY | TTCGCCGCTACGGAAACTTACAACGATATTTCGGAG |
| | QLTYVPDNLGELGSQCGI | TTCTACAAAGATGTTGAACGTCAGGGGTACAAGATT |
| | IFYVPAAYTSKIDPVTGF | GAATGGTCGTACATTTCCGAGGACGATATTAATCAG |
| | VDVEDFKAYSNAEARLDE | ATGGATCGTGACGGCCAGATTTATCTTTTTCAAATC |
| | INKLDCIRYDAPRNKFEI | TACAACAAGGATTTTGCCCCAAACTCTAAGGGCATG |
| | AFDYGNERTHHTTLAKTS | CAGAATTTACATACACTCTATTTAAAAAATATTTTT |
| | WTIFIHGDRIKKERGSYG | TCAGAGGAAAACCTCTCTGATGTCGTCATTAAACTG |
| | WKDEIIDIEARIRKLFED | AATGGCGAGGCTGAGCTCTTCTTCCGCAAGAGCTCG |
| | TDIEYADGHNLIGDINEL | ATCCAACATAAACGCGGTCATAAGAAGGGTAGTGTG |
| | ESPIQKKFVGELFDIIRE | TTGGTAAATAAGACCTATAAAACCACAGAAAAAACT |
| | TVQLRNSKSEKYDGTEKE | GAAAATGGTCAAGGCGAAATTGAAGTAATCGAGAGC |
| | YDKIISPVMDEEGVFFTT | GTGCCGGACCAGTGTTACCTGGAGCTTGTTAAGTAC |
| | DSYIRADGTELPKDADAN | TGGTCAGAGGGTGGTGTAGGTCAGTIGTCAGAAGAG |
| | GAYCIALKGLYDVLAVKK | GCTTCCAAATACAAAGATAAAGTCAGCCACTACGCT |
| | YWKEGEKFDRKLLAITNY | GCAACAATGGATATTGTCAAGGACCGGCGGTACACG |
| | NWFDFIQNRRFAAAKRPA | GAGGATAAGTTCTTTATTCACATGCCGATTACGATT |
| | ATKKAGQAKKKKASGSGA | AATTTTAAAGCTGATAACCGGAACAATGTCAACGAG |
| | GSPKKKRKVEDPKKKRKV | AAAGTGCTGAAGTTTATTGCAGAAAACGATGATCTC |
| | (SEQ ID NO: 94) | CACGTTATTGGTATTGACCGTGGGGAACGTAATCTC |
| | | CTGTACGTCTCAGTAATTGATTCACGTGGGCGTATT |
| | | GTTGAGCAGAAGTCGTTTAATATTGTTGAGAATTAC |
| | | GAGAGCAGTAAAAATGTGATCCGCCGCCATGATTAT |
| | | CGTGGGAAATTAGTAAATAAAGAGCACTATCGTAAT |
| | | GAGGCACGTAAGAGCTGGAAAGAAATCGGCAAAATC |
| | | AAGGAGATCAAAGAAGGTTATCTCAGTCAAGTTATC |
| | | CATGAGATTAGTAAGTTGGTATTAAAGTATAACGCC |
| | | ATCATCGTGATGGAAGATCTTAATTATGGCTTCAAA |
| | | CGCGGGCGGTTTAAAGTCGAGCGGCAGGTATACCAG |
| | | AAGTTCGAGACCATGCTTATTAACAAATTAGCCTAC |
| | | TTAGTGGACAAATCACGCGCGGTAGACGAACCGGGT |
| | | GGGTTATTAAAAGGCTACCAGCTGACATACGTGCCA |
| | | GATAACTTGGGTGAACTGGGGTCCCAGTGCGGGATC |
| | | ATTTTTTATGTGCCAGCAGCATACACTTCGAAAATC |
| | | GATCCTGTTACGGGCTTTGTAGACGTGTTTGATTTT |
| | | AAGGCATACTCCAATGCCGAAGCACGTTTAGATTTC |
| | | ATCAATAAACTGGACTGCATCCGGTATGACGCGCCG |
| | | CGTAACAAGTTTGAAATTGCTTTCGACTACGGTAAC |
| | | TTCCGGACTCATCATACAACCCTTGCAAAGACTAGC |
| | | TGGACTATTTTTATTCACGGCGACCGTATTAAAAAG |
| | | GAGCGCGGTTCTTACGGCTGGAAGGACGAAATTATC |
| | | GATATCGAGGCCCGTATTCGTAAGCTGTTTGAAGAC |
| | | ACAGACATCGAATACGCCGATGGTCACAATTTGATC |
| | | GGTGACATTAACGAGCTCGAGAGTCCAATTCAAAAG |
| | | AAATTCGTTGGTGAGCTGTTCGACATTATCCGTTTC |
| | | ACTGTCCAACTGCGCAACAGCAAAAGTGAGAAATAT |
| | | GACGGCACCGAAAAGGAGTATGACAAAATTATTTCG |
| | | CCGGTAATGGACGAGGAGGGGGTTTTCTTTACAACC |
| | | GACAGTTATATCCGCGCAGATGGTACTGAATTACCT |
| | | AAAGATGCTGATGCTAACGGGGCCTATTGTATCGCG |
| | | CTGAAGGGTCTTTACGACGTGCTCGCGGTAAAGAAA |
| | | TATTGGAAGGAGGGGGAGAAGTTCGATCGGAAGTTA |
| | | CTTGCCATCACCAATTACAACTGGTTTGATTTCATT |
| | | CAGAATCGTCGCTTCGCGGCCGCAAAAAGGCCGGCG |
| | | GCCACGAAAAAGGCCGGCCAGGCAAAAAAGAAAAAG |
| | | GCTAGCGGCAGCGGCGCCGGATCCCCAAAGAAGAAA |
| | | AGGAAGGTTGAAGACCCCAAGAAAAAGAGGAAGGTG |
| | | TGATAA (SEQ ID NO: 95) |
| ABW9 | MGHHHHHHSSGLVPRGSG | ATGGGGCATCACCACCACCACCACTCGTCGGGTCTT |
| | TMSDRLDVLTNQYPLSKT | GTTCCACGTGGTTCTGGTACCATGTCTGATCGCCTG |
| | LRFELKPVGATADWIRKH | GACGTGCTTACTAACCAATACCCATTATCGAAAACT |
| | NVIRYHNGKLVGKDAIRE | TTGCGCTTCGAATTGAAGCCGGTTGGAGCCACAGCT |
| | QNYKYLKKMLDEMHRLFL | GACTGGATTCGCAAACACAACGTTATCCGCTATCAT |
| | QQALVLEPNSNQAQELTA | AATGGTAAACTGGTTGGAAAGGATGCGATCCGTTTT |
| | LLRAIENNYCNNNDLLAG | CAAAATTATAAGTATCTGAAGAAAATGCTTGATGAG |
| | DYPSLSTDKTIKISNGLS | ATGCATCGCTTATTTCTTCAGCAAGCACTGGTGTTG |
| | KLTTDLFDKKFEDWAYQY | GAGCCAAATAGCAACCAGGCGCAGGAGTTGACCGCA |
| | KEDMPNFWRQDIAELEQK | CTGCTGCGTGCTATTGAGAATAATTATTGCAACAAC |
| | LQVSANAKDQKFYKGIIK | AACGACCTGCTGGCGGGCGATTATCCCAGCCTCTCT |
| | KLKNKIQKSELKAETHKG | ACCGATAAGACCATTAAAATCAGCAACGGCCTTAGC |
| | LYSPTESLQLLEWLVRRG | AAGCTGACCACGGATCTGTTCGATAAGAAGTTCGAA |
| | DIKLTYLEIGKENEKLNE | GACTGGGCATACCAATACAAAGAAGATATGCCCAAT |
| | LVPLVELKDIHRNENNFA | TTCTGGCGTCAAGATATTGCGGAATTAGAGCAAAAG |
| | TYLSGFSKNRENVYSTKE | CTTCAGGTGAGTGCGAACGCAAAAGATCAAAAGTTC |
| | DRRSGYKATSVIARTFEQ | TACAAAGGGATCATCAAGAAGCTGAAGAATAAGATC |
| | NLMFCLGNIAKWHKVTEF | CAGAAGTCTGAACTGAAAGCGGAAACGCACAAGGGC |
| | INQANNYELLQEHGIDWN | TTATACTCACCTACGGAGTCACTGCAACTGCTGGAG |
| | KQIAALEHKLDVCLAEFF | TGGCTGGTACGTCGTGGCGATATTAAACTGACTTAC |
| | ALNNFSQTLAQQGIEKYN | TTAGAGATTGGTAAAGAGAACGAGAAACTTAATGAA |
| | QVLAGIAEIAGQPKTQGL | CTGGTCCCGCTGGTCGAACTTAAGGACATTCATCGC |
| | NELINLARQKLSAKRSQL | AATTTCAATAATTTCGCCACATATCTTTCTGGCTTC |
| | PTLQLLYKQILSKGDKPF | AGCAAGAATCGTGAGAATGTGTACTCAACCAAATTT |
| | IDDEKSDQELIAELNEFV | GATCGTCGTTCGGGTTATAAAGCCACCAGTGTAATC |
| | SSQIHGEHGAIKLINHEL | GCACGCACGTTCGAACAGAATTTAATGTTCTGTCTT |
| | ESFINEARAAQQQIYVPK | GGTAACATTGCCAAGTGGCACAAGGTGACAGAATTC |
| | DKLTELSLLLTGSWQAIN | ATCAACCAGGCGAACAATTACGAGCTCCTGCAGGAG |
| | QWRYKLFDQKQLDKQQKQ | CACGGCATCGATTGGAATAAGCAAATTGCCGCGCTG |
| | YSFSLAQVERWLATEVEQ | GAACACAAACTGGACGTGTGTCTCGCAGAGTTCTTC |
| | QNFYQTEKERQQHKDTQP | GCGCTTAATAACTTCTCACAAACCCTTGCACAACAG |
| | ANVTTSSDGHSILTAFEQ | GGTATCGAAAAGTATAACCAGGTCTTGGCCGGCATC |
| | QVQTLLTNICVAAEKYRQ | GCCGAGATTGCAGGCCAACCCAAGACCCAGGGCCTG |
| | LSDNLTAIDKQRESESSK | AACGAACTCATTAACCTGGCCCGTCAGAAATTGTCT |
| | GFEQIAVIKTLLDACNEL | GCCAAACGCTCACAACTGCCTACGTTGCAACTCCTT |
| | NHFLARFTVNKKDKLPED | TACAAACAAATCTTAAGCAAGGGTGATAAGCCATTC |
| | RAEFWYEKLQAYIDAFPI | ATCGACGATTTTAAAAGCGACCAAGAGTTGATCGCC |
| | YELYNKVRNYLSKKPEST | GAATTAAATGAGTTTGTAAGCAGCCAGATTCACGGA |
| | EKVKINFDNSHFLSGWTA | GAGCATGGTGCAATCAAATTAATTAATCACGAACTT |
| | DYERHSALLEKENENYLL | GAAAGCTTTATCAATGAAGCCCGTGCAGCGCAGCAA |
| | GVVNENLSSEEEEKLKLV | CAGATTTATGTGCCCAAGGACAAGCTTACCGAATTA |
| | GGEEHAKRFIYDFQKIDN | AGTCTTCTCTTAACGGGCAGTTGGCAAGCTATTAAT |
| | SNPPRVFIRSKGSSFAPA | CAATGGCGTTACAAACTGTTCGACCAGAAACAGCTG |
| | VEKYQLPIGDIIDIYDQG | GATAAACAACAGAAACAATATTCATTTAGCCTGGCC |
| | KFKTEHKKKNEAEFKDSL | CAGGTTGAACGCTGGCTGGCAACTGAGGTTGAGCAA |
| | VRLIDYFKLGFSRHDSYK | CAAAACTTCTACCAAACCGAAAAGGAGCGCCAGCAG |
| | HYPFKWKASHQYSDIAEF | CATAAAGATACGCAGCCGGCGAACGTCACCACCAGC |
| | YAHTASFCYTLKEENINF | AGCGATGGACACAGCATTTTAACAGCATTTGAGCAA |
| | NVLRELSSAGKVYLFEIY | CAGGTGCAGACCTTATTAACCAACATCTGTGTTGCT |
| | NKDFSKNKRGQGRDNLHT | GCCGAGAAATATCGCCAATTAAGTGATAATCTCACA |
| | SYWKLLESAENLKDVVLK | GCCATCGATAAACAACGCGAGAGCGAATCAAGTAAG |
| | LNGQAEIFYRPASLAETK | GGATTCGAGCAAATCGCGGTGATTAAAACCTTGCTG |
| | AYTHKKGEVLKHKAYSKV | GACGCGTGTAACGAGCTGAATCACTTTCTGGCACGC |
| | WEALDSPIGTRLSWDDAL | TTCACGGTCAACAAGAAGGACAAACTCCCCGAAGAT |
| | KIPSITEKTNHNNQRVVQ | CGCGCAGAATTTTGGTATGAAAAGTTACAAGCGTAC |
| | YNGQEIGRKAEFAIIKNR | ATTGACGCGTTTCCGATCTACGAGCTGTATAATAAA |
| | RYSVDKFLFHCPITLNEK | GTGCGTAATTACTTAAGCAAGAAGCCGTTTAGCACT |
| | ANGQDNINARVNQFLANN | GAGAAAGTCAAAATTAATTTTGACAATTCCCATTTC |
| | KKINIIGIDRGEKHLLYI | CTGTCGGGTTGGACGGCGGACTATGAGCGTCACAGC |
| | SVINQQGEVLHQESENTI | GCCTTATTATTCAAATTTAATGAAAATTACCTGCTG |
| | TNSYQTANGEKRQVVTDY | GGTGTAGTGAATGAGAACTTAAGCAGCGAGGAAGAA |
| | HQKLDMSEDKRDKARKSW | GAAAAGCTGAAGCTCGTGGGCGGCGAAGAACATGCC |
| | STIENIKELKAGYLSHVV | AAGCGCTTCATTTATGATTTTCAGAAAATCGACAAC |
| | HRLAQLIIEFNAIVALED | TCAAACCCACCGCGCGTTTTCATTCGTAGCAAGGGG |
| | LNHGFKRGRFKIEKQVYQ | TCATCGTTCGCACCTGCGGTCGAAAAGTATCAGTTA |
| | KFEKALIDKLSYLAFKDR | CCGATTGGCGATATCATTGACATTTACGATCAGGGT |
| | TSCLETGHYLNAFQLTSK | AAATTTAAGACAGAACACAAGAAGAAGAATGAGGCC |
| | FKGFNNLGKQSGILFYVN | GAGTTTAAAGACAGTCTGGTACGTTTGATCGATTAT |
| | ADYTSTTDPLTGYIKNVY | TTTAAGCTGGGCTTCTCTCGCCATGACAGCTATAAG |
| | KTYSSVKDSTEFWQRENS | CACTACCCATTCAAGTGGAAAGCCAGTCATCAATAT |
| | IRYIASENRFEFSYDLAD | AGCGACATTGCGGAATTTTACGCTCATACCGCCTCA |
| | LKQKSLESKTKQTPLAKT | TTTTGTTACACGCTTAAGGAAGAAAACATCAATTTT |
| | QWTVSSHVTRSYYNQQTK | AACGTTCTGCGTGAGTTGTCGTCGGCGGGCAAAGTA |
| | QHELFEVTARIQQLLSKA | TATCTCTTCGAAATTTACAATAAGGATTTCTCAAAG |
| | EISYQHQNDLIPALASCQ | AACAAGCGCGGCCAAGGACGCGACAACTTGCATACC |
| | SKALHKELIWLENSILTM | AGTTATTGGAAGTTGCTGTTCTCGGCTGAGAACCTG |
| | RVTDSSKPSATSENDFIL | AAGGATGTTGTGCTGAAATTAAACGGCCAAGCGGAG |
| | SPVAPYFDSRNLNKQLPE | ATCTTTTACCGCCCAGCGTCTTTGGCCGAAACCAAG |
| | NGDANGAYNIARKGIMLL | GCCTACACCCATAAGAAAGGGGAAGTACTGAAACAT |
| | ERIGDFVPEGNKKYPDLL | AAGGCTTATAGCAAAGTGTGGGAAGCCCTGGATTCT |
| | IRNNDWQNFVQRPEMVNK | CCCATTGGCACCCGCCTGAGCTGGGACGATGCTTTA |
| | QKKKLVKLKTEYSNGSLF | AAGATCCCGTCTATTACCGAGAAGACCAATCACAAT |
| | NDLAFKAAAKRPAATKKA | AATCAGCGTGTTGTCCAGTACAACGGCCAAGAAATT |
| | GQAKKKKASGSGAGSPKK | GGCCGCAAAGCGGAGTTCGCTATTATCAAGAACCGC |
| | KRKVEDPKKKRKV (SEQ | CGTTATTCCGTCGATAAATTCCTCTTTCACTGCCCG |
| | ID NO: 107) | ATTACACTCAACTTCAAGGCGAACGGCCAGGACAAC |
| | | ATTAACGCACGCGTTAATCAATTCCTGGCAAATAAC |
| | | AAGAAGATCAACATTATTGGAATTGACCGTGGTGAA |
| | | AAGCATTTACTGTATATCAGCGTGATTAATCAACAA |
| | | GGCGAAGTCCTGCATCAGGAAAGCTTCAATACAATC |
| | | ACGAATTCATATCAGACCGCCAATGGCGAGAAACGC |
| | | CAAGTAGTCACTGACTATCACCAGAAGTTGGACATG |
| | | AGCGAGGACAAACGCGATAAAGCACGTAAGAGCTGG |
| | | AGTACAATCGAAAATATCAAAGAGCTGAAGGCGGGG |
| | | TATCTGAGCCACGTTGTACATCGCCTCGCGCAACTG |
| | | ATTATCGAATTTAATGCCATTGTTGCGTTGGAAGAT |
| | | CTTAACCACGGGTTCAAACGCGGACGTTTTAAAATC |
| | | GAAAAGCAAGTGTATCAGAAGTTCGAAAAGGCGCTG |
| | | ATCGACAAATTGAGCTACTTAGCGTTTAAGGATCGC |
| | | ACGTCGTGTCTGGAAACTGGACATTACTTGAATGCC |
| | | TTTCAATTAACCTCAAAGTTCAAAGGCTTTAACAAC |
| | | CTTGGCAAGCAATCCGGGATTTTGTTCTACGTTAAC |
| | | GCCGATTACACGAGCACCACGGATCCCTTAACAGGC |
| | | TATATTAAGAACGTATACAAAACCTACTCCTCGGTG |
| | | AAGGATTCGACCGAATTTTGGCAGCGCTTTAACTCT |
| | | ATCCGCTATATTGCGAGCGAGAACCGTTTTGAATTT |
| | | AGCTACGACTTAGCGGACCTGAAACAGAAGTCGCTC |
| | | GAGAGTAAAACCAAACAGACCCCTCTCGCCAAGACC |
| | | CAATGGACGGTCTCTAGCCACGTTACCCGTTCCTAT |
| | | TACAACCAGCAGACGAAGCAACATGAGTTATTCGAA |
| | | GTGACAGCGCGCATTCAGCAATTGCTTAGCAAAGCA |
| | | GAAATCAGCTATCAACATCAAAACGACTTGATCCCT |
| | | GCGTTAGCATCATGTCAAAGTAAGGCGTTACACAAG |
| | | GAGTTGATTTGGCTGTTCAACAGCATCCTGACTATG |
| | | CGCGTCACGGACTCAAGCAAACCGTCCGCGACCTCG |
| | | GAGAATGATTTTATCCTGAGCCCGGTAGCGCCGTAC |
| | | TTCGACTCCCGCAATCTGAATAAGCAGCTGCCGGAA |
| | | AACGGCGACGCGAACGGCGCATACAATATCGCTCGT |
| | | AAAGGTATCATGCTTCTGGAACGTATCGGGGACTTC |
| | | GTCCCGGAAGGTAACAAGAAGTACCCCGATTTACTG |
| | | ATCCGCAATAATGACTGGCAGAATTTTGTACAACGC |
| | | CCGGAGATGGTGAACAAGCAGAAGAAGAAACTCGTG |
| | | AAGTTGAAAACGGAATACTCTAATGGCAGCCTCTTC |
| | | AATGATTTGGCGTTTAAGGCCGCAGCTAAGCGCCCC |
| | | GCCGCGACTAAGAAAGCGGGTCAAGCGAAGAAGAAG |
| | | AAAGCGTCGGGGTCGGGAGCGGGCAGTCCGAAGAAG |
| | | AAGCGTAAAGTAGAGGATCCGAAGAAGAAACGCAAA |
| | | GTATAATAA (SEQ ID NO: 108) |

In some embodiments, nuclease constructs disclosed herein can have a polypeptide sequence having at least 8500 homology to the polypeptide represented by SEQ ID NO: 94 (ABW8), 29 (ABW3), 81 (ABW7), 107 (ABW9), 3 (ABW1), 16 (ABW2), 42 (ABW4), 55 (ABW5), and/or 68 (AWBW6). In some embodiments, nuclease constructs herein can have a polynucleotide sequence at least 850% homologous to the polynucleotide encoding the polypeptide having a polynucleotide represented by SEQ ID NOs: 95-104 (ABW8 variants 1-10), 30-39 (ABW3 variants 1-10), 82-91 (ABW7 variants 1-10), 108-117 (ABW9 variants 1-10), 4-13 (ABW1 variants 1-10), 17-26 (ABW2 variants 1-10), 43-52 (ABW4 variants 1-10), 56-65 (ABW5 variants 1-10), and/or 69-78 (ABW6 variants 1-10).

In some embodiments, nuclease constructs herein having a polypeptide of at least 850% homology to the polypeptide represented SEQ ID NO: 94 (ABW8) can have increased activity and/or editing accuracy compared to other nuclease constructs. In some embodiments, nuclease constructs herein having a polypeptide of at least 85% homology to the polypeptide represented by SEQ ID NO: 94 (ABW8), 29 (ABW3), 81 (ABW7) and/or 107 (ABW9) can have increased enzymatic activity and/or editing efficiency and/or accuracy compared to other nuclease constructs such as control nuclease constructs or native sequence-containing nucleases.

In some embodiments, nuclease constructs disclosed herein having a polynucleotide encoding a polypeptide having a polynucleotide of at least 85% homology to a polynucleotide represented by SEQ ID NOs: 95-104 (ABW8 variants 1-10) can have increased enzymatic activity and/or editing efficiency and/or accuracy compared to control nuclease constructs or nuclease constructs having native sequences. In some embodiments, nuclease constructs disclosed herein having a polynucleotide encoding a polypeptide of at least 85% homology to a polynucleotide represented by SEQ ID NOs: 95-104 (ABW8 variants 1-10), 30-39 (ABW3 variants 1-10) or 82-91 (ABW7 variants 1-10) can have increased activity (e.g., editing and/or efficiency) compared to control nuclease constructs or other nuclease constructs.

As used herein, a non-naturally occurring nucleic acid sequence can be an engineered sequence or engineered nucleotide sequences of synthetized variants. Such non-naturally occurring nucleic acid sequences can be amplified, cloned, assembled, synthesized, generated from synthesized oligonucleotides or dNTPs, or otherwise obtained using methods known by those skilled in the art. In certain embodiments, examples of non-naturally occurring nucleic acid-guided nucleases disclosed herein can include those nucleic acid-guided nucleases with engineered polypeptide sequences (e.g., SEQ ID NOs: 15-17).

SEQ ID NO: 15
MGHHHHHHSSGVDLGTENLYFQSPAAKKKKLDGSVDMNNGINNFQ
NFIGISSLQKTLRNALIPTETTQQFIVKNGIIKEDELRGENRQIL
KDIMDDYYRGFISETLSSIDDIDWTSLFEKMEIQLKNGDNKDTLI
KEQTEYRKAIHKKFANDDREKNMESAKLISDILPEFVIHNNNYSA
SEKEEKTQVIKLESRFATSFKDYFKNRANCESADDISSSSCHRIV
NDNAEIFFSNALVYRRIVKSLSNDDINKISGDMKDSLKEMSLEEI
YSYEKYGEFITQEGISFYNDICGKVNSEMNLYCQKNKENKNLYKL
QKLHKQILCIADTSYEVPYKFESDEEVYQSVNGELDNISSKHIVE
RLRKIGDNYNGYNLDKIYIVSKFYESVSQKTYRDWETINTALEIH
YNNILPGNGKSKADKVKKAVKNDLQKSITEINELVSNYKLCSDDN
IKAETYIHEISHILNNFEAQELKYNPEIHLVESELKASELKNVLD
VIMNAFHWCSVEMTEELVDKDNNFYAELEEIYDEIYPVISLYNLV
RNYVTQKPYSTKKIKLNFGIPTLADGWSKSKEYSNNAIILMRDNL
YYLGIFNAKNKPDKKIIEGNTSENKGDYKKMIYNLLPGPNKMIPK
VELSSKTGVETYKPSAYILEGYKQNKHIKSSKDEDITFCHDLIDY
FKNCIAIHPEWKNFGEDESDTSTYEDISGFYREVELQGYKIDWTY
ISEKDIDLLQEKGQLYLFQIYNKDESKKSTGNDNLHTMYLKNLFS
EENLKDIVLKLNGEAEIFFRKSSIKNPIIHKKGSILVNRTYEAEE
KDQFGNIQIVRKNIPENIYQELYKYENDKSDKELSDEAAKLKNVV
GHHEAATNIVKDYRYTYDKYFLHMPITINFKANKTGFINDRILQY
IAKEKDLHVIGIDRGERNLIYVSVIDTCGNIVEQKSENIVNGYDY
QIKLKQQEGARQIARKEWKEIGKIKEIKEGYLSLVIHEISKMVIK
YNAIIAMEDLSYGFKKGREKVERQVYQKFETMLINKLNYLVFKDI
SITENGGLLKGYQLTYIPDKLKNVGHQCGCIFYVPAAYTSKIDPT
TGFVNIFKEKDLTVDAKREFIKKEDSIRYDSEKNLFCFTEDYNNF
ITQNTVMSKSSWSVYTYGVRIKRRFVNGRESNESDTIDITKDMEK
TLEMTDINWRDGHDLRQDIIDYEIVQHIFEIFRLTVQMRNSLSEL
EDRDYDRLISPVLNENNIFYDSAKAGDALPKDADANGAYCIALKG
LYEIKQITENWKEDGKFSRDKLKISNKDWEDFIQNKRYLKRPAAT
KKAGQAKKKKASGSGAGSPKKKRKVEDPKKKRKVIPG
SEQ ID NO: 16
SPAAKKKKLDGSVDMNNGINNFQNFIGISSLQKTLRNALIPTETT
QQFIVKNGIIKEDELRGENRQILKDIMDDYYRGFISETLSSIDDI
DWTSLFEKMEIQLKNGDNKDTLIKEQTEYRKAIHKKFANDDRFKN
MESAKLISDILPEFVIHNNNYSASEKEEKTQVIKLESRFATSFKD
YFKNRANCESADDISSSSCHRIVNDNAEIFFSNALVYRRIVKSLS
NDDINKISGDMKDSLKEMSLEEIYSYEKYGEFITQEGISFYNDIC
GKVNSFMNLYCQKNKENKNLYKLQKLHKQILCIADTSYEVPYKFE
SDEEVYQSVNGELDNISSKHIVERLRKIGDNYNGYNLDKIYIVSK
FYESVSQKTYRDWETINTALEIHYNNILPGNGKSKADKVKKAVKN
DLQKSITEINELVSNYKLCSDDNIKAETYIHEISHILNNFEAQEL
KYNPEIHLVESELKASELKNVLDVIMNAFHWCSVEMTEELVDKDN
NFYAELEEIYDEIYPVISLYNLVRNYVTQKPYSTKKIKLNFGIPT
LADGWSKSKEYSNNAIILMRDNLYYLGIFNAKNKPDKKIIEGNTS
ENKGDYKKMIYNLLPGPNKMIPKVELSSKTGVETYKPSAYILEGY
KQNKHIKSSKDEDITFCHDLIDYFKNCIAIHPEWKNFGFDESDTS
TYEDISGFYREVELQGYKIDWTYISEKDIDLLQEKGQLYLFQIYN
KDFSKKSTGNDNLHTMYLKNLFSEENLKDIVLKLNGEAEIFFRKS
SIKNPIIHKKGSILVNRTYEAEEKDQFGNIQIVRKNIPENIYQEL
YKYENDKSDKELSDEAAKLKNVVGHHEAATNIVKDYRYTYDKYFL
HMPITINFKANKTGFINDRILQYIAKEKDLHVIGIDRGERNLIYV
SVIDTCGNIVEQKSFNIVNGYDYQIKLKQQEGARQIARKEWKEIG
KIKEIKEGYLSLVIHEISKMVIKYNAIIAMEDLSYGFKKGRFKVE
RQVYQKFETMLINKLNYLVFKDISITENGGLLKGYQLTYIPDKLK
NVGHQCGCIFYVPAAYTSKIDPTTGFVNIFKEKDLTVDAKREFIK
KEDSIRYDSEKNLFCFTEDYNNFITQNTVMSKSSWSVYTYGVRIK
RRFVNGRESNESDTIDITKDMEKTLEMTDINWRDGHDLRQDIIDY
EIVQHIFEIFRLTVQMRNSLSELEDRDYDRLISPVLNENNIFYDS
AKAGDALPKDADANGAYCIALKGLYEIKQITENWKEDGKFSRDKL
KISNKDWEDFIQNKRYLKRPAATKKAGQAKKKKASGSGAGSPKKK
RKVEDPKKKRKVIPG
SEQ ID NO: 17
PAAKKKKLDGSVDMNNGTNNFQNFIGISSLQKTLRNALIPTETTQ
QFIVKNGIIKEDELRGENRQILKDIMDDYYRGFISETLSSIDDID
WTSLFEKMEIQLKNGDNKDTLIKEQTEYRKAIHKKFANDDRFKNM
ESAKLISDILPEFVIHNNNYSASEKEEKTQVIKLESRFATSFKDY
FKNRANCESADDISSSSCHRIVNDNAEIFFSNALVYRRIVKSLSN
DDINKISGDMKDSLKEMSLEEIYSYEKYGEFITQEGISFYNDICG
KVNSFMNLYCQKNKENKNLYKLQKLHKQILCIADTSYEVPYKFES
DEEVYQSVNGFLDNISSKHIVERLRKIGDNYNGYNLDKIYIVSKF
YESVSQKTYRDWETINTALEIHYNNILPGNGKSKADKVKKAVKND
LQKSITEINELVSNYKLCSDDNIKAETYIHEISHILNNFEAQELK
YNPEIHLVESELKASELKNVLDVIMNAFHWCSVEMTEELVDKDNN
FYAELEEIYDEIYPVISLYNLVRNYVTQKPYSTKKIKLNFGIPTL
ADGWSKSKEYSNNAIILMRDNLYYLGIFNAKNKPDKKIIEGNTSE
NKGDYKKMIYNLLPGPNKMIPKVFLSSKTGVETYKPSAYILEGYK
QNKHIKSSKDEDITECHDLIDYFKNCIAIHPEWKNFGFDESDTST
YEDISGFYREVELQGYKIDWTYISEKDIDLLQEKGQLYLFQIYNK
DFSKKSTGNDNLHTMYLKNLFSEENLKDIVLKLNGEAEIFFRKSS
IKNPIIHKKGSILVNRTYEAEEKDQFGNIQIVRKNIPENIYQELY
KYENDKSDKELSDEAAKLKNVVGHHEAATNIVKDYRYTYDKYFLH
MPITINFKANKTGFINDRILQYIAKEKDLHVIGIDRGERNLIYVS
VIDTCGNIVEQKSFNIVNGYDYQIKLKQQEGARQIARKEWKEIGK
IKEIKEGYLSLVIHEISKMVIKYNAIIAMEDLSYGFKKGREKVER
QVYQKFETMLINKLNYLVFKDISITENGGLLKGYQLTYIPDKLKN
VGHQCGCIFYVPAAYTSKIDPTTGFVNIFKFKDLTVDAKREFIKK
EDSIRYDSEKNLFCFTEDYNNFITQNTVMSKSSWSVYTYGVRIKR
RFVNGRESNESDTIDITKDMEKTLEMTDINWRDGHDLRQDIIDYE
IVQHIFEIFRLTVQMRNSLSELEDRDYDRLISPVLNENNIFYDSA
KAGDALPKDADANGAYCIALKGLYEIKQITENWKEDGKFSRDKLK
ISNKDWEDFIQNKRYLKRPAATKKAGQAKKKKASGSGAGSPKKKR
KVEDPKKKRKVIPG

More type V-A Cas proteins and their corresponding naturally occurring CRISPR-Cas systems can be identified by computational and experimental methods known in the art, e.g., as described in U.S. Pat. No. 9,790,490 and Shmakov et al. (2015) MOL. CELL, 60: 385. Exemplary computational methods include analysis of putative Cas proteins by homology modeling, structural BLAST, PSI-BLAST, or HHPred, and analysis of putative CRISPR loci by identification of CRISPR arrays. Exemplary experimental methods include in vitro cleavage assays and in-cell nuclease assays (e.g., the Surveyor assay) as described in Zetsche et al. (2015) CELL, 163: 759.

In certain embodiments, the Cas protein is a Cas nuclease that directs cleavage of one or both strands at the target locus, such as the target strand (i.e., the strand having the target nucleotide sequence that hybridizes with a single guide nucleic acid or dual guide nucleic acids) and/or the non-target strand. In certain embodiments, the Cas nuclease directs cleavage of one or both strands within at least 1, 2, 3, 4, 5, 6, 7, 8, 9, 10, 15, 20, 25, 50, 100, 200, 500, or more nucleotides from the first or last nucleotide of the target nucleotide sequence or its complementary sequence. In certain embodiments, the cleavage is staggered, i.e. generating sticky ends. In certain embodiments, the cleavage generates a staggered cut with a 5′ overhang. In certain embodiments, the cleavage generates a staggered cut with a 5′ overhang of 1 to 5 nucleotides, e.g., of 4 or 5 nucleotides. In certain embodiments, the cleavage site is distant from the PAM, e.g., the cleavage occurs after the 18th nucleotide on the non-target strand and after the 23rd nucleotide on the target strand.

In certain embodiments, the engineered, non-naturally occurring system of the present invention further comprises the Cas nuclease that a complex comprising the targeter nucleic acid and the modulator nucleic acid is capable of activating. In other embodiments, the engineered, non-naturally occurring system of the present invention further comprises a Cas protein that is related to the Cas nuclease that a complex comprising the targeter nucleic acid and the modulator nucleic acid is capable of activating. For example, in certain embodiments, the Cas protein comprises an amino acid sequence at least 80% (e.g., at least 85%, at least 90%, at least 91%, at least 92%, at least 93%, at least 94%, at least 95%, at least 96%, at least 97%, at least 98%, or at least 99%) identical to the Cas nuclease. In certain embodiments, the Cas protein comprises a nuclease-inactive mutant of the Cas nuclease. In certain embodiments, the Cas protein further comprises an effector domain.

In certain embodiments, the Cas protein lacks substantially all DNA cleavage activity. Such a Cas protein can be generated by introducing one or more mutations to an active Cas nuclease (e.g., a naturally occurring Cas nuclease). A mutated Cas protein is considered to substantially lack all DNA cleavage activity when the DNA cleavage activity of the protein has no more than 25%, 10%, 5%, 1%, 0.1%, 0.01%, or less of the DNA cleavage activity of the corresponding non-mutated form, for example, nil or negligible as compared with the non-mutated form. Thus, the Cas protein may comprise one or more mutations (e.g., a mutation in the RuvC domain of a type V-A Cas protein) and be used as a generic DNA binding protein with or without fusion to an effector domain. Exemplary mutations include D908A, E993A, and D1263A with reference to the amino acid positions in AsCpf1; D832A, E925A, and D1180A with reference to the amino acid positions in LbCpf1; and D917A, E1006A, and D1255A with reference to the amino acid position numbering of the FnCpf1. More mutations can be designed and generated according to the crystal structure described in Yamano et al. (2016) CELL, 165: 949.

It is understood that the Cas protein, rather than losing nuclease activity to cleave all DNA, may lose the ability to cleave only the target strand or only the non-target strand of a double-stranded DNA, thereby being functional as a nickase (see, Gao et al. (2016) CELL RES., 26: 901). Accordingly, in certain embodiments, the Cas nuclease is a Cas nickase. In certain embodiments, the Cas nuclease has the activity to cleave the non-target strand but substantially lacks the activity to cleave the target strand, e.g., by a mutation in the Nuc domain. In certain embodiments, the Cas nuclease has the cleavage activity to cleave the target strand but substantially lacks the activity to cleave the non-target strand.

In other embodiments, the Cas nuclease has the activity to cleave a double-stranded DNA and result in a double-strand break.

Cas proteins that lack substantially all DNA cleavage activity or have the ability to cleave only one strand may also be identified from naturally occurring systems. For example, certain naturally occurring CRISPR-Cas systems may retain the ability to bind the target nucleotide sequence but lose entire or partial DNA cleavage activity in eukaryotic (e.g., mammalian or human) cells. Such type V-A proteins are disclosed, for example, in Kim et al. (2017) ACS SYNTH. BIOL. 6(7): 1273-82 and Zhang et al. (2017) CELL DISCOV. 3:17018.

The activity of the Cas protein (e.g., Cas nuclease) can be altered, thereby creating an engineered Cas protein. In certain embodiments, the altered activity of the engineered Cas protein comprises increased targeting efficiency and/or decreased off-target binding. While not wishing to be bound by theory, it is hypothesized that off-target binding can be recognized by the Cas protein, for example, by the presence of one or more mismatches between the spacer sequence and the target nucleotide sequence, which may affect the stability and/or conformation of the CRISPR-Cas complex. In certain embodiments, the altered activity comprises modified binding, e.g., increased binding to the target locus (e.g., the target strand or the non-target strand) and/or decreased binding to off-target loci. In certain embodiments, the altered activity comprises altered charge in a region of the protein that associates with a single guide nucleic acid or dual guide nucleic acids. In certain embodiments, the altered activity of the engineered Cas protein comprises altered charge in a region of the protein that associates with the target strand and/or the non-target strand. In certain embodiments, the altered activity of the engineered Cas protein comprises altered charge in a region of the protein that associates with an off-target locus. The altered charge can include decreased positive charge, decreased negative charge, increased positive charge, and increased negative charge. For example, decreased negative charge and increased positive charge may generally strengthen the binding to the nucleic acid(s) whereas decreased positive charge and increased negative charge may weaken the binding to the nucleic acid(s). In certain embodiments, the altered activity comprises increased or decreased steric hindrance between the protein and a single guide nucleic acid or dual guide nucleic acids. In certain embodiments, the altered activity comprises increased or decreased steric hindrance between the protein and the target strand and/or the non-target strand. In certain embodiments, the altered activity comprises increased or decreased steric hindrance between the protein and an off-target locus. In certain embodiments, the modification or mutation comprises a substitution of Lys, His, Arg, Glu, Asp, Ser, Gly, or Thr. In certain embodiments, the modification or mutation comprises a substitution with Gly, Ala, Ile, Glu, or Asp. In certain embodiments, the modification or mutation comprises an amino acid substitution in the groove between the WED and RuvC domain of the Cas protein (e.g., a type V-A Cas protein).

In certain embodiments, the altered activity of the engineered Cas protein comprises increased nuclease activity to cleave the target locus. In certain embodiments, the altered activity of the engineered Cas protein comprises decreased nuclease activity to cleave an off-target locus. In certain embodiments, the altered activity of the engineered Cas protein comprises altered helicase kinetics. In certain embodiments, the engineered Cas protein comprises a modification that alters formation of the CRISPR complex.

In certain embodiments, a protospacer adjacent motif (PAM) or PAM-like motif directs binding of the Cas protein complex to the target locus. Many Cas proteins have PAM specificity. The precise sequence and length requirements for the PAM differ depending on the Cas protein used. PAM sequences are typically 2-5 base pairs in length and are adjacent to (but located on a different strand of target DNA from) the target nucleotide sequence. PAM sequences can be identified using a method known in the art, such as testing cleavage, targeting, or modification of oligonucleotides having the target nucleotide sequence and different PAM sequences.

Exemplary PAM sequences are provided in Tables 10 and 11. In one embodiment, the Cas protein is MAD7 and the PAM is TTTN, wherein N is A, C, G, or T. In another embodiment, the Cas protein is MAD7 and the PAM is CTTN, wherein N is A, C, G, or T. In another embodiment, the Cas protein is AsCpf1 and the PAM is TTTN, wherein N is A, C, G, or T. In another embodiment, the Cas protein is FnCpf1 and the PAM is 5′ TTN, wherein N is A, C, G, or T. PAM sequences for certain other type V-A Cas proteins are disclosed in Zetsche et al. (2015) CELL, 163: 759 and U.S. Pat. No. 9,982,279. Further, engineering of the PAM Interacting (PI) domain of a Cas protein may allow programing of PAM specificity, improve target site recognition fidelity, and increase the versatility of the engineered, non-naturally occurring system. Exemplary approaches to alter the PAM specificity of Cpf1 is described in Gao et al. (2017) NAT. BIOTECHNOL., 35: 789.

In certain embodiments, the engineered Cas protein comprises a modification that alters the Cas protein specificity in concert with modification to targeting range. Cas mutants can be designed to have increased target specificity as well as accommodating modifications in PAM recognition, for example by choosing mutations that alter PAM specificity (e.g., in the PI domain) and combining those mutations with groove mutations that increase (or if desired, decrease) specificity for the on-target locus versus off-target loci. The Cas modifications described herein can be used to counter loss of specificity resulting from alteration of PAM recognition, enhance gain of specificity resulting from alteration of PAM recognition, counter gain of specificity resulting from alteration of PAM recognition, or enhance loss of specificity resulting from alteration of PAM recognition.

In certain embodiments, the engineered Cas protein comprises one or more nuclear localization signal (NLS) motifs. In certain embodiments, the engineered Cas protein comprises at least 2 (e.g., at least 3, at least 4, at least 5, at least 6, at least 7, at least 8, at least 9, or at least 10) NLS motifs. Non-limiting examples of NLS motifs include: the NLS of SV40 large T-antigen, having the amino acid sequence of PKKKRKV (SEQ ID NO: 35); the NLS from nucleoplasmin, e.g., the nucleoplasmin bipartite NLS having the amino acid sequence of KRPAATKKAGQAKKKK (SEQ ID NO: 36); the c-myc NLS, having the amino acid sequence of PAAKRVKLD (SEQ ID NO: 37) or RQRRNELKRSP (SEQ ID NO: 38); the hRNPA1 M9 NLS, having the amino acid sequence of NQSSNFGPMKGGNFGGRSSGPYGGGGQYFAKPRNQGGY (SEQ ID NO: 39); the importin-α IBB domain NLS, having the amino acid sequence of RMRIZFKNKGKDTAELRRRRVEVSVELRKAKKDEQILKRRNV (SEQ ID NO: 40); the myoma T protein NLS, having the amino acid sequence of VSRKRPRP (SEQ ID NO: 41) or PPKKARED (SEQ ID NO: 42); the human p53 NLS, having the amino acid sequence of PQPKKKPL (SEQ ID NO: 43); the mouse c-abl IV NLS, having the amino acid sequence of SALIKKKKKMAP (SEQ ID NO: 44); the influenza virus NS1 NLS, having the amino acid sequence of DRLRR (SEQ ID NO: 45) or PKQKKRK (SEQ ID NO: 46); the hepatitis virus S antigen NLS, having the amino acid sequence of RKLKKKIKKL (SEQ ID NO: 47); the mouse Mx1 protein NLS, having the amino acid sequence of REKKKFLKRR (SEQ ID NO: 48); the human poly(ADP-ribose) polymerase NLS, having the amino acid sequence of KRKGDEVDGVDEVAKKKSKK (SEQ ID NO: 49); the human glucocorticoid receptor NLS, having the amino acid sequence of RKCLQAGMNLEARKTKK (SEQ ID NO: 33), and synthetic NLS motifs such as PAAKKKKLD (SEQ ID NO: 34).

In general, the one or more NLS motifs are of sufficient strength to drive accumulation of the Cas protein in a detectable amount in the nucleus of a eukaryotic cell. The strength of nuclear localization activity may derive from the number of NLS motif(s) in the Cas protein, the particular NLS motif(s) used, the position(s) of the NLS motif(s), or a combination of these factors. In certain embodiments, the engineered Cas protein comprises at least 1 (e.g., at least 2, at least 3, at least 4, at least 5, at least 6, at least 7, at least 8, at least 9, or at least 10) NLS motif(s) at or near the N-terminus (e.g., within 1, 2, 3, 4, 5, 10, 15, 20, 25, 30, 40, 50, or more amino acids along the polypeptide chain from the N-terminus). In certain embodiments, the engineered Cas protein comprises at least 1 (e.g., at least 2, at least 3, at least 4, at least 5, at least 6, at least 7, at least 8, at least 9, or at least 10) NLS motif(s) at or near the C-terminus (e.g., within 1, 2, 3, 4, 5, 10, 15, 20, 25, 30, 40, 50, or more amino acids along the polypeptide chain from the C-terminus). In certain embodiments, the engineered Cas protein comprises at least 1 (e.g., at least 2, at least 3, at least 4, at least 5, at least 6, at least 7, at least 8, at least 9, or at least 10) NLS motif(s) at or near the C-terminus and at least 1 (e.g., at least 2, at least 3, at least 4, at least 5, at least 6, at least 7, at least 8, at least 9, or at least 10) NLS motif(s) at or near the N-terminus. In certain embodiments, the engineered Cas protein comprises one, two, or three NLS motifs at or near the C-terminus. In certain embodiments, the engineered Cas protein comprises one NLS motif at or near the N-terminus and one, two, or three NLS motifs at or near the C-terminus. In certain embodiments, the engineered Cas protein comprises a nucleoplasmin NLS at or near the C-terminus.

Detection of accumulation in the nucleus may be performed by any suitable technique. For example, a detectable marker may be fused to the nucleic acid-targeting protein, such that location within a cell may be visualized. Cell nuclei may also be isolated from cells, the contents of which may then be analyzed by any suitable process for detecting the protein, such as immunohistochemistry, Western blot, or enzyme activity assay. Accumulation in the nucleus may also be determined indirectly, such as by an assay that detects the effect of the nuclear import of a Cas protein complex (e.g., assay for DNA cleavage or mutation at the target locus, or assay for altered gene expression activity) as compared to a control not exposed to the Cas protein or exposed to a Cas protein lacking one or more of the NLS motifs.

In certain embodiments, the Cas protein is a chimeric Cas protein, e.g., a Cas protein having enhanced function by being a chimera. Chimeric Cas proteins may be new Cas proteins containing fragments from more than one naturally occurring Cas proteins or variants thereof. For example, fragments of multiple type V-A Cas homologs (e.g., orthologs) may be fused to form a chimeric Cas protein. In certain embodiments, the chimeric Cas protein comprises fragments of Cpf1 orthologs from multiple species and/or strains.

In certain embodiments, the Cas protein comprises one or more effector domains. The one or more effector domains may be located at or near the N-terminus of the Cas protein and/or at or near the C-terminus of the Cas protein. In certain embodiments, an effector domain comprised in the Cas protein is a transcriptional activation domain (e.g., VP64), a transcriptional repression domain (e.g., a KRAB domain or an SID domain), an exogenous nuclease domain (e.g., FokI), a deaminase domain (e.g., cytidine deaminase or adenine deaminase), or a reverse transcriptase domain (e.g., a high fidelity reverse transcriptase domain). Other activities of effector domains include but are not limited to methylase activity, demethylase activity, transcription release factor activity, translational initiation activity, translational activation activity, translational repression activity, histone modification (e.g., acetylation or demethylation) activity, single-stranded RNA cleavage activity, double-strand RNA cleavage activity, single-strand DNA cleavage activity, double-strand DNA cleavage activity, and nucleic acid binding activity.

In certain embodiments, the Cas protein comprises one or more protein domains that enhance homology-directed repair (HDR) and/or inhibit non-homologous end joining (NHEJ). Exemplary protein domains having such functions are described in Jayavaradhan et al. (2019) NAT. COMMUN. 10(1): 2866 and Janssen et al. (2019) MOL. THER. NUCLEIC ACIDS 16: 141-54. In certain embodiments, the Cas protein comprises a dominant negative version of p53-binding protein 1 (53BP1), for example, a fragment of 53BP1 comprising a minimum focus forming region (e.g., amino acids 1231-1644 of human 53BP1). In certain embodiments, the Cas protein comprises a motif that is targeted by APC-Cdhl, such as amino acids 1-110 of human Geminin, thereby resulting in degradation of the fusion protein during the HDR non-permissive G1 phase of the cell cycle.

In certain embodiments, the Cas protein comprises an inducible or controllable domain. Non-limiting examples of inducers or controllers include light, hormones, and small molecule drugs. In certain embodiments, the Cas protein comprises a light inducible or controllable domain. In certain embodiments, the Cas protein comprises a chemically inducible or controllable domain.

In certain embodiments, the Cas protein comprises a tag protein or peptide for ease of tracking or purification. Non-limiting examples of tag proteins and peptides include fluorescent proteins (e.g., green fluorescent protein (GFP), YFP, RFP, CFP, mCherry, tdTomato), HIS tags (e.g., 6× His tag, (SEQ ID NO: 789)), hemagglutinin (HA) tag, FLAG tag, and Myc tag.

In certain embodiments, the Cas protein is conjugated to a non-protein moiety, such as a fluorophore useful for genomic imaging. In certain embodiments, the Cas protein is covalently conjugated to the non-protein moiety. The terms “CRISPR-Associated protein,” “Cas protein,” “Cas,” “CRISPR-Associated nuclease,” and “Cas nuclease” are used herein to include such conjugates despite the presence of one or more non-protein moieties.

### Guide Nucleic Acids

In certain embodiments, the guide nucleic acid of the present invention is a guide nucleic acid that is capable of binding a Cas protein alone (e.g., in the absence of a tracrRNA). Such guide nucleic acid is also called a single guide nucleic acid. In certain embodiments, the single guide nucleic acid is capable of activating a Cas nuclease alone (e.g., in the absence of a tracrRNA). The present invention also provides an engineered, non-naturally occurring system comprising the single guide nucleic acid. In certain embodiments, the system further comprises the Cas protein that the single guide nucleic acid is capable of binding or the Cas nuclease that the single guide nucleic acid is capable of activating.

In other embodiments, the guide nucleic acid of the present invention is a targeter nucleic acid that, in combination with a modulator nucleic acid, is capable of binding a Cas protein. In certain embodiments, the guide nucleic acid is a targeter nucleic acid that, in combination with a modulator nucleic acid, is capable of activating a Cas nuclease. The present invention also provides an engineered, non-naturally occurring system comprising the targeter nucleic acid and the cognate modulator nucleic acid. In certain embodiments, the system further comprises the Cas protein that the targeter nucleic acid and the modulator nucleic acid are capable of binding or the Cas nuclease that the targeter nucleic acid and the modulator nucleic acid are capable of activating.

It is contemplated that the single or dual guide nucleic acids need to be the compatible with a Cas protein (e.g., Cas nuclease) to provide an operative CRISPR system. For example, the targeter stem sequence and the modulator stem sequence can be derived from a naturally occurring crRNA capable of activating a Cas nuclease in the absence of a tracrRNA. Alternatively, the targeter stem sequence and the modulator stem sequence can be derived from a naturally occurring set of crRNA and tracrRNA, respectively, that are capable of activating a Cas nuclease. In certain embodiments, the nucleotide sequences of the targeter stem sequence and the modulator stem sequence are identical to the corresponding stem sequences of a stem-loop structure in such naturally occurring crRNA.

Guide nucleic acid sequences that are operative with a type II or type V Cas protein are known in the art and are disclosed, for example, in U.S. Pat. Nos. 9,790,490, 9,896,696, 10,113,179, and 10,266,850, and U.S. Patent Application Publication No. 2014/0242664. Exemplary single guide and dual guide sequences that are operative with certain type V-A Cas proteins are provided in Tables 10 and 11, respectively. It is understood that these sequences are merely illustrative, and other guide nucleic acid sequences may also be used with these Cas proteins.

TABLE 12
Type V-A Cas Protein and Corresponding
Single Guide Nucleic Acid Sequences

| Cas Protein | Scaffold Sequence¹ | PAM² |
|---|---|---|
| MAD7 (SEQ ID | UAAUUUCUACUCUUGUAGA (SEQ ID NO: 15), | 5′ TTTN |
| NO: 1) | AUCUACAACAGUAGA (SEQ ID NO: 16), | or 5′ |
| | AUCUACAAAAGUAGA (SEQ ID NO: 17), | |
| | GGAAUUUCUACUCUUGUAGA (SEQ ID NO: 18), | CTTN |
| | UAAUUCCCACUCUUGUGGG (SEQ ID NO: 19) | |
| MAD2 (SEQ ID | AUCUACAAGAGUAGA (SEQ ID NO: 20), | 5′ TTTN |
| NO: 2) | AUCUACAACAGUAGA (SEQ ID NO: 16), | |
| | AUCUACAAAAGUAGA (SEQ ID NO: 17), | |
| | AUCUACACUAGUAGA (SEQ ID NO: 21) | |
| AsCpf1 (SEQ | UAAUUUCUACUCUUGUAGA (SEQ ID NO: 15) | 5′ TTTN |
| ID NO: 3) | | |
| LbCpf1 (SEQ | UAAUUUCUACUAAGUGUAGA (SEQ ID NO: 22) | 5′ TTTN |
| ID NO: 4) | | |
| FnCpf1 (SEQ | UAAUUUUCUACUUGUUGUAGA (SEQ ID NO: 23) | 5′ TTN |
| ID NO: 5) | | |
| PbCpf1 (SEQ | AAUUUCUACUGUUGUAGA (SEQ ID NO: 24) | 5′ TTTC |
| ID NO: 6) | | |
| PsCpf1 (SEQ | AAUUUCUACUGUUGUAGA (SEQ ID NO: 24) | 5′ TTTC |
| ID NO: 7) | | |
| As2Cpf1 (SEQ | AAUUUCUACUGUUGUAGA (SEQ ID NO: 24) | 5′ TTTC |
| ID NO: 8) | | |
| McCpf1 (SEQ | GAAUUUCUACUGUUGUAGA (SEQ ID NO: 25) | 5′ TTTC |
| ID NO: 9) | | |
| Lb3Cpf1 (SEQ | GAAUUUCUACUGUUGUAGA (SEQ ID NO: 25) | 5′ TTTC |
| ID NO: 10) | | |
| EcCpf1 (SEQ | GAAUUUCUACUGUUGUAGA (SEQ ID NO: 25) | 5′ TTTC |
| ID NO: 11) | | |
| SmCsm1 (SEQ | GAAUUUCUACUGUUGUAGA (SEQ ID NO: 25) | 5′ TTTC |
| ID NO: 12) | | |
| SsCsm1 (SEQ | GAAUUUCUACUGUUGUAGA (SEQ ID NO: 25) | 5′ TTTC |
| ID NO: 13) | | |
| MbCsm1 (SEQ | GAAUUUCUACUGUUGUAGA (SEQ ID NO: 25) | 5′ TTTC |
| ID NO: 14) | | |

¹The modulator sequence in the scaffold sequence is underlined; the targeter stem sequence in the scaffold sequence is bold-underlined. It is understood that a “scaffold sequence” listed herein constitutes a portion of a single guide nucleic acid. Additional nucleotide sequences, other than the spacer sequence, can be comprised in the single guide nucleic acid.
²In the consensus PAM sequences, N represents A, C, G, or T. Where the PAM sequence is preceded by “5′,” it means that the PAM is located immediately upstream of the target nucleotide sequence when using the non-target strand (i.e., the strand not hybridized with the spacer sequence) as the coordinate.

TABLE 13
Type V-A Cas Protein and Corresponding Dual Guide Nucleic Acid Sequences

| | | Targeter | |
|---|---|---|---|
| | | Stem | |
| Cas Protein | Modulator Sequence¹ | Sequence | PAM² |
| MAD7 (SEQ ID NO: | UAAUUUCUAC (SEQ ID NO: | GUAGA | 5′ TTTN |
| 1) | 26) | | or 5′ |
| | AUCUAC (SEQ ID NO: 27) | GUAGA | CTTN |
| | GGAAUUUCUAC (SEQ ID NO: | GUAGA | |
| | 28) | | |
| | UAAUUCCCAC (SEQ ID NO: | GUGGG | |
| | 29) | | |
| MAD2 (SEQ ID NO: | AUCUAC (SEQ ID NO: 27) | GUAGA | 5′ TTTN |
| 2) | | | |
| AsCpf1 (SEQ ID | UAAUUUCUAC (SEQ ID NO: | GUAGA | 5′ TTTN |
| NO: 3) | 26) | | |
| LbCpf1 (SEQ ID | UAAUUUCUAC (SEQ ID NO: | GUAGA | 5′ TTTN |
| NO: 4) | 26) | | |
| FnCpf1 (SEQ ID | UAAUUUUCUACU (SEQ ID NO: | GUAGA | 5′ TTN |
| NO: 5) | 30) | | |
| PbCpf1 (SEQ ID | AAUUUCUAC (SEQ ID NO: 31) | GUAGA | 5′ TTTC |
| NO: 6) | | | |
| PsCpf1 (SEQ ID | AAUUUCUAC (SEQ ID NO: 31) | GUAGA | 5′ TTTC |
| NO: 7) | | | |
| As2Cpf1 (SEQ ID | AAUUUCUAC (SEQ ID NO: 31) | GUAGA | 5′ TTTC |
| NO: 8) | | | |
| McCpf1 (SEQ ID | GAAUUUCUAC (SEQ ID NO: | GUAGA | 5′ TTTC |
| NO: 9) | 32) | | |
| Lb3Cpf1 (SEQ ID | GAAUUUCUAC (SEQ ID NO: | GUAGA | 5′ TTTC |
| NO: 10) | 32) | | |
| EcCpf1 (SEQ ID | GAAUUUCUAC (SEQ ID NO: | GUAGA | 5′ TTTC |
| NO: 11) | 32) | | |
| SmCsm1 (SEQ ID | GAAUUUCUAC (SEQ ID NO: | GUAGA | 5′ TTTC |
| NO: 12) | 32) | | |
| SsCsm1 (SEQ ID | GAAUUUCUAC (SEQ ID NO: | GUAGA | 5′ TTTC |
| NO: 13) | 32) | | |
| MbCsm1 (SEQ ID | GAAUUUCUAC (SEQ ID NO: | GUAGA | 5′ TTTC |
| NO: 14) | 32) | | |

¹It is understood that a “modulator sequence” listed herein may constitute the nucleotide sequence of a modulator nucleic acid. Alternatively, additional nucleotide sequences can be comprised in the modulator nucleic acid 5′ and/or 3′ to a “modulator sequence” listed herein.
²In the consensus PAM sequences, N represents A, C, G, or T. Where the PAM sequence is preceded by “5′,” it means that the PAM is located immediately upstream of the target nucleotide sequence when using the non-target strand (i.e., the strand not hybridized with the spacer sequence) as the coordinate.

In certain embodiments, the guide nucleic acid of the present invention, in the context of a type V-A CRISPR-Cas system, comprises a targeter stem sequence listed in Table 13. The same targeter stem sequences, as a portion of scaffold sequences, are bold-underlined in Table 12.

In certain embodiments, the guide nucleic acid is a single guide nucleic acid that comprises, from 5′ to 3′, a modulator stem sequence, a loop sequence, a targeter stem sequence, and a spacer sequence disclosed herein. In certain embodiments, the targeter stem sequence in the single guide nucleic acid is listed in Table 12 as a bold-underlined portion of scaffold sequence, and the modulator stem sequence is complementary (e.g., 100% complementary) to the targeter stem sequence. In certain embodiments, the single guide nucleic acid comprises, from 5′ to 3′, a modulator sequence listed in Table 12 as an underlined portion of a scaffold sequence, a loop sequence, a targeter stem sequence a bold-underlined portion of the same scaffold sequence, and a spacer sequence disclosed herein. In certain embodiments, an engineered, non-naturally occurring system of the present invention comprises the single guide nucleic acid comprising a scaffold sequence listed in Table 12. In certain embodiments, the system further comprises a Cas protein (e.g., Cas nuclease) comprising an amino acid sequence at least 30%, at least 40%, at least 50%, at least 60%, at least 70%, at least 75%, at least 80%, at least 85%, at least 90%, at least 91%, at least 92%, at least 93%, at least 94%, at least 95%, at least 96%, at least 97%, at least 98%, or at least 99% identical to the amino acid sequence set forth in the SEQ ID NO listed in the same line of Table 12. In certain embodiments, the system further comprises a Cas protein (e.g., Cas nuclease) comprising the amino acid sequence set forth in the SEQ ID NO listed in the same line of Table 12. In certain embodiments, the system is useful for targeting, editing, or modifying a nucleic acid comprising a target nucleotide sequence close or adjacent to (e.g., immediately downstream of) a PAM listed in the same line of Table 12 when using the non-target strand (i.e., the strand not hybridized with the spacer sequence) as the coordinate.

In certain embodiments, the guide nucleic acid is a targeter guide nucleic acid that comprises, from 5′ to 3′, a targeter stem sequence and a spacer sequence disclosed herein. In certain embodiments, the targeter stem sequence in the targeter nucleic acid is listed in Table 13. In certain embodiments, an engineered, non-naturally occurring system of the present invention comprises the targeter nucleic acid and a modulator stem sequence complementary (e.g., 100% complementary) to the targeter stem sequence. In certain embodiments, the modulator nucleic acid comprises a modulator sequence listed in the same line of Table 13. In certain embodiments, the system further comprises a Cas protein (e.g., Cas nuclease) comprising an amino acid sequence at least 30%, at least 40%, at least 50%, at least 60%, at least 70%, at least 75%, at least 80%, at least 85%, at least 90%, at least 91%, at least 92%, at least 93%, at least 94%, at least 95%, at least 96%, at least 97%, at least 98%, or at least 99% identical to the amino acid sequence set forth in the SEQ ID NO listed in the same line of Table 13. In certain embodiments, the system further comprises a Cas protein (e.g., Cas nuclease) comprising the amino acid sequence set forth in the SEQ ID NO listed in the same line of Table 13. In certain embodiments, the system is useful for targeting, editing, or modifying a nucleic acid comprising a target nucleotide sequence close or adjacent to (e.g., immediately downstream of) a PAM listed in the same line of Table 13 when using the non-target strand (i.e., the strand not hybridized with the spacer sequence) as the coordinate.

The single guide nucleic acid, the targeter nucleic acid, and/or the modulator nucleic acid can be synthesized chemically or produced in a biological process (e.g., catalyzed by an RNA polymerase in an in vitro reaction). Such reaction or process may limit the lengths of the single guide nucleic acid, targeter nucleic acid, and modulator nucleic acid. In certain embodiments, the single guide nucleic acid is no more than 100, 90, 80, 70, 60, 50, 40, 30, or 25 nucleotides in length. In certain embodiments, the single guide nucleic acid is at least 20, 25, 30, 40, 50, 60, 70, 80, or 90 nucleotides in length. In certain embodiments, the single guide nucleic acid is 20-100, 20-90, 20-80, 20-70, 20-60, 20-50, 20-40, 20-30, 20-25, 25-100, 25-90, 25-80, 25-70, 25-60, 25-50, 25-40, 25-30, 30-100, 30-90, 30-80, 30-70, 30-60, 30-50, 30-40, 40-100, 40-90, 40-80, 40-70, 40-60, 40-50, 50-100, 50-90, 50-80, 50-70, 50-60, 60-100, 60-90, 60-80, 60-70, 70-100, 70-90, 70-80, 80-100, 80-90, or 90-100 nucleotides in length. In certain embodiments, the targeter nucleic acid is no more than 100, 90, 80, 70, 60, 50, 40, 30, or 25 nucleotides in length. In certain embodiments, the targeter nucleic acid is at least 20, 25, 30, 40, 50, 60, 70, 80, or 90 nucleotides in length. In certain embodiments, the targeter nucleic acid is 20-100, 20-90, 20-80, 20-70, 20-60, 20-50, 20-40, 20-30, 20-25, 25-100, 25-90, 25-80, 25-70, 25-60, 25-50, 25-40, 25-30, 30-100, 30-90, 30-80, 30-70, 30-60, 30-50, 30-40, 40-100, 40-90, 40-80, 40-70, 40-60, 40-50, 50-100, 50-90, 50-80, 50-70, 50-60, 60-100, 60-90, 60-80, 60-70, 70-100, 70-90, 70-80, 80-100, 80-90, or 90-100 nucleotides in length. In certain embodiments, the modulator nucleic acid is no more than 100, 90, 80, 70, 60, 50, 40, 30, or 20 nucleotides in length. In certain embodiments, the modulator nucleic acid is at least 10, 15, 20, 25, 30, 40, 50, 60, 70, 80, or 90 nucleotides in length. In certain embodiments, the modulator nucleic acid is 10-100, 10-90, 10-80, 10-70, 10-60, 10-50, 10-40, 10-30, 10-20, 15-100, 15-90, 15-80, 15-70, 15-60, 15-50, 15-40, 15-30, 15-20, 20-100, 20-90, 20-80, 20-70, 20-60, 20-50, 20-40, 20-30, 25-100, 25-90, 25-80, 25-70, 25-60, 25-50, 25-40, 25-30, 30-100, 30-90, 30-80, 30-70, 30-60, 30-50, 30-40, 40-100, 40-90, 40-80, 40-70, 40-60, 40-50, 50-100, 50-90, 50-80, 50-70, 50-60, 60-100, 60-90, 60-80, 60-70, 70-100, 70-90, 70-80, 80-100, 80-90, or 90-100 nucleotides in length.

In naturally occurring type V-A CRISPR-Cas systems, the crRNA comprises a scaffold sequence (also called direct repeat sequence) and a spacer sequence that hybridizes with the target nucleotide sequence. In certain naturally occurring type V-A CRISPR-Cas systems, the scaffold sequence forms a stem-loop structure in which the stem consists of five consecutive base pairs. A dual guide type V-A CRISPR-Cas system may be derived from a naturally occurring type V-A CRISPR-Cas system, or a variant thereof in which the Cas protein is guided to the target nucleotide sequence by a crRNA alone, such system referred to herein as a “single guide type V-A CRISPR-Cas system.” In certain modified dual guide type V-A CRISPR-Cas systems disclosed herein, the targeter nucleic acid comprises the chain of the stem sequence between the spacer and the loop (the “targeter stem sequence”) and the spacer sequence, and the modulator nucleic acid comprises the other chain of the stem sequence (the “modulator stem sequence”) and the 5′ sequence, e.g., a tail sequence, positioned 5′ to the modulator stem sequence. The targeter stem sequence is 100% complementary to the modulator stem sequence. As such, the double-stranded complex of the targeter nucleic acid and the modulator nucleic acid retains the orientation of the 5′ sequence, e.g., a tail sequence, the modulator stem sequence, the targeter stem sequence, and the spacer sequence of a single guide type V-A CRISPR-Cas system but lacks the loop structure between the modulator stem sequence and the targeter stem sequence. A schematic representation of an exemplary double-stranded complex is shown in FIG. 1.

Notwithstanding the general structural similarity, it has been discovered that the stem-loop structure of the crRNA in a naturally occurring type V-A CRISPR complex is dispensable for the functionality of the CRISPR system. This discovery is surprising because the prior art has suggested that the stem-loop structure is critical (see, Zetsche et al. (2015) Cell, 163: 759) and that removal of the loop structure by “splitting” the crRNA abrogated the activity of a AsCpf1 CRISPR system (see, Li et al. (2017) Nat. Biomed. Eng., 1: 0066).

It is contemplated that the length of the duplex formed within the single guide nucleic acid or formed between the targeter nucleic acid and the modulator nucleic acid may be a factor in providing an operative CRISPR system. In certain embodiments, the targeter stem sequence and the modulator stem sequence each consist of 4-10 nucleotides that base pair with each other. In certain embodiments, the targeter stem sequence and the modulator stem sequence each consist of 4-9, 4-8, 4-7, 4-6, 4-5, 5-10, 5-9, 5-8, 5-7, or 5-6 nucleotides that base pair with each other. In certain embodiments, the targeter stem sequence and the modulator stem sequence each consist of 4, 5, 6, 7, 8, 9, or 10 nucleotides. It is understood that the composition of the nucleotides in each sequence affects the stability of the duplex, and a C-G base pair confers greater stability than an A-U base pair. In certain embodiments, 20%-80%, 20%-70%, 20%-60%, 20%-50%, 20%-40%, 20%-30%, 30%-80%, 30%-70%, 30%-60%, 30%-50%, 30%-40%, 40%-80%, 40%-70%, 40%-60%, 40%-50%, 50%-80%, 50%-70%, 50%-60%, 60%-80%, 60%-70%, or 70%-80% of the base pairs are C-G base pairs. In certain embodiments, the targeter stem sequence and the modulator stem share at least 80%, 85%, 90%, 95%, 99%, 99.5%, or 100% sequence complementarity. In a preferred embodiment, the target stem sequence and the modulator stem sequence share at 80-100% sequence complementarity.

In certain embodiments, the targeter stem sequence and the modulator stem sequence each consist of 5 nucleotides. As such, the targeter stem sequence and the modulator stem sequence form a duplex of 5 base pairs. In certain embodiments, 0-4, 0-3, 0-2, 0-1, 1-5, 1-4, 1-3, 1-2, 2-5, 2-4, 2-3, 3-5, 3-4, or 4-5 out of the 5 base pairs are C-G base pairs. In certain embodiments, 0, 1, 2, 3, 4, or 5 out of the 5 base pairs are C-G base pairs. In certain embodiments, the targeter stem sequence consists of 5′-GUAGA-3′ and the modulator stem sequence consists of 5′-UCUAC-3′. In certain embodiments, the targeter stem sequence consists of 5′-GUGGG-3′ and the modulator stem sequence consists of 5′-CCCAC-3′.

It is also contemplated that the compatibility of the duplex for a given Cas nuclease may be a factor in providing an operative modified dual guide CRISPR system. For example, the targeter stem sequence and the modulator stem sequence can be derived from a naturally occurring crRNA capable of activating a Cas nuclease in the absence of a tracrRNA. In certain embodiments, the nucleotide sequences of the targeter stem sequence and the modulator stem sequence are identical to the corresponding stem sequences of a stem-loop structure in such naturally occurring crRNA.

In certain embodiments, in a type V-A system, the 3′ end of the targeter stem sequence is linked by no more than 1, 2, 3, 4, 5, 6, 7, 8, 9, or 10 nucleotides to the 5′ end of the spacer sequence. In certain embodiments, the targeter stem sequence and the spacer sequence are adjacent to each other, directly linked by an internucleotide bond. In certain embodiments, the targeter stem sequence and the spacer sequence are linked by one nucleotide, e.g., a uridine. In certain embodiments, the targeter stem sequence and the spacer sequence are linked by two or more nucleotides. In certain embodiments, the targeter stem sequence and the spacer sequence are linked by 1, 2, 3, 4, 5, 6, 7, 8, 9, or 10 nucleotides.

In certain embodiments, the targeter nucleic acid further comprises an additional nucleotide sequence 5′ to the targeter stem sequence. In certain embodiments, the additional nucleotide sequence comprises at least 1 (e.g., at least 2, at least 3, at least 4, at least 5, at least 6, at least 7, at least 8, at least 9, at least 10, at least 15, at least 20, at least 25, at least 30, at least 35, at least 40, at least 45, or at least 50) nucleotides. In certain embodiments, the additional nucleotide sequence consists of 1, 2, 3, 4, 5, 6, 7, 8, 9, 10, 15, 20, 25, 30, 35, 40, 45, or 50 nucleotides. In certain embodiments, the additional nucleotide sequence consists of 2 nucleotides. In certain embodiments, the additional nucleotide sequence is reminiscent to the loop or a fragment thereof (e.g., one, two, three, or four nucleotides at or near the 3′ end of the loop) in a crRNA of a corresponding single guide CRISPR-Cas system. It is understood that an additional nucleotide sequence 5′ to the targeter stem sequence is dispensable. Accordingly, in certain embodiments, the targeter nucleic acid does not comprise any additional nucleotide 5′ to the targeter stem sequence.

In certain embodiments, the targeter nucleic acid or the single guide nucleic acid further comprises an additional nucleotide sequence containing one or more nucleotides at or near the 3′ end that does not hybridize with the target nucleotide sequence. The additional nucleotide sequence may protect the targeter nucleic acid from degradation by 3′-5′ exonuclease. In certain embodiments, the additional nucleotide sequence is no more than 100 nucleotides in length. In certain embodiments, the additional nucleotide sequence is no more than 90, 80, 70, 60, 50, 40, 30, 20, or 10 nucleotides in length. In certain embodiments, the additional nucleotide sequence is at least 1, 2, 3, 4, 5, 6, 7, 8, 9, 10, 15, 20, 25, 30, 35, 40, 45, or 50 nucleotides in length. In certain embodiments, the additional nucleotide sequence is 5-100, 5-50, 5-40, 5-30, 5-25, 5-20, 5-15, 5-10, 10-100, 10-50, 10-40, 10-30, 10-25, 10-20, 10-15, 15-100, 15-50, 15-40, 15-30, 15-25, 15-20, 20-100, 20-50, 20-40, 20-30, 20-25, 25-100, 25-50, 25-40, 25-30, 30-100, 30-50, 30-40, 40-100, 40-50, or 50-100 nucleotides in length.

In certain embodiments, the additional nucleotide sequence forms a hairpin with the spacer sequence. Such secondary structure may increase the specificity of guide nucleic acid or the engineered, non-naturally occurring system (see, Kocak et al. (2019) NAT. BIOTECH. 37: 657-66). In certain embodiments, the free energy change during the hairpin formation is greater than or equal to −20 kcal/mol, −15 kcal/mol, −14 kcal/mol, −13 kcal/mol, −12 kcal/mol, −11 kcal/mol, or −10 kcal/mol. In certain embodiments, the free energy change during the hairpin formation is greater than or equal to −5 kcal/mol, −6 kcal/mol, −7 kcal/mol, −8 kcal/mol, −9 kcal/mol, −10 kcal/mol, −11 kcal/mol, −12 kcal/mol, −13 kcal/mol, −14 kcal/mol, or −15 kcal/mol. In certain embodiments, the free energy change during the hairpin formation is in the range of −20 to −10 kcal/mol, −20 to −11 kcal/mol, −20 to −12 kcal/mol, −20 to −13 kcal/mol, −20 to −14 kcal/mol, −20 to −15 kcal/mol, −15 to −10 kcal/mol, −15 to −11 kcal/mol, −15 to −12 kcal/mol, −15 to −13 kcal/mol, −15 to −14 kcal/mol, −14 to −10 kcal/mol, −14 to −11 kcal/mol, −14 to −12 kcal/mol, −14 to −13 kcal/mol, −13 to −10 kcal/mol, −13 to −11 kcal/mol, −13 to −12 kcal/mol, −12 to −10 kcal/mol, −12 to −11 kcal/mol, or −11 to −10 kcal/mol. In other embodiments, the targeter nucleic acid or the single guide nucleic acid does not comprise any nucleotide 3′ to the spacer sequence.

In certain embodiments, the modulator nucleic acid further comprises an additional nucleotide sequence 3′ to the modulator stem sequence. In certain embodiments, the additional nucleotide sequence comprises at least 1 (e.g., at least 2, at least 3, at least 4, at least 5, at least 6, at least 7, at least 8, at least 9, at least 10, at least 15, at least 20, at least 25, at least 30, at least 35, at least 40, at least 45, or at least 50) nucleotides. In certain embodiments, the additional nucleotide sequence consists of 1, 2, 3, 4, 5, 6, 7, 8, 9, 10, 15, 20, 25, 30, 35, 40, 45, or 50 nucleotides. In certain embodiments, the additional nucleotide sequence consists of 1 nucleotide (e.g., uridine). In certain embodiments, the additional nucleotide sequence consists of 2 nucleotides. In certain embodiments, the additional nucleotide sequence is reminiscent to the loop or a fragment thereof (e.g., one, two, three, or four nucleotides at or near the 5′ end of the loop) in a crRNA of a corresponding single guide CRISPR-Cas system. It is understood that an additional nucleotide sequence 3′ to the modulator stem sequence is dispensable. Accordingly, in certain embodiments, the modulator nucleic acid does not comprise any additional nucleotide 3′ to the modulator stem sequence.

It is understood that the additional nucleotide sequence 5′ to the targeter stem sequence and the additional nucleotide sequence 3′ to the modulator stem sequence, if present, may interact with each other. For example, although the nucleotide immediately 5′ to the targeter stem sequence and the nucleotide immediately 3′ to the modulator stem sequence do not form a Watson-Crick base pair (otherwise they would constitute part of the targeter stem sequence and part of the modulator stem sequence, respectively), other nucleotides in the additional nucleotide sequence 5′ to the targeter stem sequence and the additional nucleotide sequence 3′ to the modulator stem sequence may form one, two, three, or more base pairs (e.g., Watson-Crick base pairs). Such interaction may affect the stability of the complex comprising the targeter nucleic acid and the modulator nucleic acid.

The stability of a complex comprising a targeter nucleic acid and a modulator nucleic acid can be assessed by the Gibbs free energy change (ΔG) during the formation of the complex, either calculated or actually measured. Where all the predicted base pairing in the complex occurs between a base in the targeter nucleic acid and a base in the modulator nucleic acid, i.e., there is no intra-strand secondary structure, the ΔG during the formation of the complex correlates generally with the ΔG during the formation of a secondary structure within the corresponding single guide nucleic acid. Methods of calculating or measuring the ΔG are known in the art. An exemplary method is RNAfold (rna.tbi.univie.ac.at/cgi-bin/RNAWebSuite/RNAfold.cgi) as disclosed in Gruber et al. (2008) NUCLEIC ACIDS RES., 36(Web Server issue): W70-W74. Unless indicated otherwise, the ΔG values in the present disclosure are calculated by RNAfold for the formation of a secondary structure within a corresponding single guide nucleic acid. In certain embodiments, the ΔG is lower than or equal to −1 kcal/mol, e.g., lower than or equal to −2 kcal/mol, lower than or equal to −3 kcal/mol, lower than or equal to −4 kcal/mol, lower than or equal to −5 kcal/mol, lower than or equal to −6 kcal/mol, lower than or equal to −7 kcal/mol, lower than or equal to −7.5 kcal/mol, or lower than or equal to −8 kcal/mol. In certain embodiments, the ΔG is greater than or equal to −10 kcal/mol, e.g., greater than or equal to −9 kcal/mol, greater than or equal to −8.5 kcal/mol, or greater than or equal to −8 kcal/mol. In certain embodiments, the ΔG is in the range of −10 to −4 kcal/mol. In certain embodiments, the ΔG is in the range of −8 to −4 kcal/mol, −7 to −4 kcal/mol, −6 to −4 kcal/mol, −5 to −4 kcal/mol, −8 to −4.5 kcal/mol, −7 to −4.5 kcal/mol, −6 to −4.5 kcal/mol, or −5 to −4.5 kcal/mol, for example −8 kcal/mol, −7 kcal/mol, −6 kcal/mol, −5 kcal/mol, −4.9 kcal/mol, −4.8 kcal/mol, −4.7 kcal/mol, −4.6 kcal/mol, −4.5 kcal/mol, −4.4 kcal/mol, −4.3 kcal/mol, −4.2 kcal/mol, −4.1 kcal/mol, or −4 kcal/mol.

It is understood that the ΔG may be affected by a sequence in the targeter nucleic acid that is not within the targeter stem sequence, and/or a sequence in the modulator nucleic acid that is not within the modulator stem sequence. For example, one or more base pairs (e.g., Watson-Crick base pair) between an additional sequence 5′ to the targeter stem sequence and an additional sequence 3′ to the modulator stem sequence may reduce the ΔG, i.e., stabilize the nucleic acid complex. In certain embodiments, the nucleotide immediately 5′ to the targeter stem sequence comprises a uracil or is a uridine, and the nucleotide immediately 3′ to the modulator stem sequence comprises a uracil or is a uridine, thereby forming a nonconventional U-U base pair.

In certain embodiments, the modulator nucleic acid or the single guide nucleic acid comprises a nucleotide sequence referred to herein as a “5′ sequence”, e.g., a tail sequence, positioned 5′ to the modulator stem sequence. In a naturally occurring type V-A CRISPR-Cas system, the 5′ sequence, e.g., a tail sequence, is a nucleotide sequence positioned 5′ to the stem-loop structure of the crRNA. A 5′ sequence, e.g., a tail sequence, in an engineered type V-A CRISPR-Cas system, whether single guide or dual guide, can be reminiscent to the 5′ sequence, e.g., a tail sequence, in a corresponding naturally occurring type V-A CRISPR-Cas system.

Without being bound by theory, it is contemplated that the 5′ sequence, e.g., a tail sequence, may participate in the formation of the CRISPR-Cas complex. For example, in certain embodiments, the 5′ sequence, e.g., a tail sequence, forms a pseudoknot structure with the modulator stem sequence, which is recognized by the Cas protein (see, Yamano et al. (2016) CELL, 165: 949). In certain embodiments, the 5′ sequence, e.g., a tail sequence, is at least 3 (e.g., at least 4 or at least 5) nucleotides in length. In certain embodiments, the 5′ sequence, e.g., a tail sequence, is 3, 4, or 5 nucleotides in length. In certain embodiments, the nucleotide at the 3′ end of the 5′ sequence, e.g., a tail sequence, comprises a uracil or is a uridine. In certain embodiments, the second nucleotide in the 5′ sequence, e.g., a tail sequence, the position counted from the 3′ end, comprises a uracil or is a uridine. In certain embodiments, the third nucleotide in the 5′ sequence, e.g., a tail sequence, the position counted from the 3′ end, comprises an adenine or is an adenosine. This third nucleotide may form a base pair (e.g., a Watson-Crick base pair) with a nucleotide 5′ to the modulator stem sequence. Accordingly, in certain embodiments, the modulator nucleic acid comprises a uridine or a uracil-containing nucleotide 5′ to the modulator stem sequence. In certain embodiments, the 5′ sequence, e.g., a tail sequence, comprises the nucleotide sequence of 5′-AUU-3′. In certain embodiments, the 5′ sequence, e.g., a tail sequence, comprises the nucleotide sequence of 5′-AAUU-3′. In certain embodiments, the 5′ sequence, e.g., a tail sequence, comprises the nucleotide sequence of 5′-UAAUU-3′. In certain embodiments, the 5′ sequence, e.g., a tail sequence, is positioned immediately 5′ to the modulator stem sequence.

In certain embodiments, the single guide nucleic acid, the targeter nucleic acid, and/or the modulator nucleic acid are designed to reduce the degree of secondary structure other than the hybridization between the targeter stem sequence and the modulator stem sequence. In certain embodiments, no more than 75%, 50%, 40%, 30%, 25%, 20%, 15%, 10%, 5%, 1%, or fewer of the nucleotides of the single guide nucleic acid other than the targeter stem sequence and the modulator stem sequence participate in self-complementary base pairing when optimally folded. In certain embodiments, no more than 75%, 50%, 40%, 30%, 25%, 20%, 15%, 10%, 5%, 1%, or fewer of the nucleotides of the targeter nucleic acid and/or the modulator nucleic acid participate in self-complementary base pairing when optimally folded. Optimal folding may be determined by any suitable polynucleotide folding algorithm. Some programs are based on calculating the minimal Gibbs free energy. An example of one such algorithm is mFold, as described by Zuker and Stiegler (Nucleic Acids Res. 9 (1981), 133-148). Another example folding algorithm is the online webserver RNAfold, developed at Institute for Theoretical Chemistry at the University of Vienna, using the centroid structure prediction algorithm (see e.g., A. R. Gruber et al., 2008, Cell 106(1): 23-24; and P A Carr and G M Church, 2009, Nature Biotechnology 27(12): 1151-62).

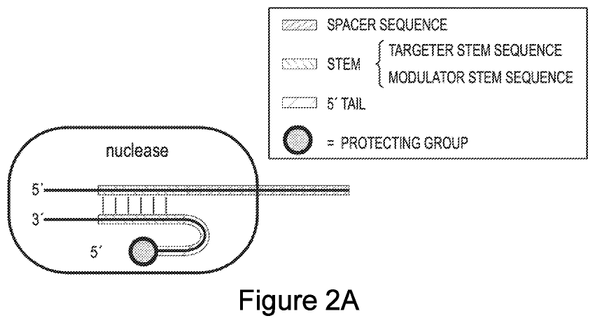

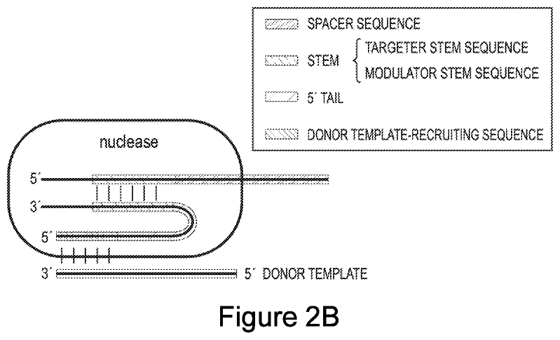

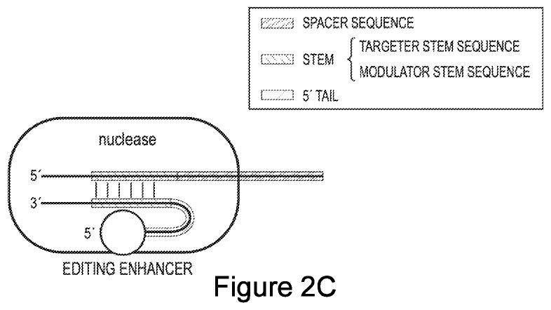

The targeter nucleic acid is directed to a specific target nucleotide sequence, and a donor template can be designed to modify the target nucleotide sequence or a sequence nearby. It is understood, therefore, that association of the single guide nucleic acid, the targeter nucleic acid, or the modulator nucleic acid with a donor template can increase editing efficiency and reduce off-targeting. Accordingly, in certain embodiments, the single guide nucleic acid or the modulator nucleic acid further comprises a donor template-recruiting sequence capable of hybridizing with a donor template (see FIG. 2B). Donor templates are described in the “Donor Templates” subsection of section II infra. The donor template and donor template-recruiting sequence can be designed such that they bear sequence complementarity. In certain embodiments, the donor template-recruiting sequence is at least 90% (e.g., at least 91%, at least 92%, at least 93%, at least 94%, at least 95%, at least 96%, at least 97%, at least 98%, or at least 99%) complementary to at least a portion of the donor template. In certain embodiments, the donor template-recruiting sequence is 100% complementary to at least a portion of the donor template. In certain embodiments, where the donor template comprises an engineered sequence not homologous to the sequence to be repaired, the donor template-recruiting sequence is capable of hybridizing with the engineered sequence in the donor template. In certain embodiments, the donor template-recruiting sequence is at least 20, 25, 30, 35, 40, 45, 50, 55, 60, 65, 70, 75, 80, 85, 90, 95, or 100 nucleotides in length. In certain embodiments, the donor template-recruiting sequence is positioned at or near the 5′ end of the single guide nucleic acid or at or near the 5′ end of the modulator nucleic acid. In certain embodiments, the donor template-recruiting sequence is linked to the 5′ sequence, e.g., tail sequence, if present, or to the modulator stem sequence, of the single guide nucleic acid or the modulator nucleic acid through an internucleotide bond or a nucleotide linker.

In certain embodiments, a guide nucleic acid as described herein is associated with a donor template comprising a single strand oligodeoxynucleotide (ssODN).

In certain embodiments, the single guide nucleic acid or the modulator nucleic acid further comprises an editing enhancer sequence, which increases the efficiency of gene editing and/or homology-directed repair (HDR) (see FIG. 2C). Exemplary editing enhancer sequences are described in Park et al. (2018) NAT. COMMUN. 9: 3313. In certain embodiments, the editing enhancer sequence is positioned 5′ to the 5′ sequence, e.g., a tail sequence, if present, or 5′ to the single guide nucleic acid or the modulator stem sequence. In certain embodiments, the editing enhancer sequence is 1-50, 4-50, 9-50, 15-50, 25-50, 1-25, 4-25, 9-25, 15-25, 1-15, 4-15, 9-15, 1-9, 4-9, or 1-4 nucleotides in length. In certain embodiments, the editing enhancer sequence is at least 1, 2, 3, 4, 5, 6, 7, 8, 9, 10, 15, 20, 25, 30, 35, 40, 45, 50, or 55 nucleotides in length. The editing enhancer sequence is designed to minimize homology to the target nucleotide sequence or any other sequence that the engineered, non-naturally occurring system may be contacted to, e.g., the genome sequence of a cell into which the engineered, non-naturally occurring system is delivered. In certain embodiments, the editing enhancer is designed to minimize the presence of hairpin structure. The editing enhancer can comprise one or more of the chemical modifications disclosed herein.

The single guide nucleic acid, the modulator nucleic acid, and/or the targeter nucleic acid can further comprise a protective nucleotide sequence that prevents or reduces nucleic acid degradation. In certain embodiments, the protective nucleotide sequence is at least 5 (e.g., at least 10, at least 15, at least 20, at least 25, at least 30, at least 35, at least 40, at least 45, or at least 50) nucleotides in length. The length of the protective nucleotide sequence increases the time for an exonuclease to reach the 5′ sequence, e.g., a tail sequence, modulator stem sequence, targeter stem sequence, and/or spacer sequence, thereby protecting these portions of the single guide nucleic acid, the modulator nucleic acid, and/or the targeter nucleic acid from degradation by an exonuclease. In certain embodiments, the protective nucleotide sequence forms a secondary structure, such as a hairpin or a tRNA structure, to reduce the speed of degradation by an exonuclease (see, for example, Wu et al. (2018) CELL. MOL. LIFE SCI., 75(19): 3593-3607). Secondary structures can be predicted by methods known in the art, such as the online webserver RNAfold developed at University of Vienna using the centroid structure prediction algorithm (see, Gruber et al. (2008) NUCLEIC ACIDS RES., 36: W70). Certain chemical modifications, which may be present in the protective nucleotide sequence, can also prevent or reduce nucleic acid degradation, as disclosed in the “RNA Modifications” subsection infra.

A protective nucleotide sequence is typically located at or near the 5′ or 3′ end of the single guide nucleic acid, the modulator nucleic acid, and/or the targeter nucleic acid. In certain embodiments, the single guide nucleic acid comprises a protective nucleotide sequence at or near the 5′ end, at or near the 3′ end, or at or near both ends, optionally through a nucleotide linker. In certain embodiments, the modulator nucleic acid comprises a protective nucleotide sequence at or near the 5′ end, at or near the 3′ end, or at or near both ends, optionally through a nucleotide linker. In particular embodiments, the modulator nucleic acid comprises a protective nucleotide sequence at or near the 5′ end (see FIG. 2A). In certain embodiments, the targeter nucleic acid comprises a protective nucleotide sequence at or near the 5′ end, at or near the 3′ end, or at or near both ends, optionally through a nucleotide linker.

As described above, various nucleotide sequences can be present in the 5′ portion of a single nucleic acid or a modulator nucleic acid, including but not limited to a donor template-recruiting sequence, an editing enhancer sequence, a protective nucleotide sequence, and a linker connecting such sequence to the 5′ sequence, e.g., tail sequence, if present, or to the modulator stem sequence. It is understood that the functions of donor template recruitment, editing enhancement, protection against degradation, and linkage are not exclusive to each other, and one nucleotide sequence can have one or more of such functions. For example, in certain embodiments, the single guide nucleic acid or the modulator nucleic acid comprises a nucleotide sequence that is both a donor template-recruiting sequence and an editing enhancer sequence. In certain embodiments, the single guide nucleic acid or the modulator nucleic acid comprises a nucleotide sequence that is both a donor template-recruiting sequence and a protective sequence. In certain embodiments, the single guide nucleic acid or the modulator nucleic acid comprises a nucleotide sequence that is both an editing enhancer sequence and a protective sequence. In certain embodiments, the single guide nucleic acid or the modulator nucleic acid comprises a nucleotide sequence that is a donor template-recruiting sequence, an editing enhancer sequence, and a protective sequence. In certain embodiments, the nucleotide sequence 5′ to the 5′ sequence, e.g., a tail sequence, if present, or 5′ to the modulator stem sequence is 1-90, 1-80, 1-70, 1-60, 1-50, 1-40, 1-30, 1-20, 1-10, 10-90, 10-80, 10-70, 10-60, 10-50, 10-40, 10-30, 10-20, 20-90, 20-80, 20-70, 20-60, 20-50, 20-40, 20-30, 30-90, 30-80, 30-70, 30-60, 30-50, 30-40, 40-90, 40-80, 40-70, 40-60, 40-50, 50-90, 50-80, 50-70, 50-60, 60-90, 60-80, 60-70, 70-90, 70-80, or 80-90 nucleotides in length.

In certain embodiments, the engineered, non-naturally occurring system further comprises one or more compounds (e.g., small molecule compounds) that enhance HDR and/or inhibit NHEJ. Exemplary compounds having such functions are described in Maruyama et al. (2015) NAT BIOTECHNOL. 33(5): 538-42; Chu et al. (2015) NAT BIOTECHNOL. 33(5): 543-48; Yu et al. (2015) CELL STEM CELL 16(2): 142-47; Pinder et al. (2015) NUCLEIC ACIDS RES. 43(19): 9379-92; and Yagiz et al. (2019) COMMUN. BIOL. 2: 198. In certain embodiments, the engineered, non-naturally occurring system further comprises one or more compounds selected from the group consisting of DNA ligase IV antagonists (e.g., SCR7 compound, Ad4 ElB55K protein, and Ad4 E4orf6 protein), RAD51 agonists (e.g., RS-1), DNA-dependent protein kinase (DNA-PK) antagonists (e.g., NU7441 and KU0060648), β3-adrenergic receptor agonists (e.g., L755507), inhibitors of intracellular protein transport from the ER to the Golgi apparatus (e.g., brefeldin A), and any combinations thereof.

In certain embodiments, the engineered, non-naturally occurring system comprising a targeter nucleic acid and a modulator nucleic acid is tunable or inducible. For example, in certain embodiments, the targeter nucleic acid, the modulator nucleic acid, and/or the Cas protein can be introduced to the target nucleotide sequence at different times, the system becoming active only when all components are present. In certain embodiments, the amounts of the targeter nucleic acid, the modulator nucleic acid, and/or the Cas protein can be titrated to achieve desired efficiency and specificity. In certain embodiments, excess amount of a nucleic acid comprising the targeter stem sequence or the modulator stem sequence can be added to the system, thereby dissociating the complex of the targeter nucleic and modulator nucleic acid and turning off the system.

### B. RNA Modifications

The guide nucleic acids disclosed herein, including a single guide nucleic acid, a targeter nucleic acid, and/or a modulator nucleic acid, may comprise a DNA (e.g., modified DNA), an RNA (e.g., modified RNA), or a combination thereof. In certain embodiments, the single guide nucleic acid comprises a DNA (e.g., modified DNA), an RNA (e.g., modified RNA), or a combination thereof. In certain embodiments, the targeter nucleic acid comprises a DNA (e.g., modified DNA), an RNA (e.g., modified RNA), or a combination thereof. In certain embodiments, the modulator nucleic acid comprises a DNA (e.g., modified DNA), an RNA (e.g., modified RNA), or a combination thereof. The spacer sequences disclosed herein are presented as DNA sequences by including thymidines (T) rather than uridines (U). It is understood that corresponding RNA sequences and DNA/RNA chimeric sequences are also contemplated. For example, where the spacer sequence is an RNA, its sequence can be derived from a DNA sequence disclosed herein by replacing each T with U. As a result, for the purpose of describing a nucleotide sequence, T and U are used interchangeably herein.

In certain embodiments, the single guide nucleic acid is an RNA. A single guide nucleic acid in the form of an RNA is also called a single guide RNA. In certain embodiments, the targeter nucleic acid is an RNA and the modulator nucleic acid is an RNA. A targeter nucleic acid in the form of an RNA is also called targeter RNA, and a modulator nucleic acid in the form of an RNA is also called modulator RNA.

In certain embodiments some or all of the gNA is RNA, e.g., a gRNA. In certain embodiments, 5-100%, 10-100%, 20-100%, 30-100%, 40-100%, 50-100%, 60-100%, 70-100%, 80-100%, 90-100%, 95-100%, 99-100%, 99.5-100% of the gNA is gRNA. In certain embodiments, 20%-80%, 20%-70%, 20%-60%, 20%-50%, 20%-40%, 20%-30%, 30%-80%, 30%-70%, 30%-60%, 30%-50%, 30%-40%, 40%-80%, 40%-70%, 40%-60%, 40%-50%, 50%-80%, 50%-70%, 50%-60%, 60%-80%, 60%-70%, or 70%-80% of gNA is RNA. In certain embodiments, 50% of the gNA is RNA. In certain embodiments, 70% of the gNA is RNA. In certain embodiments, 90% of the gNA is RNA. In certain embodiments, 100% of the gNA is RNA, e.g., a gRNA.

In certain embodiments the stem sequences are 1-20, 2-19, 3-18, 4-17, 5-16, 6-15, 7-14, 8-13, 9-12, 10-11, 1-9, 2-8, 3-7, 4-6, or 2-9 nucleotides in length. In a preferred embodiment, the stem sequences are 4-6 nucleotides in length. In certain embodiments, the stem sequence of the modulator and targeter nucleic acids share 5-100%, 10-100%, 20-100%, 30-100%, 40-100%, 50-100%, 60-100%, 70-100%, 80-100%, 90-100%, 95-100%, 99-100%, 99.5-100% of the gNA is gRNA. In certain embodiments, 20%-80%, 20%-70%, 20%-60%, 20%-50%, 20%-40%, 20%-30%, 30%-80%, 30%-70%, 30%-60%, 30%-50%, 30%-40%, 40%-80%, 40%-70%, 40%-60%, 40%-50%, 50%-80%, 50%-70%, 50%-60%, 60%-80%, 60%-70%, or 70%-80% sequence complementarity. In certain embodiments, the stem sequence of the modulator and targeter nucleic acids share 80, 90, 95, or 100% sequence complementarity. In a preferred embodiment, the stem sequence of the modulator and targeter nucleic acids share 80-100% sequence complementarity.

In certain embodiments, the single guide nucleic acid, the targeter nucleic acid, and/or the modulator nucleic acid are RNAs with one or more modifications in a ribose group, one or more modifications in a phosphate group, one or more modifications in a nucleobase, one or more terminal modifications, or a combination thereof. Exemplary modifications are disclosed in U.S. Pat. Nos. 10,900,034 and 10,767,175, U.S. Patent Application Publication No. 2018/0119140, Watts et al. (2008) Drug Discov. Today 13: 842-55, and Hendel et al. (2015) NAT. BIOTECHNOL. 33: 985.

Modifications in a ribose group include but are not limited to modifications at the 2′ position or modifications at the 4′ position. For example, in certain embodiments, the ribose comprises 2′-O—C1-4alkyl, such as 2′-O-methyl (2′-OMe). In certain embodiments, the ribose comprises 2′-O—C1-3alkyl-O—C1-3alkyl, such as 2′-methoxyethoxy (2′-O—CH₂CH₂OCH₃) also known as 2′-O-(2-methoxyethyl) or 2′-MOE. In certain embodiments, the ribose comprises 2′-O-allyl. In certain embodiments, the ribose comprises 2′-O-2,4-Dinitrophenol (DNP). In certain embodiments, the ribose comprises 2′-halo, such as 2′-F, 2′-Br, 2′-Cl, or 2′-I. In certain embodiments, the ribose comprises 2′-NH₂. In certain embodiments, the ribose comprises 2′-H (e.g., a deoxynucleotide). In certain embodiments, the ribose comprises 2′-arabino or 2′-F-arabino. In certain embodiments, the ribose comprises 2′-LNA or 2′-ULNA. In certain embodiments, the ribose comprises a 4′-thioribosyl.

Modifications can also include a deoxy group, for example a 2′-deoxy-3′-phosphonoacetate (DP), a 2′-deoxy-3′-thiophosphonoacetate (DSP).

Modifications in a phosphate group include but are not limited to a phosphorothioate, a chiral phosphorothioate, a phosphorodithioate, a boranophosphonate, a C₁₋₄alkyl phosphonate such as a methylphosphonate, a boranophosphonate, a phosphonocarboxylate such as a phosphonoacetate, a phosphonocarboxylate ester such as a phosphonoacetate ester, an amide linkage, a thiophosphonocarboxylate such as a thiophosphonoacetate, a thiophosphonocarboxylate ester such as a thiophosphonoacetate ester, and a 2′,5′-linkage having a phosphodiester linker or any of the linkers above. Various salts, mixed salts and free acid forms are also included.

Modifications in a nucleobase include but are not limited to 2-thiouracil, 2-thiocytosine, 4-thiouracil, 6-thioguanine, 2-aminoadenine, 2-aminopurine, pseudouracil, hypoxanthine, 7-deazaguanine, 7-deaza-8-azaguanine, 7-deazaadenine, 7-deaza-8-azaadenine, 5-methylcytosine, 5-methyluracil, 5-hydroxymethylcytosine, 5-hydroxymethyluracil, 5,6-dehydrouracil, 5-propynylcytosine, 5-propynyluracil, 5-ethynylcytosine, 5-ethynyluracil, 5-allyluracil, 5-allylcytosine, 5-aminoallyluracil, 5-aminoallyl-cytosine, 5-bromouracil, 5-iodouracil, diaminopurine, difluorotoluene, dihydrouracil, an abasic nucleotide, Z base, P base, Unstructured Nucleic Acid, isoguanine, isocytosine (see, Piccirilli et al. (1990) NATURE, 343: 33), 5-methyl-2-pyrimidine (see, Rappaport (1993) BIOCHEMISTRY, 32: 3047), x(A,G,C,T), and y(A,G,C,T).

Terminal modifications include but are not limited to polyethyleneglycol (PEG), hydrocarbon linkers (such as heteroatom (O,S,N)-substituted hydrocarbon spacers; halo-substituted hydrocarbon spacers; keto-, carboxyl-, amido-, thionyl-, carbamoyl-, thionocarbamaoyl-containing hydrocarbon spacers), spermine linkers, dyes such as fluorescent dyes (for example, fluoresceins, rhodamines, cyanines), quenchers (for example, dabcyl, BHQ), and other labels (for example biotin, digoxigenin, acridine, streptavidin, avidin, peptides and/or proteins). In certain embodiments, a terminal modification comprises a conjugation (or ligation) of the RNA to another molecule comprising an oligonucleotide (such as deoxyribonucleotides and/or ribonucleotides), a peptide, a protein, a sugar, an oligosaccharide, a steroid, a lipid, a folic acid, a vitamin and/or other molecule. In certain embodiments, a terminal modification incorporated into the RNA is located internally in the RNA sequence via a linker such as 2-(4-butylamidofluorescein)propane-1,3-diol bis(phosphodiester) linker, which is incorporated as a phosphodiester linkage and can be incorporated anywhere between two nucleotides in the RNA.

The modifications disclosed above can be combined in the targeter nucleic acid and/or the modulator nucleic acid that are in the form of RNA. In certain embodiments, the modification in the RNA is selected from the group consisting of incorporation of 2′-O-methyl-3′phosphorothioate (MS), 2′-O-methyl-3′-phosphonoacetate (MP), 2′-O-methyl-3′-thiophosphonoacetate (MSP), 2′-halo-3′-phosphorothioate (e.g., 2′-fluoro-3′-phosphorothioate), 2′-halo-3′-phosphonoacetate (e.g., 2′-fluoro-3′-phosphonoacetate), and 2′-halo-3′-thiophosphonoacetate (e.g., 2′-fluoro-3′-thiophosphonoacetate).

In certain embodiments, modifications can include 2′-O-methyl (M), a phosphorothioate (S), a phosphonoacetate (P), a thiophosphonoacetate (SP), a 2′-O-methyl-3′-phosphorothioate (MS), a 2′-O-methyl-3′-phosphonoacetate (MP), a 2′-O-methyl-3′-thiophosphonoacetate (MSP), a 2′-deoxy-3′-phosphonoacetate (DP), a 2′-deoxy-3′-thiophosphonoacetate (DSP), or a combination thereof, at or near either the 3′ or 5′ end of either the targeter or modulator nucleic acid, as appropriate for single or dual gNA.

In certain embodiments, modifications can include either a 5′ or a 3′ propanediol or C3 linker modification.

The modifications disclosed above can be combined in the single guide RNA, the targeter RNA, and/or the modulator RNA. In certain embodiments, the modification in the RNA is selected from the group consisting of incorporation of 2′-O-methyl-3′phosphorothioate, 2′-O-methyl-3′-phosphonoacetate, 2′-O-methyl-3′-thiophosphonoacetate, 2′-halo-3′-phosphorothioate (e.g., 2′-fluoro-3′-phosphorothioate), 2′-halo-3′-phosphonoacetate (e.g., 2′-fluoro-3′-phosphonoacetate), and 2′-halo-3′-thiophosphonoacetate (e.g., 2′-fluoro-3′-thiophosphonoacetate).

In certain embodiments, the modification alters the stability of the RNA. In certain embodiments, the modification enhances the stability of the RNA, e.g., by increasing nuclease resistance of the RNA relative to a corresponding RNA without the modification. Stability-enhancing modifications include but are not limited to incorporation of 2′-O-methyl, a 2′-O—C₁₋₄alkyl, 2′-halo (e.g., 2′-F, 2′-Br, 2′-Cl, or 2′-I), 2′MOE, a 2′-O—C₁₋₃alkyl-O—C₁₋₃alkyl, 2′-NH₂, 2′-H (or 2′-deoxy), 2′-arabino, 2′-F-arabino, 4′-thioribosyl sugar moiety, 3′-phosphorothioate, 3′-phosphonoacetate, 3′-thiophosphonoacetate, 3′-methylphosphonate, 3′-boranophosphate, 3′-phosphorodithioate, locked nucleic acid (“LNA”) nucleotide which comprises a methylene bridge between the 2′ and 4′ carbons of the ribose ring, and unlocked nucleic acid (“ULNA”) nucleotide. Such modifications are suitable for use as a protecting group to prevent or reduce degradation of the 5′ sequence, e.g., a tail sequence, modulator stem sequence, targeter stem sequence, and/or spacer sequence (see, the “Guide Nucleic Acids” subsection supra).

In certain embodiments, the modification alters the specificity of the engineered, non-naturally occurring system. In certain embodiments, the modification enhances the specification of the engineered, non-naturally occurring system, e.g., by enhancing on-target binding and/or cleavage, or reducing off-target binding and/or cleavage, or a combination thereof. Specificity-enhancing modifications include but are not limited to 2-thiouracil, 2-thiocytosine, 4-thiouracil, 6-thioguanine, 2-aminoadenine, and pseudouracil.

In certain embodiments, the modification alters the immunostimulatory effect of the RNA relative to a corresponding RNA without the modification. For example, in certain embodiments, the modification reduces the ability of the RNA to activate TLR7, TLR8, TLR9, TLR3, RIG-I, and/or MDA5.

In certain embodiments, the single guide nucleic acid, the targeter nucleic acid, and/or the modulator nucleic acid comprise at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 modified nucleotides. The modification can be made at one or more positions in the single guide nucleic acid, the targeter nucleic acid, and/or the modulator nucleic acid such that these nucleic acids retain functionality. For example, the modified nucleic acids can still direct the Cas protein to the target nucleotide sequence and allow the Cas protein to exert its effector function. It is understood that the particular modification(s) at a position may be selected based on the functionality of the nucleotide at the position. For example, a specificity-enhancing modification may be suitable for one or more nucleotides or internucleotide linkages in the spacer sequence, the targeter stem sequence, or the modulator stem sequence. A stability-enhancing modification may be suitable for one or more terminal nucleotides or internucleotide linkages in the single guide nucleic acid, the targeter nucleic acid, and/or the modulator nucleic acid. In certain embodiments, at least 1 (e.g., at least 2, at least 3, at least 4, or at least 5) terminal nucleotides or internucleotide linkages at the 5′ end and/or at least 1 (e.g., at least 2, at least 3, at least 4, or at least 5) terminal nucleotides or internucleotide linkages at the 3′ end of the single guide nucleic acid are modified. In certain embodiments, 5 or fewer (e.g., 1 or fewer, 2 or fewer, 3 or fewer, or 4 or fewer) terminal nucleotides or internucleotide linkages at the 5′ end and/or 5 or fewer (e.g., 1 or fewer, 2 or fewer, 3 or fewer, or 4 or fewer) terminal nucleotides or internucleotide linkages at the 3′ end of the single guide nucleic acid are modified. In certain embodiments, at least 1 (e.g., at least 2, at least 3, at least 4, or at least 5) terminal nucleotides or internucleotide linkages at the 5′ end and/or at least 1 (e.g., at least 2, at least 3, at least 4, or at least 5) terminal nucleotides or internucleotide linkages at the 3′ end of the targeter nucleic acid are modified. In certain embodiments, 5 or fewer (e.g., 1 or fewer, 2 or fewer, 3 or fewer, or 4 or fewer) terminal nucleotides or internucleotide linkages at the 5′ end and/or 5 or fewer (e.g., 1 or fewer, 2 or fewer, 3 or fewer, or 4 or fewer) terminal nucleotides or internucleotide linkages at the 3′ end of the targeter nucleic acid are modified. In certain embodiments, at least 1 (e.g., at least 2, at least 3, at least 4, or at least 5) terminal nucleotides or internucleotide linkages at the 5′ end and/or at least 1 (e.g., at least 2, at least 3, at least 4, or at least 5) terminal nucleotides internucleotide linkages at the 3′ end of the modulator nucleic acid are modified. In certain embodiments, 5 or fewer (e.g., 1 or fewer, 2 or fewer, 3 or fewer, or 4 or fewer) terminal nucleotides at the 5′ end and/or 5 or fewer (e.g., 1 or fewer, 2 or fewer, 3 or fewer, or 4 or fewer) terminal nucleotides or internucleotide linkages at the 3′ end of the modulator nucleic acid are modified. Selection of positions for modifications is described in U.S. Pat. Nos. 10,900,034 and 10,767,175. As used in this paragraph, where the targeter or modulator nucleic acid is a combination of DNA and RNA, the nucleic acid as a whole is considered as an RNA, and the DNA nucleotide(s) are considered as modification(s) of the RNA, including a 2′-H modification of the ribose and optionally a modification of the nucleobase. Exemplary modifications are disclosed in Dang et al. (2015) Genome Biol. 16: 280, Kocaz et al. (2019) Nature Biotech. 37: 657-66, Liu et al. (2019) Nucleic Acids Res. 47(8): 4169-4180, Schubert et al. (2018) J. Cytokine Biol. 3(1): 121, Teng et al. (2019) Genome Biol. 20(1): 15, Watts et al. (2008) Drug Discov. Today 13(19-20): 842-55, and Wu et al. (2018) Cell Mol. Life. Sci. 75(19): 3593-607.

It is understood that the targeter nucleic acid and the modulator nucleic acid, while not in the same nucleic acids, i.e., not linked end-to-end through a traditional internucleotide bond, can be covalently conjugated to each other through one or more chemical modifications introduced into these nucleic acids, thereby increasing the stability of the double-stranded complex and/or improving other characteristics of the system.

### II. METHODS OF TARGETING, EDITING, AND/OR MODIFYING GENOMIC DNA

The engineered, non-naturally occurring system disclosed herein are useful for targeting, editing, and/or modifying a target nucleic acid, such as a DNA (e.g., genomic DNA) in a cell or organism. For example, in certain embodiments, with respect to a given target gene listed in Tables 1-9, an engineered, non-naturally occurring system disclosed herein that comprises a guide nucleic acid comprising a corresponding spacer sequence, when delivered into a population of human cells (e.g., Jurkat cells) ex vivo, edits the genomic sequence at the locus of the target gene in at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, or at least 99% of the cells.

The present invention provides a method of cleaving a target nucleic acid (e.g., DNA) comprising the sequence of a preselected target gene or a portion thereof, the method comprising contacting the target DNA with an engineered, non-naturally occurring system disclosed herein, thereby resulting in cleavage of the target DNA.

In addition, the present invention provides a method of binding a target nucleic acid (e.g., DNA) comprising the sequence of a preselected target gene or a portion thereof, the method comprising contacting the target DNA with an engineered, non-naturally occurring system disclosed herein, thereby resulting in binding of the system to the target DNA. This method is useful for detecting the presence and/or location of the preselected target gene, for example, if a component of the system (e.g., the Cas protein) comprises a detectable marker.

In addition, the present invention provides a method of modifying a target nucleic acid (e.g., DNA) comprising the sequence of a preselected target gene or a portion thereof, or a structure (e.g., protein) associated with the target DNA (e.g., a histone protein in a chromosome), the method comprising contacting the target DNA with an engineered, non-naturally occurring system disclosed herein, wherein the Cas protein comprises an effector domain or is associated with an effector protein, thereby resulting in modification of the target DNA or the structure associated with the target DNA. The modification corresponds to the function of the effector domain or effector protein. Exemplary functions described in the “Cas Proteins” subsection in Section I supra are applicable hereto.

The engineered, non-naturally occurring system can be contacted with the target nucleic acid as a complex. Accordingly, in certain embodiments, the method comprises contacting the target nucleic acid with a CRISPR-Cas complex comprising a targeter nucleic acid, a modulator nucleic acid, and a Cas protein disclosed herein. In certain embodiments, the Cas protein is a type V-A, type V-C, or type V-D Cas protein (e.g., Cas nuclease). In certain embodiments, the Cas protein is a type V-A Cas protein (e.g., Cas nuclease).

The preselected target genes include human APLNR, BBS1, CALR, CD247, CD3D, CD38, CD3E, CD3G, CD40LG, CD52, CD58, COL17A1, CSF2, DEFB134, ERAP1, ERAP2, IFNGR1, IFNGR2, JAK1, JAK2, mir-101-2, MLANA, NLRC5 PSMB5, PSMB8, PSMB9, PTCD2, RFX5, RFXANK, RFXAP, RPL23, SOX10, SRP54, STAT1, Tap1, TAP2, TAPBP, TRBC1, TRBC1_2 (or TRBC1+2), TRBC2, or TWF1 genes. Accordingly, the present invention also provides a method of editing a human genomic sequence at one of these preselected target gene loci, the method comprising delivering the engineered, non-naturally occurring system disclosed herein into a human cell, thereby resulting in editing of the genomic sequence at the target gene locus in the human cell. In addition, the present invention provides a method of detecting a human genomic sequence at one of these preselected target gene loci, the method comprising delivering the engineered, non-naturally occurring system disclosed herein into a human cell, wherein a component of the system (e.g., the Cas protein) comprises a detectable marker, thereby detecting the target gene locus in the human cell. In addition, the present invention provides a method of modifying a human chromosome at one of these preselected target gene loci, the method comprising delivering the engineered, non-naturally occurring system disclosed herein into a human cell, wherein the Cas protein comprises an effector domain or is associated with an effector protein, thereby resulting in modification of the chromosome at the target gene locus in the human cell.

The CRISPR-Cas complex may be delivered to a cell by introducing a pre-formed ribonucleoprotein (RNP) complex into the cell. Alternatively, one or more components of the CRISPR-Cas complex may be expressed in the cell. Exemplary methods of delivery are known in the art and described in, for example, U.S. Pat. Nos. 10,113,167, 8,697,359, 10,570,418, 11,125,739, 10,829,787, and 11,118,194, and U.S. Patent Application Publication Nos. 2015/0344912, 2018/0119140, and 2018/0282763.

It is understood that contacting a DNA (e.g., genomic DNA) in a cell with a CRISPR-Cas complex does not require delivery of all components of the complex into the cell. For examples, one or more of the components may be pre-existing in the cell. In certain embodiments, the cell (or a parental/ancestral cell thereof) has been engineered to express the Cas protein, and the single guide nucleic acid (or a nucleic acid comprising a regulatory element operably linked to a nucleotide sequence encoding the single guide nucleic acid), the targeter nucleic acid (or a nucleic acid comprising a regulatory element operably linked to a nucleotide sequence encoding the targeter nucleic acid), and/or the modulator nucleic acid (or a nucleic acid comprising a regulatory element operably linked to a nucleotide sequence encoding the modulator nucleic acid) are delivered into the cell. In certain embodiments, the cell (or a parental/ancestral cell thereof) has been engineered to express the modulator nucleic acid, and the Cas protein (or a nucleic acid comprising a regulatory element operably linked to a nucleotide sequence encoding the Cas protein) and the targeter nucleic acid (or a nucleic acid comprising a regulatory element operably linked to a nucleotide sequence encoding the targeter nucleic acid) are delivered into the cell. In certain embodiments, the cell (or a parental/ancestral cell thereof) has been engineered to express the Cas protein and the modulator nucleic acid, and the targeter nucleic acid (or a nucleic acid comprising a regulatory element operably linked to a nucleotide sequence encoding the targeter nucleic acid) is delivered into the cell.

In certain embodiments, the target DNA is in the genome of a target cell. Accordingly, the present invention also provides a cell comprising the non-naturally occurring system or a CRISPR expression system described herein. In addition, the present invention provides a cell whose genome has been modified by the CRISPR-Cas system or complex disclosed herein.

The target cells can be mitotic or post-mitotic cells from any organism, such as a bacterial cell, an archaeal cell, a cell of a single-cell eukaryotic organism, a plant cell, an algal cell, e.g., Botryococcus braunii, Chlamydomonas reinhardtii, Nannochloropsis gaditana, Chlorella pyrenoidosa, Sargassum patens C. Agardh, and the like, a fungal cell (e.g., a yeast cell), an animal cell, a cell from an invertebrate animal (e.g., fruit fly, enidarian, echinoderm, nematode, etc.), a cell from a vertebrate animal (e.g., fish, amphibian, reptile, bird, mammal), a cell from a mammal, a cell from a rodent, or a cell from a human. The types of target cells include but are not limited to a stem cell (e.g., an embryonic stem (ES) cell, an induced pluripotent stem (iPS) cell, a germ cell), a somatic cell (e.g., a fibroblast, a hematopoietic cell, a T lymphocyte (e.g., CD8⁺ T lymphocyte), an NK cell, a neuron, a muscle cell, a bone cell, a hepatocyte, a pancreatic cell), an in vitro or in vivo embryonic cell of an embryo at any stage (e.g., a 1-cell, 2-cell, 4-cell, 8-cell; stage zebrafish embryo). Cells may be from established cell lines or may be primary cells (i.e., cells and cells cultures that have been derived from a subject and allowed to grow in vitro for a limited number of passages of the culture). For example, primary cultures are cultures that may have been passaged within 0 times, 1 time, 2 times, 4 times, 5 times, 10 times, or 15 times, but not enough times to go through the crisis stage. Typically, the primary cell lines of the present invention are maintained for fewer than 10 passages in vitro. If the cells are primary cells, they may be harvest from an individual by any suitable method. For example, leukocytes may be harvested by apheresis, leukocytapheresis, or density gradient separation, while cells from tissues such as skin, muscle, bone marrow, spleen, liver, pancreas, lung, intestine, or stomach can be harvested by biopsy. The harvested cells may be used immediately, or may be stored under frozen conditions with a cryopreservative and thawed at a later time in a manner as commonly known in the art.

### A. Ribonucleonrotein (RNP) Delivery and “Cas RNA” Delivery

The engineered, non-naturally occurring system disclosed herein can be delivered into a cell by suitable methods known in the art, including but not limited to ribonucleoprotein (RNP) delivery and “Cas RNA” delivery described below.

In certain embodiments, a CRISPR-Cas system including a single guide nucleic acid and a Cas protein, or a CRISPR-Cas system including a targeter nucleic acid, a modulator nucleic acid, and a Cas protein, can be combined into a RNP complex and then delivered into the cell as a pre-formed complex. This method is suitable for active modification of the genetic or epigenetic information in a cell during a limited time period. For example, where the Cas protein has nuclease activity to modify the genomic DNA of the cell, the nuclease activity only needs to be retained for a period of time to allow DNA cleavage, and prolonged nuclease activity may increase off-targeting. Similarly, certain epigenetic modifications can be maintained in a cell once established and can be inherited by daughter cells.

A “ribonucleoprotein” or “RNP,” as used herein, can include a complex comprising a nucleoprotein and a ribonucleic acid. A “nucleoprotein” as provided herein can include a protein capable of binding a nucleic acid (e.g., RNA, DNA). Where the nucleoprotein binds a ribonucleic acid it is referred to as “ribonucleoprotein.” The interaction between the ribonucleoprotein and the ribonucleic acid may be direct, e.g., by covalent bond, or indirect, e.g., by non-covalent bond (e.g., electrostatic interactions (e.g., ionic bond, hydrogen bond, halogen bond), van der Waals interactions (e.g., dipole-dipole, dipole-induced dipole, London dispersion), ring stacking (pi effects), hydrophobic interactions, and the like). In certain embodiments, the ribonucleoprotein includes an RNA-binding motif non-covalently bound to the ribonucleic acid. For example, positively charged aromatic amino acid residues (e.g., lysine residues) in the RNA-binding motif may form electrostatic interactions with the negative nucleic acid phosphate backbones of the RNA.

To ensure efficient loading of the Cas protein, the single guide nucleic acid, or the combination of the targeter nucleic acid and the modulator nucleic acid, can be provided in excess molar amount (e.g., at least 1 fold, at least 1.5 fold, at least 2 fold, at least 3 fold, at least 4 fold, or at least 5 fold) relative to the Cas protein. In certain embodiments, the targeter nucleic acid and the modulator nucleic acid are annealed under suitable conditions prior to complexing with the Cas protein. In other embodiments, the targeter nucleic acid, the modulator nucleic acid, and the Cas protein are directly mixed together to form an RNP.

A variety of delivery methods can be used to introduce an RNP disclosed herein into a cell. Exemplary delivery methods or vehicles include but are not limited to microinjection, liposomes (see, e.g., U.S. Pat. No. 10,829,787) such as molecular trojan horses liposomes that delivers molecules across the blood brain barrier (see, Pardridge et al. (2010) COLD SPRING HARB. PROTOC., doi:10.1101/pdb.prot5407), immunoliposomes, virosomes, microvesicles (e.g., exosomes and ARMMs), polycations, lipid:nucleic acid conjugates, electroporation, cell permeable peptides (see, U.S. Pat. No. 11,118,194), nanoparticles, nanowires (see, Shalek et al. (2012) NANO LETTERS, 12: 6498), exosomes, and perturbation of cell membrane (e.g., by passing cells through a constriction in a microfluidic system, see, U.S. Pat. No. 11,125,739). Where the target cell is a proliferating cell, the efficiency of RNP delivery can be enhanced by cell cycle synchronization (see, U.S. Pat. No. 10,570,418).

In other embodiments, the dual guide CRISPR-Cas system is delivered into a cell in a “Cas RNA” approach, i.e., delivering (a) a single guide nucleic acid, or a combination of a targeter nucleic acid and a modulator nucleic acid, and (b) an RNA (e.g., messenger RNA (mRNA)) encoding a Cas protein. The RNA encoding the Cas protein can be translated in the cell and form a complex with the single guide nucleic acid or combination of the targeter nucleic acid and the modulator nucleic acid intracellularly. Similar to the RNP approach, RNAs have limited half-lives in cells, even though stability-increasing modification(s) can be made in one or more of the RNAs. Accordingly, the “Cas RNA” approach is suitable for active modification of the genetic or epigenetic information in a cell during a limited time period, such as DNA cleavage, and has the advantage of reducing off-targeting.

The mRNA can be produced by transcription of a DNA comprising a regulatory element operably linked to a Cas coding sequence. Given that multiple copies of Cas protein can be generated from one mRNA, the targeter nucleic acid and the modulator nucleic acid are generally provided in excess molar amount (e.g., at least 5 fold, at least 10 fold, at least 20 fold, at least 30 fold, at least 50 fold, or at least 100 fold) relative to the mRNA. In certain embodiments, the targeter nucleic acid and the modulator nucleic acid are annealed under suitable conditions prior to delivery into the cells. In other embodiments, the targeter nucleic acid and the modulator nucleic acid are delivered into the cells without annealing in vitro.

A variety of delivery systems can be used to introduce an “Cas RNA” system into a cell. Non-limiting examples of delivery methods or vehicles include microinjection, biolistic particles, liposomes (see, e.g., U.S. Pat. No. 10,829,787) such as molecular trojan horses liposomes that delivers molecules across the blood brain barrier (see, Pardridge et al. (2010) COLD SPRING HARB. PROTOC., doi:10.1101/pdb.prot5407), immunoliposomes, virosomes, polycations, lipid:nucleic acid conjugates, electroporation, nanoparticles, nanowires (see, Shalek et al. (2012) NANO LETTERS, 12: 6498), exosomes, and perturbation of cell membrane (e.g., by passing cells through a constriction in a microfluidic system, see, U.S. Pat. No. 11,125,739). Specific examples of the “nucleic acid only” approach by electroporation are described in International (PCT) Publication No. WO2016/164356.

In other embodiments, the CRISPR-Cas system is delivered into a cell in the form of (a) a single guide nucleic acid or a combination of a targeter nucleic acid and a modulator nucleic acid, and (b) a DNA comprising a regulatory element operably linked to a Cas coding sequence. The DNA can be provided in a plasmid, viral vector, or any other form described in the “CRISPR Expression Systems” subsection. Such delivery method may result in constitutive expression of Cas protein in the target cell (e.g., if the DNA is maintained in the cell in an episomal vector or is integrated into the genome), and may increase the risk of off-targeting which is undesirable when the Cas protein has nuclease activity. Notwithstanding, this approach is useful when the Cas protein comprises a non-nuclease effector (e.g., a transcriptional activator or repressor). It is also useful for research purposes and for genome editing of plants.

### B. CRISPR Expression Systems

The present invention also provides a nucleic acid comprising a regulatory element operably linked to a nucleotide sequence encoding a guide nucleic acid disclosed herein. In certain embodiments, the nucleic acid comprises a regulatory element operably linked to a nucleotide sequence encoding a single guide nucleic acid disclosed herein; this nucleic acid alone can constitute a CRISPR expression system. In certain embodiments, the nucleic acid comprises a regulatory element operably linked to a nucleotide sequence encoding a targeter nucleic acid disclosed herein. In certain embodiments, the nucleic acid further comprises a nucleotide sequence encoding a modulator nucleic acid disclosed herein, wherein the nucleotide sequence encoding the modulator nucleic acid is operably linked to the same regulatory element as the nucleotide sequence encoding the targeter nucleic acid or a different regulatory element; this nucleic acid alone can constitute a CRISPR expression system.

In addition, the present invention provides a CRISPR expression system comprising: (a) a nucleic acid comprising a first regulatory element operably linked to a nucleotide sequence encoding a targeter nucleic acid disclosed herein and (b) a nucleic acid comprising a second regulatory element operably linked to a nucleotide sequence encoding a modulator nucleic acid disclosed herein.

In certain embodiments, the CRISPR expression system disclosed herein further comprises a nucleic acid comprising a third regulatory element operably linked to a nucleotide sequence encoding a Cas protein disclosed herein. In certain embodiments, the Cas protein is a type V-A, type V-C, or type V-D Cas protein (e.g., Cas nuclease). In certain embodiments, the Cas protein is a type V-A Cas protein (e.g., Cas nuclease).

As used in this context, the term “operably linked” is intended to mean that the nucleotide sequence of interest is linked to the regulatory element in a manner that allows for expression of the nucleotide sequence (e.g., in an in vitro transcription/translation system or in a host cell when the vector is introduced into the host cell).

The nucleic acids of the CRISPR expression system described above may be independently selected from various nucleic acids such as DNA (e.g., modified DNA) and RNA (e.g., modified RNA). In certain embodiments, the nucleic acids comprising a regulatory element operably linked to one or more nucleotide sequences encoding the guide nucleic acids are in the form of DNA. In certain embodiments, the nucleic acid comprising a third regulatory element operably linked to a nucleotide sequence encoding the Cas protein is in the form of DNA. The third regulatory element can be a constitutive or inducible promoter that drives the expression of the Cas protein. In other embodiments, the nucleic acid comprising a third regulatory element operably linked to a nucleotide sequence encoding the Cas protein is in the form of RNA (e.g., mRNA).

The nucleic acids of the CRISPR expression system can be provided in one or more vectors. The term “vector,” as used herein, refers to a nucleic acid molecule capable of transporting another nucleic acid to which it has been linked. Conventional viral and non-viral based gene transfer methods can be used to introduce nucleic acids in cells, such as prokaryotic cells, eukaryotic cells, mammalian cells, or target tissues. Non-viral vector delivery systems include DNA plasmids, RNA (e.g., a transcript of a vector described herein), naked nucleic acid, and nucleic acid complexed with a delivery vehicle, such as a liposome. Viral vector delivery systems include DNA and RNA viruses, which have either episomal or integrated genomes after delivery to the cell. Gene therapy procedures are known in the art and disclosed in Van Brunt (1988) BIOTECHNOLOGY, 6: 1149; Anderson (1992) SCIENCE, 256: 808; Nabel & Feigner (1993) TIBTECH, 11: 211; Mitani & Caskey (1993) TIBTECH, 11: 162; Dillon (1993) TIBTECH, 11: 167; Miller (1992) NATURE, 357: 455; Vigne, (1995) RESTORATIVE NEUROLOGY AND NEUROSCIENCE, 8: 35; Kremer & Perricaudet (1995) BRITISH MEDICAL BULLETIN, 51: 31; Haddada et al. (1995) CURRENT TOPICS IN MICROBIOLOGY AND IMMUNOLOGY, 199: 297; Yu et al. (1994) GENE THERAPY, 1: 13; and Doerfler and Bohm (Eds.) (2012) The Molecular Repertoire of Adenoviruses II: Molecular Biology of Virus-Cell Interactions. In certain embodiments, at least one of the vectors is a DNA plasmid. In certain embodiments, at least one of the vectors is a viral vector (e.g., retrovirus, adenovirus, or adeno-associated virus).

Certain vectors are capable of autonomous replication in a host cell into which they are introduced (e.g., bacterial vectors having a bacterial origin of replication and episomal mammalian vectors). Other vectors (e.g., non-episomal mammalian vectors and replication defective viral vectors) do not autonomously replicate in the host cell. Certain vectors, however, may be integrated into the genome of the host cell and thereby are replicated along with the host genome. A skilled person in the art will appreciate that different vectors may be suitable for different delivery methods and have different host tropism, and will be able to select one or more vectors suitable for the use.

The term “regulatory element,” as used herein, refers to a transcriptional and/or translational control sequence, such as a promoter, enhancer, transcription termination signal (e.g., polyadenylation signal), internal ribosomal entry sites (IRES), protein degradation signal, and the like, that provide for and/or regulate transcription of a non-coding sequence (e.g., a targeter nucleic acid or a modulator nucleic acid) or a coding sequence (e.g., a Cas protein) and/or regulate translation of an encoded polypeptide. Such regulatory elements are described, for example, in Goeddel, GENE EXPRESSION TECHNOLOGY: METHODS IN ENZYMOLOGY, 185, Academic Press, San Diego, Calif. (1990). Regulatory elements include those that direct constitutive expression of a nucleotide sequence in many types of host cell and those that direct expression of the nucleotide sequence only in certain host cells (e.g., tissue-specific regulatory sequences). A tissue-specific promoter may direct expression primarily in a desired tissue of interest, such as muscle, neuron, bone, skin, blood, specific organs (e.g., liver, pancreas), or particular cell types (e.g., lymphocytes). Regulatory elements may also direct expression in a temporal-dependent manner, such as in a cell-cycle dependent or developmental stage-dependent manner, which may or may not also be tissue or cell-type specific. In certain embodiments, a vector comprises one or more pol III promoter (e.g., 1, 2, 3, 4, 5, or more pol III promoters), one or more pol II promoters (e.g., 1, 2, 3, 4, 5, or more pol II promoters), one or more pol I promoters (e.g., 1, 2, 3, 4, 5, or more pol I promoters), or combinations thereof. Examples of pol III promoters include, but are not limited to, U6 and H1 promoters. Examples of pol II promoters include, but are not limited to, the retroviral Rous sarcoma virus (RSV) LTR promoter (optionally with the RSV enhancer), the cytomegalovirus (CMV) promoter (optionally with the CMV enhancer), the SV40 promoter, the dihydrofolate reductase promoter, the β-actin promoter, the phosphoglycerol kinase (PGK) promoter, and the EF1α promoter. Also encompassed by the term “regulatory element” are enhancer elements, such as WPRE; CMV enhancers; the R-U5′ segment in LTR of HTLV-I (see, Takebe et al. (1988) MOL. CELL. BIOL., 8: 466); SV40 enhancer; and the intron sequence between exons 2 and 3 of rabbit pi-globin (see, O'Hare et al. (1981) PROC. NATL. ACAD. SCI. USA., 78: 1527). It will be appreciated by those skilled in the art that the design of the expression vector can depend on factors such as the choice of the host cell to be transformed, the level of expression desired, etc. A vector can be introduced into host cells to produce transcripts, proteins, or peptides, including fusion proteins or peptides, encoded by nucleic acids as described herein (e.g., CRISPR transcripts, proteins, enzymes, mutant forms thereof, or fusion proteins thereof).

In certain embodiments, the nucleotide sequence encoding the Cas protein is codon optimized for expression in a eukaryotic host cell, e.g., a yeast cell, a mammalian cell (e.g., a mouse cell, a rat cell, or a human cell), or a plant cell. Various species exhibit particular bias for certain codons of a particular amino acid. Codon bias (differences in codon usage between organisms) often correlates with the efficiency of translation of messenger RNA (mRNA), which is in turn believed to be dependent on, among other things, the properties of the codons being translated and the availability of particular transfer RNA (tRNA) molecules. The predominance of selected tRNAs in a cell is generally a reflection of the codons used most frequently in peptide synthesis. Accordingly, genes can be tailored for optimal gene expression in a given organism based on codon optimization. Codon usage tables are readily available, for example, at the “Codon Usage Database” available at kazusa.or.jp/codon/and these tables can be adapted in a number of ways (see, Nakamura et al. (2000) NUCL. ACIDS RES., 28: 292). Computer algorithms for codon optimizing a particular sequence for expression in a particular host cell, such as Gene Forge (Aptagen; Jacobus, Pa.), are also available. In certain embodiments, the codon optimization facilitates or improves expression of the Cas protein in the host cell.

### C. Donor Templates

Cleavage of a target nucleotide sequence in the genome of a cell by the CRISPR-Cas system or complex disclosed herein can activate the DNA damage pathways, which may rejoin the cleaved DNA fragments by NHEJ or HDR. HDR requires a repair template, either endogenous or exogenous, to transfer the sequence information from the repair template to the target.

In certain embodiments, the engineered, non-naturally occurring system or CRISPR expression system further comprises a donor template. As used herein, the term “donor template” refers to a nucleic acid designed to serve as a repair template at or near the target nucleotide sequence upon introduction into a cell or organism. In certain embodiments, the donor template is complementary to a polynucleotide comprising the target nucleotide sequence or a portion thereof. When optimally aligned, a donor template may overlap with one or more nucleotides of a target nucleotide sequences (e.g., at least 1, 5, 10, 15, 20, 25, 30, 35, 40, 50, 100, 500 or more nucleotides). The nucleotide sequence of the donor template is typically not identical to the genomic sequence that it replaces. Rather, the donor template may contain one or more substitutions, insertions, deletions, inversions or rearrangements with respect to the genomic sequence, so long as sufficient homology is present to support homology-directed repair. In certain embodiments, the donor template comprises a non-homologous sequence flanked by two regions of homology (i.e., homology arms), such that homology-directed repair between the target DNA region and the two flanking sequences results in insertion of the non-homologous sequence at the target region. In certain embodiments, the donor template comprises a non-homologous sequence 10-100 nucleotides, 50-500 nucleotides, 100-1,000 nucleotides, 200-2,000 nucleotides, or 500-5,000 nucleotides in length positioned between two homology arms.

Generally, the homologous region(s) of a donor template has at least 50% sequence identity to a genomic sequence with which recombination is desired. The homology arms are designed or selected such that they are capable of recombining with the nucleotide sequences flanking the target nucleotide sequence under intracellular conditions. In certain embodiments, where HDR of the non-target strand is desired, the donor template comprises a first homology arm homologous to a sequence 5′ to the target nucleotide sequence and a second homology arm homologous to a sequence 3′ to the target nucleotide sequence. In certain embodiments, the first homology arm is at least 50% (e.g., at least 60%, at least 70%, at least 75%, at least 80%, at least 85%, at least 90%, at least 91%, at least 92%, at least 93%, at least 94%, at least 95%, at least 96%, at least 97%, at least 98%, at least 99%, or 100%) identical to a sequence 5′ to the target nucleotide sequence. In certain embodiments, the second homology arm is at least 50% (e.g., at least 60%, at least 70%, at least 75%, at least 80%, at least 85%, at least 90%, at least 91%, at least 92%, at least 93%, at least 94%, at least 95%, at least 96%, at least 97%, at least 98%, at least 99%, or 100%) identical to a sequence 3′ to the target nucleotide sequence. In certain embodiments, when the donor template sequence and a polynucleotide comprising a target nucleotide sequence are optimally aligned, the nearest nucleotide of the donor template is within 1, 5, 10, 15, 20, 25, 50, 75, 100, 200, 300, 400, 500, 1000, 2000, 3000, 4000, or more nucleotides from the target nucleotide sequence.

In certain embodiments, the donor template further comprises an engineered sequence not homologous to the sequence to be repaired. Such engineered sequence can harbor a barcode and/or a sequence capable of hybridizing with a donor template-recruiting sequence disclosed herein.

In certain embodiments, the donor template further comprises one or more mutations relative to the genomic sequence, wherein the one or more mutations reduce or prevent cleavage, by the same CRISPR-Cas system, of the donor template or of a modified genomic sequence with at least a portion of the donor template sequence incorporated. In certain embodiments, in the donor template, the PAM adjacent to the target nucleotide sequence and recognized by the Cas nuclease is mutated to a sequence not recognized by the same Cas nuclease. In certain embodiments, in the donor template, the target nucleotide sequence (e.g., the seed region) is mutated. In certain embodiments, the one or more mutations are silent with respect to the reading frame of a protein-coding sequence encompassing the mutated sites.

The donor template can be provided to the cell as single-stranded DNA, single-stranded RNA, double-stranded DNA, or double-stranded RNA. It is understood that the CRISPR-Cas system disclosed herein may possess nuclease activity to cleave the target strand, the non-target strand, or both. When HDR of the target strand is desired, a donor template having a nucleic acid sequence complementary to the target strand is also contemplated.

The donor template can be introduced into a cell in linear or circular form. If introduced in linear form, the ends of the donor template may be protected (e.g., from exonucleolytic degradation) by methods known to those of skill in the art. For example, one or more dideoxynucleotide residues are added to the 3′ terminus of a linear molecule and/or self-complementary oligonucleotides are ligated to one or both ends (see, for example, Chang et al. (1987) PROC. NATL. ACAD SC USA, 84: 4959; Nehls et al. (1996) SCIENCE, 272: 886; see also the chemical modifications for increasing stability and/or specificity of RNA disclosed supra). Additional methods for protecting exogenous polynucleotides from degradation include, but are not limited to, addition of terminal amino group(s) and the use of modified internucleotide linkages such as, for example, phosphorothioates, phosphoramidates, and O-methyl ribose or deoxyribose residues. As an alternative to protecting the termini of a linear donor template, additional lengths of sequence may be included outside of the regions of homology that can be degraded without impacting recombination.

A donor template can be a component of a vector as described herein, contained in a separate vector, or provided as a separate polynucleotide, such as an oligonucleotide, linear polynucleotide, or synthetic polynucleotide. In certain embodiments, the donor template is a DNA. In certain embodiments, a donor template is in the same nucleic acid as a sequence encoding the single guide nucleic acid, a sequence encoding the targeter nucleic acid, a sequence encoding the modulator nucleic acid, and/or a sequence encoding the Cas protein, where applicable. In certain embodiments, a donor template is provided in a separate nucleic acid. A donor template polynucleotide may be of any suitable length, such as 50, 75, 100, 150, 200, 500, 1000, 2000, 3000, 4000, or more nucleotides in length.

A donor template can be introduced into a cell as an isolated nucleic acid. Alternatively, a donor template can be introduced into a cell as part of a vector (e.g., a plasmid) having additional sequences such as, for example, replication origins, promoters and genes encoding antibiotic resistance, that are not intended for insertion into the DNA region of interest. Alternatively, a donor template can be delivered by viruses (e.g., adenovirus, adeno-associated virus (AAV)). In certain embodiments, the donor template is introduced as an AAV, e.g., a pseudotyped AAV. The capsid proteins of the AAV can be selected by a person skilled in the art based upon the tropism of the AAV and the target cell type. For example, in certain embodiments, the donor template is introduced into a hepatocyte as AAV8 or AAV9. In certain embodiments, the donor template is introduced into a hematopoietic stem cell, a hematopoietic progenitor cell, or a T lymphocyte (e.g., CD8⁺ T lymphocyte) as AAV6 or an AAVHSC (see, U.S. Pat. No. 9,890,396). It is understood that the sequence of a capsid protein (VP1, VP2, or VP3) may be modified from a wild-type AAV capsid protein, for example, having at least 50% (e.g., at least 60%, at least 70%, at least 75%, at least 80%, at least 85%, at least 90%, at least 91%, at least 92%, at least 93%, at least 94%, at least 95%, at least 96%, at least 97%, at least 98%, or at least 99%) sequence identity to a wild-type AAV capsid sequence.

The donor template can be delivered to a cell (e.g., a primary cell) by various delivery methods, such as a viral or non-viral method disclosed herein. In certain embodiments, a non-viral donor template is introduced into the target cell as a naked nucleic acid or in complex with a liposome or poloxamer. In certain embodiments, a non-viral donor template is introduced into the target cell by electroporation. In other embodiments, a viral donor template is introduced into the target cell by infection. The engineered, non-naturally occurring system can be delivered before, after, or simultaneously with the donor template (see, International (PCT) Application Publication No. WO2017/053729). A skilled person in the art will be able to choose proper timing based upon the form of delivery (consider, for example, the time needed for transcription and translation of RNA and protein components) and the half-life of the molecule(s) in the cell. In particular embodiments, where the CRISPR-Cas system including the Cas protein is delivered by electroporation (e.g., as an RNP), the donor template (e.g., as an AAV) is introduced into the cell within 4 hours (e.g., within 1, 2, 3, 4, 5, 6, 7, 8, 9, 10, 11, 12, 13, 14, 15, 16, 17, 18, 19, 20, 25, 30, 35, 40, 45, 50, 55, 60, 90, 120, 150, 180, 210, or 240 minutes) after the introduction of the engineered, non-naturally occurring system.

In certain embodiments, the donor template is conjugated covalently to the modulator nucleic acid. Covalent linkages suitable for this conjugation are known in the art and are described, for example, in U.S. Pat. No. 9,982,278 and Savic et al. (2018) ELIFE 7:e33761. In certain embodiments, the donor template is covalently linked to the modulator nucleic acid (e.g., the 5′ end of the modulator nucleic acid) through an internucleotide bond. In certain embodiments, the donor template is covalently linked to the modulator nucleic acid (e.g., the 5′ end of the modulator nucleic acid) through a linker.

### D. Efficiency and Specificity

The engineered, non-naturally occurring system of the present invention has the advantage of high efficiency and/or high specificity in nucleic acid targeting, cleavage, or modification.

In certain embodiments, the engineered, non-naturally occurring system has high efficiency. For example, in certain embodiments, at least 1%, at least 1.5%, at least 2%, at least 2.5%, at least 3%, at least 4%, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, or at least 99% of a population of nucleic acids having the target nucleotide sequence and a cognate PAM, when contacted with the engineered, non-naturally occurring system, is targeted, cleaved, or modified. In certain embodiments, the genomes of at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, or at least 99% of a population of cells, when the engineered, non-naturally occurring system is delivered into the cells, are targeted, cleaved, or modified.

In certain embodiments, where the engineered, non-naturally occurring system comprises a guide nucleic acid comprising a spacer sequence listed in any of the Tables 1-9 or a portion thereof, the genomes of at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, or at least 99% of a population of human cells are targeted, cleaved, edited, or modified when the engineered, non-naturally occurring system is delivered into the cells. In certain embodiments, where the engineered, non-naturally occurring system comprises a guide nucleic acid comprising a spacer sequence listed in any of the Tables 1-9 or a portion thereof, the genomes of at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, or at least 99% of a population of human cells are edited when the engineered, non-naturally occurring system is delivered into the cells.

In certain embodiments, where the engineered, non-naturally occurring system comprises a guide nucleic acid comprising a spacer sequence listed in any one of Tables 1-9 or a portion thereof, the genomes of at least 1%, at least 1.5%, at least 2%, at least 2.5%, at least 3%, at least 4%, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, or at least 99% of a population of human cells are targeted, cleaved, edited, or modified when the engineered, non-naturally occurring system is delivered into the cells. In certain embodiments, where the engineered, non-naturally occurring system comprises a guide nucleic acid comprising a spacer sequence listed in any one of Tables 1-9 or a portion thereof, the genomes of at least 1%, at least 1.5%, at least 2%, at least 2.5%, at least 3%, at least 4%, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, or at least 99% of a population of human cells are edited when the engineered, non-naturally occurring system is delivered into the cells.

In certain embodiments, when an engineered, non-naturally occurring system comprising a guide nucleic acid comprising a spacer sequence set forth in SEQ ID NOs: 201-253 is delivered into a population of human cells ex vivo, the genome sequence at the CSF2 gene locus is edited in at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, or at least 99% of the cells.

In certain embodiments, when an engineered, non-naturally occurring system comprising a guide nucleic acid comprising a spacer sequence set forth in SEQ ID NOs: 254-313 is delivered into a population of human cells ex vivo, the genome sequence at the CD40LG gene locus is edited in at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, or at least 99% of the cells.

In certain embodiments, when an engineered, non-naturally occurring system comprising a guide nucleic acid comprising a spacer sequence set forth in SEQ ID NOs: 314-319 and 329-332 is delivered into a population of human cells ex vivo, the genome sequence at the TRBC1gene locus is edited in at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, or at least 99% of the cells.

In certain embodiments, when an engineered, non-naturally occurring system comprising a guide nucleic acid comprising a spacer sequence set forth in SEQ ID NOs: 320-328 and 329-332 is delivered into a population of human cells ex vivo, the genome sequence at the TRBC2 gene locus is edited in at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, or at least 99% of the cells.

In certain embodiments, when an engineered, non-naturally occurring system comprising a guide nucleic acid comprising a spacer sequence set forth in SEQ ID NOs: 329-332 is delivered into a population of human cells ex vivo, the genome sequence at both the human TRBC1 gene and the human TRBC2 gene (TRBC1_2) locus is edited in at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, or at least 99% of the cells.

In certain embodiments, when an engineered, non-naturally occurring system comprising a guide nucleic acid comprising a spacer sequence set forth in SEQ ID NOs: 333-374 is delivered into a population of human cells ex vivo, the genome sequence at the CD3E gene locus is edited in at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, or at least 99% of the cells.

In certain embodiments, when an engineered, non-naturally occurring system comprising a guide nucleic acid comprising a spacer sequence set forth in SEQ ID NOs: 375-411 is delivered into a population of human cells ex vivo, the genome sequence at the CD38 gene locus is edited in at least 1%, at least 1.5%, at least 5%, at least 10%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, or at least 99% of the cells.

In certain embodiments, when an engineered, non-naturally occurring system comprising a guide nucleic acid comprising a spacer sequence set forth in SEQ ID NOs: 412-421 is delivered into a population of human cells ex vivo, the genome sequence at the APLNR gene locus is edited in at least 1%, at least 1.5%, at least 5%, at least 10%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, or at least 99% of the cells.

In certain embodiments, when an engineered, non-naturally occurring system comprising a guide nucleic acid comprising a spacer sequence set forth in SEQ ID NOs: 422-431 is delivered into a population of human cells ex vivo, the genome sequence at the BBS1 gene locus is edited in at least 1%, at least 1.5%, at least 5%, at least 10%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, or at least 99% of the cells.

In certain embodiments, when an engineered, non-naturally occurring system comprising a guide nucleic acid comprising a spacer sequence set forth in SEQ ID NOs: 432-441 is delivered into a population of human cells ex vivo, the genome sequence at the CALR gene locus is edited in at least 1%, at least 1.5%, at least 5%, at least 10%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, or at least 99% of the cells.

In certain embodiments, when an engineered, non-naturally occurring system comprising a guide nucleic acid comprising a spacer sequence set forth in SEQ ID NOs: 442-451 is delivered into a population of human cells ex vivo, the genome sequence at the CD247 gene locus is edited in at least 1%, at least 1.5%, at least 5%, at least 10%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, or at least 99% of the cells.

In certain embodiments, when an engineered, non-naturally occurring system comprising a guide nucleic acid comprising a spacer sequence set forth in SEQ ID NOs: 452-461 is delivered into a population of human cells ex vivo, the genome sequence at the CD3G gene locus is edited in at least 1%, at least 1.5%, at least 5%, at least 10%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, or at least 99% of the cells.

In certain embodiments, when an engineered, non-naturally occurring system comprising a guide nucleic acid comprising a spacer sequence set forth in SEQ ID NOs: 462-465 is delivered into a population of human cells ex vivo, the genome sequence at the CD52 gene locus is edited in at least 1%, at least 1.5%, at least 5%, at least 10%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, or at least 99% of the cells.

In certain embodiments, when an engineered, non-naturally occurring system comprising a guide nucleic acid comprising a spacer sequence set forth in SEQ ID NOs: 466-475 is delivered into a population of human cells ex vivo, the genome sequence at the CD58 gene locus is edited in at least 1%, at least 1.5%, at least 5%, at least 10%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, or at least 99% of the cells.

In certain embodiments, when an engineered, non-naturally occurring system comprising a guide nucleic acid comprising a spacer sequence set forth in SEQ ID NOs: 476-485 is delivered into a population of human cells ex vivo, the genome sequence at the COL17A1 gene locus is edited in at least 1%, at least 1.5%, at least 5%, at least 10%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, or at least 99% of the cells.

In certain embodiments, when an engineered, non-naturally occurring system comprising a guide nucleic acid comprising a spacer sequence set forth in SEQ ID NOs: 486-495 is delivered into a population of human cells ex vivo, the genome sequence at the DEFB134 gene locus is edited in at least 1%, at least 1.5%, at least 5%, at least 10%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, or at least 99% of the cells.

In certain embodiments, when an engineered, non-naturally occurring system comprising a guide nucleic acid comprising a spacer sequence set forth in SEQ ID NOs: 496-505 is delivered into a population of human cells ex vivo, the genome sequence at the ERAP1 gene locus is edited in at least 1%, at least 1.5%, at least 5%, at least 10%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, or at least 99% of the cells.

In certain embodiments, when an engineered, non-naturally occurring system comprising a guide nucleic acid comprising a spacer sequence set forth in SEQ ID NOs: 506-515 is delivered into a population of human cells ex vivo, the genome sequence at the ERAP2 gene locus is edited in at least 1%, at least 1.5%, at least 5%, at least 10%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, or at least 99% of the cells.

In certain embodiments, when an engineered, non-naturally occurring system comprising a guide nucleic acid comprising a spacer sequence set forth in SEQ ID NOs: 516-525 is delivered into a population of human cells ex vivo, the genome sequence at the IFNGR1 gene locus is edited in at least 1%, at least 1.5%, at least 5%, at least 10%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, or at least 99% of the cells.

In certain embodiments, when an engineered, non-naturally occurring system comprising a guide nucleic acid comprising a spacer sequence set forth in SEQ ID NOs: 526-535 is delivered into a population of human cells ex vivo, the genome sequence at the IFNGR2 gene locus is edited in at least 1%, at least 1.5%, at least 5%, at least 10%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, or at least 99% of the cells.

In certain embodiments, when an engineered, non-naturally occurring system comprising a guide nucleic acid comprising a spacer sequence set forth in SEQ ID NOs: 536-545 is delivered into a population of human cells ex vivo, the genome sequence at the JAK1 gene locus is edited in at least 1%, at least 1.5%, at least 5%, at least 10%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, or at least 99% of the cells.

In certain embodiments, when an engineered, non-naturally occurring system comprising a guide nucleic acid comprising a spacer sequence set forth in SEQ ID NOs: 546-555 is delivered into a population of human cells ex vivo, the genome sequence at the JAK2 gene locus is edited in at least 1%, at least 1.5%, at least 5%, at least 10%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, or at least 99% of the cells.

In certain embodiments, when an engineered, non-naturally occurring system comprising a guide nucleic acid comprising a spacer sequence set forth in SEQ ID NOs: 556-558 is delivered into a population of human cells ex vivo, the genome sequence at the mir-101-2 gene locus is edited in at least 1%, at least 1.5%, at least 5%, at least 10%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, or at least 99% of the cells.

In certain embodiments, when an engineered, non-naturally occurring system comprising a guide nucleic acid comprising a spacer sequence set forth in SEQ ID NOs: 559-568 is delivered into a population of human cells ex vivo, the genome sequence at the MLANA gene locus is edited in at least 1%, at least 1.5%, at least 5%, at least 10%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, or at least 99% of the cells.

In certain embodiments, when an engineered, non-naturally occurring system comprising a guide nucleic acid comprising a spacer sequence set forth in SEQ ID NOs: 569-578 is delivered into a population of human cells ex vivo, the genome sequence at the PSMB5 gene locus is edited in at least 1%, at least 1.5%, at least 5%, at least 10%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, or at least 99% of the cells.

In certain embodiments, when an engineered, non-naturally occurring system comprising a guide nucleic acid comprising a spacer sequence set forth in SEQ ID NOs: 579-588 is delivered into a population of human cells ex vivo, the genome sequence at the PSMB8 gene locus is edited in at least 1%, at least 1.5%, at least 5%, at least 10%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, or at least 99% of the cells.

In certain embodiments, when an engineered, non-naturally occurring system comprising a guide nucleic acid comprising a spacer sequence set forth in SEQ ID NOs: 589-598 is delivered into a population of human cells ex vivo, the genome sequence at the PSMB9 gene locus is edited in at least 1%, at least 1.5%, at least 5%, at least 10%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, or at least 99% of the cells.

In certain embodiments, when an engineered, non-naturally occurring system comprising a guide nucleic acid comprising a spacer sequence set forth in SEQ ID NOs: 599-608 is delivered into a population of human cells ex vivo, the genome sequence at the PTCD2 gene locus is edited in at least 1%, at least 1.5%, at least 5%, at least 10%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, or at least 99% of the cells.

In certain embodiments, when an engineered, non-naturally occurring system comprising a guide nucleic acid comprising a spacer sequence set forth in SEQ ID NOs: 609-618 is delivered into a population of human cells ex vivo, the genome sequence at the RFX5 gene locus is edited in at least 1%, at least 1.5%, at least 5%, at least 10%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, or at least 99% of the cells.

In certain embodiments, when an engineered, non-naturally occurring system comprising a guide nucleic acid comprising a spacer sequence set forth in SEQ ID NOs: 619-628 is delivered into a population of human cells ex vivo, the genome sequence at the RFXANK gene locus is edited in at least 1%, at least 1.5%, at least 5%, at least 10%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, or at least 99% of the cells.

In certain embodiments, when an engineered, non-naturally occurring system comprising a guide nucleic acid comprising a spacer sequence set forth in SEQ ID NOs: 629-638 is delivered into a population of human cells ex vivo, the genome sequence at the RFXAP gene locus is edited in at least 1%, at least 1.5%, at least 5%, at least 10%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, or at least 99% of the cells.

In certain embodiments, when an engineered, non-naturally occurring system comprising a guide nucleic acid comprising a spacer sequence set forth in SEQ ID NOs: 639-648 is delivered into a population of human cells ex vivo, the genome sequence at the RPL23 gene locus is edited in at least 1%, at least 1.5%, at least 5%, at least 10%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, or at least 99% of the cells.

In certain embodiments, when an engineered, non-naturally occurring system comprising a guide nucleic acid comprising a spacer sequence set forth in SEQ ID NOs: 649-654 is delivered into a population of human cells ex vivo, the genome sequence at the SOX10 gene locus is edited in at least 1%, at least 1.5%, at least 5%, at least 10%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, or at least 99% of the cells.

In certain embodiments, when an engineered, non-naturally occurring system comprising a guide nucleic acid comprising a spacer sequence set forth in SEQ ID NOs: 655-665 is delivered into a population of human cells ex vivo, the genome sequence at the SRP54 gene locus is edited in at least 1%, at least 1.5%, at least 5%, at least 10%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, or at least 99% of the cells.

In certain embodiments, when an engineered, non-naturally occurring system comprising a guide nucleic acid comprising a spacer sequence set forth in SEQ ID NOs: 666-675 is delivered into a population of human cells ex vivo, the genome sequence at the STAT1 gene locus is edited in at least 1%, at least 1.5%, at least 5%, at least 10%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, or at least 99% of the cells.

In certain embodiments, when an engineered, non-naturally occurring system comprising a guide nucleic acid comprising a spacer sequence set forth in SEQ ID NOs: 676-685 is delivered into a population of human cells ex vivo, the genome sequence at the Tap1 gene locus is edited in at least 1%, at least 1.5%, at least 5%, at least 10%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, or at least 99% of the cells.

In certain embodiments, when an engineered, non-naturally occurring system comprising a guide nucleic acid comprising a spacer sequence set forth in SEQ ID NOs: 686-695 is delivered into a population of human cells ex vivo, the genome sequence at the TAP2 gene locus is edited in at least 1%, at least 1.5%, at least 5%, at least 10%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, or at least 99% of the cells.

In certain embodiments, when an engineered, non-naturally occurring system comprising a guide nucleic acid comprising a spacer sequence set forth in SEQ ID NOs: 696-705 is delivered into a population of human cells ex vivo, the genome sequence at the TAPBP gene locus is edited in at least 1%, at least 1.5%, at least 5%, at least 10%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, or at least 99% of the cells.

In certain embodiments, when an engineered, non-naturally occurring system comprising a guide nucleic acid comprising a spacer sequence set forth in SEQ ID NOs: 706-715 is delivered into a population of human cells ex vivo, the genome sequence at the TWF1 gene locus is edited in at least 1%, at least 1.5%, at least 5%, at least 10%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, or at least 99% of the cells.

In certain embodiments, when an engineered, non-naturally occurring system comprising a guide nucleic acid comprising a spacer sequence set forth in SEQ ID NOs: 716-725 is delivered into a population of human cells ex vivo, the genome sequence at the CD3D gene locus is edited in at least 1%, at least 1.5%, at least 5%, at least 10%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, or at least 99% of the cells.

In certain embodiments, when an engineered, non-naturally occurring system comprising a guide nucleic acid comprising a spacer sequence set forth in SEQ ID NOs: 726-744 is delivered into a population of human cells ex vivo, the genome sequence at the NLRC5 gene locus is edited in at least 1%, at least 1.5%, at least 5%, at least 10%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, or at least 99% of the cells.

In certain embodiments, the genome edit is an insertion or a deletion, ie., an INDEL.

In certain embodiments, when an engineered, non-naturally occurring system comprising a guide nucleic acid comprising a spacer sequence of any one of Tables 1-9 is delivered into a one or more cells ex vivo, the edited cell demonstrates less than 80% (e.g., less than 70%, less than 60%, less than 50%, less than 40%, less than 30%, less than 20%, less than 10%, or less than 5%) of the expression of the endogenous gene relative to a corresponding unmodified or parental cell.

It has been observed that for a given spacer sequence, the occurrence of on-target events and the occurrence of off-target events are generally correlated. For certain therapeutic purposes, lower on-target efficiency can be tolerated and low off-target frequency is more desirable. For example, when editing or modifying a proliferating cell that will be delivered to a subject and proliferate in vivo, tolerance to off-target events is low. Prior to delivery, it is possible to assess the on-target and off-target events, thereby selecting one or more colonies that have the desired edit or modification and lack any undesired edit or modification. Notwithstanding, the on-target efficiency needs to meet a certain standard to be suitable for therapeutic use. The high editing efficiency observed with the spacer sequences disclosed herein in a standard CRISPR-Cas system allows tuning of the system, for example, by reducing the binding of the guide nucleic acids to the Cas protein, without losing therapeutic applicability.

In certain embodiments, when a population of nucleic acids having the target nucleotide sequence and a cognate PAM is contacted with the engineered, non-naturally occurring system disclosed herein, the frequency of off-target events (e.g., targeting, cleavage, or modification, depending on the function of the CRISPR-Cas system) is reduced. Methods of assessing off-target events were summarized in Lazzarotto et al. (2018) NAT PROTOC. 13(11): 2615-42, and include discovery of in situ Cas off-targets and verification by sequencing (DISCOVER-seq) as disclosed in Wienert et al. (2019) SCIENCE 364(6437): 286-89; genome-wide unbiased identification of double-stranded breaks (DSBs) enabled by sequencing (GUIDE-seq) as disclosed in Kleinstiver et al. (2016) NAT. BIOTECH. 34: 869-74; circularization for in vitro reporting of cleavage effects by sequencing (CIRCLE-seq) as described in Kocak et al. (2019) NAT. BIOTECH. 37: 657-66. In certain embodiments, the off-target events include targeting, cleavage, or modification at a given off-target locus (e.g., the locus with the highest occurrence of off-target events detected). In certain embodiments, the off-target events include targeting, cleavage, or modification at all the loci with detectable off-target events, collectively.

In certain embodiments, genomic mutations are detected in no more than 0.0001%, 0.0002%, 0.0003%, 0.0004%, 0.0005%, 0.0006%, 0.0007%, 0.0008%, 0.0009%, 0.001%, 0.002%, 0.003%, 0.004%, 0.005%, 0.006%, 0.007%, 0.008%, 0.009%, 0.01%, 0.02%, 0.03%, 0.04%, 0.05%, 0.06%, 0.07%, 0.08%, 0.09%, 0.1%, 0.2%, 0.3%, 0.4%, 0.5%, 0.6%, 0.7%, 0.8%, 0.9%, 1%, 2%, 3%, 4%, or 5% of the cells at any off-target loci (in aggregate). In certain embodiments, the ratio of the percentage of cells having an on-target event to the percentage of cells having any off-target event (e.g., the ratio of the percentage of cells having an on-target editing event to the percentage of cells having a mutation at any off-target loci) is at least 10, 20, 30, 40, 50, 60, 70, 80, 90, 100, 200, 300, 400, 500, 600, 700, 800, 900, 1000, 2000, 3000, 4000, 5000, 6000, 7000, 8000, 9000, or 10000. It is understood that genetic variation may be present in a population of cells, for example, by spontaneous mutations, and such mutations are not included as off-target events.

### E. Multiplex Methods

The method of targeting, editing, and/or modifying a genomic DNA disclosed herein can be conducted in multiplicity. For example, a library of targeter nucleic acids can be used to target multiple genomic loci; a library of donor templates can also be used to generate multiple insertions, deletions, and/or substitutions. The multiplex assay can be conducted in a screening method wherein each separate cell culture (e.g., in a well of a 96-well plate or a 384-well plate) is exposed to a different guide nucleic acid having a different targeter stem sequence and/or a different donor template. The multiplex assay can also be conducted in a selection method wherein a cell culture is exposed to a mixed population of different guide nucleic acids and/or donor templates, and the cells with desired characteristics (e.g., functionality) are enriched or selected by advantageous survival or growth, resistance to a certain agent, expression of a detectable protein (e.g., a fluorescent protein that is detectable by flow cytometry), etc.

In certain embodiments, the plurality of guide nucleic acids and/or the plurality of donor templates are designed for saturation editing. For example, in certain embodiments, each nucleotide position in a sequence of interest is systematically modified with each of all four traditional bases, A, T, G and C. In other embodiments, at least one sequence in each gene from a pool of genes of interest is modified, for example, according to a CRISPR design algorithm. In certain embodiments, each sequence from a pool of exogenous elements of interest (e.g., protein coding sequences, non-protein coding genes, regulatory elements) is inserted into one or more given loci of the genome.

It is understood that the multiplex methods suitable for the purpose of carrying out a screening or selection method, which is typically conducted for research purposes, may be different from the methods suitable for therapeutic purposes. For example, constitutive expression of certain elements (e.g., a Cas nuclease and/or a guide nucleic acid) may be undesirable for therapeutic purposes due to the potential of increased off-targeting. Conversely, for research purposes, constitutive expression of a Cas nuclease and/or a guide nucleic acid may be desirable. For example, the constitutive expression provides a large window during which other elements can be introduced. When a stable cell line is established for the constitutive expression, the number of exogenous elements that need to be co-delivered into a single cell is also reduced. Therefore, constitutive expression of certain elements can increase the efficiency and reduce the complexity of a screening or selection process. Inducible expression of certain elements of the system disclosed herein may also be used for research purposes given similar advantages. Expression may be induced by an exogenous agent (e.g., a small molecule) or by an endogenous molecule or complex present in a particular cell type (e.g., at a particular stage of differentiation). Methods known in the art, such as those described in the “CRISPR Expression Systems” subsection supra, can be used for constitutively or inducibly expressing one or more elements.

It is further understood that despite the need to introduce multiple elements—the single guide nucleic acid and the Cas protein; or the targeter nucleic acid, the modulator nucleic acid, and the Cas protein—these elements can be delivered into the cell as a single complex of pre-formed RNP. Therefore, the efficiency of the screening or selection process can also be achieved by pre-assembling a plurality of RNP complexes in a multiplex manner.

In certain embodiments, the method disclosed herein further comprises a step of identifying a guide nucleic acid, a Cas protein, a donor template, or a combination of two or more of these elements from the screening or selection process. A set of barcodes may be used, for example, in the donor template between two homology arms, to facilitate the identification. In specific embodiments, the method further comprises harvesting the population of cells; selectively amplifying a genomic DNA or RNA sample including the target nucleotide sequence(s) and/or the barcodes; and/or sequencing the genomic DNA or RNA sample and/or the barcodes that has been selectively amplified.

In addition, the present invention provides a library comprising a plurality of guide nucleic acids disclosed herein. In another aspect, the present invention provides a library comprising a plurality of nucleic acids each comprising a regulatory element operably linked to a different guide nucleic acid disclosed herein. These libraries can be used in combination with one or more Cas proteins or Cas-coding nucleic acids disclosed herein, and/or one or more donor templates as disclosed herein for a screening or selection method.

### III. PHARMACEUTICAL COMPOSITIONS

The present invention provides a composition (e.g., pharmaceutical composition) comprising a guide nucleic acid, an engineered, non-naturally occurring system, or a eukaryotic cell disclosed herein. In certain embodiments, the composition comprises an RNP comprising a guide nucleic acid disclosed herein and a Cas protein (e.g., Cas nuclease). In certain embodiments, the composition comprises a complex of a targeter nucleic acid and a modulator nucleic acid disclosed herein. In certain embodiments, the composition comprises an RNP comprising the targeter nucleic acid, the modulator nucleic acid, and a Cas protein (e.g., Cas nuclease).

In addition, the present invention provides a method of producing a composition, the method comprising incubating a single guide nucleic acid disclosed herein with a Cas protein, thereby producing a complex of the single guide nucleic acid and the Cas protein (e.g., an RNP). In certain embodiments, the method further comprises purifying the complex (e.g., the RNP).

In addition, the present invention provides a method of producing a composition, the method comprising incubating a targeter nucleic acid and a modulator nucleic acid disclosed herein under suitable conditions, thereby producing a composition (e.g., pharmaceutical composition) comprising a complex of the targeter nucleic acid and the modulator nucleic acid. In certain embodiments, the method further comprises incubating the targeter nucleic acid and the modulator nucleic acid with a Cas protein (e.g., the Cas nuclease that the targeter nucleic acid and the modulator nucleic acid are capable of activating or a related Cas protein), thereby producing a complex of the targeter nucleic acid, the modulator nucleic acid, and the Cas protein (e.g., an RNP). In certain embodiments, the method further comprises purifying the complex (e.g., the RNP).

For therapeutic use, a guide nucleic acid, an engineered, non-naturally occurring system, a CRISPR expression system, or a cell comprising such system or modified by such system disclosed herein is combined with a pharmaceutically acceptable carrier. The term “pharmaceutically acceptable” as used herein refers to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit-to-risk ratio.

The term “pharmaceutically acceptable carrier” as used herein refers to buffers, carriers, and excipients suitable for use in contact with the tissues of human beings and animals without excessive toxicity, irritation, allergic response, or other problem or complication, commensurate with a reasonable benefit/risk ratio. Pharmaceutically acceptable carriers include any of the standard pharmaceutical carriers, such as a phosphate buffered saline solution, water, emulsions (e.g., such as an oil/water or water/oil emulsions), and various types of wetting agents. The compositions also can include stabilizers and preservatives. For examples of carriers, stabilizers and adjuvants, see, e.g., Martin, Remington's Pharmaceutical Sciences, 15th Ed., Mack Publ. Co., Easton, PA (1975). Pharmaceutically acceptable carriers include buffers, solvents, dispersion media, coatings, isotonic and absorption delaying agents, and the like, that are compatible with pharmaceutical administration. The use of such media and agents for pharmaceutically active substances is known in the art.

In certain embodiments, a pharmaceutical composition disclosed herein comprises a salt, e.g., NaCl, MgCl₂, KCl, MgSO₄, etc.; a buffering agent, e.g., a Tris buffer, N-(2-Hydroxyethyl)piperazine-N′-(2-ethanesulfonic acid) (HEPES), 2-(N-Morpholino)ethanesulfonic acid (MES), MES sodium salt, 3-(N-Morpholino)propanesulfonic acid (MOPS), N-tris[Hydroxymethyl]methyl-3-aminopropanesulfonic acid (TAPS), etc.; a solubilizing agent; a detergent, e.g., a non-ionic detergent such as Tween-20, etc.; a nuclease inhibitor; and the like. For example, in certain embodiments, a subject composition comprises a subject DNA-targeting RNA and a buffer for stabilizing nucleic acids.

In certain embodiments, a pharmaceutical composition may contain formulation materials for modifying, maintaining or preserving, for example, the pH, osmolarity, viscosity, clarity, color, isotonicity, odor, sterility, stability, rate of dissolution or release, adsorption or penetration of the composition. In such embodiments, suitable formulation materials include, but are not limited to, amino acids (such as glycine, glutamine, asparagine, arginine or lysine); antimicrobials; antioxidants (such as ascorbic acid, sodium sulfite or sodium hydrogen-sulfite); buffers (such as borate, bicarbonate, Tris-HCl, citrates, phosphates or other organic acids); bulking agents (such as mannitol or glycine); chelating agents (such as ethylenediamine tetraacetic acid (EDTA)); complexing agents (such as caffeine, polyvinylpyrrolidone, beta-cyclodextrin or hydroxypropyl-beta-cyclodextrin); fillers; monosaccharides; disaccharides; and other carbohydrates (such as glucose, mannose or dextrins); proteins (such as serum albumin, gelatin or immunoglobulins); coloring, flavoring and diluting agents; emulsifying agents; hydrophilic polymers (such as polyvinylpyrrolidone); low molecular weight polypeptides; salt-forming counterions (such as sodium); preservatives (such as benzalkonium chloride, benzoic acid, salicylic acid, thimerosal, phenethyl alcohol, methylparaben, propylparaben, chlorhexidine, sorbic acid or hydrogen peroxide); solvents (such as glycerin, propylene glycol or polyethylene glycol); sugar alcohols (such as mannitol or sorbitol); suspending agents; surfactants or wetting agents (such as pluronics, PEG, sorbitan esters, polysorbates such as polysorbate 20, polysorbate, triton, tromethamine, lecithin, cholesterol, tyloxapal); stability enhancing agents (such as sucrose or sorbitol); tonicity enhancing agents (such as alkali metal halides, preferably sodium or potassium chloride, mannitol sorbitol); delivery vehicles; diluents; excipients and/or pharmaceutical adjuvants (see, Remington's Pharmaceutical Sciences, 18th ed. (Mack Publishing Company, 1990).

In certain embodiments, a pharmaceutical composition may contain nanoparticles, e.g., polymeric nanoparticles, liposomes, or micelles (See Anselmo et al. (2016) BIOENG. TRANSL. MED. 1: 10-29). In certain embodiment, the pharmaceutical composition comprises an inorganic nanoparticle. Exemplary inorganic nanoparticles include, e.g., magnetic nanoparticles (e.g., Fe₃MnO₂) or silica. The outer surface of the nanoparticle can be conjugated with a positively charged polymer (e.g., polyethylenimine, polylysine, polyserine) which allows for attachment (e.g., conjugation or entrapment) of payload. In certain embodiment, the pharmaceutical composition comprises an organic nanoparticle (e.g., entrapment of the payload inside the nanoparticle). Exemplary organic nanoparticles include, e.g., SNALP liposomes that contain cationic lipids together with neutral helper lipids which are coated with polyethylene glycol (PEG) and protamine and nucleic acid complex coated with lipid coating. In certain embodiment, the pharmaceutical composition comprises a liposome, for example, a liposome disclosed in International (PCT) Publication No. WO2015/148863.

In certain embodiments, the pharmaceutical composition comprises a targeting moiety to increase target cell binding or update of nanoparticles and liposomes. Exemplary targeting moieties include cell specific antigens, monoclonal antibodies, single chain antibodies, aptamers, polymers, sugars, and cell penetrating peptides. In certain embodiments, the pharmaceutical composition comprises a fusogenic or endosome-destabilizing peptide or polymer.

In certain embodiments, a pharmaceutical composition may contain a sustained- or controlled-delivery formulation. Techniques for formulating sustained- or controlled-delivery means, such as liposome carriers, bio-erodible microparticles or porous beads and depot injections, are also known to those skilled in the art. Sustained-release preparations may include, e.g., porous polymeric microparticles or semipermeable polymer matrices in the form of shaped articles, e.g., films, or microcapsules. Sustained release matrices may include polyesters, hydrogels, polylactides, copolymers of L-glutamic acid and gamma ethyl-L-glutamate, poly (2-hydroxyethyl-inethacrylate), ethylene vinyl acetate, or poly-D(−)-3-hydroxybutyric acid. Sustained release compositions may also include liposomes that can be prepared by any of several methods known in the art.

A pharmaceutical composition of the invention can be administered by a variety of methods known in the art. The route and/or mode of administration vary depending upon the desired results. Administration can be intravenous, intramuscular, intraperitoneal, or subcutaneous, or administered proximal to the site of the target. The pharmaceutically acceptable carrier should be suitable for intravenous, intramuscular, subcutaneous, parenteral, spinal or epidermal administration (e.g., by injection or infusion). Depending on the route of administration, the active compound (e.g., the guide nucleic acid, engineered, non-naturally occurring system, or CRISPR expression system of the invention) may be coated in a material to protect the compound from the action of acids and other natural conditions that may inactivate the compound.

Formulation components suitable for parenteral administration include a sterile diluent such as water for injection, saline solution, fixed oils, polyethylene glycols, glycerin, propylene glycol or other synthetic solvents; antibacterial agents such as benzyl alcohol or methyl parabens; antioxidants such as ascorbic acid or sodium bisulfite; chelating agents such as EDTA; buffers such as acetates, citrates or phosphates; and agents for the adjustment of tonicity such as sodium chloride or dextrose.

For intravenous administration, suitable carriers include physiological saline, bacteriostatic water, Cremophor EL™ (BASF, Parsippany, NJ) or phosphate buffered saline (PBS). The carrier should be stable under the conditions of manufacture and storage, and should be preserved against microorganisms. The carrier can be a solvent or dispersion medium containing, for example, water, ethanol, polyol (for example, glycerol, propylene glycol, and liquid polyetheylene glycol), and suitable mixtures thereof.

Pharmaceutical formulations preferably are sterile. Sterilization can be accomplished by any suitable method, e.g., filtration through sterile filtration membranes. Where the composition is lyophilized, filter sterilization can be conducted prior to or following lyophilization and reconstitution. In certain embodiments, the pharmaceutical composition is lyophilized, and then reconstituted in buffered saline, at the time of administration.

Pharmaceutical compositions of the invention can be prepared in accordance with methods well known and routinely practiced in the art. See, e.g., Remington: The Science and Practice of Pharmacy, Mack Publishing Co., 20th ed., 2000; and Sustained and Controlled Release Drug Delivery Systems, J. R. Robinson, ed., Marcel Dekker, Inc., New York, 1978. Pharmaceutical compositions are preferably manufactured under GMP conditions. Typically, a therapeutically effective dose or efficacious dose of the guide nucleic acid, engineered, non-naturally occurring system, or CRISPR expression system of the invention is employed in the pharmaceutical compositions of the invention. The multispecific antibodies of the invention are formulated into pharmaceutically acceptable dosage forms by conventional methods known to those of skill in the art. Dosage regimens are adjusted to provide the optimum desired response (e.g., a therapeutic response). For example, a single bolus may be administered, several divided doses may be administered over time or the dose may be proportionally reduced or increased as indicated by the exigencies of the therapeutic situation. It is especially advantageous to formulate parenteral compositions in dosage unit form for ease of administration and uniformity of dosage. Dosage unit form as used herein refers to physically discrete units suited as unitary dosages for the subjects to be treated; each unit contains a predetermined quantity of active compound calculated to produce the desired therapeutic effect in association with the required pharmaceutical carrier.

Actual dosage levels of the active ingredients in the pharmaceutical compositions of the invention can be varied so as to obtain an amount of the active ingredient which is effective to achieve the desired therapeutic response for a particular patient, composition, and mode of administration, without being toxic to the patient. The selected dosage level depends upon a variety of pharmacokinetic factors including the activity of the particular compositions of the present invention employed, or the ester, salt or amide thereof, the route of administration, the time of administration, the rate of excretion of the particular compound being employed, the duration of the treatment, other drugs, compounds and/or materials used in combination with the particular compositions employed, the age, sex, weight, condition, general health and prior medical history of the patient being treated, and like factors.

### IV. THERAPEUTIC USES

The guide nucleic acids, the engineered, non-naturally occurring systems, and the CRISPR expression systems disclosed herein are useful for targeting, editing, and/or modifying the genomic DNA in a cell or organism. These guide nucleic acids and systems, as well as a cell comprising one of the systems or a cell whose genome has been modified by one of the systems, can be used to treat a disease or disorder in which modification of genetic or epigenetic information is desirable. Accordingly, the present invention provides a method of treating a disease or disorder, the method comprising administering to a subject in need thereof a guide nucleic acid, a non-naturally occurring system, a CRISPR expression system, or a cell disclosed herein.

The term “subject” includes human and non-human animals. Non-human animals include all vertebrates, e.g., mammals and non-mammals, such as non-human primates, sheep, dog, cow, chickens, amphibians, and reptiles. Except when noted, the terms “patient” or “subject” are used herein interchangeably.

The terms “treatment”, “treating”, “treat”, “treated”, and the like, as used herein, include obtaining a desired pharmacologic and/or physiologic effect. The effect may be therapeutic in terms of a partial or complete cure for a disease and/or adverse effect attributable to the disease or delaying the disease progression. “Treatment”, as used herein, covers any treatment of a disease in a mammal, e.g., in a human, and includes: (a) inhibiting the disease, i.e., arresting its development; and (b) relieving the disease, i.e., causing regression of the disease. It is understood that a disease or disorder may be identified by genetic methods and treated prior to manifestation of any medical symptom.

For minimization of toxicity and off-target effect, it is important to control the concentration of the CRISPR-Cas system delivered. Optimal concentrations can be determined by testing different concentrations in a cellular, tissue, or non-human eukaryote animal model and using deep sequencing to analyze the extent of modification at potential off-target genomic loci. The concentration that gives the highest level of on-target modification while minimizing the level of off-target modification should be selected for ex vivo or in vivo delivery.

It is understood that the guide nucleic acid, the engineered, non-naturally occurring system, and the CRISPR expression system disclosed herein can be used to treat any disease or disorder that can be improved by editing or modifying human APLNR, BBS1, CALR, CD247, CD3D, CD38, CD3E, CD3G, CD40LG, CD52, CD58, COL17A1, CSF2, DEFB134, ERAP1, ERAP2, IFNGR1, IFNGR2, JAK1, JAK2, mir-101-2, MLANA, NLRC5 PSMB5, PSMB8, PSMB9, PTCD2, RFX5, RFXANK, RFXAP, RPL23, SOX10, SRP54, STAT1, Tap1, TAP2, TAPBP, TRBC1, TRBC1_2 (or TRBC1+2), TRBC2, or TWF1 gene in a cell. In certain embodiments, the guide nucleic acid, the engineered, non-naturally occurring system, and the CRISPR expression system disclosed herein can be used to engineer an immune cell. Immune cells include but are not limited to lymphocytes (e.g., B lymphocytes or B cells, T lymphocytes or T cells, and natural killer cells), myeloid cells (e.g., monocytes, macrophages, eosinophils, mast cells, basophils, and granulocytes), and the stem and progenitor cells that can differentiate into these cell types (e.g., hematopoietic stem cells, hematopoietic progenitor cells, and lymphoid progenitor cells). The cells can include autologous cells derived from a subject to be treated, or alternatively allogenic cells derived from a donor.

In certain embodiments, the immune cell is a T cell, which can be, for example, a cultured T cell, a primary T cell, a T cell from a cultured T cell line (e.g., Jurkat, SupTi), or a T cell obtained from a mammal, for example, from a subject to be treated. If obtained from a mammal, the T cell can be obtained from numerous sources, including but not limited to blood, bone marrow, lymph node, the thymus, or other tissues or fluids. T cells can also be enriched or purified. The T cell can be any type of T cell and can be of any developmental stage, including but not limited to, CD4⁺/CD8⁺ double positive T cells, CD4⁺ helper T cells (e.g., Th1 and Th2 cells), CD8⁺ T cells (e.g., cytotoxic T cells), tumor infiltrating lymphocytes (TILs), memory T cells (e.g., central memory T cells and effector memory T cells), regulatory T cells, naive T cells, and the like.

In certain embodiments, an immune cell, e.g., a T cell, is engineered to express an exogenous gene. For example, in certain embodiments, the guide nucleic acid, the engineered, non-naturally occurring system, and the CRISPR expression system disclosed herein may be used to engineer an immune cell to express an exogenous gene at the locus of a human APLNR, BBS1, CALR, CD247, CD3D, CD38, CD3E, CD3G, CD40LG, CD52, CD58, COL17A1, CSF2, DEFB134, ERAP1, ERAP2, IFNGR1, IFNGR2, JAK1, JAK2, mir-101-2, MLANA, NLRC5 PSMB5, PSMB8, PSMB9, PTCD2, RFX5, RFXANK, RFXAP, RPL23, SOX10, SRP54, STAT1, Tap1, TAP2, TAPBP, TRBC1, TRBC1_2 (or TRBC1+2), TRBC2, or TWF1 gene. For example, in certain embodiments, an engineered CRISPR system disclosed herein may catalyze DNA cleavage at the gene locus, allowing for site-specific integration of the exogenous gene at the gene locus by HDR.

In certain embodiments, an immune cell, e.g., a T cell, is engineered to express a chimeric antigen receptor (CAR), i.e., the T cell comprises an exogenous nucleotide sequence encoding a CAR. As used herein, the term “chimeric antigen receptor” or “CAR” refers to any artificial receptor including an antigen-specific binding moiety and one or more signaling chains derived from an immune receptor. CARs can comprise a single chain fragment variable (scFv) of an antibody specific for an antigen coupled via hinge and transmembrane regions to cytoplasmic domains of T cell signaling molecules, e.g., a T cell costimulatory domain (e.g., from CD28, CD137, OX40, ICOS, or CD27) in tandem with a T cell triggering domain (e.g., from CD3ζ). A T cell expressing a chimeric antigen receptor is referred to as a CAR T cell. Exemplary CAR T cells include CD19 targeted CTL019 cells (see, Grupp et al. (2015) BLOOD, 126: 4983), 19-28z cells (see, Park et al. (2015) J. CLIN. ONCOL., 33: 7010), and KTE-C19 cells (see, Locke et al. (2015) BLOOD, 126: 3991). Additional exemplary CAR T cells are described in U.S. Pat. Nos. 8,399,645, 8,906,682, 7,446,190, 9,181,527, 9,272,002, 9,266,960, 10,253,086, 10,808,035, and 10,640,569, and International (PCT) Publication Nos. WO2013/142034, WO2015/120180, WO2015/188141, WO2016/120220, and WO2017/040945. Exemplary approaches to express CARs using CRISPR systems are described in Hale et al. (2017) MOL THER METHODS CLIN DEV., 4: 192, MacLeod et al. (2017) MOL THER, 25: 949, and Eyquem et al. (2017) NATURE, 543: 113.

In certain embodiments, an immune cell, e.g., a T cell, binds an antigen, e.g., a cancer antigen, through an endogenous T cell receptor (TCR). In certain embodiments, an immune cell, e.g., a T cell, is engineered to express an exogenous TCR, e.g., an exogenous naturally occurring TCR or an exogenous engineered TCR. T cell receptors comprise two chains referred to as the α- and β-chains, that combine on the surface of a T cell to form a heterodimeric receptor that can recognize MHC-restricted antigens. Each of α- and β-chain comprises a constant region and a variable region. Each variable region of the α- and β-chains defines three loops, referred to as complementary determining regions (CDRs) known as CDR₁, CDR₂, and CDR₃ that confer the T cell receptor with antigen binding activity and binding specificity.

In certain embodiments, a CAR or TCR binds a cancer antigen selected from B-cell maturation antigen (BCMA), mesothelin, prostate specific membrane antigen (PSMA), prostate stem cell antigen (PCSA), carbonic anhydrase IX (CAIX), carcinoembryonic antigen (CEA), CD5, CD7, CD10, CD19, CD20, CD22, CD30, CD33, CD34, CD38, CD41, CD44, CD49f, CD56, CD70, CD74, CD123, CD133, CD138, epithelial glycoprotein2 (EGP 2), epithelial glycoprotein-40 (EGP-40), epithelial cell adhesion molecule (EpCAM), receptor-type tyrosine-protein kinase (FLT3), folate-binding protein (FBP), fetal acetylcholine receptor (AChR), folate receptor-α and β (FRa and β), Ganglioside G2 (GD2), Ganglioside G3 (GD3), epidermal growth factor receptor 2 (HER-2/ERB2), epidermal growth factor receptor vIII (EGFRvIII), ERB3, ERB4, human telom erase reverse transcriptase (hTERT), Interleukin-13 receptor subunit alpha-2 (IL-13Ra2), K-light chain, kinase insert domain receptor (KDR), Lewis A (CA19.9), Lewis Y (LeY), LI cell adhesion molecule (LICAM), melanoma-associated antigen 1 (melanoma antigen family A1, MAGE-A1), Mucin 16 (MUC-16), Mucin 1 (MUC-1; e.g., a truncated MUC-1), KG2D ligands, cancer-testis antigen NY-ESO-1, oncofetal antigen (h5T4), tumor-associated glycoprotein 72 (TAG-72), vascular endothelial growth factor R2 (VEGF-R2), Wilms tumor protein (WT-1), type 1 tyrosine-protein kinase transmembrane receptor (ROR1), B7-H3 (CD276), B7-H6 (Nkp30), Chondroitin sulfate proteoglycan-4 (CSPG4), DNAX Accessory Molecule (DNAM-1), Ephrin type A Receptor 2 (EpHA2), Fibroblast Associated Protein (FAP), Gp100/HLA-A2, Glypican 3 (GPC3), HA-IH, HERK-V, IL-1 IRa, Latent Membrane Protein 1 (LMP1), Neural cell-adhesion molecule (N-CAM/CD56), and Trail Receptor (TRAIL-R).

Genetic loci suitable for insertion of a CAR- or exogenous TCR-encoding sequence include but are not limited to TCR subunit loci (e.g., the TCRα constant (TRAC) locus, the TCRβ constant 1 (TRBC1) locus, and the TCRβ constant 2 (TRBC2) locus). It is understood that insertion in the TRAC locus reduces tonic CAR signaling and enhances T cell potency (see, Eyquem et al. (2017) NATURE, 543: 113). Furthermore, inactivation of the endogenous TRAC, TRBC1, or TRBC2 gene may reduce a graft-versus-host disease (GVHD) response, thereby allowing use of allogeneic T cells as starting materials for preparation of CAR-T cells. Accordingly, in certain embodiments, an immune cell, e.g., a T cell, is engineered to have reduced expression of an endogenous TCR or TCR subunit, e.g., TRAC, TRBC1, and/or TRBC2. The cell may be engineered to have partially reduced or no expression of the endogenous TCR or TCR subunit. For example, in certain embodiments, the immune cell, e.g., a T cell, is engineered to have less than 80% (e.g., less than 70%, less than 60%, less than 50%, less than 40%, less than 30%, less than 20%, less than 10%, or less than 5%) of the expression of the endogenous TCR or TCR subunit relative to a corresponding unmodified or parental cell. In certain embodiments, the immune cell, e.g., a T cell, is engineered to have no detectable expression of the endogenous TCR or TCR subunit. Exemplary approaches to reduce expression of TCRs using CRISPR systems are described in U.S. Pat. No. 9,181,527, Liu et al. (2017) CELL RES, 27: 154, Ren et al. (2017) CLIN CANCER RES, 23: 2255, Cooper et al. (2018) LEUKEMIA, 32: 1970, and Ren et al. (2017) ONCOTARGET, 8: 17002.

It is understood that certain immune cells, such as T cells, also express major histocompatibility complex (MHC) or human leukocyte antigen (HLA) genes, and inactivation of these endogenous gene may reduce a GVHD response, thereby allowing use of allogeneic T cells as starting materials for preparation of CAR-T cells. Accordingly, in certain embodiments, an immune cell, e.g., a T-cell, is engineered to have reduced expression of one or more endogenous class I or class II MHCs or HLAs (e.g., beta 2-microglobulin (B2M), class II major histocompatibility complex transactivator (CIITA), HLA-E, and/or HLA-G). The cell may be engineered to have partially reduced or no expression of an endogenous MHC or HLA. For example, in certain embodiments, the immune cell, e.g., a T-cell, is engineered to have less than less than 80% (e.g., less than 70%, less than 60%, less than 50%, less than 40%, less than 30%, less than 20%, less than 10%, or less than 5%) of the expression of endogenous MHC (e.g., B2M, CIITA, HLA-E, or HLA-G) relative to a corresponding unmodified or parental cell. In certain embodiments, the immune cell, e.g., a T cell, is engineered to have no detectable expression of an endogenous MHC (e.g., B2M, CIITA, HLA-E, or HLA-G). Exemplary approaches to reduce expression of MHCs using CRISPR systems are described in Liu et al. (2017) CELL RES, 27: 154, Ren et al. (2017) CLIN CANCER RES, 23: 2255, and Ren et al. (2017) ONCOTARGET, 8: 17002. Additional gene targets include but are not limited to B2M, CD247, CD3D, CD3E, CD3G, CIITA, NLRC5, TRAC, and TRBC1/2.

Other genes that may be inactivated to reduce a GVHD response include but are not limited to CD3, CD52, and deoxycytidine kinase (DCK). For example, inactivation of DCK may render the immune cells (e.g., T cells) resistant to purine nucleotide analogue (PNA) compounds, which are often used to compromise the host immune system in order to reduce a GVHD response during an immune cell therapy. In certain embodiments, the immune cell, e.g., a T-cell, is engineered to have less than less than 80% (e.g., less than 70%, less than 60%, less than 50%, less than 40%, less than 30%, less than 20%, less than 10%, or less than 5%) of the expression of endogenous CD52 or DCK relative to a corresponding unmodified or parental cell.

It is understood that the activity of an immune cell (e.g., T cell) may be enhanced by inactivating or reducing the expression of an immune suppressor such as an immune checkpoint protein. Accordingly, in certain embodiments, an immune cell, e.g., a T cell, is engineered to have reduced expression of an immune checkpoint protein. Exemplary immune checkpoint proteins expressed by wild-type T cells include but are not limited to PDCD1 (PD-1), CTLA4, ADORA2A (A2AR), B7-H3, B7-H4, BTLA, KIR, LAG3, HAVCR2 (TIM3), TIGIT, VISTA, PTPN6 (SHP-1), and FAS. The cell may be modified to have partially reduced or no expression of the immune checkpoint protein. For example, in certain embodiments, the immune cell, e.g., a T cell, is engineered to have less than 80% (e.g., less than 70%, less than 60%, less than 50%, less than 40%, less than 30%, less than 20%, less than 10%, or less than 5%) of the expression of the immune checkpoint protein relative to a corresponding unmodified or parental cell. In certain embodiments, the immune cell, e.g., a T cell, is engineered to have no detectable expression of the immune checkpoint protein. Exemplary approaches to reduce expression of immune checkpoint proteins using CRISPR systems are described in International (PCT) Publication No. WO2017/017184, Cooper et al. (2018) LEUKEMIA, 32: 1970, Su et al. (2016) ONCOIMMUNOLOGY, 6: e1249558, and Zhang et al. (2017) FRONT MED, 11: 554.

The immune cell can be engineered to have reduced expression of an endogenous gene, e.g., an endogenous genes described above, by gene editing or modification. For example, in certain embodiments, an engineered CRISPR system disclosed herein may result in DNA cleavage at a gene locus, thereby inactivating the targeted gene. In other embodiments, an engineered CRISPR system disclosed herein may be fused to an effector domain (e.g., a transcriptional repressor or histone methylase) to reduce the expression of the target gene.

The immune cell can also be engineered to express an exogenous protein (besides an antigen-binding protein described above) at the locus of a human APLNR, BBS1, CALR, CD247, CD3D, CD38, CD3E, CD3G, CD40LG, CD52, CD58, COL17A1, CSF2, DEFB134, ERAP1, ERAP2, IFNGR1, IFNGR2, JAK1, JAK2, mir-101-2, MLANA, NLRC5 PSMB5, PSMB8, PSMB9, PTCD2, RFX5, RFXANK, RFXAP, RPL23, SOX10, SRP54, STAT1, Tap1, TAP2, TAPBP, TRBC1, TRBC1_2 (or TRBC1+2), TRBC2, or TWF1 gene.

In certain embodiments, an immune cell, e.g., a T cell, is modified to express a dominant-negative form of an immune checkpoint protein. In certain embodiments, the dominant-negative form of the checkpoint inhibitor can act as a decoy receptor to bind or otherwise sequester the natural ligand that would otherwise bind and activate the wild-type immune checkpoint protein. Examples of engineered immune cells, for example, T cells containing dominant-negative forms of an immune suppressor are described, for example, in International (PCT) Publication No. WO2017/040945.

In certain embodiments, an immune cell, e.g., a T cell, is modified to express a gene (e.g., a transcription factor, a cytokine, or an enzyme) that regulates the survival, proliferation, activity, or differentiation (e.g., into a memory cell) of the immune cell. In certain embodiments, the immune cell is modified to express TET2, FOXO1, IL-12, IL-15, IL-18, IL-21, IL-7, GLUT1, GLUT3, HK1, HK2, GAPDH, LDHA, PDK1, PKM2, PFKFB3, PGK1, ENO1, GYS1, and/or ALDOA. In certain embodiments, the modification is an insertion of a nucleotide sequence encoding the protein operably linked to a regulatory element. In certain embodiments, the modification is a substitution of a single nucleotide polymorphism (SNP) site in the endogenous gene. In certain embodiments, an immune cell, e.g., a T cell, is modified to express a variant of a gene, for example, a variant that has greater activity than the respective wild-type gene. In certain embodiments, the immune cell is modified to express a variant of CARD11, CD247, IL7R, LCK, or PLCG1. For example, certain gain-of-function variants of IL7R were disclosed in Zenatti et al., (2011) NAT. GENET. 43(10):932-39. The variant can be expressed from the native locus of the respective wild-type gene by delivering an engineered system described herein for targeting the native locus in combination with a donor template that carries the variant or a portion thereof.

In certain embodiments, an immune cell, e.g., a T cell, is modified to express a protein (e.g., a cytokine or an enzyme) that regulates the microenvironment that the immune cell is designed to migrate to (e.g., a tumor microenvironment). In certain embodiments, the immune cell is modified to express CA9, CA12, a V-ATPase subunit, NHE1, and/or MCT-1.

### V. KITS

It is understood that the guide nucleic acid, the engineered, non-naturally occurring system, the CRISPR expression system, and the library disclosed herein can be packaged in a kit suitable for use by a medical provider. Accordingly, in another aspect, the invention provides kits containing any one or more of the elements disclosed in the above systems, libraries, methods, and compositions. In certain embodiments, the kit comprises an engineered, non-naturally occurring system as disclosed herein and instructions for using the kit. The instructions may be specific to the applications and methods described herein. In certain embodiments, one or more of the elements of the system are provided in a solution. In certain embodiments, one or more of the elements of the system are provided in lyophilized form, and the kit further comprises a diluent. Elements may be provided individually or in combinations, and may be provided in any suitable container, such as a vial, a bottle, a tube, or immobilized on the surface of a solid base (e.g., chip or microarray). In certain embodiments, the kit comprises one or more of the nucleic acids and/or proteins described herein. In certain embodiments, the kit provides all elements of the systems of the invention.

In certain embodiments of a kit comprising the engineered, non-naturally occurring dual guide system, the targeter nucleic acid and the modulator nucleic acid are provided in separate containers. In other embodiments, the targeter nucleic acid and the modulator nucleic acid are pre-complexed, and the complex is provided in a single container.

In certain embodiments, the kit comprises a Cas protein or a nucleic acid comprising a regulatory element operably linked to a nucleic acid encoding a Cas protein provided in a separate container. In other embodiments, the kit comprises a Cas protein pre-complexed with the single guide nucleic acid or a combination of the targeter nucleic acid and the modulator nucleic acid, and the complex is provided in a single container.

In certain embodiments, the kit further comprises one or more donor templates provided in one or more separate containers. In certain embodiments, the kit comprises a plurality of donor templates as disclosed herein (e.g., in separate tubes or immobilized on the surface of a solid base such as a chip or a microarray), one or more guide nucleic acids disclosed herein, and optionally a Cas protein or a regulatory element operably linked to a nucleic acid encoding a Cas protein as disclosed herein. Such kits are useful for identifying a donor template that introduces optimal genetic modification in a multiplex assay. The CRISPR expression systems as disclosed herein are also suitable for use in a kit.

In certain embodiments, a kit further comprises one or more reagents and/or buffers for use in a process utilizing one or more of the elements described herein. Reagents may be provided in any suitable container and may be provided in a form that is usable in a particular assay, or in a form that requires addition of one or more other components before use (e.g., in concentrate or lyophilized form). A buffer may be a reaction or storage buffer, including but not limited to a sodium carbonate buffer, a sodium bicarbonate buffer, a borate buffer, a Tris buffer, a MOPS buffer, a HEPES buffer, and combinations thereof. In some embodiments, the buffer is alkaline. In certain embodiments, the buffer has a pH from 6-9, 6.5-8.5, 7-8, 6.5-7.5, 6-8, 7.5-8.5, 7-9, 6.5-9.5, 6-10, 8-9, 7.5-9.5, 7-10, for example 7-8, such as 7.5. In certain embodiments, the kit further comprises a pharmaceutically acceptable carrier. In certain embodiments, the kit further comprises one or more devices or other materials for administration to a subject.

Throughout the description, where compositions are described as having, including, or comprising specific components, or where processes and methods are described as having, including, or comprising specific steps, it is contemplated that, additionally, there are compositions of the present invention that consist essentially of, or consist of, the recited components, and that there are processes and methods according to the present invention that consist essentially of, or consist of, the recited processing steps.

In the application, where an element or component is said to be included in and/or selected from a list of recited elements or components, it should be understood that the element or component can be any one of the recited elements or components, or the element or component can be selected from a group consisting of two or more of the recited elements or components.

Further, it should be understood that elements and/or features of a composition or a method described herein can be combined in a variety of ways without departing from the spirit and scope of the present invention, whether explicit or implicit herein. For example, where reference is made to a particular compound, that compound can be used in various embodiments of compositions of the present invention and/or in methods of the present invention, unless otherwise understood from the context. In other words, within this application, embodiments have been described and depicted in a way that enables a clear and concise application to be written and drawn, but it is intended and will be appreciated that embodiments may be variously combined or separated without parting from the present teachings and invention(s). For example, it will be appreciated that all features described and depicted herein can be applicable to all aspects of the invention(s) described and depicted herein.

The terms “a” and “an” and “the” and similar references in the context of describing the invention (especially in the context of the following claims) are to be construed to cover both the singular and the plural, unless otherwise indicated herein or clearly contradicted by context. For example, the term “a cell” includes a plurality of cells, including mixtures thereof. Where the plural form is used for compounds, salts, and the like, this is taken to mean also a single compound, salt, or the like.

It should be understood that the expression “at least one of” includes individually each of the recited objects after the expression and the various combinations of two or more of the recited objects unless otherwise understood from the context and use. The expression “and/or” in connection with three or more recited objects should be understood to have the same meaning unless otherwise understood from the context.

The use of the term “include,” “includes,” “including,” “have,” “has,” “having,” “contain,” “contains,” or “containing,” including grammatical equivalents thereof, should be understood generally as open-ended and non-limiting, for example, not excluding additional unrecited elements or steps, unless otherwise specifically stated or understood from the context.

Where the use of the term “about” is before a quantitative value, the present invention also includes the specific quantitative value itself, unless specifically stated otherwise. As used herein, the term “about” refers to a ±10% variation from the nominal value unless otherwise indicated or inferred.

It should be understood that the order of steps or order for performing certain actions is immaterial so long as the present invention remain operable. Moreover, two or more steps or actions may be conducted simultaneously.

The use of any and all examples, or exemplary language herein, for example, “such as” or “including,” is intended merely to illustrate better the present invention and does not pose a limitation on the scope of the invention unless claimed. No language in the specification should be construed as indicating any non-claimed element as essential to the practice of the present invention.

### Embodiments

In embodiment 1 provided herein is a guide nucleic acid comprising a targeter stem sequence and a spacer sequence, wherein the spacer sequence comprises a nucleotide sequence listed in Table 1, 2, 3, 4, 5, 6, 7, 8, or 9. In embodiment 2 provided herein is the guide nucleic acid of embodiment 1, wherein the targeter stem sequence comprises a nucleotide sequence of GUAGA. In embodiment 3 provided herein is the guide nucleic acid of embodiment 1 or 2, wherein the targeter stem sequence is 5′ to the spacer sequence, optionally wherein the targeter stem sequence is linked to the spacer sequence by a linker consisting of 1, 2, 3, 4, or 5 nucleotides. In embodiment 4 provided herein is the guide nucleic acid of any one of embodiments 1-3, wherein the guide nucleic acid is capable of activating a CRISPR Associated (Cas) nuclease in the absence of a tracrRNA. In embodiment 5 provided herein is the guide nucleic acid of embodiment 4, wherein the guide nucleic acid comprises from 5′ to 3′ a modulator stem sequence, a loop sequence, a targeter stem sequence, and the spacer sequence. In embodiment 6 provided herein is the guide nucleic acid of any one of embodiments 1-3, wherein the guide nucleic acid is a targeter nucleic acid that, in combination with a modulator nucleic acid, is capable of activating a Cas nuclease. In embodiment 7 provided herein is the guide nucleic acid of embodiment 6, wherein the guide nucleic acid comprises from 5′ to 3′ a targeter stem sequence and the spacer sequence. In embodiment 8 provided herein is the guide nucleic acid of any one of embodiments 4-7, wherein the Cas nuclease is a type V Cas nuclease. In embodiment 9 provided herein is the guide nucleic acid of embodiment 8, wherein the Cas nuclease is a type V-A Cas nuclease. In embodiment 10 provided herein is the guide nucleic acid of embodiment 9, wherein the Cas nuclease comprises an amino acid sequence at least 80% identical to SEQ ID NO: 1. In embodiment 11 provided herein is the guide nucleic acid of embodiment 9, wherein the Cas nuclease is Cpf1. In embodiment 12 provided herein is the guide nucleic acid of any one of embodiments 4-11, wherein the Cas nuclease recognizes a protospacer adjacent motif (PAM) consisting of the nucleotide sequence of TTTN or CTTN. In embodiment 13 provided herein is the guide nucleic acid of any one of the proceeding embodiments, wherein the guide nucleic acid comprises a ribonucleic acid (RNA). In embodiment 14 provided herein is the guide nucleic acid of embodiment 13, wherein the guide nucleic acid comprises a modified RNA. In embodiment 15 provided herein is the guide nucleic acid of embodiment 13 or 14, wherein the guide nucleic acid comprises a combination of RNA and DNA. In embodiment 16 provided herein is the guide nucleic acid of any one of embodiments 13-15, wherein the guide nucleic acid comprises a chemical modification. In embodiment 17 provided herein is the guide nucleic acid of embodiment 16, wherein the chemical modification is present in one or more nucleotides at the 5′ end of the guide nucleic acid. In embodiment 18 provided herein is the guide nucleic acid of embodiment 16 or 17, wherein the chemical modification is present in one or more nucleotides at the 3′ end of the guide nucleic acid. In embodiment 19 provided herein is the guide nucleic acid of any one of embodiments 16-18, wherein the chemical modification is selected from the group consisting of 2′-O-methyl, 2′-fluoro, 2′-O-methoxyethyl, phosphorothioate, phosphorodithioate, pseudouridine, and any combinations thereof. In embodiment 20 provided herein is an engineered, non-naturally occurring system comprising the guide nucleic acid of any one of embodiments 4-5 and 8-19. In embodiment 21 provided herein is the engineered, non-naturally occurring system of embodiment 20, further comprising the Cas nuclease. In embodiment 22 provided herein is the engineered, non-naturally occurring system of embodiment 21, wherein the guide nucleic acid and the Cas nuclease are present in a ribonucleoprotein (RNP) complex. In embodiment 23 provided herein is an engineered, non-naturally occurring system comprising the guide nucleic acid of any one of embodiments 6-19, further comprising the modulator nucleic acid. In embodiment 24 provided herein is the engineered, non-naturally occurring system of embodiment 23, further comprising the Cas nuclease. In embodiment 25 provided herein is the engineered, non-naturally occurring system of embodiment 24, wherein the guide nucleic acid, the modulator nucleic acid, and the Cas nuclease are present in an RNP complex. In embodiment 26 provided herein is the engineered, non-naturally occurring system of any one of embodiments 1-25, wherein the spacer sequence comprises a nucleotide sequence selected from the group consisting of SEQ ID NOs: 201-253, and wherein the spacer sequence is capable of hybridizing with the human CSF2 gene. In embodiment 27 provided herein is the engineered, non-naturally occurring system of embodiment 26, wherein, when the system is delivered into a population of human cells ex vivo, the genomic sequence at the CSF2 gene locus is edited in at least 1.5% of the cells. In embodiment 28 provided herein is the engineered, non-naturally occurring system of any one of embodiments 1-25, wherein the spacer sequence comprises a nucleotide sequence selected from the group consisting of SEQ ID NOs: 254-313, and wherein the spacer sequence is capable of hybridizing with the human CD40LG gene. In embodiment 29 provided herein is the engineered, non-naturally occurring system of embodiment 28, wherein, when the system is delivered into a population of human cells ex vivo, the genomic sequence at the CD40LG gene locus is edited in at least 1.5% of the cells. In embodiment 30 provided herein is the engineered, non-naturally occurring system of any one of embodiments 1-25, wherein the spacer sequence comprises a nucleotide sequence selected from the group consisting of SEQ ID NOs: 314-319 and 329-332, and wherein the spacer sequence is capable of hybridizing with the human TRBC1 gene. In embodiment 31 provided herein is the engineered, non-naturally occurring system of embodiment 30, wherein, when the system is delivered into a population of human cells ex vivo, the genomic sequence at the TRBC1 gene locus is edited in at least 1.5% of the cells. In embodiment 32 provided herein is the engineered, non-naturally occurring system of any one of embodiments 1-25, wherein the spacer sequence comprises a nucleotide sequence selected from the group consisting of SEQ ID NOs: 320-328 and 329-332, and wherein the spacer sequence is capable of hybridizing with the human TRBC2 gene. In embodiment 33 provided herein is the engineered, non-naturally occurring system of embodiment 32, wherein, when the system is delivered into a population of human cells ex vivo, the genomic sequence at the TRBC2 gene locus is edited in at least 1.5% of the cells. In embodiment 34 provided herein is the engineered, non-naturally occurring system of any one of embodiments 1-25, wherein the spacer sequence comprises a nucleotide sequence selected from the group consisting of SEQ ID NOs: 329-332, and wherein the spacer sequence is capable of hybridizing with both the human TRBC1 gene and the human TRBC2 gene. In embodiment 35 provided herein is the engineered, non-naturally occurring system of embodiment 34, wherein, when the system is delivered into a population of human cells ex vivo, the genomic sequence at both the human TRBC1 gene and the human TRBC2 gene locus is edited in at least 1.5% of the cells. In embodiment 36 provided herein is the engineered, non-naturally occurring system of any one of embodiments 1-25, wherein the spacer sequence comprises a nucleotide sequence selected from the group consisting of SEQ ID NOs: 333-374 and wherein the spacer sequence is capable of hybridizing with the human CD3E gene. In embodiment 37 provided herein is the engineered, non-naturally occurring system of embodiment 36, wherein, when the system is delivered into a population of human cells ex vivo, the genomic sequence at the CD3E gene locus is edited in at least 1.5% of the cells. In embodiment 38 provided herein is the engineered, non-naturally occurring system of any one of embodiments 1-25, wherein the spacer sequence comprises a nucleotide sequence selected from the group consisting of SEQ ID NOs: 375-411, and wherein the spacer sequence is capable of hybridizing with the human CD38 gene. In embodiment 39 provided herein is the engineered, non-naturally occurring system of embodiment 38, wherein, when the system is delivered into a population of human cells ex vivo, the genomic sequence at the CD38 gene locus is edited in at least 1.5% of the cells. In embodiment 40 provided herein is the engineered, non-naturally occurring system of any one of embodiments 1-25, wherein the spacer sequence comprises a nucleotide sequence selected from the group consisting of SEQ ID NOs: 412-421, and wherein the spacer sequence is capable of hybridizing with the human APLNR gene. In embodiment 41 provided herein is the engineered, non-naturally occurring system of embodiment 40, wherein, when the system is delivered into a population of human cells ex vivo, the genomic sequence at the APLNR gene locus is edited in at least 1.5% of the cells. In embodiment 42 provided herein is the engineered, non-naturally occurring system of any one of embodiments 1-25, wherein the spacer sequence comprises a nucleotide sequence selected from the group consisting of SEQ ID NOs: 422-431, and wherein the spacer sequence is capable of hybridizing with the human BBS1 gene. In embodiment 43 provided herein is the engineered, non-naturally occurring system of embodiment 42, wherein, when the system is delivered into a population of human cells ex vivo, the genomic sequence at the BBS1 gene locus is edited in at least 1.5% of the cells. In embodiment 44 provided herein is the engineered, non-naturally occurring system of any one of embodiments 1-25, wherein the spacer sequence comprises a nucleotide sequence selected from the group consisting of SEQ ID NOs: 432-441, and wherein the spacer sequence is capable of hybridizing with the human CALR gene. In embodiment 45 provided herein is the engineered, non-naturally occurring system of embodiment 44, wherein, when the system is delivered into a population of human cells ex vivo, the genomic sequence at the CALR gene locus is edited in at least 1.5% of the cells. In embodiment 46 provided herein is the engineered, non-naturally occurring system of any one of embodiments 1-25, wherein the spacer sequence comprises a nucleotide sequence selected from the group consisting of SEQ ID NOs: 442-451, and wherein the spacer sequence is capable of hybridizing with the human CD247 gene. In embodiment 47 provided herein is the engineered, non-naturally occurring system of embodiment 46, wherein, when the system is delivered into a population of human cells ex vivo, the genomic sequence at the CD247 gene locus is edited in at least 1.5% of the cells. In embodiment 48 provided herein is the engineered, non-naturally occurring system of any one of embodiments 1-25, wherein the spacer sequence comprises a nucleotide sequence selected from the group consisting of SEQ ID NOs: 452-461, and wherein the spacer sequence is capable of hybridizing with the human CD3G gene. In embodiment 49 provided herein is the engineered, non-naturally occurring system of embodiment 48, wherein, when the system is delivered into a population of human cells ex vivo, the genomic sequence at the CD3G locus is edited in at least 1.5% of the cells. In embodiment 50 provided herein is the engineered, non-naturally occurring system of any one of embodiments 1-25, wherein the spacer sequence comprises a nucleotide sequence selected from the group consisting of SEQ ID NOs: 462-465, and wherein the spacer sequence is capable of hybridizing with the human CD52 gene. In embodiment 51 provided herein is the engineered, non-naturally occurring system of embodiment 50, wherein, when the system is delivered into a population of human cells ex vivo, the genomic sequence at the CD52 locus is edited in at least 1.5% of the cells. In embodiment 52 provided herein is the engineered, non-naturally occurring system of any one of embodiments 1-25, wherein the spacer sequence comprises a nucleotide sequence selected from the group consisting of SEQ ID NOs: 466-475, and wherein the spacer sequence is capable of hybridizing with the human CD58 gene. In embodiment 53 provided herein is the engineered, non-naturally occurring system of embodiment 52, wherein, when the system is delivered into a population of human cells ex vivo, the genomic sequence at the CD58 locus is edited in at least 1.5% of the cells. In embodiment 54 provided herein is the engineered, non-naturally occurring system of any one of embodiments 1-25, wherein the spacer sequence comprises a nucleotide sequence selected from the group consisting of SEQ ID NOs: 476-485, and wherein the spacer sequence is capable of hybridizing with the human COL17A1 gene. In embodiment 55 provided herein is the engineered, non-naturally occurring system of embodiment 54, wherein, when the system is delivered into a population of human cells ex vivo, the genomic sequence at the COL17A1 locus is edited in at least 1.5% of the cells. In embodiment 56 provided herein is the engineered, non-naturally occurring system of any one of embodiments 1-25, wherein the spacer sequence comprises a nucleotide sequence selected from the group consisting of SEQ ID NOs: 486-495, and wherein the spacer sequence is capable of hybridizing with the human DEFB134 gene. In embodiment 57 provided herein is the engineered, non-naturally occurring system of embodiment 56, wherein, when the system is delivered into a population of human cells ex vivo, the genomic sequence at the DEFB134 locus is edited in at least 1.5% of the cells. In embodiment 58 provided herein is the engineered, non-naturally occurring system of any one of embodiments 1-25, wherein the spacer sequence comprises a nucleotide sequence selected from the group consisting of SEQ ID NOs: 496-505, and wherein the spacer sequence is capable of hybridizing with the human ERAP1 gene. In embodiment 59 provided herein is the engineered, non-naturally occurring system of embodiment 58, wherein, when the system is delivered into a population of human cells ex vivo, the genomic sequence at the ERAP1 locus is edited in at least 1.5% of the cells. In embodiment 60 provided herein is the engineered, non-naturally occurring system of any one of embodiments 1-25, wherein the spacer sequence comprises a nucleotide sequence selected from the group consisting of SEQ ID NOs: 506-515, and wherein the spacer sequence is capable of hybridizing with the human ERAP2 gene. In embodiment 61 provided herein is the engineered, non-naturally occurring system of embodiment 60, wherein, when the system is delivered into a population of human cells ex vivo, the genomic sequence at the ERAP2 locus is edited in at least 1.5% of the cells. In embodiment 62 provided herein is the engineered, non-naturally occurring system of any one of embodiments 1-25, wherein the spacer sequence comprises a nucleotide sequence selected from the group consisting of SEQ ID NOs: 516-525, and wherein the spacer sequence is capable of hybridizing with the human IFNGR1 gene. In embodiment 63 provided herein is the engineered, non-naturally occurring system of embodiment 62, wherein, when the system is delivered into a population of human cells ex vivo, the genomic sequence at the IFNGR1 locus is edited in at least 1.5% of the cells. In embodiment 64 provided herein is the engineered, non-naturally occurring system of any one of embodiments 1-25, wherein the spacer sequence comprises a nucleotide sequence selected from the group consisting of SEQ ID NOs: 526-535, and wherein the spacer sequence is capable of hybridizing with the human IFNGR2 gene. In embodiment 65 provided herein is the engineered, non-naturally occurring system of embodiment 64, wherein, when the system is delivered into a population of human cells ex vivo, the genomic sequence at the IFNGR2 locus is edited in at least 1.5% of the cells. In embodiment 66 provided herein is the engineered, non-naturally occurring system of any one of embodiments 1-25, wherein the spacer sequence comprises a nucleotide sequence selected from the group consisting of SEQ ID NOs: 536-545, and wherein the spacer sequence is capable of hybridizing with the human JAK1 gene. In embodiment 67 provided herein is the engineered, non-naturally occurring system of embodiment 66, wherein, when the system is delivered into a population of human cells ex vivo, the genomic sequence at the JAK1 locus is edited in at least 1.5% of the cells. In embodiment 68 provided herein is the engineered, non-naturally occurring system of any one of embodiments 1-25, wherein the spacer sequence comprises a nucleotide sequence selected from the group consisting of SEQ ID NOs: 546-555, and wherein the spacer sequence is capable of hybridizing with the human JAK2 gene. In embodiment 69 provided herein is the engineered, non-naturally occurring system of embodiment 68, wherein, when the system is delivered into a population of human cells ex vivo, the genomic sequence at the JAK2 locus is edited in at least 1.5% of the cells. In embodiment 70 provided herein is the engineered, non-naturally occurring system of any one of embodiments 1-25, wherein the spacer sequence comprises a nucleotide sequence selected from the group consisting of SEQ ID NOs: 556-558, and wherein the spacer sequence is capable of hybridizing with the human mir-101-2 gene. In embodiment 71 provided herein is the engineered, non-naturally occurring system of embodiment 70, wherein, when the system is delivered into a population of human cells ex vivo, the genomic sequence at the mir-101-2 locus is edited in at least 1.5% of the cells. In embodiment 72 provided herein is the engineered, non-naturally occurring system of any one of embodiments 1-25, wherein the spacer sequence comprises a nucleotide sequence selected from the group consisting of SEQ ID NOs: 559-568, and wherein the spacer sequence is capable of hybridizing with the human MLANA gene. In embodiment 73 provided herein is the engineered, non-naturally occurring system of embodiment 72, wherein, when the system is delivered into a population of human cells ex vivo, the genomic sequence at the MLANA locus is edited in at least 1.5% of the cells. In embodiment 74 provided herein is the engineered, non-naturally occurring system of any one of embodiments 1-25, wherein the spacer sequence comprises a nucleotide sequence selected from the group consisting of SEQ ID NOs: 569-578, and wherein the spacer sequence is capable of hybridizing with the human PSMB5 gene. In embodiment 75 provided herein is the engineered, non-naturally occurring system of embodiment 74, wherein, when the system is delivered into a population of human cells ex vivo, the genomic sequence at the PSMB5 locus is edited in at least 1.5% of the cells. In embodiment 76 provided herein is the engineered, non-naturally occurring system of any one of embodiments 1-25, wherein the spacer sequence comprises a nucleotide sequence selected from the group consisting of SEQ ID NOs: 579-588, and wherein the spacer sequence is capable of hybridizing with the human PSMB8 gene. In embodiment 77 provided herein is the engineered, non-naturally occurring system of embodiment 76, wherein, when the system is delivered into a population of human cells ex vivo, the genomic sequence at the PSMB8 locus is edited in at least 1.5% of the cells. In embodiment 78 provided herein is the engineered, non-naturally occurring system of any one of embodiments 1-25, wherein the spacer sequence comprises a nucleotide sequence selected from the group consisting of SEQ ID NOs: 589-598, and wherein the spacer sequence is capable of hybridizing with the human PSMB9 gene. In embodiment 79 provided herein is the engineered, non-naturally occurring system of embodiment 78, wherein, when the system is delivered into a population of human cells ex vivo, the genomic sequence at the PSMB9 locus is edited in at least 1.5% of the cells. In embodiment 80 provided herein is the engineered, non-naturally occurring system of any one of embodiments 1-25, wherein the spacer sequence comprises a nucleotide sequence selected from the group consisting of SEQ ID NOs: 599-608, and wherein the spacer sequence is capable of hybridizing with the human PTCD2 gene. In embodiment 81 provided herein is the engineered, non-naturally occurring system of embodiment 80, wherein, when the system is delivered into a population of human cells ex vivo, the genomic sequence at the PTCD2 locus is edited in at least 1.5% of the cells. In embodiment 82 provided herein is the engineered, non-naturally occurring system of any one of embodiments 1-25, wherein the spacer sequence comprises a nucleotide sequence selected from the group consisting of SEQ ID NOs: 609-618, and wherein the spacer sequence is capable of hybridizing with the human RFX5 gene. In embodiment 83 provided herein is the engineered, non-naturally occurring system of embodiment 82, wherein, when the system is delivered into a population of human cells ex vivo, the genomic sequence at the RFX5 locus is edited in at least 1.5% of the cells. In embodiment 84 provided herein is the engineered, non-naturally occurring system of any one of embodiments 1-25, wherein the spacer sequence comprises a nucleotide sequence selected from the group consisting of SEQ ID NOs: 619-628, and wherein the spacer sequence is capable of hybridizing with the human RFXANK gene. In embodiment 85 provided herein is the engineered, non-naturally occurring system of embodiment 84, wherein, when the system is delivered into a population of human cells ex vivo, the genomic sequence at the RFXANK locus is edited in at least 1.5% of the cells. In embodiment 86 provided herein is the engineered, non-naturally occurring system of any one of embodiments 1-25, wherein the spacer sequence comprises a nucleotide sequence selected from the group consisting of SEQ ID NOs: 629-638, and wherein the spacer sequence is capable of hybridizing with the human RFXAP gene. In embodiment 87 provided herein is the engineered, non-naturally occurring system of embodiment 86, wherein, when the system is delivered into a population of human cells ex vivo, the genomic sequence at the RFXAP locus is edited in at least 1.5% of the cells. In embodiment 88 provided herein is the engineered, non-naturally occurring system of any one of embodiments 1-25, wherein the spacer sequence comprises a nucleotide sequence selected from the group consisting of SEQ ID NOs: 639-648, and wherein the spacer sequence is capable of hybridizing with the human RPL23 gene. In embodiment 89 provided herein is the engineered, non-naturally occurring system of embodiment 88, wherein, when the system is delivered into a population of human cells ex vivo, the genomic sequence at the RPL23 locus is edited in at least 1.5% of the cells. In embodiment 90 provided herein is the engineered, non-naturally occurring system of any one of embodiments 1-25, wherein the spacer sequence comprises a nucleotide sequence selected from the group consisting of SEQ ID NOs: 649-654, and wherein the spacer sequence is capable of hybridizing with the human SOX10 gene. In embodiment 91 provided herein is the engineered, non-naturally occurring system of embodiment 90, wherein, when the system is delivered into a population of human cells ex vivo, the genomic sequence at the SOX10 locus is edited in at least 1.5% of the cells. In embodiment 92 provided herein is the engineered, non-naturally occurring system of any one of embodiments 1-25, wherein the spacer sequence comprises a nucleotide sequence selected from the group consisting of SEQ ID NOs: 655-665, and wherein the spacer sequence is capable of hybridizing with the human SRP54 gene. In embodiment 93 provided herein is the engineered, non-naturally occurring system of embodiment 92, wherein, when the system is delivered into a population of human cells ex vivo, the genomic sequence at the SRP54 locus is edited in at least 1.5% of the cells. In embodiment 94 provided herein is the engineered, non-naturally occurring system of any one of embodiments 1-25, wherein the spacer sequence comprises a nucleotide sequence selected from the group consisting of SEQ ID NOs: 666-675, and wherein the spacer sequence is capable of hybridizing with the human STAT1 gene. In embodiment 95 provided herein is the engineered, non-naturally occurring system of embodiment 94, wherein, when the system is delivered into a population of human cells ex vivo, the genomic sequence at the STAT1 locus is edited in at least 1.5% of the cells. In embodiment 96 provided herein is the engineered, non-naturally occurring system of any one of embodiments 1-25, wherein the spacer sequence comprises a nucleotide sequence selected from the group consisting of SEQ ID NOs: 676-685, and wherein the spacer sequence is capable of hybridizing with the human Tap1 gene. In embodiment 97 provided herein is the engineered, non-naturally occurring system of embodiment 96, wherein, when the system is delivered into a population of human cells ex vivo, the genomic sequence at the Tap1 locus is edited in at least 1.5% of the cells. In embodiment 98 provided herein is the engineered, non-naturally occurring system of any one of embodiments 1-25, wherein the spacer sequence comprises a nucleotide sequence selected from the group consisting of SEQ ID NOs: 686-695, and wherein the spacer sequence is capable of hybridizing with the human Tap2 gene. In embodiment 99 provided herein is the engineered, non-naturally occurring system of embodiment 98, wherein, when the system is delivered into a population of human cells ex vivo, the genomic sequence at the Tap2 locus is edited in at least 1.5% of the cells. In embodiment 100 provided herein is the engineered, non-naturally occurring system of any one of embodiments 1-25, wherein the spacer sequence comprises a nucleotide sequence selected from the group consisting of SEQ ID NOs: 696-705, and wherein the spacer sequence is capable of hybridizing with the human TAPBP gene. In embodiment 101 provided herein is the engineered, non-naturally occurring system of embodiment 100, wherein, when the system is delivered into a population of human cells ex vivo, the genomic sequence at the TAPBP locus is edited in at least 1.5% of the cells. In embodiment 102 provided herein is the engineered, non-naturally occurring system of any one of embodiments 1-25, wherein the spacer sequence comprises a nucleotide sequence selected from the group consisting of SEQ ID NOs: 706-715, and wherein the spacer sequence is capable of hybridizing with the human TFW1 gene. In embodiment 103 provided herein is the engineered, non-naturally occurring system of embodiment 102, wherein, when the system is delivered into a population of human cells ex vivo, the genomic sequence at the TFW1 locus is edited in at least 1.5% of the cells. In embodiment 104 provided herein is the engineered, non-naturally occurring system of any one of embodiments 1-25, wherein the spacer sequence comprises a nucleotide sequence selected from the group consisting of SEQ ID NOs: 716-725, and wherein the spacer sequence is capable of hybridizing with the human CD3D gene. In embodiment 105 provided herein is the engineered, non-naturally occurring system of embodiment 104, wherein, when the system is delivered into a population of human cells ex vivo, the genomic sequence at the CD3D locus is edited in at least 1.5% of the cells. In embodiment 106 provided herein is the engineered, non-naturally occurring system of any one of embodiments 1-25, wherein the spacer sequence comprises a nucleotide sequence selected from the group consisting of SEQ ID NOs: 726-744, and wherein the spacer sequence is capable of hybridizing with the human NLRC5 gene. In embodiment 107 provided herein is the engineered, non-naturally occurring system of embodiment 106, wherein, when the system is delivered into a population of human cells ex vivo, the genomic sequence at the NLRC5 locus is edited in at least 1.5% of the cells. In embodiment 108 provided herein is the engineered, non-naturally occurring system of any one of embodiments 20-107, wherein genomic mutations are detected in no more than 2% of the cells at any off-target loci by CIRCLE-Seq. In embodiment 109 provided herein is the engineered, non-naturally occurring system of embodiment 108, wherein genomic mutations are detected in no more than 1% of the cells at any off-target loci by CIRCLE-Seq. In embodiment 110 provided herein is a human cell comprising the engineered, non-naturally occurring system of any one of embodiments 20-109. In embodiment 111 provided herein is a composition comprising the guide nucleic acid of any one of embodiments 1-19, the engineered, non-naturally occurring system of any one of embodiments 20-109, or the human cell of embodiment 110. In embodiment 112 provided herein is a method of cleaving a target DNA comprising the sequence of a preselected target gene or a portion thereof, the method comprising contacting the target DNA with the engineered, non-naturally occurring system of any one of embodiments 20-109, thereby resulting in cleavage of the target DNA. In embodiment 113 provided herein is the method of embodiment 112, wherein the contacting occurs in vitro. In embodiment 114 provided herein is the method of embodiment 112, wherein the contacting occurs in a cell ex vivo. In embodiment 115 provided herein is the method of embodiment 114, wherein the target DNA is genomic DNA of the cell. In embodiment 116 provided herein is a method of editing human genomic sequence at a preselected target gene locus, the method comprising delivering the engineered, non-naturally occurring system of any one of embodiments 20-109 into a human cell, thereby resulting in editing of the genomic sequence at the target gene locus in the human cell. In embodiment 117 provided herein is the method of any one of embodiments 114-116, wherein the cell is an immune cell. In embodiment 118 provided herein is the method of embodiment 117, wherein the immune cell is a T lymphocyte. In embodiment 119 provided herein is the method of embodiment 116, the method comprising delivering the engineered, non-naturally occurring system of any one of embodiments 20-109 into a population of human cells, thereby resulting in editing of the genomic sequence at the target gene locus in at least a portion of the human cells. In embodiment 120 provided herein is the method of embodiment 119, wherein the population of human cells comprises human immune cells. In embodiment 121 provided herein is the method of embodiment 119 or 120, wherein the population of human cells is an isolated population of human immune cells. In embodiment 122 provided herein is the method of embodiment 120 or 121, wherein the immune cells are T lymphocytes. In embodiment 123 provided herein is the method of any one of embodiments 119-122, wherein editing of the genomic sequence at the target gene locus results lowered expression of the target gene. In embodiment 124 provided herein is the method of embodiment 123, wherein the edited cell demonstrates less than 80% of the expression of the endogenous gene relative to a corresponding unmodified or parental cell. In embodiment 125 provided herein is the method of embodiment 123, wherein the edited cell demonstrates less than 70% of the expression of the endogenous gene relative to a corresponding unmodified or parental cell. In embodiment 126 provided herein is the method of embodiment 123, wherein the edited cell demonstrates less than 60% of the expression of the endogenous gene relative to a corresponding unmodified or parental cell. In embodiment 127 provided herein is the method of embodiment 123, wherein the edited cell demonstrates less than 50% of the expression of the endogenous gene relative to a corresponding unmodified or parental cell. In embodiment 128 provided herein is the method of any one of embodiments 116-127, wherein the engineered, non-naturally occurring system is delivered into the cell(s) as a pre-formed RNP complex. In embodiment 129 provided herein is the method of embodiment 128, wherein the pre-formed RNP complex is delivered into the cell(s) by electroporation. In embodiment 130 provided herein is the method of any one of embodiments 116-129, wherein the target gene is human CSF2 gene, and wherein the spacer sequence comprises a nucleotide sequence selected from the group consisting of SEQ ID NOs: 201-253. In embodiment 131 provided herein is the method of any one of embodiments 119-130, wherein the genomic sequence at the CSF2 gene locus is edited in at least 1.5% of the human cells. In embodiment 132 provided herein is the method of any one of embodiments 116-129, wherein the target gene is human CD40LG gene, and wherein the spacer sequence comprises a nucleotide sequence selected from the group consisting of SEQ ID NOs: 254-313. In embodiment 133 provided herein is the method of any one of embodiments 119-129 and 132, wherein the genomic sequence at the CD40LG gene locus is edited in at least 1.5% of the human cells. In embodiment 134 provided herein is the method of any one of embodiments 116-129, wherein the target gene is human TRBC1 gene, and wherein the spacer sequence comprises a nucleotide sequence selected from the group consisting of SEQ ID NOs: 314-319 and 329-332. In embodiment 135 provided herein is the method of any one of embodiments 119-129 and 134, wherein the genomic sequence at the TRBC1 gene locus is edited in at least 1.5% of the human cells. In embodiment 136 provided herein is the method of any one of embodiments 116-129, wherein the target gene is human TRBC2 gene, and wherein the spacer sequence comprises a nucleotide sequence selected from the group consisting of SEQ ID NOs: 320-328 and 329-332. In embodiment 137 provided herein is the method of any one of embodiments 119-129 and 136, wherein the genomic sequence at the TRBC2 gene locus is edited in at least 1.5% of the human cells. In embodiment 138 provided herein is the method of any one of embodiments 116-129, wherein the target gene is both the human TRBC1 gene and the human TRBC2 gene, and wherein the spacer sequence comprises a nucleotide sequence selected from the group consisting of SEQ ID NOs: 329-332. In embodiment 139 provided herein is the method of any one of embodiments 119-129 and 138, wherein the genomic sequence at both the human TRBC1 gene and the human TRBC2 gene locus is edited in at least 1.5% of the human cells. In embodiment 140 provided herein is the method of any one of embodiments 116-129, wherein the target gene is human CD3E gene, and wherein the spacer sequence comprises a nucleotide sequence selected from the group consisting of SEQ ID NOs: 333-374. In embodiment 141 provided herein is the method of any one of embodiments 119-129 and 140, wherein the genomic sequence at the CD3E gene locus is edited in at least 1.5% of the human cells. In embodiment 142 provided herein is the method of any one of embodiments 116-129, wherein the target gene is human CD38 gene, and wherein the spacer sequence comprises a nucleotide sequence selected from the group consisting of SEQ ID NOs: 375-411. In embodiment 143 provided herein is the method of any one of embodiments 119-129 and 142, wherein the genomic sequence at the CD38 gene locus is edited in at least 1.5% of the human cells. In embodiment 144 provided herein is the method of any one of embodiments 116-129, wherein the target gene is human APLNR gene, and wherein the spacer sequence comprises a nucleotide sequence selected from the group consisting of SEQ ID NOs: 412-421. In embodiment 145 provided herein is the method of any one of embodiments 119-129 and 144, wherein the genomic sequence at the APLNR gene locus is edited in at least 1.5% of the human cells. In embodiment 146 provided herein is the method of any one of embodiments 116-129, wherein the target gene is human BBS1 gene, and wherein the spacer sequence comprises a nucleotide sequence selected from the group consisting of SEQ ID NOs: 422-431. In embodiment 147 provided herein is the method of any one of embodiments 119-129 and 146, wherein the genomic sequence at the BBS1 gene locus is edited in at least 1.5% of the human cells. In embodiment 148 provided herein is the method of any one of embodiments 116-129, wherein the target gene is human CALR gene, and wherein the spacer sequence comprises a nucleotide sequence selected from the group consisting of SEQ ID NOs: 432-441. In embodiment 149 provided herein is the method of any one of embodiments 119-129 and 148, wherein the genomic sequence at the CD247 gene locus is edited in at least 1.5% of the human cells. In embodiment 150 provided herein is the method of any one of embodiments 116-129, wherein the target gene is human CALR gene, and wherein the spacer sequence comprises a nucleotide sequence selected from the group consisting of SEQ ID NOs: 442-451. In embodiment 151 provided herein is the method of any one of embodiments 119-129 and 150, wherein the genomic sequence at the CD247 gene locus is edited in at least 1.5% of the human cells. In embodiment 152 provided herein is the method of any one of embodiments 116-129, wherein the target gene is human CD3G gene, and wherein the spacer sequence comprises a nucleotide sequence selected from the group consisting of SEQ ID NOs: 452-461. In embodiment 153 provided herein is the method of any one of embodiments 119-129 and 152, wherein the genomic sequence at the CD3G gene locus is edited in at least 1.5% of the human cells. In embodiment 154 provided herein is the method of any one of embodiments 116-129, wherein the target gene is human CD52 gene, and wherein the spacer sequence comprises a nucleotide sequence selected from the group consisting of SEQ ID NOs: 462-465. In embodiment 155 provided herein is the method of any one of embodiments 119-129 and 154, wherein the genomic sequence at the CD52 gene locus is edited in at least 1.5% of the human cells. In embodiment 156 provided herein is the method of any one of embodiments 116-129, wherein the target gene is human CD58 gene, and wherein the spacer sequence comprises a nucleotide sequence selected from the group consisting of SEQ ID NOs: 466-475. In embodiment 157 provided herein is the method of any one of embodiments 119-129 and 156, wherein the genomic sequence at the CD58 gene locus is edited in at least 1.5% of the human cells. In embodiment 158 provided herein is the method of any one of embodiments 116-129, wherein the target gene is human COL17A1 gene, and wherein the spacer sequence comprises a nucleotide sequence selected from the group consisting of SEQ ID NOs: 476-485. In embodiment 159 provided herein is the method of any one of embodiments 119-129 and 158, wherein the genomic sequence at the COL17A1 gene locus is edited in at least 1.5% of the human cells. In embodiment 160 provided herein is the method of any one of embodiments 116-129, wherein the target gene is human DEFB134 gene, and wherein the spacer sequence comprises a nucleotide sequence selected from the group consisting of SEQ ID NOs: 486-495. In embodiment 161 provided herein is the method of any one of embodiments 119-129 and 160, wherein the genomic sequence at the DEFB134 gene locus is edited in at least 1.5% of the human cells. In embodiment 162 provided herein is the method of any one of embodiments 116-129, wherein the target gene is human ERAP1 gene, and wherein the spacer sequence comprises a nucleotide sequence selected from the group consisting of SEQ ID NOs: 496-505. In embodiment 163 provided herein is the method of any one of embodiments 119-129 and 162, wherein the genomic sequence at the ERAP1 gene locus is edited in at least 1.5% of the human cells. In embodiment 164 provided herein is the method of any one of embodiments 116-129, wherein the target gene is human ERAP2 gene, and wherein the spacer sequence comprises a nucleotide sequence selected from the group consisting of SEQ ID NOs: 506-515. In embodiment 165 provided herein is the method of any one of embodiments 119-129 and 164, wherein the genomic sequence at the ERAP2 gene locus is edited in at least 1.5% of the human cells. In embodiment 166 provided herein is the method of any one of embodiments 116-129, wherein the target gene is human IFNGR1 gene, and wherein the spacer sequence comprises a nucleotide sequence selected from the group consisting of SEQ ID NOs: 516-525. In embodiment 167 provided herein is the method of any one of embodiments 119-129 and 166, wherein the genomic sequence at the IFNGR1 gene locus is edited in at least 1.5% of the human cells. In embodiment 168 provided herein is the method of any one of embodiments 116-129, wherein the target gene is human IFNGR2 gene, and wherein the spacer sequence comprises a nucleotide sequence selected from the group consisting of SEQ ID NOs: 526-535. In embodiment 169 provided herein is the method of any one of embodiments 119-129 and 168, wherein the genomic sequence at the IFNGR2 gene locus is edited in at least 1.5% of the human cells. In embodiment 170 provided herein is the method of any one of embodiments 116-129, wherein the target gene is human JAK1 gene, and wherein the spacer sequence comprises a nucleotide sequence selected from the group consisting of SEQ ID NOs: 536-545. In embodiment 171 provided herein is the method of any one of embodiments 119-129 and 170, wherein the genomic sequence at the JAK1 gene locus is edited in at least 1.5% of the human cells. In embodiment 172 provided herein is the method of any one of embodiments 116-129, wherein the target gene is human JAK2 gene, and wherein the spacer sequence comprises a nucleotide sequence selected from the group consisting of SEQ ID NOs: 546-555. In embodiment 173 provided herein is the method of any one of embodiments 119-129 and 172, wherein the genomic sequence at the JAK2 gene locus is edited in at least 1.5% of the human cells. In embodiment 174 provided herein is the method of any one of embodiments 116-129, wherein the target gene is human mir-101-2 gene, and wherein the spacer sequence comprises a nucleotide sequence selected from the group consisting of SEQ ID NOs: 556-558. In embodiment 175 provided herein is the method of any one of embodiments 119-129 and 174, wherein the genomic sequence at the mir-101-2 gene locus is edited in at least 1.5% of the human cells. In embodiment 176 provided herein is the method of any one of embodiments 116-129, wherein the target gene is human MLANA gene, and wherein the spacer sequence comprises a nucleotide sequence selected from the group consisting of SEQ ID NOs: 559-568. In embodiment 177 provided herein is the method of any one of embodiments 119-129 and 176, wherein the genomic sequence at the PSMB5 gene locus is edited in at least 1.5% of the human cells. In embodiment 178 provided herein is the method of any one of embodiments 116-129, wherein the target gene is human PSMB5 gene, and wherein the spacer sequence comprises a nucleotide sequence selected from the group consisting of SEQ ID NOs: 569-578. In embodiment 179 provided herein is the method of any one of embodiments 119-129 and 178, wherein the genomic sequence at the PSMB5 gene locus is edited in at least 1.5% of the human cells. In embodiment 180 provided herein is the method of any one of embodiments 116-129, wherein the target gene is human PSMB8 gene, and wherein the spacer sequence comprises a nucleotide sequence selected from the group consisting of SEQ ID NOs: 579-588. In embodiment 181 provided herein is the method of any one of embodiments 119-129 and 180, wherein the genomic sequence at the PSMB8 gene locus is edited in at least 1.5% of the human cells. In embodiment 182 provided herein is the method of any one of embodiments 116-129, wherein the target gene is human PSMB9 gene, and wherein the spacer sequence comprises a nucleotide sequence selected from the group consisting of SEQ ID NOs: 589-598. In embodiment 183 provided herein is the method of any one of embodiments 119-129 and 182, wherein the genomic sequence at the PSMB9 gene locus is edited in at least 1.5% of the human cells. In embodiment 184 provided herein is the method of any one of embodiments 116-129, wherein the target gene is human PTCD2 gene, and wherein the spacer sequence comprises a nucleotide sequence selected from the group consisting of SEQ ID NOs: 599-608. In embodiment 185 provided herein is the method of any one of embodiments 119-129 and 184, wherein the genomic sequence at the PTCD2 gene locus is edited in at least 1.5% of the human cells. In embodiment 186 provided herein is the method of any one of embodiments 116-129, wherein the target gene is human RFX5 gene, and wherein the spacer sequence comprises a nucleotide sequence selected from the group consisting of SEQ ID NOs: 609-618. In embodiment 187 provided herein is the method of any one of embodiments 119-129 and 186, wherein the genomic sequence at the RFX5 gene locus is edited in at least 1.5% of the human cells. In embodiment 188 provided herein is the method of any one of embodiments 116-129, wherein the target gene is human RFXANK gene, and wherein the spacer sequence comprises a nucleotide sequence selected from the group consisting of SEQ ID NOs: 619-628. In embodiment 189 provided herein is the method of any one of embodiments 119-129 and 188, wherein the genomic sequence at the RFXANK gene locus is edited in at least 1.5% of the human cells. In embodiment 190 provided herein is the method of any one of embodiments 116-129, wherein the target gene is human RFXAP gene, and wherein the spacer sequence comprises a nucleotide sequence selected from the group consisting of SEQ ID NOs: 629-638. In embodiment 191 provided herein is the method of any one of embodiments 119-129 and 190, wherein the genomic sequence at the RFXAP gene locus is edited in at least 1.5% of the human cells. In embodiment 192 provided herein is the method of any one of embodiments 116-129, wherein the target gene is human RPL23 gene, and wherein the spacer sequence comprises a nucleotide sequence selected from the group consisting of SEQ ID NOs: 639-648. In embodiment 193 provided herein is the method of any one of embodiments 119-129 and 192, wherein the genomic sequence at the RPL23 gene locus is edited in at least 1.5% of the human cells. In embodiment 194 provided herein is the method of any one of embodiments 116-129, wherein the target gene is human SOX10 gene, and wherein the spacer sequence comprises a nucleotide sequence selected from the group consisting of SEQ ID NOs: 649-654. In embodiment 195 provided herein is the method of any one of embodiments 119-129 and 194, wherein the genomic sequence at the SOX10 gene locus is edited in at least 1.5% of the human cells. In embodiment 196 provided herein is the method of any one of embodiments 116-129, wherein the target gene is human SRP54 gene, and wherein the spacer sequence comprises a nucleotide sequence selected from the group consisting of SEQ ID NOs: 655-665. In embodiment 197 provided herein is the method of any one of embodiments 119-129 and 196, wherein the genomic sequence at the SRP54 gene locus is edited in at least 1.5% of the human cells. In embodiment 198 provided herein is the method of any one of embodiments 116-129, wherein the target gene is human STAT1 gene, and wherein the spacer sequence comprises a nucleotide sequence selected from the group consisting of SEQ ID NOs: 666-675. In embodiment 199 provided herein is the method of any one of embodiments 119-129 and 198, wherein the genomic sequence at the STAT1 gene locus is edited in at least 1.5% of the human cells. In embodiment 200 provided herein is the method of any one of embodiments 116-129, wherein the target gene is human Tap1 gene, and wherein the spacer sequence comprises a nucleotide sequence selected from the group consisting of SEQ ID NOs: 676-685. In embodiment 201 provided herein is the method of any one of embodiments 119-129 and 200, wherein the genomic sequence at the Tap1 gene locus is edited in at least 1.5% of the human cells. In embodiment 202 provided herein is the method of any one of embodiments 116-129, wherein the target gene is human TAP2 gene, and wherein the spacer sequence comprises a nucleotide sequence selected from the group consisting of SEQ ID NOs: 686-695. In embodiment 203 provided herein is the method of any one of embodiments 119-129 and 202, wherein the genomic sequence at the TAP2 gene locus is edited in at least 1.5% of the human cells. In embodiment 204 provided herein is the method of any one of embodiments 116-129, wherein the target gene is human TAPBP gene, and wherein the spacer sequence comprises a nucleotide sequence selected from the group consisting of SEQ ID NOs: 696-705. In embodiment 205 provided herein is the method of any one of embodiments 119-129 and 204, wherein the genomic sequence at the TAPBP gene locus is edited in at least 1.5% of the human cells. In embodiment 206 provided herein is the method of any one of embodiments 116-129, wherein the target gene is human TWF1 gene, and wherein the spacer sequence comprises a nucleotide sequence selected from the group consisting of SEQ ID NOs: 706-715. In embodiment 207 provided herein is the method of any one of embodiments 119-129 and 206, wherein the genomic sequence at the TWF1 gene locus is edited in at least 1.5% of the human cells. In embodiment 208 provided herein is the method of any one of embodiments 116-129, wherein the target gene is human CD3D gene, and wherein the spacer sequence comprises a nucleotide sequence selected from the group consisting of SEQ ID NOs: 716-725. In embodiment 209 provided herein is the method of any one of embodiments 119-129 and 208, wherein the genomic sequence at the CD3D gene locus is edited in at least 1.5% of the human cells. In embodiment 210 provided herein is the method of any one of embodiments 116-129, wherein the target gene is human NLRC2 gene, and wherein the spacer sequence comprises a nucleotide sequence selected from the group consisting of SEQ ID NOs: 726-744. In embodiment 211 provided herein is the method of any one of embodiments 119-129 and 210, wherein the genomic sequence at the NLRC2 gene locus is edited in at least 1.5% of the human cells. In embodiment 212 provided herein is the method of any one of embodiments 119-211, wherein genomic mutations are detected in no more than 2% of the cells at any off-target loci by CIRCLE-Seq. In embodiment 213 provided herein is the method of any one of embodiments 119-211, wherein genomic mutations are detected in no more than 1% of the cells at any off-target loci by CIRCLE-Seq.

### VII. EXAMPLES

The following Examples are merely illustrative and are not intended to limit the scope or content of the invention in any way.

### Example 1. Cleavage of Genomic DNA by Single Guide MAD7 CRISPR-Cas Systems

MAD7 is a type V-A Cas protein that has endonuclease activity when complexed with a single guide RNA, also known as a crRNA in a type V-A system (see, U.S. Pat. No. 9,982,279). This example describes cleavage of the genomic DNA of Jurkat cells using MAD7 in complex with single guide nucleic acids targeting human CSF2, CD40LG, TRBC1, TRBC2, TRBC1_2, CD3E, CD38, DHODH, MVD, PLK1, TUBB, or U6 gene.

Briefly, Jurkat cells were grown in RPMI 1640 medium (Thermo Fisher Scientific, A1049101) supplemented with 10% fetus bovine serum at 37° C. in a 5% CO₂ environment, and split every 2-3 days to a density of 100,000 cells/mL. MAD7 protein, which contained a nucleoplasmin NLS at the C-terminus, was expressed in E. coli and purified by fast protein liquid chromatography (FPLC). RNP complexes were prepared by incubating 100 pmol MAD7 protein with 100 pmol chemically synthesized single guide RNA for 10 minutes at room temperature. The RNPs were mixed with 200,000 Jurkat cells in a final volume of 25 μL. Electroporation was carried out on a 4D-Nucleofector (Lonza) using program CA-137. Following electroporation, the cells were cultured for three days.

Genomic DNA of the cells was extracted using the Quick Extract DNA extraction solution 1.0 (Epicentre). The genes were amplified from the genomic DNA samples in a PCR reaction with primers with or without overhang adaptors and processed using the Nextera XT Index Kit v2 Set A (Illumina, FC-131-2001) or the KAPA HyperPlus kit (Roche, cat. no. KK8514), respectively. The final PCR products were analyzed by next-generation sequencing, and the data were analyzed with the AmpliCan package (see, Labun et al. (2019), Accurate analysis of genuine CRISPR editing events with ampliCan, Genome Res., electronically published in advance). Editing efficiency was determined by the number of edited reads relative to the total number of reads obtained under each condition.

The nucleotide sequence of each single guide RNA used in this example consisted of, from 5′ to 3′, UAAUUUCUACUCUUGUAGAU (SEQ ID NO: 50) and a spacer sequence. In SEQ ID NO: 50, the modulator stem sequence (UCUAC) and the targeter stem sequence (GUAGA) are underlined. The editing efficiency of each single guide RNA was measured as the percentage of cells having one or more insertion or deletion at the target site (% indel). The spacer sequences tested for targeting human CSF2, CD40LG, TRBC1, TRBC2, TRBC1_2, CD3E, CD38, DHODH, MVD, PLK1, TUBB, or U6 gene and the editing efficiency of each single guide RNA are shown in Tables 14-20.

TABLE 14
Selected Spacer Sequences
Targeting Human CSF2 Genes

| | | SEQ | 8 | % | INDEL |
|---|---|---|---|---|---|
| | Spacer | ID | INDEL | INDEL | 1 |
| crRNA | Sequence | NO | control | rep1 | rep2 |
| gCSF2_ | TGAGATGACT | 201 | 0.005 | 1.5 | 0.16 |
| 001 | TCTACTGTTT | | | | |
| | C | | | | |
| gCSF2_ | CCTTTTCTAC | 202 | 0.006 | 0.0077 | 0.038 |
| 002 | AGAATGAAAC | | | | |
| | A | | | | |
| gCSF2_ | CTTTTCTACA | 203 | 0.003 | 22.4 | 6 |
| 003 | GAATGAAACA | | | | |
| | G | | | | |
| gCSF2_ | CTACAGAATG | 204 | 0.003 | 0.019 | 0.018 |
| 004 | AAACAGTAGA | | | | |
| | A | | | | |
| gCSF2_ | TACAGAATGA | 205 | 0.003 | 29 | 26 |
| 005 | AACAGTAGAA | | | | |
| | G | | | | |
| gCSF2_ | CCACAGGAGC | 206 | 0.007 | 2.4 | 0.021 |
| 006 | CGACCTGCCT | | | | |
| | A | | | | |
| gCSF2_ | CACAGGAGCC | 207 | 0.007 | 27 | 34.7 |
| 007 | GACCTGCCTA | | | | |
| | C | | | | |
| gCSF2_ | ttatttttct | 208 | 0.91 | 0.12 | 0.78 |
| 008 | ttttttAAAG | | | | |
| | G | | | | |
| gCSF2_ | tatttttctt | 209 | 0.91 | 0.14 | 0.10 |
| 009 | tttttAAAGG | | | | |
| | A | | | | |
| gCSF2_ | atttttcttt | 210 | 0.91 | 0.15 | 0.15 |
| 010 | ttttAAAGGA | | | | |
| | A | | | | |
| gCSF2_ | tttttctttt | 211 | 0.91 | 0 | 0.16 |
| 011 | tttAAAGGAA | | | | |
| | A | | | | |
| gCSF2_ | tctttttttA | 212 | 0.024 | 0.046 | 0.051 |
| 012 | AAGGAAACTT | | | | |
| | C | | | | |
| gCSF2_ | ctttttttAA | 213 | 0.022 | 0.038 | 0.035 |
| 013 | AGGAAACTTC | | | | |
| | C | | | | |
| gCSF2_ | tttttttAAA | 214 | 0.011 | 0.011 | 0.016 |
| 014 | GGAAACTTCC | | | | |
| | T | | | | |
| gCSF2_ | tttAAAGGAA | 215 | 0.004 | 0.035 | 0.005 |
| 015 | ACTTCCTGTG | | | | |
| | C | | | | |
| gCSF2_ | ttAAAGGAAA | 216 | 0.004 | 0.28 | 0.005 |
| 016 | CTTCCTGTGC | | | | |
| | A | | | | |
| gCSF2_ | tAAAGGAAAC | 217 | 0.004 | 0.019 | 0.88 |
| 017 | TTCCTGTGCA | | | | |
| | A | | | | |
| gCSF2_ | AAAGGTGATA | 218 | 0.01 | 0.01 | 0.01 |
| 018 | ATCTGGGTTG | | | | |
| | C | | | | |
| gCSF2_ | AAAGGAAACT | 219 | 0.004 | 0.0078 | 0.01 |
| 019 | TCCTGTGCAA | | | | |
| | C | | | | |
| gCSF2_ | AAGGAAACTT | 220 | 0.003 | 7 | 6.6 |
| 020 | CCTGTGCAAC | | | | |
| | C | | | | |
| gCSF2_ | AAACTTTCAA | 221 | 0.008 | 0.007 | 0.014 |
| 021 | AGGTGATAAT | | | | |
| | C | | | | |
| gCSF2_ | AAAGTTTCAA | 222 | 0.017 | 0.016 | 0.029 |
| 022 | AGAGAACCTG | | | | |
| | A | | | | |
| gCSF2_ | AAAGAGAACC | 223 | 0.006 | 0.007 | 3.5 |
| 023 | TGAAGGACTT | | | | |
| | T | | | | |
| gCSF2_ | TGCTTGTCAT | 224 | 0.029 | 7.9 | 9.4 |
| 024 | CCCCTTTGAC | | | | |
| | T | | | | |
| gCSF2_ | ACTGCTGGGA | 225 | 0.005 | 0.099 | 1.5 |
| 025 | GCCAGTCCAG | | | | |
| | G | | | | |

TABLE 15
Selected Spacer Sequences
Targeting Human CD40LG Genes

| | | SEQ | % | % | 8 |
|---|---|---|---|---|---|
| | Spacer | ID | INDEL_ | INDEL_ | INDEL_ |
| crRNA | Sequence | NO | control | rep1 | rep2 |
| gCD40LG_ | GTTGTATGTT | 254 | 0.009 | 20.6 | 9.7 |
| 001 | TCGATCATGC | | | | |
| | T | | | | |
| gCD40LG_ | AACTTTAACA | 255 | 0.01 | 0.004 | 3.3 |
| 002 | CAGCATGATC | | | | |
| | G | | | | |
| gCD40LG_ | ACACAGCATG | 256 | 0.017 | 1.06 | 1.5 |
| 003 | ATCGAAACAT | | | | |
| | A | | | | |
| gCD40LG_ | ATGCTGATGG | 257 | 0.012 | 6.6 | 10.9 |
| 004 | GCAGTCCAGT | | | | |
| | G | | | | |
| gCD40LG_ | CATGCTGATG | 258 | 0.012 | 0.007 | 0.45 |
| 005 | GGCAGTCCAG | | | | |
| | T | | | | |
| gCD40LG_ | TATGTATTTA | 259 | 0.045 | 0.06 | 0.05 |
| 006 | CTTACTGTTT | | | | |
| | T | | | | |
| gCD40LG_ | ATGTATTTAC | 260 | 0.045 | 0.05 | 0.05 |
| 007 | TTACTGTTTT | | | | |
| | T | | | | |
| gCD40LG_ | TGTATTTACT | 261 | 0.049 | 0.059 | 0.02 |
| 008 | TACTGTTTTT | | | | |
| | C | | | | |
| gCD40LG_ | CTTACTGTTT | 262 | 0.05 | 0.029 | 0.02 |
| 009 | TTCTTATCAC | | | | |
| | C | | | | |
| gCD40LG_ | TCTTATCACC | 263 | 0.025 | 0.029 | 0.06 |
| 010 | CAGATGATTG | | | | |
| | G | | | | |
| gCD40LG_ | CTTATCACCC | 264 | 0.099 | 0.034 | 0.14 |
| 011 | AGATGATTGG | | | | |
| | G | | | | |
| gCD40LG_ | TTATCACCCA | 265 | 0.10 | 0.37 | 0.11 |
| 012 | GATGATTGGG | | | | |
| | T | | | | |
| gCD40LG_ | TGCTGTGTAT | 266 | 0.02 | 0.019 | 0.014 |
| 013 | CTTCATAGAA | | | | |
| | G | | | | |
| gCD40LG_ | GCTGTGTATC | 267 | 0.02 | 4.6 | 4 |
| 014 | TTCATAGAAG | | | | |
| | G | | | | |
| gCD40LG_ | CTGTGTATCT | 268 | 0.017 | 9.2 | 12.45 |
| 015 | TCATAGAAGG | | | | |
| | T | | | | |
| gCD40LG_ | ATGAATACAA | 269 | 0.019 | 0.004 | 0.018 |
| 016 | AATCTTCATG | | | | |
| | A | | | | |
| gCD40LG_ | CATGAATACA | 270 | 0.021 | 0.009 | 0.005 |
| 017 | AAATCTTCAT | | | | |
| | G | | | | |
| gCD40LG_ | TCCTGTGTTG | 271 | 0.009 | 1.19 | 0.07 |
| 018 | CATCTCTGTA | | | | |
| | T | | | | |
| gCD40LG_ | GTATTCATGA | 272 | 0.023 | 7 | 2 |
| 019 | AAACGATACA | | | | |
| | G | | | | |
| gCD40LG_ | TATTCATGAA | 273 | 0.023 | 1.5 | 1.4 |
| 020 | AACGATACAG | | | | |
| | A | | | | |
| gCD40LG_ | ATCTCCTCAC | 274 | 0.035 | 65 | 63.5 |
| 021 | AGTTCAGTAA | | | | |
| | G | | | | |
| gCD40LG_ | AATCTCCTCA | 275 | 0.035 | 0.26 | 0.29 |
| 022 | CAGTTCAGTA | | | | |
| | A | | | | |
| gCD40LG_ | CCAGTAATTA | 276 | 0.021 | 93 | 74.9 |
| 023 | AGCTGCTTAC | | | | |
| | C | | | | |
| gCD40LG_ | ACCAGTAATT | 277 | 0.023 | 0.53 | 0.019 |
| _ | AAGCTGCTTA | | | | |
| 024 | C | | | | |
| gCD40LG_ | AAGGCTTTGT | 278 | 0.033 | 9.7 | 13 |
| 025 | GAAGGTAAGC | | | | |
| | A | | | | |
| gCD40LG_ | TTCGTCTCCT | 279 | 0.019 | 0.028 | 0.04 |
| 026 | CTTTGTTTAA | | | | |
| | C | | | | |
| gCD40LG_ | TTTCTTCGTC | 280 | 0.026 | 0.013 | 0.25 |
| 027 | TCCTCTTTGT | | | | |
| | T | | | | |
| gCD40LG_ | CTTTCTTCGT | 281 | 0.028 | 0.033 | 0.045 |
| 028 | CTCCTCTTTG | | | | |
| | T | | | | |
| gCD40LG_ | AGGATATAAT | 282 | 0.034 | 1.14 | 0.57 |
| 029 | GTTAAACAAA | | | | |
| | G | | | | |
| gCD40LG_ | GGATATAATG | 283 | 0.034 | 63.5 | 59.9 |
| 030 | TTAAACAAAG | | | | |
| | A | | | | |
| gCD40LG_ | AAAGCTGTTT | 284 | 0.028 | 0.115 | 0.023 |
| 031 | TCTTTCTTCG | | | | |
| | T | | | | |
| gCD40LG_ | CATTTCAAAG | 285 | 0.016 | 0.17 | 0.020 |
| 032 | CTGTTTTCTT | | | | |
| | T | | | | |
| gCD40LG_ | GCATTTCAAA | 286 | 0.016 | 0.015 | 0.021 |
| 033 | GCTGTTTTCT | | | | |
| | T | | | | |
| gCD40LG_ | TGCATTTCAA | 287 | 0.016 | 0.006 | 0.016 |
| 034 | AGCTGTTTTC | | | | |
| | T | | | | |
| gCD40LG_ | AGGATTCTGA | 288 | 0.119 | 80.7 | 59 |
| 035 | TCACCTGAAA | | | | |
| | T | | | | |
| gCD40LG_ | TGGTTCCATT | 289 | 0.078 | 0.25 | 1.3 |
| 036 | TCAGGTGATC | | | | |
| | A | | | | |
| gCD40LG_ | GGTTCCATTT | 290 | 0.073 | 0.13 | 0.33 |
| 037 | CAGGTGATCA | | | | |
| | G | | | | |
| gCD40LG_ | GTTCCATTTC | 291 | 0.073 | 0.017 | 4.9 |
| 038 | AGGTGATCAG | | | | |
| | A | | | | |
| gCD40LG_ | AGGTGATCAG | 292 | 0.021 | 0.009 | 0.009 |
| 039 | AATCCTCAAA | | | | |
| | T | | | | |
| gCD40LG_ | CTGCTGGCCT | 293 | 0.011 | 90.7 | 87 |
| 040 | CACTTATGAC | | | | |
| | A | | | | |
| gCD40LG_ | AGCCCACTGT | 294 | 0.053 | 86.8 | 91.8 |
| 041 | AACACTGTTA | | | | |
| | C | | | | |
| gCD40LG_ | CAGCCCACTG | 295 | 0.053 | 3.7 | 9.1 |
| 042 | TAACACTGTT | | | | |
| | A | | | | |
| gCD40LG_ | TCAGCCCACT | 296 | 0.049 | 17.7 | 5.5 |
| 043 | GTAACACTGT | | | | |
| | T | | | | |
| gCD40LG_ | CCTTTCTTTG | 297 | 0.022 | 22 | 15 |
| 044 | TAACAGTGTT | | | | |
| | A | | | | |
| gCD40LG_ | TTTGTAACAG | 298 | 0.25 | 20 | 14.9 |
| 045 | TGTTACAGTG | | | | |
| | G | | | | |
| gCD40LG_ | TAACAGTGTT | 299 | 0.24 | 37.6 | 42.5 |
| 046 | ACAGTGGGCT | | | | |
| | G | | | | |
| gCD40LG_ | CAGGGTTACC | 300 | 0.013 | 0.23 | 0 |
| 047 | AAGTTGTTGC | | | | |
| | T | | | | |
| gCD40LG_ | CCAGGGTTAC | 301 | 0.008 | 2 | 1.07 |
| 048 | CAAGTTGTTG | | | | |
| | C | | | | |
| gCD40LG_ | CCATTTTCCA | 302 | 0.017 | 24 | 0 |
| 049 | GGGTTACCAA | | | | |
| | G | | | | |
| gCD40LG_ | ACGGTCAGCT | 303 | 0.101 | 5.3 | 0 |
| 050 | GTTTCCCATT | | | | |
| | T | | | | |
| gCD40LG_ | AACGGTCAGC | 304 | 0.101 | 0 | 0 |
| 051 | TGTTTCCCAT | | | | |
| | T | | | | |
| gCD40LG_ | GGCAGAGGCT | 305 | 0.062 | 78.4 | 85 |
| 052 | GGCTATAAAT | | | | |
| | G | | | | |
| gCD40LG_ | TAGCCAGCCT | 306 | 0.090 | 73.6 | 86.6 |
| 053 | CTGCCTAAAG | | | | |
| | T | | | | |
| gCD40LG_ | CAGCTCTGAG | 307 | 0.017 | 4 | 28.6 |
| 054 | TAAGATTCTC | | | | |
| | T | | | | |
| gCD40LG_ | GCGGAACTGT | 308 | 0.015 | 23 | 16.9 |
| 055 | GGGTATTTGC | | | | |
| | A | | | | |
| gCD40LG_ | AATTGCAACC | 309 | 0.020 | 0 | 0.005 |
| 056 | AGGTGCTTCG | | | | |
| | G | | | | |
| gCD40LG_ | TCAATGTGAC | 310 | 0.005 | 9 | 5.9 |
| 057 | TGATCCAAGC | | | | |
| | C | | | | |
| gCD40LG_ | AGTAAGCCAA | 311 | 0.002 | 73 | 70.9 |
| 058 | AGGACGTGAA | | | | |
| | G | | | | |
| gCD40LG_ | GCTTACTCAA | 312 | 0.017 | 2 | 2 |
| 059 | ACTCTGAACA | | | | |
| | G | | | | |

TABLE 16
Selected Spacer Sequences
Targeting Human TRBC1 Genes

| | | SEQ | % | % | % |
|---|---|---|---|---|---|
| | Spacer | ID | INDEL_ | INDEL_ | INDEL_ |
| crRNA | Sequence | NO | control | rep1 | rep2 |
| gTRBC1_ | CAGAGGACCTG | 314 | 0.022 | 1.1 | 0.87 |
| 001 | AACAAGGTGT | | | | |
| gTRBC1_ | CCTCTCCCTGC | 315 | 0.014 | 0.36 | 0.019 |
| 002 | TTTCTTTCAG | | | | |
| gTRBC1_ | CTCTCCCTGCT | 316 | 0.014 | 4 | 2 |
| 003 | TTCTTTCAGA | | | | |
| gTRBC1_ | TTTCAGACTGT | 317 | 0.034 | 1 | 0.31 |
| 004 | GGCTTTACCT | | | | |
| gTRBC1_ | AGACTGTGGCT | 318 | 0.029 | 93.6 | 27.6 |
| 005 | TTACCTCGGG | | | | |
| gTRBC1_ | TCTTCTGCAGG | 319 | 0.028 | 19 | 13 |
| 006 | TCAAGAGAAA | | | | |

TABLE 17
Selected Spacer Sequences Targeting
Human TRBC2 Genes

| | | SEQ | % | % | % |
|---|---|---|---|---|---|
| | Spacer | ID | INDEL_ | INDEL_ | INDEL_ |
| crRNA | Sequence | NO | control | rep1 | rep2 |
| gTRBC2_ | CAGAGGACCTG | 320 | 0.058 | 0.053 | 0.026 |
| 001 | AAAAACGTGT | | | | |
| gTRBC2_ | TCTTCCCCTGT | 321 | 0.019 | 0.022 | 0.021 |
| 002 | TTTCTTTCAG | | | | |
| gTRBC2_ | CTTCCCCTGTT | 322 | 0.021 | 0.021 | 0.018 |
| 003 | TTCTTTCAGA | | | | |
| gTRBC2_ | TTCCCCTGTTT | 323 | 0.021 | 7.5 | 8 |
| 004 | TCTTTCAGAC | | | | |
| gTRBC2_ | CTTTCAGACTG | 324 | 0.028 | 0.045 | 0.038 |
| 005 | TGGCTTCACC | | | | |
| gTRBC2_ | TTTCAGACTGT | 325 | 0.025 | 0.48 | 0.72 |
| 006 | GGCTTCACCT | | | | |
| gTRBC2_ | AGACTGTGGCT | 326 | 0.023 | 29 | 18.6 |
| 007 | TCACCTCCGG | | | | |
| gTRBC2_ | GAGCTAGCCTC | 327 | 0.016 | 17 | 4.5 |
| 008 | TGGAATCCTT | | | | |
| gTRBC2_ | GGAGCTAGCCT | 328 | 0.019 | 67 | 53.7 |
| 009 | CTGGAATCCT | | | | |

TABLE 18
Selected Spacer Sequences
Targeting Human TRBC1_2 Genes

| | | SEQ | % | % | % |
|---|---|---|---|---|---|
| | Spacer | ID | INDEL_ | INDEL_ | INDEL_ |
| crRNA | Sequence | NO | control | rep1 | rep2 |
| gTRBC1_ | GGTGTGGGAGA | 329 | 0.0053 | 93.5 | 58 |
| 2_001 | TCTCTGCTTC | | | | |
| gTRBC1_ | GGTGTGGGAGA | 329 | 0.0063 | 88.6 | 87 |
| 2_001 | TCTCTGCTTC | | | | |
| gTRBC1_ | GGGTGTGGGAG | 330 | 0.0053 | 9.8 | 3.5 |
| 2_002 | ATCTCTGCTT | | | | |
| gTRBC1_ | GGGTGTGGGAG | 330 | 0.0063 | 14 | 6 |
| 2_002 | ATCTCTGCTT | | | | |
| gTRBC1_ | AGCCATCAGAA | 331 | 0.019 | 71.8 | 72 |
| 2_003 | GCAGAGATCT | | | | |
| gTRBC1_ | AGCCATCAGAA | 331 | 0.023 | 66 | 60 |
| 2_003 | GCAGAGATCT | | | | |

TABLE 19
Selected Spacer Sequences Targeting Human CD3E Genes

| | | SEQ | % | % | % |
|---|---|---|---|---|---|
| | | ID | INDEL_ | INDEL_ | INDEL_ |
| crRNA | Spacer Sequence | NO | control | rep1 | rep2 |
| gCD3E_1 | CACTCCATCCTACTCACCIGA | 333 | 0.012 | 26.9 | 26.8 |
| gCD3E_2 | tttttCTTATTTATTTTCTAG | 334 | 0.022 | 0.028 | 0.035 |
| gCD3E_3 | ttttCTTATTTATTTTCTAGT | 335 | 0.022 | 0.018 | 0.02 |
| gCD3E_4 | tttCTTATTTATTTTCTAGTT | 336 | 0.016 | 0.01 | 0.016 |
| gCD3E_5 | ttCTTATTTATTTTCTAGTTG | 337 | 0.016 | 0.007 | 0.02 |
| gCD3E_6 | tCTTATTTATTTTCTAGTTGG | 338 | 0.016 | 0.015 | 0.019 |
| gCD3E_7 | CTTATTTATTTTCTAGTTGGC | 339 | 0.088 | 0.058 | 0.037 |
| gCD3E_8 | TTATTTATTTTCTAGTTGGCG | 340 | 0.088 | 0.088 | 0.061 |
| gCD3E_9 | TTTTCTAGTTGGCGTTTGGGG | 341 | 0.084 | 0.086 | 0.049 |
| gCD3E_ | CTAGTTGGCGTTTGGGGGCAA | 342 | 0.081 | 0.51 | 0.29 |
| 10 | | | | | |
| gCD3E_ | TAGTTGGCGTTTGGGGGCAAG | 343 | 0.081 | 5.96 | 1.97 |
| 11 | | | | | |
| gCD3E_ | CTTTTCAGGTAATGAAGAAAT | 344 | 0.041 | 38.5 | 31.9 |
| 12 | | | | | |
| gCD3E_ | CAGGTAATGAAGAAATGGGTA | 345 | 0.042 | 1.5 | 1.66 |
| 13 | | | | | |
| gCD3E_ | AGGTAATGAAGAAATGGGTAA | 346 | 0.042 | 68 | 75 |
| 14 | | | | | |
| gCD3E_ | CTTTTTTCATTTTCAGGTGGT | 347 | 0.059 | 0.17 | 0.15 |
| 15 | | | | | |
| gCD3E_ | TTCATTTTCAGGTGGTATTAC | 348 | 0.019 | 31 | 0.05 |
| 16 | | | | | |
| gCD3E_ | TCATTTTCAGGTGGTATTACA | 349 | 0.019 | 0.031 | 0.01 |
| 17 | | | | | |
| gCD3E_ | CATTTTCAGGTGGTATTACAC | 350 | 0.015 | 0.032 | 0.66 |
| 18 | | | | | |
| gCD3E_ | ATTTTCAGGTGGTATTACACA | 351 | 0.0149 | 50.6 | 41 |
| 19 | | | | | |
| gCD3E_ | CAGGTGGTATTACACAGACAC | 352 | 0.027 | 69.5 | 43.8 |
| 20 | | | | | |
| gCD3E_ | AGGTGGTATTACACAGACACG | 353 | 0.020 | 90.5 | 87.3 |
| 21 | | | | | |
| gCD3E_ | CCTTCTTTCTCCCCAGCATAT | 354 | 0.083 | 24 | 14 |
| 22 | | | | | |
| gCD3E_ | TCCCCAGCATATAAAGTCTCC | 355 | 0.041 | 0.61 | 10 |
| 23 | | | | | |
| gCD3E_ | AGATCCAGGATACTGAGGGCA | 356 | 0.039 | 76.6 | 59 |
| 24 | | | | | |
| gCD3E_ | tcatTGTGTTGCCATAGTATT | 357 | 0.0029 | 44.8 | 43.5 |
| 25 | | | | | |
| gCD3E_ | atcatTGTGTTGCCATAGTAT | 358 | 0.0029 | 3.85 | 0.02 |
| 26 | | | | | |
| gCD3E_ | tatcatTGTGTTGCCATAGTA | 359 | 0.0059 | 0 | 0.03 |
| 27 | | | | | |
| gCD3E_ | tcatcctcatcaccgcctatg | 360 | 0.050 | 0 | 70 |
| 28 | | | | | |
| gCD3E_ | atcatcctcatcaccgcctat | 361 | 0.050 | 30 | 17.8 |
| 29 | | | | | |
| gCD3E_ | tatcatcctcatcaccgccta | 362 | 0.050 | 5 | 1.39 |
| 30 | | | | | |
| CD33E_ | CTCCAATTCTGAAAATTCCTT | 363 | 0.014 | 0 | 0.017 |
| 31 | | | | | |
| CD33E_ | CAGAATTGGAGCAAAGTGGTT | 364 | 0.021 | 0.065 | 0.20 |
| 32 | | | | | |
| CD33E_ | AGAATTGGAGCAAAGTGGTTA | 365 | 0.021 | 22.8 | 23 |
| 33 | | | | | |
| CD33E_ | CTTCCTCTGGGGTAGCAGACA | 366 | 0.020 | 99.9 | 84.6 |
| 34 | | | | | |
| CD33E_ | ATCTCTACCTGAGGGCAAGAG | 367 | 0.055 | 0.30 | 1.69 |
| 35 | | | | | |
| CD33E_ | TCTCTACCTGAGGGCAAGAGG | 368 | 0.055 | 32.9 | 36.8 |
| 36 | | | | | |
| CD33E_ | TATTCTTGCTCCAGTAGTAAA | 369 | 0.027 | 2 | 3.5 |
| 37 | | | | | |
| CD33E_ | CTACTGGAGCAAGAATAGAAA | 370 | 0.013 | 81 | 75 |
| 38 | | | | | |
| CD33E_ | CCTGCCGCCAGCACCCGCTCC | 371 | 0.008 | 32.6 | 28.9 |
| 39 | | | | | |
| gCD3E_ | CCCTCCTTCCTCCGCAGGACA | 372 | 0.031 | 77.9 | 67 |
| 40 | | | | | |
| gCD3E_ | TATCCCACGTTACCTCATAGT | 373 | 0.015 | 35.2 | 19 |
| 41 | | | | | |
| gCD3E_ | ACCCCCAGCCCATCCGGAAAG | 374 | 0.029 | 79 | 82 |
| 42 | | | | | |

TABLE 20
Tested crRNAs Targeting Certain
Other Human Genes

| | | SEQ | |
|---|---|---|---|
| | | ID | % |
| crRNA | Spacer Sequence | NO | Indel |
| gDHODH_1 | TTGCAGAAGCGGGCCCAGGAT | 770 | 0.60 |
| gDHODH_2 | TTGCAGAAGCGGGCCCAGGAT | 771 | 0.59 |
| gDHODH_3 | TATGCTGAACACCTGATGCCG | 772 | 74.94 |
| gPLK1_1 | CCAGGGTCGGCCGGTGCCCGT | 773 | 29.06 |
| gPLK1_2 | GCCGGTGGAGCCGCCGCCGGA | 774 | 2.01 |
| gPLK1_3 | TGGGCAAGGGCGGCTTTGCCA | 775 | 2.26 |
| gPLK1_4 | GGGCAAGGGCGGCTTTGCCAA | 776 | 28.24 |
| gPLK1_5 | GGCAAGGGCGGCTTTGCCAAG | 777 | 28.41 |
| gPLK1_6 | CCAAGTGCTTCGAGATCTCGG | 778 | 2.07 |
| gPLK1_7 | CATGGACATCTTCTCCCTCTG | 779 | 90.07 |
| gPLK1_8 | TCGAGGACAACGACTTCGTGT | 780 | 0.16 |
| gPLK1_9 | CGAGGACAACGACTTCGTGTT | 781 | 6.84 |
| gPLK1_10 | GAGGACAACGACTTCGTGTTC | 782 | 8.52 |
| gMVD_1 | CAGTTAAAAACCACCACAACA | 783 | 1.42 |
| gMVD_2 | GCTGAATGGCCGGGAGGAGGA | 784 | 14.06 |
| gMVD_3 | TGGAGTGGCAGATGGGAGAGC | 785 | 63.22 |
| gTUBB_1 | AACCATGAGGGAAATCGTGCA | 786 | 2.61 |
| gTUBB_2 | ACCATGAGGGAAATCGTGCAC | 787 | 68.40 |
| gTUBB_3 | TTCTCTGTAGGTGGCAAATAT | 788 | 18.67 |
| gU6_1 | GTCCTTTCCACAAGATATATA | 763 | 68.1 |
| gU6_2 | GATTTCTTGGCTTTATATATC | 764 | 0.71 |
| gU6_3 | TTGGCTTTATATATCTTGTGG | 765 | 2.83 |
| gU6_4 | GCTTTATATATCTTGTGGAAA | 766 | 0.37 |
| gU6_5 | ATATATCTTGTGGAAAGGACG | 767 | 0.39 |
| gU6_6 | TATATCTTGTGGAAAGGACGA | 768 | 0.39 |
| gU6_7 | TGGAAAGGACGAAACACCGTG | 769 | 0.24 |

### Example 2. Knock Out of Human CD38 by Single Guide MAD7 CRISPR-Cas Systems

MAD7 is a type V-A Cas protein that has endonuclease activity when complexed with a single guide RNA, also known as a crRNA in a type V-A system (see, U.S. Pat. No. 9,982,279). This example describes cleavage of the genomic DNA of primary Pan T-cells using MAD7 in complex with single guide nucleic acids targeting human CD38 gene and analysis on a genome and functional level. CD38 is a surface marker expressed on natural killer cells. Given CD38 is a target for multiple myeloma, anti-CD38 or CD38-CAR cells target CD38 expressing natural killer cells. Therefore, knockout of CD38 in natural killer cells protect them from anti-CD38 treatment.

Briefly, Pan T-cells were isolated from Leukopaks (StemCell Technology) using EasySep Direct Human T cell Isolation Kit (StemCell Technology Catalog #19661) and cryopreserved using CryoStor CS10 (StemCell Technology Catalog #07930). The cells were thawed and activated with ImmunoCult Human CD3/CD28 T Cell Activator (StemCell Technology Catalog #10991) and cultivated in ImmunoCult-XF T Cell Expansion Medium (StemCell Technology, Catalog #10981) supplemented with IL2 (StemCell Technology Catalog #78036.3) at 37° C. in a 5% CO₂ environment, and transfected after approximately 48 hours with RNPs, consisting of MAD7 protein and synthetic gRNA. MAD7 protein, which contained a nucleoplasmin NLS at the C-terminus, was expressed in E. coli and purified by fast protein liquid chromatography (FPLC). RNP complexes were prepared by incubating 100 pmol MAD7 protein with 100 pmol chemically synthesized single guide RNA for 10 minutes at room temperature. The RNPs were mixed with 1,000,000 Pan T-cells resuspended in nucleofection buffer P3 (Lonza) in a final volume of 25 μL. Electroporation was carried out on a 4D-Nucleofector (Lonza) using program EO-115. Following electroporation, the cells were cultured for 2-3 days.

Genomic DNA of the cells was extracted using the Quick Extract DNA extraction solution 1.0 (Epicentre). The genes fragments were amplified from the genomic DNA samples in a PCR reaction with primers with overhang adaptors and processed using the Nextera XT designed primers (IDT). The final PCR products were analyzed by next-generation sequencing, and the data were analyzed with the Crispresso (see, Clement et al. (2019), CRISPResso2 provides accurate and rapid genome editing sequence analysis. Nat Biotechnol. 2019 March; 37(3):224-226. doi: 10.1038/s41587-019-0032-3. PubMed PMID: 30809026). Editing efficiency was determined by the number of edited reads relative to the total number of reads obtained under each condition.

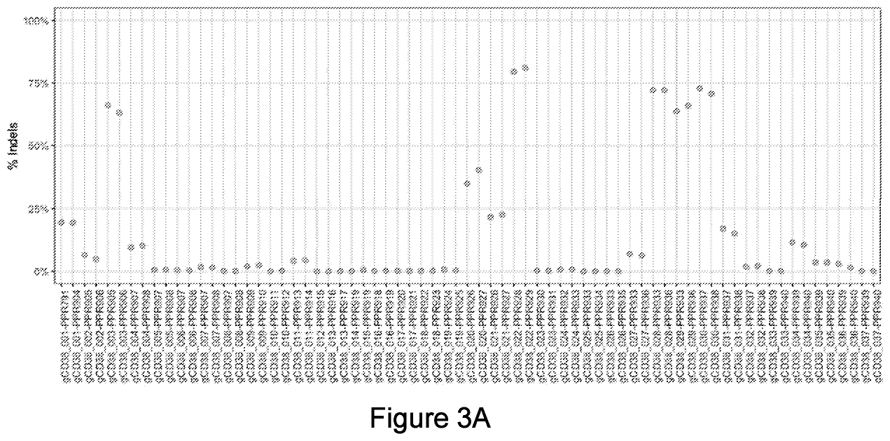

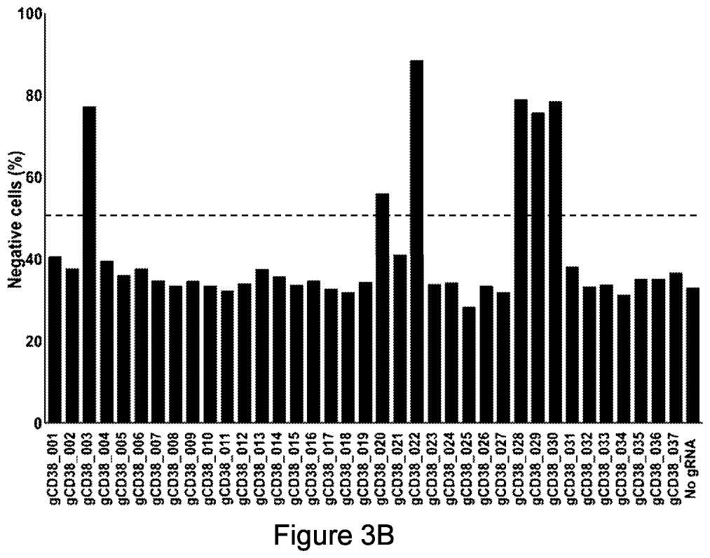

The nucleotide sequence of each single guide RNA used in this example consisted of, from 5′ to 3′, UAAUUUCUACUCUUGUAGAU (SEQ ID NO: 50) and a spacer sequence. In SEQ ID NO: 50, the modulator stem sequence (UCUAC) and the targeter stem sequence (GUAGA) are underlined. The editing efficiency of each single guide RNA was measured as the percentage of cells having one or more insertion or deletion at the target site (% indel). The spacer sequences tested for targeting human CD38 are shown in Table 7. The editing efficiency of each single guide RNA targeting human CD38 is shown in FIG. 3A. Six spacer sequences in particular demonstrate high (>30%) gene editing efficiency: gCD38_003 (SEQ ID NO: 377), gCD38_020 (SEQ ID NO: 394), gCD38_022 (SEQ ID NO: 396), gCD38_028 (SEQ ID NO: 402), gCD38_029 (SEQ ID NO: 403), gCD38_030 (SEQ ID NO: 404).

To functional analyze the editing outcome we used antibody staining of the cells and flowcytometry to determine the negative cell population of the edited protein coding gene. Briefly, 1,000,000 cells/ml were harvested and washed with Cell Staining Buffer (Biolegend, catalog #420201), incubated with a fluorophore tagged antibody against the protein of interest or an indirect marker for the protein of interest, washed with Cell Staining Buffer (Biolegend, catalog #420201), resuspended in 1×PBS and analyzed by Flow cytometry. The data were analyzed using Flowjo, gated for viable, single cells and the negative cell population of the stained protein were determined. The percent of negative cells in a population is plotted against each single guide RNA tested in FIG. 3B. A no gRNA control sample was also tested resulting in a negative cell population of 37%. The same six spacer sequences demonstrating high gene editing efficiency in FIG. 3A demonstrate high negative cell populations (>50%): gCD38_003 (SEQ ID NO: 377), gCD38_020 (SEQ ID NO: 394), gCD38_022 (SEQ ID NO: 396), gCD38_028 (SEQ ID NO: 402), gCD38_029 (SEQ ID NO: 403), gCD38_030 (SEQ ID NO: 404).

### Example 3. Knock Out of Other Human Genes by Single Guide MAD7 CRISPR-Cas Systems

MAD7 is a type V-A Cas protein that has endonuclease activity when complexed with a single guide RNA, also known as a crRNA in a type V-A system (see, U.S. Pat. No. 9,982,279). This example describes cleavage of the genomic DNA of primary Pan T-cells using MAD7 in complex with single guide nucleic acids targeting various human genomic targets to identify factors to generate allogenic cells by reducing the surface levels of HLA class I and II proteins.

Briefly, Pan T-cells were isolated from Leukopaks (StemCell Technology) using EasySep Direct Human T cell Isolation Kit (StemCell Technology Catalog #19661) and cryopreserved using CryoStor CS10 (StemCell Technology Catalog #07930). The cells were thawed and activated with ImmunoCult Human CD3/CD28 T Cell Activator (StemCell Technology Catalog #10991) and cultivated in ImmunoCult-XF T Cell Expansion Medium (StemCell Technology, Catalog #10981) supplemented with IL2 (StemCell Technlogy Catalog #78036.3) at 37° C. in a 5% CO₂ environment, and transfected after approximately 48 hours with RNPs, consisting of MAD7 protein and synthetic gRNA. MAD7 protein, which contained a nucleoplasmin NLS at the C-terminus, was expressed in E. coli and purified by fast protein liquid chromatography (FPLC). RNP complexes were prepared by incubating 100 pmol MAD7 protein with 100 pmol chemically synthesized single guide RNA for 10 minutes at room temperature. The RNPs were mixed with 1,000,000 Pan T-cells resuspended in nucleofection buffer P3 (Lonza) in a final volume of 25 μL. Electroporation was carried out on a 4D-Nucleofector (Lonza) using program EO-115. Following electroporation, the cells were cultured for 2-3 days.

Genomic DNA of the cells was extracted using the Quick Extract DNA extraction solution 1.0 (Epicentre). The genes fragments were amplified from the genomic DNA samples in a PCR reaction with primers with overhang adaptors and processed using the Nextera XT designed primers (IDT). The final PCR products were analyzed by next-generation sequencing, and the data were analyzed with the Crispresso (see, Clement et al. (2019), CRISPResso2 provides accurate and rapid genome editing sequence analysis. Nat Biotechnol. 2019 March; 37(3):224-226. doi: 10.1038/s41587-019-0032-3. PubMed PMID: 30809026). Editing efficiency was determined by the number of edited reads relative to the total number of reads obtained under each condition.

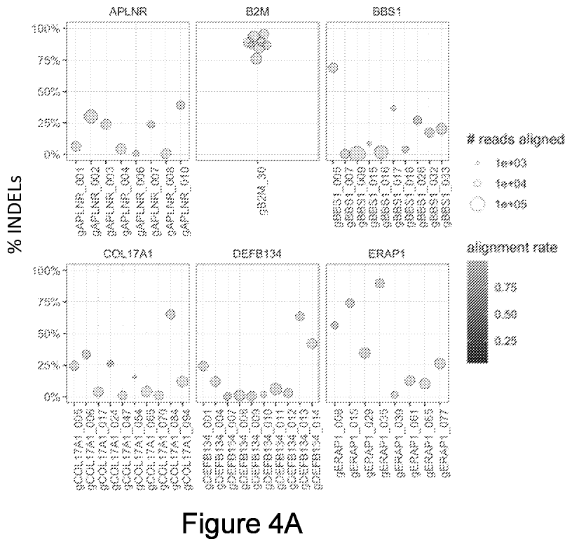

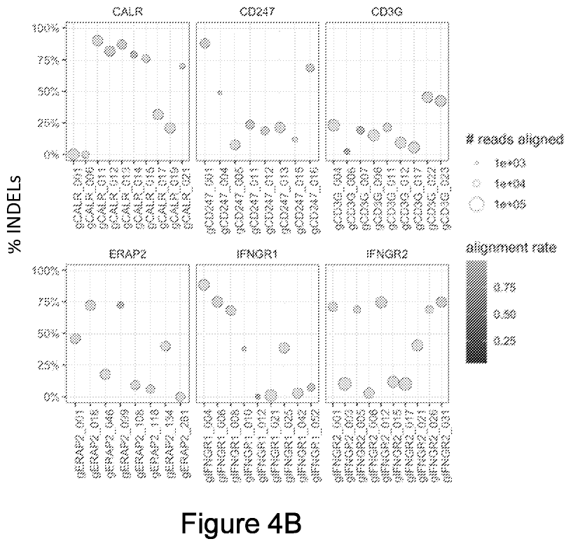

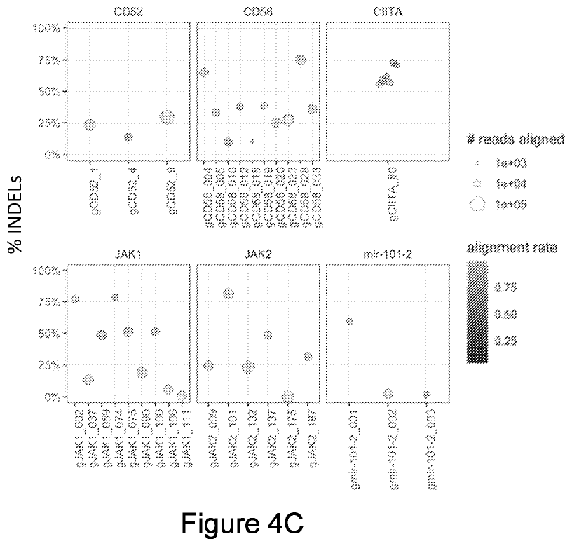

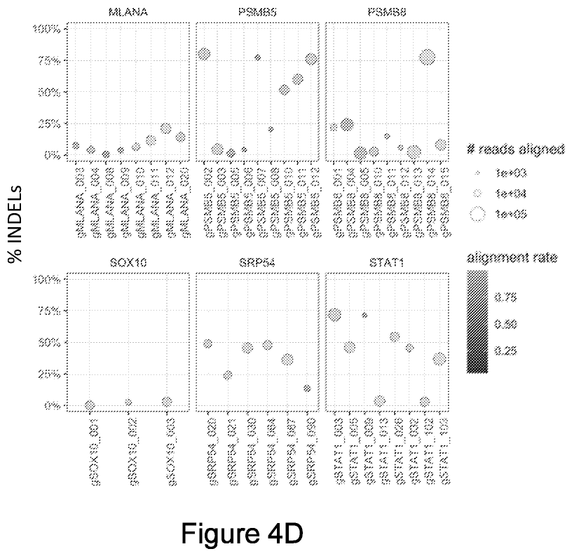

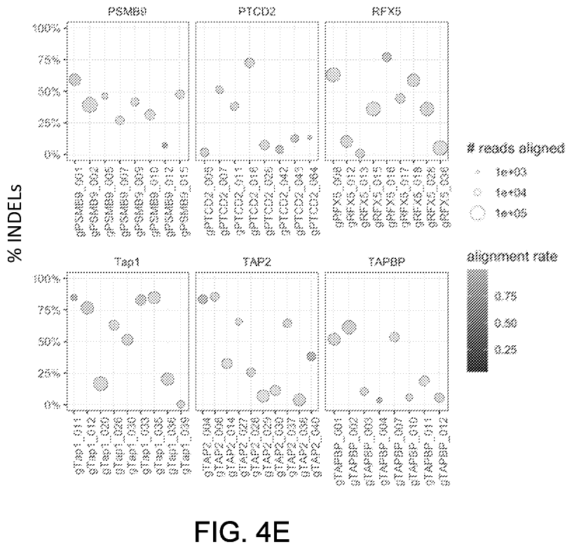

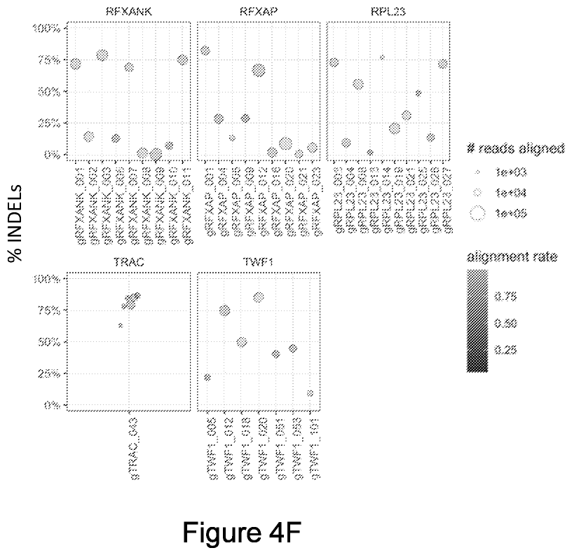

The nucleotide sequence of each single guide RNA used in this example consisted of, from 5′ to 3′, UAAUUUCUACUCUUGUAGAU (SEQ ID NO: 50) and a spacer sequence. In SEQ ID NO: 50, the modulator stem sequence (UCUAC) and the targeter stem sequence (GUAGA) are underlined. The editing efficiency of each single guide RNA was measured as the percentage of cells having one or more insertion or deletion at the target site (% indel). The spacer sequences tested are shown in Table 8. The editing efficiency of each single guide RNA for each gene target (separate subplots) is shown in FIGS. 4 A-F, with the editing efficiency as measured by INDEL formation on the y-axis and the spacer sequence on the x-axis.

### Example 4. Knock Out of Human CD3D and NLRC5 Genes by Single Guide MAD7 CRISPR-Cas Systems

MAD7 is a type V-A Cas protein that has endonuclease activity when complexed with a single guide RNA, also known as a crRNA in a type V-A system (see, U.S. Pat. No. 9,982,279). This example describes cleavage of the genomic DNA of primary Pan T-cells using MAD7 in complex with single guide nucleic acids targeting human CD3D and NLRC5 to identify factors to generate allogenic cells by reducing the surface levels of HLA class I and II proteins.

Briefly, Pan T-cells were isolated from Leukopaks (StemCell Technology) using EasySep Direct Human T cell Isolation Kit (StemCell Technology Catalog #19661) and cryopreserved using CryoStor CS10 (StemCell Technology Catalog #07930). The cells were thawed and activated with ImmunoCult Human CD3/CD28 T Cell Activator (StemCell Technology Catalog #10991) and cultivated in ImmunoCult-XF T Cell Expansion Medium (StemCell Technology, Catalog #10981) supplemented with IL2 (StemCell Technlogy Catalog #78036.3) at 37° C. in a 5% CO₂ environment, and transfected after approximately 48 hours with RNPs, consisting of MAD7 protein and synthetic gRNA. MAD7 protein, which contained a nucleoplasmin NLS at the C-terminus, was expressed in E. coli and purified by fast protein liquid chromatography (FPLC). RNP complexes were prepared by incubating 100 pmol MAD7 protein with 100 pmol chemically synthesized single guide RNA for 10 minutes at room temperature. The RNPs were mixed with 1,000,000 Pan T-cells resuspended in nucleofection buffer P3 (Lonza) in a final volume of 25 μL. Electroporation was carried out on a 4D-Nucleofector (Lonza) using program EO-115. Following electroporation, the cells were cultured for 2-3 days.

The nucleotide sequence of each single guide RNA used in this example consisted of, from 5′ to 3′, UAAUUUCUACUCUU\vec{GUAGA}U (SEQ ID NO: 50) and a spacer sequence. In SEQ ID NO: 50, the modulator stem sequence (UCUAC) and the targeter stem sequence (GUAGA) are underlined. The editing efficiency of each single guide RNA was measured as the percentage of cells having one or more insertion or deletion at the target site (% indel). The spacer sequences tested for targeting human CD3D and NLRC5 are shown in Table 8. The spacer sequence for gB2M_30 was 5′ AGTGGGGGTGAATTCAGTGTA 3′, for gCIITA_80 was 5′ CAAGGACTTCAGCTGGGGGAA 3′, and for gTRAC_043 was 5′ GAGTCTCTCAGCTGGTACACG 3′.

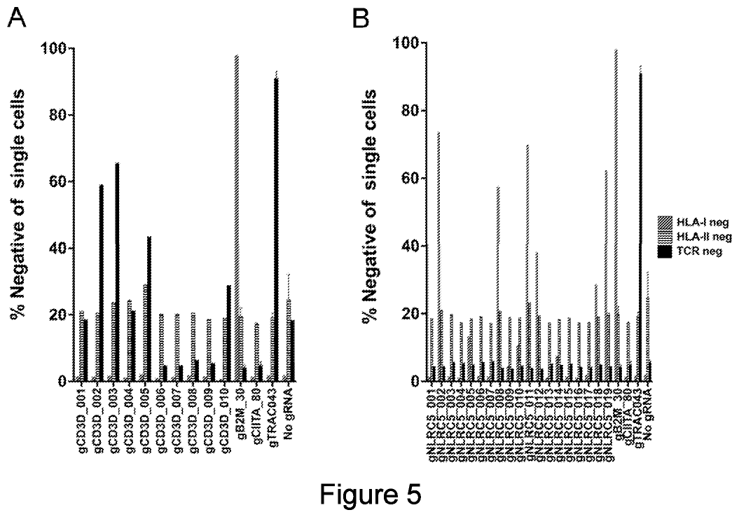

To functionally analyze the editing outcome we used antibody staining of the cells and flowcytometry to determine the negative cell population of the edited protein coding gene. Briefly, 1,000,000 cells/ml were harvested and washed with Cell Staining Buffer (Biolegend, catalog #420201), incubated with a fluorophore tagged antibody against the protein of interest or an indirect marker for the protein of interest, washed with Cell Staining Buffer (Biolegend, catalog #420201), resuspended in 1×PBS and analyzed by Flowcytometry. The data were analyzed using Flowjo, gated for viable, single cells and the negative cell population of the stained protein were determined. The percent of negative cells in a population is plotted against each CD3D and NLRC5 single guide RNA tested for TCR, HLA-I, and HLA-II surface markers in FIGS. 5A and B respectively. A no gRNA control sample was also tested for each of the three surface markers shown as the far right bar.

As shown in FIG. 5A black bars, four sgRNAs demonstrated reduced TCR surface marker expression (higher % negative cells) compared the no sgRNA control: gCD3D_002 (SEQ ID NO: 717), gCD3D_003 (SEQ ID NO: 718), gCD3D_005 (SEQ ID NO: 720), and gCD3D_010 (SEQ ID NO: 725).

As show in FIG. 5B gray bars, nine sgRNAs demonstrated reduced HLA-I surface marker expression (higher % negative cells) compared to the no sgRNA control: gNLRC5_002 (SEQ ID NO: 727), gNLRC5_005 (SEQ ID NO: 730), gNLRC5_008 (SEQ ID NO: 733), gNLRC5_010 (SEQ ID NO: 735), gNLRC5_011 (SEQ ID NO: 736), gNLRC5_012 (SEQ ID NO: 737), gNLRC5_014 (SEQ ID NO: 739), gNLRC5_018 (SEQ ID NO: 743), gNLRC5_019 (SEQ ID NO: 744).

### Example 5. Knock in of DSG3 CAAR into TRBC1/2 or CD3E Loci

This example demonstrates the use of the TRBC1/2 and CD3E loci for knock in of one or more heterologous genes, specifically a DSG3 CAAR. A CAAR (chimeric autoantibody receptor) is a CAR-like protein, wherein instead of comprising a extracellularly-displayed binding domain as for a CAR, a CAAR comprises an extracellularly-displayed antigen. When bound by a B-cell, a CAAR triggers an intracellular cascade that results in the eventual death of the B-cell, thereby demonstrating utility to treat autoimmune disease. Further the example demonstrates the utility of the TRBC1/2 and CD3E loci for knock in in both Pan T-cells and Jurkat cells.

Briefly, Pan T-cells were isolated from Leukopaks (StemCell Technology) using EasySep Direct Human T cell Isolation Kit (StemCell Technology Catalog #19661) and cryopreserved using CryoStor CS10 (StemCell Technology Catalog #07930). The cells were thawed and activated with ImmunoCult Human CD3/CD28 T Cell Activator (StemCell Technology Catalog #10991) and cultivated in ImmunoCult-XF T Cell Expansion Medium (StemCell Technology, Catalog #10981) supplemented with IL2 (StemCell Technlogy Catalog #78036.3) at 37° C. in a 5% CO₂ environment, and transfected after approximately 48 hours with RNPs, consisting of MAD7 protein and synthetic gRNA. MAD7 protein, which contained a nucleoplasmin NLS at the C-terminus, was expressed in E. coli and purified by fast protein liquid chromatography (FPLC). RNP complexes were prepared by incubating 100 pmol MAD7 protein with 100 pmol chemically synthesized single guide RNA for 10 minutes at room temperature. The RNPs were mixed with 1,000,000 Pan T-cells resuspended in nucleofection buffer P3 (Lonza) in a final volume of 25 μL. Electroporation was carried out on a 4D-Nucleofector (Lonza) using program EO-115. Following electroporation, the cells were cultured for 3 days prior to passaging at 1:1 v:v dilution.

Briefly, Jurkat cells were thawed from a glycerol stock stored at −80° C. and seeded into RPMI with 10% FBS at concentration of 1E5 cells/mL. The cells were grown at at 37° C. in a 5% CO₂ environment, and transfected after approximately 48 hours with RNPs, consisting of MAD7 protein and synthetic gRNA. MAD7 protein, which contained a nucleoplasmin NLS at the C-terminus, was expressed in E. coli and purified by fast protein liquid chromatography (FPLC). RNP complexes were prepared by incubating 100 pmol MAD7 protein with 100 pmol chemically synthesized single guide RNA for 10 minutes at room temperature along with 0.3, 0.6, or 0.9 ug of donor template. The RNPs were mixed with 1,000,000 Jurkat cells resuspended in nucleofection buffer P3 (Lonza) in a final volume of 25 μL. Electroporation was carried out on a 4D-Nucleofector (Lonza) using program EO-115. Following electroporation, the cells were cultured for 1 day prior to passaging at 1:1 v:v dilution.

For the TRBC1/2 and CD3E, synthetic guides comprising spacer sequences gTRBC1_2_003 (SEQ ID NO: 331) and gCD3E_34 (SEQ ID NO: 366) were used respectively. ART-21-100 and ART-21-101 plasmids comprising the DSG3 CAAR were used as donor templates.

The ART-21-100_pUCmu-gCD3e34-DSG3-EC1-3 donor template for knock in of the CAAR at the CD3E locus is shown below with the DSG3 CAAR sequence in bold:

CGCGTATTGGGATCCTCAGCGTTCCAAATAGGGACTTCTGTGGGT
TTTTCTTTACATCCATCTTACCCTTCCCAAGTCCCCATGTCCCTG
CGTAAACCCTAAAGCCACCTCTCAAAAGGTTCTCTAGTTCCCTTC
AAGGTTCTCTAGTTCCCTTCATTCCACATATCTCCTCTTCCACAC
CCTCTAGCCAGTAGAGCTCCCTTCTGACAAGCAAGTCTAAGATCT
AGATGACAGATGACTTCCTGCATTTGGGTGGTTCTTTTGTCACTA
ATTTGCCTTTTCTAAAATTGTCCTGGTTTCTTCTGCCAATTTCCC
TTCTTTCTCCCCAGCATATAAAGTCTCCATCTCTGGAACCACAGT
AATATTGACATGCCCTCAGTATCCTGGATCTGAAATACTATGGCA
ACACAATGATAAAAACATAGGCGGTGATGAGGATGATAAAAACAT
AGGCAGTGATGAGGATCACCTGTCACTGAAGGAATTTTCAGAATT
GGAGCAAAGTGGTTATTATGTCTGCCGTGAGGCTCCGGTGCCCGT
CAGTGGGCAGAGCGCACATCGCCCACAGTCCCCGAGAAGTTGGGG
GGAGGGGTCGGCAATTGAACCGGTGCCTAGAGAAGGTGGCGCGGG
GTAAACTGGGAAAGTGATGTCGTGTACTGGCTCCGCCTTTTTCCC
GAGGGTGGGGGAGAACCGTATATAAGTGCAGTAGTCGCCGTGAAC
GTTCTTTTTCGCAACGGGTTTGCCGCCAGAACACAGGTAAGTGCC
GTGTGTGGTTCCCGCGGGCCTGGCCTCTTTACGGGTTATGGCCCT
TGCGTGCCTTGAATTACTTCCACCTGGCTGCAGTACGTGATTCTT
GATCCCGAGCTTCGGGTTGGAAGTGGGTGGGAGAGTTCGAGGCCT
TGCGCTTAAGGAGCCCCTTCGCCTCGTGCTTGAGTTGAGGCCTGG
CCTGGGCGCTGGGGCCGCCGCGTGCGAATCTGGTGGCACCTTCGC
GCCTGTCTCGCTGCTTTCGATAAGTCTCTAGCCATTTAAAATTTT
TGATGACCTGCTGCGACGCTTTTTTTCTGGCAAGATAGTCTTGTA
AATGCGGGCCAAGATCTGCACACTGGTATTTCGGTTTTTGGGGCC
GCGGGCGGCGACGGGGCCCGTGCGTCCCAGCGCACATGTTCGGCG
AGGCGGGGCCTGCGAGCGCGGCCACCGAGAATCGGACGGGGGTAG
TCTCAAGCTGGCCGGCCTGCTCTGGTGCCTGGCCTCGCGCCGCCG
TGTATCGCCCCGCCCTGGGCGGCAAGGCTGGCCCGGTCGGCACCA
GTTGCGTGAGCGGAAAGATGGCCGCTTCCCGGCCCTGCTGCAGGG
AGCTCAAAATGGAGGACGCGGCGCTCGGGAGAGCGGGCGGGTGAG
TCACCCACACAAAGGAAAAGGGCCTTTCCGTCCTCAGCCGTCGCT
TCATGTGACTCCACTGAGTACCGGGCGCCGTCCAGGCACCTCGAT
TAGTTCTCGTGCTTTTGGAGTACGTCGTCTTTAGGTTGGGGGGAG
GGGTTTTATGCGATGGAGTTTCCCCACACTGAGTGGGTGGAGACT
GAAGTTAGGCCAGCTTGGCACTTGATGTAATTCTCCTTGGAATTT
GCCCTTTTTGAGTTTGGATCTTGGTTCATTCTCAAGCCTCAGACA
GTGGTTCAAAGTTTTTTTCTTCCATTTCAGGTGTCGTGAGCTAGA
GCCACCATGGAGTTTGGGCTGAGCTGGCTTTTTCTTGTGGCTATT
TTAAAAGGTGTCCAGTGCGGATCCGAGCTGCGGATCGAGACAAAG
GGCCAGTACGACGAGGAAGAGATGACAATGCAGCAGGCCAAGCGG
CGGCAGAAACGCGAGTGGGTCAAGTTCGCCAAGCCCTGCAGAGAG
GGCGAGGACAACAGCAAGCGGAACCCTATCGCCAAGATCACCAGC
GACTACCAGGCCACCCAGAAGATCACCTACCGGATCAGCGGCGTG
GGCATCGACCAGCCCCCTTTCGGCATCTTCGTGGTGGACAAGAAC
ACCGGCGACATCAACATCACCGCCATCGTGGACAGAGAGGAAACC
CCCAGCTTCCTGATCACCTGTCGGGCCCTGAATGCCCAGGGCCTG
GACGTGGAAAAGCCCCTGATCCTGACCGTGAAGATCCTGGACATC
AACGACAACCCCCCCGTGTTCAGCCAGCAGATCTTCATGGGCGAG
ATCGAGGAAAACAGCGCCAGCAACAGCCTCGTGATGATCCTGAAC
GCCACCGACGCCGACGAGCCCAACCACCTGAATAGCAAGATCGCC
TTCAAGATCGTGTCCCAGGAACCCGCCGGAACCCCCATGTTCCTG
CTGAGCAGAAATACCGGCGAAGTGCGGACCCTGACCAACAGCCTG
GATAGAGAGCAGGCCAGCAGCTACCGGCTGGTGGTGTCTGGCGCT
GACAAGGATGGCGAGGGCCTGAGCACACAGTGCGAGTGCAACATC
AAAGTGAAGGACGTGAACGACAACTTCCCTATGTTCCGGGACAGC
CAGTACAGCGCCCGGATCGAAGAGAACATCCTGAGCAGCGAGCTG
CTGCGGTTCCAAGTGACCGACCTGGACGAAGAGTACACCGACAAC
TGGCTGGCCGTGTACTTCTTCACCAGCGGCAACGAGGGCAATTGG
TTCGAGATCCAGACCGACCCCCGGACCAATGAGGGCATCCTGAAG
GTCGTGAAGGCCCTGGACTACGAGCAGCTGCAGAGCGTGAAGCTG
TCTATCGCCGTGAAGAACAAGGCCGAGTTCCACCAGTCCGTGATC
AGCCGGTACAGAGTGCAGAGCACCCCCGTGACCATCCAAGTGATC
AACGTGCGCGAGGGCATTGCCTTCGCTAGCGGTGGCGGAGGTTCT
GGAGGTGGAGGTTCCTCCGGAATCTACATCTGGGCGCCCTTGGCC
GGGACTTGTGGGGTCCTTCTCCTGTCACTGGTTATCACCCTTTAC
TGCAAACGGGGCAGAAAGAAACTCCTGTATATATTCAAACAACCA
TTTATGAGACCAGTACAAACTACTCAAGAGGAAGATGGCTGTAGC
TGCCGATTTCCAGAAGAAGAAGAAGGAGGATGTGAACTGAGAGTG
AAGTTCAGCAGGAGCGCAGACGCCCCCGCGTACCAGCAGGGCCAG
AACCAGCTCTATAACGAGCTCAATCTAGGACGAAGAGAGGAGTAC
GATGTTTTGGACAAGAGACGTGGCCGGGACCCTGAGATGGGGGGA
AAGCCGAGAAGGAAGAACCCTCAGGAAGGCCTGTACAATGAACTG
CAGAAAGATAAGATGGCGGAGGCCTACAGTGAGATTGGGATGAAA
GGCGAGCGCCGGAGGGGCAAGGGGCACGATGGCCTTTACCAGGGT
CTCAGTACAGCCACCAAGGACACCTACGACGCCCTTCACATGCAG
GCCCTGCCCCCTCGCTAAGTCGACAATCAACCTCTGGATTACAAA
ATTTGTGAAAGATTGACTGGTATTCTTAACTATGTTGCTCCTTTT
ACGCTATGTGGATACGCTGCTTTAATGCCTTTGTATCATGCTATT
GCTTCCCGTATGGCTTTCATTTTCTCCTCCTTGTATAAATCCTGG
TTGCTGTCTCTTTATGAGGAGTTGTGGCCCGTTGTCAGGCAACGT
GGCGTGGTGTGCACTGTGTTTGCTGACGCAACCCCCACTGGTTGG
GGCATTGCCACCACCTGTCAGCTCCTTTCCGGGACTTTCGCTTTC
CCCCTCCCTATTGCCACGGCGGAACTCATCGCCGCCTGCCTTGCC
CGCTGCTGGACAGGGGCTCGGCTGTTGGGCACTGACAATTCCGTG
GTGTTGTCGGGGAAGCTGACGTCCTTTCCTTGGCTGCTCGCCTGT
GTTGCCACCTGGATTCTGCGCGGGACGTCCTTCTGCTACGTCCCT
TCGGCCCTCAATCCAGCGGACCTTCCTTCCCGCGGCCTGCTGCCG
GCTCTGCGGCCTCTTCCGCGTCTTCGCCTTCGCCCTCAGACGAGT
CGGATCTCCCTTTGGGCCGCCTCCCCGCCTGCGACTGTGCCTTCT
AGTTGCCAGCCATCTGTTGTTTGCCCCTCCCCCGTGCCTTCCTTG
ACCCTGGAAGGTGCCACTCCCACTGTCCTTTCCTAATAAAATGAG
GAAATTGCATCGCATTGTCTGAGTAGGTGTCATTCTATTCTGGGG
GGTGGGGTGGGGCAGGACAGCAAGGGGGAGGATTGGGAAGACAAT
AGCAGGCATGCTGGGGATGCGGTGGGCTCTATGGTACCCCAGAGG
AAGCAAACCAGAAGATGCGAACTTTTATCTCTACCTGAGGGCAAG
AGGTAATCCAGGTCTCCAGAACAGGTACCACCGGCTCTTTAGGGA
GGACCATTCAAAAGGGCATTCTCAGTGATTTTCCCTAACCCAGCT
CACAGTGCCCAGGCGTCTTTGCGCTTCCTCCCACACTCAATCCTG
GGACTCTCTGGTACCACACGGCATCAGTGTTTTCTGGAATATAGA
TTAAACACCAATATGAGGCTTCTGGGTAACCCCAGTCTGTGCGAG
ATCTAAAATAGCAACTCCCTAAGAGACAGGACTGGGTCATTTGCA
CCGCATCACACCCAGGTTCATAGCACACCAACATGAGTTTATCTA
ATGCTTCCTCCAGAGATAAATTTTTCAGAAAGGTTTGCAAAAAAC
ACTCAAGGCCACTATAGTAAAATGGCATAAGCTAAGGTATAATAA
TAAAATAATAACAATACTTAACATTTATTGAGTGCTTATGCGGCC
GCTGTCTGCTACCCCAGAGGAAGCAAACAGGTCGACTCTAGAGGA
TCCCGGGTACCGAGCTCGAATTCGGATATCCTCGAGACTAGTGGG
CCCGTTTAAACACATGTGTTTTTCCATAGGCTCCGCCCCCCTGAC
GAGCATCACAAAAATCGACGCTCAAGTCAGAGGTGGCGAAACCCG
ACAGGACTATAAAGATACCAGGCGTTTCCCCCTGGAAGCTCCCTC
GTGCGCTCTCCTGTTCCGACCCTGCCGCTTACCGGATACCTGTCC
GCCTTTCTCCCTTCGGGAAGCGTGGCGCTTTCTCATAGCTCACGC
TGTAGGTATCTCAGTTCGGTGTAGGTCGTTCGCTCCAAGCTGGGC
TGTGTGCACGAACCCCCCGTTCAGCCCGACCGCTGCGCCTTATCC
GGTAACTATCGTCTTGAGTCCAACCCGGTAAGACACGACTTATCG
CCACTGGCAGCAGCCACTGGTAACAGGATTAGCAGAGCGAGGTAT
GTAGGCGGTGCTACAGAGTTCTTGAAGTGGTGGCCTAACTACGGC
TACACTAGAAGAACAGTATTTGGTATCTGCGCTCTGCTGAAGCCA
GTTACCTTCGGAAAAAGAGTTGGTAGCTCTTGATCCGGCAAACAA
ACCACCGCTGGTAGCGGTGGTTTTTTTGTTTGCAAGCAGCAGATT
ACGCGCAGAAAAAAAGGATCTCAAGAAGATCCTTTGATCTTTTCT
ACTACCAATGCTTAATCAGTGAGGCACCTATCTCAGCGATCTGTC
TATTTCGTTCATCCATAGTTGCCTGACTCCCCGTCGTGTAGATAA
CTACGATACGGGAGGGCTTACCATCTGGCCCCAGTGCTGCAATGA
TACCGCGAGACCCACGCTCACCGGCTCCAGATTTATCAGCAATAA
ACCAGCCAGCCGGAAGGGCCGAGCGCAGAAGTGGTCCTGCAACTT
TATCCGCCTCCATCCAGTCTATTAATTGTTGCCGGGAAGCTAGAG
TAAGTAGTTCGCCAGTTAATAGTTTGCGCAACGTTGTTGCCATTG
CTACAGGCATCGTGGTGTCACGCTCGTCGTTTGGTATGGCTTCAT
TCAGCTCCGGTTCCCAACGATCAAGGCGAGTTACATGATCCCCCA
TGTTGTGCAAAAAAGCGGTTAGCTCCTTCGGTCCTCCGATCGTTG
TCAGAAGTAAGTTGGCCGCAGTGTTATCACTCATGGTTATGGCAG
CACTGCATAATTCTCTTACTGTCATGCCATCCGTAAGATGCTTTT
CTGTGACTGGTGAGTACTCAACCAAGTCATTCTGAGAATAGTGTA
TGCGGCGACCGAGTTGCTCTTGCCCGGCGTCAATACGGGATAATA
CCGCGCCACATAGCAGAACTTTAAAAGTGCTCATCATTGGAAAAC
GTTCTTCGGGGCGAAAACTCTCAAGGATCTTACCGCTGTTGAGAT
CCAGTTCGATGTAACCCACTCGTGCACCCAACTGATCTTCAGCAT
CTTTTACTTTCACCAGCGTTTCTGGGTGAGCAAAAACAGGAAGGC
AAAATGCCGCAAAAAAGGGAATAAGGGCGACACGGAAATGTTGAA
TACTCATACTCTTCCTTTTTCAATATTATTGAAGCATTTATCAGG
GTTATTGTCTCATGAGCGGATACATACGCGAGGCCATATGGGTTA
ACTTTGCTTCCTCTGGGGTAGCAGACACCTCAGCA

The ART-21-101_pUCmu-gTRBC1-DSG3-EC1-3 donor template for knock in of the CAAR at the TRBC1/2 locus is shown below with the DSG3 CAAR sequence in bold:

CGCGTATTGGGATCCTCAGCAAAGGAAAATTATAATTAGAAAAAG
TCAATTTAGTTATTGTAATTATACCACTAATGAGAGTTTCCTACC
TCGAGTTTCAGGATTACATAGCCATGCACCAAGCAAGGCTTTGAA
AAATAAAGATACACAGATAAATTATTTGGATAGATGATCAGACAA
GCCTCAGTAAAAACAGCCAAGACAATCAGGATATAATGTGACCAT
AGGAAGCTGGGGAGACAGTAGGCAATGTGCATCCATGGGACAGCA
TAGAAAGGAGGGGCAAAGTGGAGAGAGAGCAACAGACACTGGGAT
GGTGACCCCAAAACAATGAGGGCCTAGAATGACATAGTTGTGCTT
CATTACGGCCCATTCCCAGGGCTCTCTCTCACACACACAGAGCCC
CTACCAGAACCAGACAGCTCTCAGAGCAACCCTGGCTCCAACCCC
TCTTCCCTTTCCAGAGGACCTGAACAAGGTGTTCCCACCCGAGGT
CGCTGTGTTTGAGCCATCAGAAGCACGTGAGGCTCCGGTGCCCGT
CAGTGGGCAGAGCGCACATCGCCCACAGTCCCCGAGAAGTTGGGG
GGAGGGGTCGGCAATTGAACCGGTGCCTAGAGAAGGTGGCGCGGG
GTAAACTGGGAAAGTGATGTCGTGTACTGGCTCCGCCTTTTTCCC
GAGGGTGGGGGAGAACCGTATATAAGTGCAGTAGTCGCCGTGAAC
GTTCTTTTTCGCAACGGGTTTGCCGCCAGAACACAGGTAAGTGCC
GTGTGTGGTTCCCGCGGGCCTGGCCTCTTTACGGGTTATGGCCCT
TGCGTGCCTTGAATTACTTCCACCTGGCTGCAGTACGTGATTCTT
GATCCCGAGCTTCGGGTTGGAAGTGGGTGGGAGAGTTCGAGGCCT
TGCGCTTAAGGAGCCCCTTCGCCTCGTGCTTGAGTTGAGGCCTGG
CCTGGGCGCTGGGGCCGCCGCGTGCGAATCTGGTGGCACCTTCGC
GCCTGTCTCGCTGCTTTCGATAAGTCTCTAGCCATTTAAAATTTT
TGATGACCTGCTGCGACGCTTTTTTTCTGGCAAGATAGTCTTGTA
AATGCGGGCCAAGATCTGCACACTGGTATTTCGGTTTTTGGGGCC
GCGGGCGGCGACGGGGCCCGTGCGTCCCAGCGCACATGTTCGGCG
AGGCGGGGCCTGCGAGCGCGGCCACCGAGAATCGGACGGGGGTAG
TCTCAAGCTGGCCGGCCTGCTCTGGTGCCTGGCCTCGCGCCGCCG
TGTATCGCCCCGCCCTGGGCGGCAAGGCTGGCCCGGTCGGCACCA
GTTGCGTGAGCGGAAAGATGGCCGCTTCCCGGCCCTGCTGCAGGG
AGCTCAAAATGGAGGACGCGGCGCTCGGGAGAGCGGGCGGGTGAG
TCACCCACACAAAGGAAAAGGGCCTTTCCGTCCTCAGCCGTCGCT
TCATGTGACTCCACTGAGTACCGGGCGCCGTCCAGGCACCTCGAT
TAGTTCTCGTGCTTTTGGAGTACGTCGTCTTTAGGTTGGGGGGAG
GGGTTTTATGCGATGGAGTTTCCCCACACTGAGTGGGTGGAGACT
GAAGTTAGGCCAGCTTGGCACTTGATGTAATTCTCCTTGGAATTT
GCCCTTTTTGAGTTTGGATCTTGGTTCATTCTCAAGCCTCAGACA
GTGGTTCAAAGTTTTTTTCTTCCATTTCAGGTGTCGTGAGCTAGA
GCCACCATGGAGTTTGGGCTGAGCTGGCTTTTTCTTGTGGCTATT
TTAAAAGGTGTCCAGTGCGGATCCGAGCTGCGGATCGAGACAAAG
GGCCAGTACGACGAGGAAGAGATGACAATGCAGCAGGCCAAGCGG
CGGCAGAAACGCGAGTGGGTCAAGTTCGCCAAGCCCTGCAGAGAG
GGCGAGGACAACAGCAAGCGGAACCCTATCGCCAAGATCACCAGC
GACTACCAGGCCACCCAGAAGATCACCTACCGGATCAGCGGCGTG
GGCATCGACCAGCCCCCTTTCGGCATCTTCGTGGTGGACAAGAAC
ACCGGCGACATCAACATCACCGCCATCGTGGACAGAGAGGAAACC
CCCAGCTTCCTGATCACCTGTCGGGCCCTGAATGCCCAGGGCCTG
GACGTGGAAAAGCCCCTGATCCTGACCGTGAAGATCCTGGACATC
AACGACAACCCCCCCGTGTTCAGCCAGCAGATCTTCATGGGCGAG
ATCGAGGAAAACAGCGCCAGCAACAGCCTCGTGATGATCCTGAAC
GCCACCGACGCCGACGAGCCCAACCACCTGAATAGCAAGATCGCC
TTCAAGATCGTGTCCCAGGAACCCGCCGGAACCCCCATGTTCCTG
CTGAGCAGAAATACCGGCGAAGTGCGGACCCTGACCAACAGCCTG
GATAGAGAGCAGGCCAGCAGCTACCGGCTGGTGGTGTCTGGCGCT
GACAAGGATGGCGAGGGCCTGAGCACACAGTGCGAGTGCAACATC
AAAGTGAAGGACGTGAACGACAACTTCCCTATGTTCCGGGACAGC
CAGTACAGCGCCCGGATCGAAGAGAACATCCTGAGCAGCGAGCTG
CTGCGGTTCCAAGTGACCGACCTGGACGAAGAGTACACCGACAAC
TGGCTGGCCGTGTACTTCTTCACCAGCGGCAACGAGGGCAATTGG
TTCGAGATCCAGACCGACCCCCGGACCAATGAGGGCATCCTGAAG
GTCGTGAAGGCCCTGGACTACGAGCAGCTGCAGAGCGTGAAGCTG
TCTATCGCCGTGAAGAACAAGGCCGAGTTCCACCAGTCCGTGATC
AGCCGGTACAGAGTGCAGAGCACCCCCGTGACCATCCAAGTGATC
AACGTGCGCGAGGGCATTGCCTTCGCTAGCGGTGGCGGAGGTTCT
GGAGGTGGAGGTTCCTCCGGAATCTACATCTGGGCGCCCTTGGCC
GGGACTTGTGGGGTCCTTCTCCTGTCACTGGTTATCACCCTTTAC
TGCAAACGGGGCAGAAAGAAACTCCTGTATATATTCAAACAACCA
TTTATGAGACCAGTACAAACTACTCAAGAGGAAGATGGCTGTAGC
TGCCGATTTCCAGAAGAAGAAGAAGGAGGATGTGAACTGAGAGTG
AAGTTCAGCAGGAGCGCAGACGCCCCCGCGTACCAGCAGGGCCAG
AACCAGCTCTATAACGAGCTCAATCTAGGACGAAGAGAGGAGTAC
GATGTTTTGGACAAGAGACGTGGCCGGGACCCTGAGATGGGGGGA
AAGCCGAGAAGGAAGAACCCTCAGGAAGGCCTGTACAATGAACTG
CAGAAAGATAAGATGGCGGAGGCCTACAGTGAGATTGGGATGAAA
GGCGAGCGCCGGAGGGGCAAGGGGCACGATGGCCTTTACCAGGGT
CTCAGTACAGCCACCAAGGACACCTACGACGCCCTTCACATGCAG
GCCCTGCCCCCTCGCTAAGTCGACAATCAACCTCTGGATTACAAA
ATTTGTGAAAGATTGACTGGTATTCTTAACTATGTTGCTCCTTTT
ACGCTATGTGGATACGCTGCTTTAATGCCTTTGTATCATGCTATT
GCTTCCCGTATGGCTTTCATTTTCTCCTCCTTGTATAAATCCTGG
TTGCTGTCTCTTTATGAGGAGTTGTGGCCCGTTGTCAGGCAACGT
GGCGTGGTGTGCACTGTGTTTGCTGACGCAACCCCCACTGGTTGG
GGCATTGCCACCACCTGTCAGCTCCTTTCCGGGACTTTCGCTTTC
CCCCTCCCTATTGCCACGGCGGAACTCATCGCCGCCTGCCTTGCC
CGCTGCTGGACAGGGGCTCGGCTGTTGGGCACTGACAATTCCGTG
GTGTTGTCGGGGAAGCTGACGTCCTTTCCTTGGCTGCTCGCCTGT
GTTGCCACCTGGATTCTGCGCGGGACGTCCTTCTGCTACGTCCCT
TCGGCCCTCAATCCAGCGGACCTTCCTTCCCGCGGCCTGCTGCCG
GCTCTGCGGCCTCTTCCGCGTCTTCGCCTTCGCCCTCAGACGAGT
CGGATCTCCCTTTGGGCCGCCTCCCCGCCTGCGACTGTGCCTTCT
AGTTGCCAGCCATCTGTTGTTTGCCCCTCCCCCGTGCCTTCCTTG
ACCCTGGAAGGTGCCACTCCCACTGTCCTTTCCTAATAAAATGAG
GAAATTGCATCGCATTGTCTGAGTAGGTGTCATTCTATTCTGGGG
GGTGGGGTGGGGCAGGACAGCAAGGGGGAGGATTGGGAAGACAAT
AGCAGGCATGCTGGGGATGCGGTGGGCTCTATGGGAGATCTCCCA
CACCCAAAAGGCCACACTGGTGTGCCTGGCCACAGGCTTCTTCCC
TGACCACGTGGAGCTGAGCTGGTGGGTGAATGGGAAGGAGGTGCA
CAGTGGGGTCAGCACGGACCCGCAGCCCCTCAAGGAGCAGCCCGC
CCTCAATGACTCCAGATACTGCCTGAGCAGCCGCCTGAGGGTCTC
GGCCACCTTCTGGCAGAACCCCCGCAACCACTTCCGCTGTCAAGT
CCAGTTCTACGGGCTCTCGGAGAATGACGAGTGGACCCAGGATAG
GGCCAAACCCGTCACCCAGATCGTCAGCGCCGAGGCCTGGGGTAG
AGCAGGTGAGTGGGGCCTGGGGAGATGCCTGGAGGAGATTAGGTG
AGACCAGCTACCAGGGAAAATGGAAAGATCCAGGTAGCAGACAAG
ACTAGATCCAAAAAGAAAGGAACCAGCGCACACCATGAAGGAGAA
TTGGGCACCTGTGGTTCATTCTTCTCCCAGATTCTCAGCGCGGCC
GCAGATCTCTGCTTCTGATGGCTCAAACAGGTCGACTCTAGAGGA
TCCCGGGTACCGAGCTCGAATTCGGATATCCTCGAGACTAGTGGG
CCCGTTTAAACACATGTGTTTTTCCATAGGCTCCGCCCCCCTGAC
GAGCATCACAAAAATCGACGCTCAAGTCAGAGGTGGCGAAACCCG
ACAGGACTATAAAGATACCAGGCGTTTCCCCCTGGAAGCTCCCTC
GTGCGCTCTCCTGTTCCGACCCTGCCGCTTACCGGATACCTGTCC
GCCTTTCTCCCTTCGGGAAGCGTGGCGCTTTCTCATAGCTCACGC
TGTAGGTATCTCAGTTCGGTGTAGGTCGTTCGCTCCAAGCTGGGC
TGTGTGCACGAACCCCCCGTTCAGCCCGACCGCTGCGCCTTATCC
GGTAACTATCGTCTTGAGTCCAACCCGGTAAGACACGACTTATCG
CCACTGGCAGCAGCCACTGGTAACAGGATTAGCAGAGCGAGGTAT
GTAGGCGGTGCTACAGAGTTCTTGAAGTGGTGGCCTAACTACGGC
TACACTAGAAGAACAGTATTTGGTATCTGCGCTCTGCTGAAGCCA
GTTACCTTCGGAAAAAGAGTTGGTAGCTCTTGATCCGGCAAACAA
ACCACCGCTGGTAGCGGTGGTTTTTTTGTTTGCAAGCAGCAGATT
ACGCGCAGAAAAAAAGGATCTCAAGAAGATCCTTTGATCTTTTCT
ACTACCAATGCTTAATCAGTGAGGCACCTATCTCAGCGATCTGTC
TATTTCGTTCATCCATAGTTGCCTGACTCCCCGTCGTGTAGATAA
CTACGATACGGGAGGGCTTACCATCTGGCCCCAGTGCTGCAATGA
TACCGCGAGACCCACGCTCACCGGCTCCAGATTTATCAGCAATAA
ACCAGCCAGCCGGAAGGGCCGAGCGCAGAAGTGGTCCTGCAACTT
TATCCGCCTCCATCCAGTCTATTAATTGTTGCCGGGAAGCTAGAG
TAAGTAGTTCGCCAGTTAATAGTTTGCGCAACGTTGTTGCCATTG
CTACAGGCATCGTGGTGTCACGCTCGTCGTTTGGTATGGCTTCAT
TCAGCTCCGGTTCCCAACGATCAAGGCGAGTTACATGATCCCCCA
TGTTGTGCAAAAAAGCGGTTAGCTCCTTCGGTCCTCCGATCGTTG
TCAGAAGTAAGTTGGCCGCAGTGTTATCACTCATGGTTATGGCAG
CACTGCATAATTCTCTTACTGTCATGCCATCCGTAAGATGCTTTT
CTGTGACTGGTGAGTACTCAACCAAGTCATTCTGAGAATAGTGTA
TGCGGCGACCGAGTTGCTCTTGCCCGGCGTCAATACGGGATAATA
CCGCGCCACATAGCAGAACTTTAAAAGTGCTCATCATTGGAAAAC
GTTCTTCGGGGCGAAAACTCTCAAGGATCTTACCGCTGTTGAGAT
CCAGTTCGATGTAACCCACTCGTGCACCCAACTGATCTTCAGCAT
CTTTTACTTTCACCAGCGTTTCTGGGTGAGCAAAAACAGGAAGGC
AAAATGCCGCAAAAAAGGGAATAAGGGCGACACGGAAATGTTGAA
TACTCATACTCTTCCTTTTTCAATATTATTGAAGCATTTATCAGG
GTTATTGTCTCATGAGCGGATACATACGCGAGGCCATATGGGTTA
ACTTTGAGCCATCAGAAGCAGAGATCTCCTCAGCA

Five controls were used for the experiment: (1) wild-type Jurkat cells (WT Jurkat, negative control), (2) Pan T-cells transfected with no donor template (No Cargo Ctrl, negative control), (3) Pan T-cells without electroporation (No NF Ctrl, negative control); (4) DSG3-displaying Jurkat cells (DSG3-Jurkat, positive control); and (5) PDS-20-010 cells displaying DSG3 (positive control).

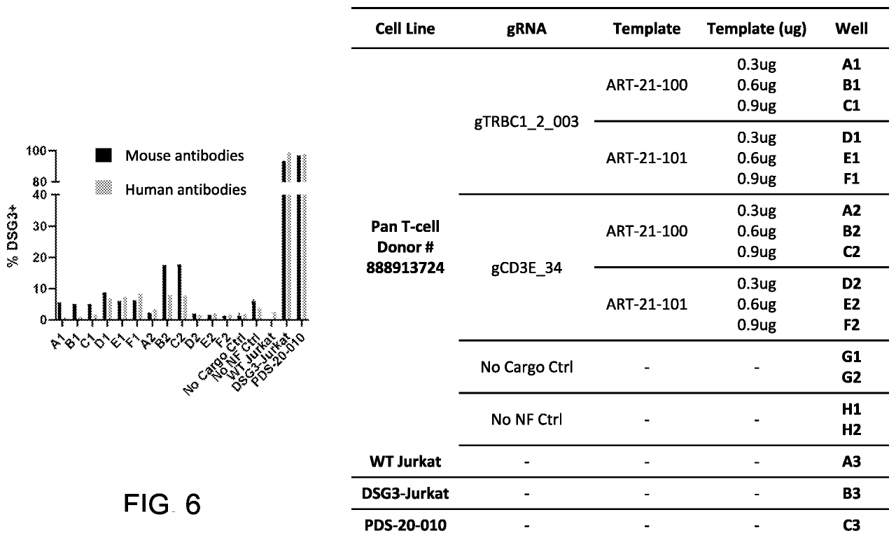

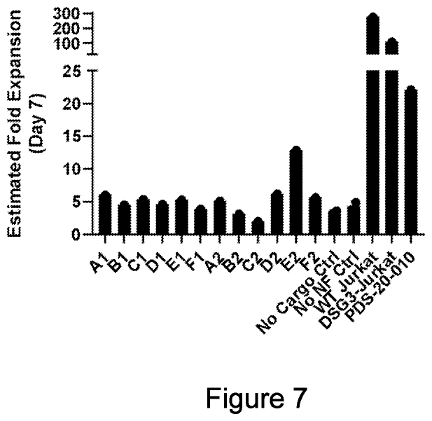

To functionally analyze the editing outcome, we used antibody staining of the cells and flowcytometry to determine the negative cell population of the edited protein coding gene. Briefly, 1,000,000 cells/ml were harvested and washed with Cell Staining Buffer (Biolegend, catalog #420201), incubated with a fluorophore tagged antibody (either primary human anti-DSG3 diluted to 1:100 and secondary anti-human IgG-AG647 diluted 1:1000 or primary mouse anti-DSG3 diluted to 1:50 and secondary anti-mouse IgG-PE diluted 1:1000) against the protein of interest or an indirect marker for the protein of interest, washed with Cell Staining Buffer (Biolegend, catalog #420201), resuspended in 1×PBS and analyzed by Flowcytometry. The data were analyzed using Flowjo, gated for viable, single cells and the negative cell population of the stained protein were determined. The percent of DSG3 positive cells (comprising the CAAR) in a population is plotted for each treatment condition as shown in FIG. 6, with the mouse primary and secondary shown in black and the human primary and second shown in gray. A no gRNA control sample was also tested for each of the three surface markers shown as the far right bar. KI efficiency of DSG3 CAAR as measured by the percentage of the recovered population of using MAD7 in combination with gTRBC1_2_003/ART-21-101 and gCD3E_34/ART-21-100 was between ˜5-20%. Cell counts were further measured daily after nucleofection. Day 7 expansion data is shown in FIG. 7 for each treatment condition. Notably, the fold expansion was on average similar across Nucleofected samples. High DSG3 CAAR expressing treatment conditions (B2 and C2 using gCD3_34/ART-21-100) demonstrates lower fold expansion than those treatment conditions showing lower DSG3 CAAR expression.

This example further demonstrates the use of the TRBC1/2 and CD3E sites for integration of heterologous genes.

### EQUIVALENTS

The invention may be embodied in other specific forms without departing from the spirit or essential characteristics thereof. The foregoing embodiments are therefore to be considered in all respects illustrative rather than limiting on the invention described herein. Scope of the invention is thus indicated by the appended claims rather than by the foregoing description, and all changes that come within the meaning and range of equivalency of the claims are intended to be embraced therein.

1. A guide nucleic acid comprising a targeter stem sequence and a spacer sequence, wherein the spacer sequence comprises a nucleotide sequence listed in Table 1, 2, 3, 4, 5, 6, 7, 8, 9, 14, 15, 16, 17, 18, 19, or 20.

2. The guide nucleic acid of claim 1, wherein the targeter stem sequence comprises a nucleotide sequence of GUAGA.

3. The guide nucleic acid of claim 1, wherein the targeter stem sequence is 5′ to the spacer sequence, optionally wherein the targeter stem sequence is linked to the spacer sequence by a linker consisting of 1, 2, 3, 4, or 5 nucleotides.

4. The guide nucleic acid of claim 1, wherein the guide nucleic acid is capable of activating a CRISPR Associated (Cas) nuclease in the absence of a tracrRNA, optionally wherein the Cas nuclease recognizes a protospacer adjacent motif (PAM) consisting of the nucleotide sequence of TTTN or CTTN.

5. The guide nucleic acid of claim 4, wherein the guide nucleic acid comprises from 5′ to 3′ a modulator stem sequence, a loop sequence, a targeter stem sequence, and the spacer sequence.

6. The guide nucleic acid of claim 1, wherein the guide nucleic acid is a targeter nucleic acid that, in combination with a modulator nucleic acid, is capable of activating a Cas nuclease.

7. The guide nucleic acid of claim 6, wherein the guide nucleic acid comprises from 5′ to 3′ a targeter stem sequence and the spacer sequence.

8. The guide nucleic acid of claim 4, wherein the Cas nuclease is a type V Cas nuclease, optionally a type V-A Cas nuclease.

9. (canceled)

10. The guide nucleic acid of claim 9, wherein the Cas nuclease comprises an amino acid sequence at least 80% identical to SEQ ID NO: 1 or wherein the Cas nuclease is Cpf1.

11.-12. (canceled)

13. The guide nucleic acid of claim 1, wherein the guide nucleic acid comprises a ribonucleic acid (RNA) or a combination of RNA and DNA, optionally wherein the RNA is modified RNA.

14.-15. (canceled)

16. The guide nucleic acid of claim 13, wherein the guide nucleic acid comprises a chemical modification, optionally wherein:
the chemical modification is present in one or more nucleotides at the 5′ end of the guide nucleic acid and/or in one or more nucleotides at the 3′ end of the guide nucleic acid; and/or
the chemical modification is selected from the group consisting of 2′-O-methyl, 2′-fluoro, 2′-O-methoxyethyl, phosphorothioate, phosphorodithioate, pseudouridine, and any combinations thereof.

17.-19. (canceled)

20. An engineered, non-naturally occurring system comprising the guide nucleic acid of claim 4.

21. The engineered, non-naturally occurring system of claim 20, further comprising the Cas nuclease, optionally wherein the guide nucleic acid and the Cas nuclease are present in a ribonucleoprotein (RNP) complex.

22. (canceled)

23. An engineered, non-naturally occurring system comprising the guide nucleic acid of claim 6, further comprising the modulator nucleic acid.

24. The engineered, non-naturally occurring system of claim 23, further comprising the Cas nuclease, optionally wherein the guide nucleic acid, the modulator nucleic acid, and the Cas nuclease are present in an RNP complex.

25. (canceled)

26. The engineered, non-naturally occurring system of claim 20, wherein the spacer sequence comprises a nucleotide sequence selected from the group consisting of:
SEQ ID NOs: 201-253, wherein the spacer sequence is capable of hybridizing with the human CSF2 gene;
SEQ ID NOs: 254-313, wherein the spacer sequence is capable of hybridizing with the human CD40LG gene;
SEQ ID NOs: 314-319 and 329-332, wherein the spacer sequence is capable of hybridizing with the human TRBC1 gene;
SEQ ID NOs: 320-328 and 329-332, wherein the spacer sequence is capable of hybridizing with the human TRBC2 gene;
SEQ ID NOs: 329-332, wherein the spacer sequence is capable of hybridizing with both the human TRBC1 gene and the human TRBC2 gene;
SEQ ID NOs: 333-374, wherein the spacer sequence is capable of hybridizing with the human CD3E gene;
SEQ ID NOs: 375-411, wherein the spacer sequence is capable of hybridizing with the human CD38 gene;
SEQ ID NOs: 412-421, wherein the spacer sequence is capable of hybridizing with the human APLNR gene;
SEQ ID NOs: 422-431, wherein the spacer sequence is capable of hybridizing with the human BBS1 gene;
SEQ ID NOs: 432-441, wherein the spacer sequence is capable of hybridizing with the human CALR gene;
SEQ ID NOs: 442-451, wherein the spacer sequence is capable of hybridizing with the human CD247 gene;
SEQ ID NOs: 452-461, wherein the spacer sequence is capable of hybridizing with the human CD3G gene;
SEQ ID NOs: 462-465, wherein the spacer sequence is capable of hybridizing with the human CD52 gene;
SEQ ID NOs: 476-485, wherein the spacer sequence is capable of hybridizing with the human COL17A1;
SEQ ID NOs: 486-495, wherein the spacer sequence is capable of hybridizing with the human DEFB134 gene;
SEQ ID NOs: 496-505, wherein the spacer sequence is capable of hybridizing with the human ERAP1 gene;
SEQ ID NOs: 506-515, wherein the spacer sequence is capable of hybridizing with the human ERAP2 gene;
SEQ ID NOs: 516-525, wherein the spacer sequence is capable of hybridizing with the human IFNGR1 gene;
SEQ ID NOs: 526-535, wherein the spacer sequence is capable of hybridizing with the human IFNGR2 gene;
SEQ ID NOs: 536-545, wherein the spacer sequence is capable of hybridizing with the human JAK1 gene;
SEQ ID NOs: 546-555, wherein the spacer sequence is capable of hybridizing with the human JAK2 gene;
SEQ ID NOs: 556-558, wherein the spacer sequence is capable of hybridizing with the human mir-101-2 gene;
SEQ ID NOs: 559-568, wherein the spacer sequence is capable of hybridizing with the human MLANA gene;
SEQ ID NOs: 569-578, wherein the spacer sequence is capable of hybridizing with the human PSMB5 gene;
SEQ ID NOs: 579-588, wherein the spacer sequence is capable of hybridizing with the human PSMB8 gene;
SEQ ID NOs: 589-598, and wherein the spacer sequence is capable of hybridizing with the human PSMB9 gene;
SEQ ID NOs: 599-608, wherein the spacer sequence is capable of hybridizing with the human PTCD2 gene;
SEQ ID NOs: 609-618, wherein the spacer sequence is capable of hybridizing with the human RFX5 gene;
SEQ ID NOs: 619-628, wherein the spacer sequence is capable of hybridizing with the human RFXANK gene;
SEQ ID NOs: 629-638, wherein the spacer sequence is capable of hybridizing with the human RFXAP gene;
SEQ ID NOs: 639-648, wherein the spacer sequence is capable of hybridizing with the human RPL23 gene;
SEQ ID NOs: 649-654, wherein the spacer sequence is capable of hybridizing with the human SOX10 gene;
SEQ ID NOs: 655-665, wherein the spacer sequence is capable of hybridizing with the human SRP54 gene;
SEQ ID NOs: 666-675, wherein the spacer sequence is capable of hybridizing with the human STAT1 gene;
SEQ ID NOs: 676-685, wherein the spacer sequence is capable of hybridizing with the human Tap1 gene;
SEQ ID NOs: 686-695, wherein the spacer sequence is capable of hybridizing with the human Tap2 gene;
SEQ ID NOs: 696-705, wherein the spacer sequence is capable of hybridizing with the human TAPBP gene;
SEQ ID NOs: 706-715, wherein the spacer sequence is capable of hybridizing with the human TFW1 gene;
SEQ ID NOs: 716-725, wherein the spacer sequence is capable of hybridizing with the human CD3D gene; and
SEQ ID NOs: 726-744, wherein the spacer sequence is capable of hybridizing with the human NLRC5 gene.

27. The engineered, non-naturally occurring system of claim 26, wherein, when the system is delivered into a population of human cells ex vivo, the genomic sequence at the CSF2 gene locus, the CD40LG gene locus, the TRBC1 gene locus, the TRBC2 gene locus, both the TRBC1 and TRBC2 gene locus, the CD3E gene locus, the CD38 gene locus, the APLNR gene locus, the BBS1 gene locus, the CALR gene locus, the CD247 gene locus, the CD3G gene locus, the CD52 gene locus, the CD58 gene locus, the COL17A1 gene locus, the DEFB134 gene locus, the ERAP1 gene locus, the ERAP2 gene locus, the IFNGR1 gene locus, the IFNGR2 gene locus, the JAK1 gene locus, the JAK2 gene locus, the mir-101-2 gene locus, the MLANA gene locus, the PSMB5 gene locus, the PSMB8 gene locus, the PSMB9 gene locus, the PTCD2 gene locus, the RFX5 gene locus, the RFXANK gene locus, the RFXAP gene locus, the RPL23 gene locus, the SOX10 gene locus, the SRP54 gene locus, the STAT1 gene locus, the Tap1 gene locus, the Tap2 gene locus, the TAPBP gene locus, the TFW1 gene locus, the CD3D gene locus, or the NLRC5 gene locus, is edited in at least 1.5% of the cells.

28.-107. (canceled)

108. The engineered, non-naturally occurring system of claim 20, wherein genomic mutations are detected in no more than 2%, optionally no more than 1%, of the cells at any off-target loci by CIRCLE-Seq.

109.-115. (canceled)

116. A method of editing human genomic sequence at a preselected target gene locus, the method comprising delivering the engineered, non-naturally occurring system of claim 20 into a human cell, thereby resulting in editing of the genomic sequence at the target gene locus in the human cell.

117. The method of claim 116, wherein the cell is an immune cell, optionally wherein the immune cell is a T lymphocyte.

118. (canceled)

119. The method of claim 116, the method comprising delivering the engineered, non-naturally occurring system comprising a guide nucleic acid that is capable of activating a CRISPR Associated (Cas) nuclease in the absence of a tracrRNA, optionally wherein the Cas nuclease recognizes a protospacer adjacent motif (PAM) consisting of the nucleotide sequence of TTTN or CTTN of into a population of human cells, thereby resulting in editing of the genomic sequence at the target gene locus in at least a portion of the human cells.

120. The method of claim 119, wherein the population of human cells comprises human immune cells, optionally wherein:
the population of human cells is an isolated population of human immune cells; and/or
the immune cells are T lymphocytes.

121.-122. (canceled)

123. The method of claim 119, wherein editing of the genomic sequence at the target gene locus results in lowered expression of the target gene, optionally less than 80%, 70%, 60%, or 50% of the expression of the endogenous gene relative to a corresponding unmodified or parental cell.

124.-127. (canceled)

128. The method of claim 116, wherein the engineered, non-naturally occurring system is delivered into the cell(s) as a pre-formed RNP complex, optionally wherein the pre-formed RNP complex is delivered into the cell(s) by electroporation.

129. (canceled)

130. The method of claim 116, wherein the target gene is selected from the group consisting of:
human CSF2 gene, and the spacer sequence comprises a nucleotide sequence selected from the group consisting of SEQ ID NOs: 201-253;
human CD40LG gene, and the spacer sequence comprises a nucleotide sequence selected from the group consisting of SEQ ID NOs: 254-313;
human TRBC1 gene, and the spacer sequence comprises a nucleotide sequence selected from the group consisting of SEQ ID NOs: 314-319 and 329-332;
human TRBC2 gene, and the spacer sequence comprises a nucleotide sequence selected from the group consisting of SEQ ID NOs: 320-328 and 329-332;
both the human TRBC1 gene and the human TRBC2 gene, and the spacer sequence comprises a nucleotide sequence selected from the group consisting of SEQ ID NOs: 329-332;
human CD3E gene, and the spacer sequence comprises a nucleotide sequence selected from the group consisting of SEQ ID NOs: 333-374;
human CD38 gene, and the spacer sequence comprises a nucleotide sequence selected from the group consisting of SEQ ID NOs: 375-411;
human APLNR gene, and the spacer sequence comprises a nucleotide sequence selected from the group consisting of SEQ ID NOs: 412-421;
human BBS1 gene, and the spacer sequence comprises a nucleotide sequence selected from the group consisting of SEQ ID NOs: 422-431;
human CALR gene, and the spacer sequence comprises a nucleotide sequence selected from the group consisting of SEQ ID NOs: 432-441;
human CALR gene, and the spacer sequence comprises a nucleotide sequence selected from the group consisting of SEQ ID NOs: 432-441;
human CD247 gene, and the spacer sequence comprises a nucleotide sequence selected from the group consisting of SEQ ID NOs: 442-451;
human CD3G gene, and the spacer sequence comprises a nucleotide sequence selected from the group consisting of SEQ ID NOs: 452-461;
human CD52 gene, and the spacer sequence comprises a nucleotide sequence selected from the group consisting of SEQ ID NOs: 462-465;
human CD58 gene, and the spacer sequence comprises a nucleotide sequence selected from the group consisting of SEQ ID NOs: 466-475;
human COL17A1 gene, and the spacer sequence comprises a nucleotide sequence selected from the group consisting of SEQ ID NOs: 476-485;
human DEFB134 gene, and the spacer sequence comprises a nucleotide sequence selected from the group consisting of SEQ ID NOs: 486-495;
human ERAP1 gene, and the spacer sequence comprises a nucleotide sequence selected from the group consisting of SEQ ID NOs: 496-505;
human ERAP2 gene, and the spacer sequence comprises a nucleotide sequence selected from the group consisting of SEQ ID NOs: 506-515;
human IFNGR1 gene, and the spacer sequence comprises a nucleotide sequence selected from the group consisting of SEQ ID NOs: 516-525;
human IFNGR2 gene, and the spacer sequence comprises a nucleotide sequence selected from the group consisting of SEQ ID NOs: 526-535;
human JAK1 gene, and the spacer sequence comprises a nucleotide sequence selected from the group consisting of SEQ ID NOs: 536-545;
human JAK2 gene, and the spacer sequence comprises a nucleotide sequence selected from the group consisting of SEQ ID NOs: 546-555;
human mir-101-2 gene, and the spacer sequence comprises a nucleotide sequence selected from the group consisting of SEQ ID NOs: 556-558;
human MLANA gene, and the spacer sequence comprises a nucleotide sequence selected from the group consisting of SEQ ID NOs: 559-568;
human PSMB5 gene, and the spacer sequence comprises a nucleotide sequence selected from the group consisting of SEQ ID NOs: 569-578;
PSMB8 gene, and the spacer sequence comprises a nucleotide sequence selected from the group consisting of SEQ ID NOs: 579-588;
PSMB9 gene, and the spacer sequence comprises a nucleotide sequence selected from the group consisting of SEQ ID NOs: 589-598;
human PTCD2 gene, and the spacer sequence comprises a nucleotide sequence selected from the group consisting of SEQ ID NOs: 599-608;
human RFX5 gene, and the spacer sequence comprises a nucleotide sequence selected from the group consisting of SEQ ID NOs: 609-618;
human RFXANK gene, and the spacer sequence comprises a nucleotide sequence selected from the group consisting of SEQ ID NOs: 619-628;
human RFXAP gene, and the spacer sequence comprises a nucleotide sequence selected from the group consisting of SEQ ID NOs: 629-638;
human RPL23 gene, and the spacer sequence comprises a nucleotide sequence selected from the group consisting of SEQ ID NOs: 639-648;
SOX10 gene, and the spacer sequence comprises a nucleotide sequence selected from the group consisting of SEQ ID NOs: 649-654;
human SRP54 gene, and the spacer sequence comprises a nucleotide sequence selected from the group consisting of SEQ ID NOs: 655-665;
human STAT1 gene, and the spacer sequence comprises a nucleotide sequence selected from the group consisting of SEQ ID NOs: 666-675;
human Tap1 gene, and the spacer sequence comprises a nucleotide sequence selected from the group consisting of SEQ ID NOs: 676-685;
human TAP2 gene, and the spacer sequence comprises a nucleotide sequence selected from the group consisting of SEQ ID NOs: 686-695;
human TAPBP gene, and the spacer sequence comprises a nucleotide sequence selected from the group consisting of SEQ ID NOs: 696-705;
human TWF1 gene, and the spacer sequence comprises a nucleotide sequence selected from the group consisting of SEQ ID NOs: 706-715;
human CD3D gene, and the spacer sequence comprises a nucleotide sequence selected from the group consisting of SEQ ID NOs: 716-725; and
human NLRC2 gene, and the spacer sequence comprises a nucleotide sequence selected from the group consisting of SEQ ID NOs: 726-744.

131.-211. (canceled)

212. The method of claim 116, wherein genomic mutations are detected in no more than 2%, optionally no more than 1%, of the cells at any off-target loci by CIRCLE-Seq.

213. (canceled)
